(19) United States
(12) Patent Application Publication
(10) Pub. No.: US 2025/0043299 A1
(43) Pub. Date: Feb. 6, 2025

(54) POINT MUTATIONS THAT BOOST AROMATIC AMINO ACID PRODUCTION AND CO2 ASSIMILATION IN PLANTS

(71) Applicant: Wisconsin Alumni Research Foundation, Madison, WI (US)

(72) Inventors: Hiroshi MAEDA, Madison, WI (US); Ryo YOKOYAMA, Madison, WI (US); Marcos Vinicius Viana DE OLIVEIRA, Madison, WI (US)

(21) Appl. No.: 18/717,257

(22) PCT Filed: Dec. 7, 2022
(86) PCT No.: PCT/US2022/081110

§ 371 Date: Jun. 6, 2024

Related U.S. Application Data
(60) Provisional application No. 63/286,811, filed on Dec. 7, 2021.

(51) Int. Cl. C12N 15/82; C12N 9/10

(52) U.S. Cl. CPC C12N 15/8251; C12N 9/1085; C12Y 205/01054

(57) ABSTRACT

The present invention provides engineered 3-deoxy-D-arabino-heptulosonate 7-phosphate synthase (DHS) polypeptides comprising mutations that deregulate the shikimate pathway, resulting in increased production of aromatic amino acids and enhanced carbon assimilation in plants. Also provided are polynucleotides, constructs, and vectors that encode the engineered polypeptides; cells, seeds, and plants that express the engineered polypeptides; and methods for generating and using plants that express the engineered polypeptides.

### CROSS-REFERENCE TO RELATED APPLICATIONS

This application claims priority to U.S. Provisional Application No. 63/286,811 filed on Dec. 7, 2021, the contents of which are incorporated by reference in their entireties.

### STATEMENT REGARDING FEDERALLY SPONSORED RESEARCH

This invention was made with government support under 1818040 awarded by the National Science Foundation. The government has certain rights in this invention.

### SEQUENCE LISTING

A Sequence Listing accompanies this application and is submitted as an XML file named “960296.04348.xml” which is 216,433 bytes in size and was created on Dec. 6, 2022. The sequence listing is electronically submitted via Patent Center with the application and is incorporated herein by reference in its entirety.

### BACKGROUND

Plants can directly convert atmospheric carbon dioxide (CO₂) into diverse aromatic natural products, which are primarily derived from the aromatic amino acids tyrosine, phenylalanine, and tryptophan. Aromatic compounds have unusual stability due to their aromaticity (i.e., electron delocalization). As a result, aromatic compounds have potential to be used as a carbon sink for reducing atmospheric CO₂ (1). Aromatic compounds are also key precursors for pharmaceuticals, commodity chemicals, and industrial materials, for which there is rapidly growing global demand (2, 6). However, the chemical conversion of CO₂ into aromatic compounds remains challenging, and fossil fuels remain the primary source of aromatic compounds (3). Thus, there remains a need in the art for improved methods for harvesting aromatic compounds from renewable sources, such as plants.

### SUMMARY

In a first aspect, the present invention provides engineered 3-deoxy-D-arabino-heptulosonate 7-phosphate synthase (DHS) polypeptides. The polypeptides comprise at least one mutation at a position corresponding to amino acid residue 109, 114, 159, 240, 244, 245, 247, 248, 319, 322, or 348 of the Arabidopsis DHS1 polypeptide (SEQ ID NO:1).

In a second aspect, the present invention provides polynucleotides encoding the engineered polypeptides disclosed herein.

In a third aspect, the present invention provides constructs comprising a promoter operably linked to one of the polynucleotides described herein.

In a fourth aspect, the present invention provides vectors comprising one of the polynucleotides or constructs described herein.

In a fifth aspect, the present invention provides cells comprising one of the engineered polypeptides, polynucleotides, constructs, or vectors described herein.

In a sixth aspect, the present invention provides seeds comprising one of the engineered polypeptides, polynucleotides, constructs, vectors, or cells described herein.

In a seventh aspect, the present invention provides plants grown from the seeds described herein and plants comprising one of the engineered polypeptides, polynucleotides, constructs, vectors, or cells described herein.

In an eighth aspect, the present invention provides methods for improving a plant by (1) increasing production of aromatic amino acids in the plant, and/or (2) increasing the amount of carbon dioxide (CO₂) sequestered by the plant. The methods comprise: introducing one of the engineered polypeptides, polynucleotides, constructs, or vectors described herein into the plant.

In a ninth aspect, the present invention provides methods for using the plants described herein to (1) produce aromatic amino acids or derivatives thereof, or (2) sequester CO₂. Both sets of methods comprise growing the plants described herein. The methods for producing aromatic amino acids or derivatives thereof further comprise purifying the aromatic amino acids or derivatives thereof produced by the plant.

### BRIEF DESCRIPTION OF THE DRAWINGS

FIG. 1 shows that multiple suppressor of tyra2 (sota) mutations rescued tyra2 growth inhibition and enhanced tyrosine (Tyr) and phenylalanine (Phe) accumulation. (A) A simplified diagram of the shikimate and AAA biosynthetic pathways. DHS, 3-deoxy-d-arabino-heptulosonate 7-phosphate synthase; E4P, erythrose-4-phosphate; PEP, phosphoenolpyruvate; TyrA, TyrA arogenate dehydrogenase. (B) Plant pictures of 4-week-old Col-0 wild-type (WT), tyra2, and two representative sota mutants of Arabidopsis thaliana. The remaining sota mutant plants are shown in FIG. 11. (C) Soluble metabolite profiling and shoot area of the 3-week-old Col-0, tyra2, and sota mutants. Dark and light bars represent that each sota mutant line showed Col-0-like fully mature green leaves and tyra2-like reticulated leaves, respectively. All the metabolic sota mutants exhibited significantly larger shoot area than tyra2 [one-way analysis of variance (ANOVA) with Dunnett's multiple comparisons test, P<0.001]. Data are means±SEM (n=4 independent plant samples). (D) Relative amounts of Tyr and Phe against Col-0 shown in (C) were plotted for metabolic sota (circles), response sota (triangles), and tyra2 (a square). (E) Plant pictures of representative complementation lines at T₂ generation that were generated by introducing either WT DHS (e.g., DHS1^{WT}) or sota-mutated DHS (e.g., DHS1^{B4}) genes, driven by the respective endogenous promoter, into the Arabidopsis tyra2 background. Scale bars, 1 cm. The remaining lines are shown in FIG. 16.

FIG. 2 shows that the sota mutations biochemically deregulate effector-mediated DHS negative feedback inhibition. (A) A structural model of A. thaliana DHS2 (AtDHS2, purple) generated from the P. aeruginosa DHS (PaDHS, white) with Trp (magenta) bound. Residues corresponding to the sota mutations mapped onto the AtDHS2 model are highlighted in yellow. The entire model is shown in FIG. 17. (B) Selected regions of the amino acid sequence alignment of PaDHS and AtDHS enzymes, with the positions of the sota mutations indicated by blue, red, green arrows for AtDHS1, AtDHS2, and AtDHS3, respectively. The entire alignment is shown in FIG. 18. (C) Enzymatic assay of DHS2 WT (DHS2^{WT}) and DHS2 with a sota mutation (DHS2^{A4}, DHS2^{A11}, and DHS2^{F1}) in the presence of Tyr, Trp, or mixture of all AAAs at 1 mM. ****P≤0.0001; significant differences by one-way ANOVA with Dunnett's multiple comparisons test against the corresponding DHS2^{WT} samples. Data are means±SEM (n=3). (D) Screening of AAAs and AAA-derived metabolites as potential inhibitors of DHS1^{W}T and DHS2^{WT}. *P≤0.05, **P≤0.01, ***P≤0.001, and ****P≤0.0001 denote significant differences by one-way ANOVA with Dunnett's test against the corresponding “No effector” samples. Data are means±SEM (n=3). The dotted horizontal lines separate four sets of independent experiments. (E and F) IC₅₀ curves of WT and sota mutant enzymes of DHS1 (left) and DHS2 (right) with varied concentrations of HGA (E) and indole-3-pyruvate (IPA) (F). Data are means±SEM (n=3). (G) Plant picture (left) and fresh weight measurement (right) of 3-week-old Col-0, sotaB4, and sotaA4 mutants (Col-0 background) on the media containing ILA at 0, 250, 500, or 1000 μM. *P≤0.05 and **P≤0.01 denote significant differences by one-way ANOVA with Dunnett's test against the corresponding Col-0 samples (n=12 to 16 independent plant samples).

FIG. 3 shows that increased carbon flux elevates the levels of AAAs but not all AAA-derived compounds in the sota mutants. (A)¹³CO₂ labeling experiment of Col-0, sotaB4, and sotaA4 (tyra2 background), followed by quantification of ¹³C-labeled Tyr and Phe by GC-MS and ¹³C-labeled Trp and shikimate by liquid chromatography (LC)-MS. Data are means±SEM (n=3 independent biological samples except for 0-hour time point having two replicates). (B) Targeted metabolomics analysis of AAAs and AAA-derived metabolites in 4-week-old Col-0, sotaB4, and sotaA4 (Col-0 background) grown on soil (also see FIG. 27 for data of the sota mutants in the tyra2 background). Actual values are shown in Table 3. Different letters indicate statistically significant differences among genotypes (one-way ANOVA with Tukey-Kramer test, P<0.05). Data are means±SEM (n=5 to 6 independent plant samples). K3GR7R, kaempferol-3-O-(2″-O-rhamnosyl)glucoside-7-O-rhamnoside; PAL, Phe ammonia lyase. (C) The correlations between the levels of AAAs and their representative derivates shown in (B). The correlations of Phe versus phenyllactate or phenylacetate are shown in FIG. 31.

FIG. 4 shows that carbon fixation is accelerated to support high AAA production in the sota mutants. (A to C) The levels of AAA and shikimate (A), starch (B), and glucose and sucrose (C) of Col-0, sotaB4, and sotaA4 (Col-0 background) harvested at the indicated time points under the 12-hour light/12-hour dark cycle (white/black bars above each graph). Starch is expressed as micromoles of glucose (Glc) equivalents per gram FW. *P≤0.05 and ****P≤0.0001; significant differences by one-way ANOVA with Dunnett's multiple comparisons test against the corresponding Col-0 samples. Data are means±SEM (n=4 to 6 independent plant samples). (D and E) The response curves of CO₂ assimilation rate (A) to light intensity and CO₂ concentration in intercellular air spaces (Cᵢ) of Arabidopsis Col-0 and the sota mutants (Col-0 background). Data are means±SEM (n=5 to 6 independent plant samples). (F) The sota mutations eliminate or attenuate feedback regulation by certain effector molecules (open diamonds) of AtDHS1/3 and AtDHS2 (blue and red lines, respectively).

FIG. 5 shows a sequence alignment of DHS proteins from crop species (SEQ ID NO:1-37; see Table 11). DHS orthologs were obtained from Arabidopsis, tomato (Solanum lycopersicum), tobacco (Nicotiana benthamiana), soybean (Glycine max), cotton (Gossypium raimondii), poplar (Populus trichocarpa), sorghum (Sorghum bicolor), rice (Oryza sativa), corn (Zea mays), and bacteria (Mycobacterium tuberculosis and Pseudomonas aeruginosa). The red and yellow colors represent the sota mutations we confirmed genetically, and the remaining mutations that we identified by sequencing, respectively. A condensed alignment showing only the mutated portions of the DHS proteins (A) and an alignment of the full-length DHS sequences (B) are shown.

FIG. 6 shows a sequence identity matrix of crop DHSs. The pairwise sequence identity of crop DHSs were shown as a heat map. DHS orthologs were obtained from Arabidopsis, tomato (Solanum lycopersicum), tobacco (Nicotiana benthamiana), soybean (Glycine max), cotton (Gossypium raimondii), poplar (Populus trichocarpa), sorghum (Sorghum bicolor), rice (Oryza sativa) and corn (Zea mays). Each sequence identity was calculated from Clustal Omega multiple sequence alignment.

FIG. 7 shows a phylogenetic tree of crop DHSs. DHS orthologs were obtained from Arabidopsis, tomato (Solanum lycopersicum), tobacco (Nicotiana benthamiana), soybean (Glycine max), cotton (Gossypium raimondii), poplar (Populus trichocarpa), sorghum (Sorghum bicolor), rice (Oryza sativa) and corn (Zea mays). The sequences were aligned by the MUSCLE algorithm and then constructed into the tree based on the maximum-likelihood method with 1,000 bootstrap replicates in MEGA X. The sequence identities of each DHS sequence against Arabidopsis DHS1 were shown next to the phylogenetic tree.

FIG. 8 shows the sota mutations on an Arabidopsis DHS2 protein model. Overlaid structures of Pseudomonas aeruginosa DHS (PaDHS, 5uxm, white) with Trp (orange) bound and AtDHS2 WT (purple) predicted based on PaDHS2. The red and yellow colors represent the residues corresponding to the sota mutations we confirmed genetically, and the remaining mutations that we identified by sequencing, respectively.

FIG. 9 shows that transient expression of the mutated Arabidopsis DHS1 in tobacco leads to elevated production of tyrosine and phenylalanine. (A) Schematic diagram of the experiment. (B) Levels of tyrosine (Tyr), phenylalanine (Phe), and tryptophan (Trp) in tobacco samples transiently expressing empty vector (EV), Arabidopsis DHS1 wild-type (WT), or mutated DHS1 sotaB4. Means±SD (n=7-8, P<0.05)

FIG. 10 shows that introducing sota mutations into DHS genes from sorghum and poplar also dramatically enhances AAA production in plants. The sotaB4 and sotaF1 mutations were introduced into the Sorghum bicolor gene SbDHS (Sobic.007G225700.1.p) and the Populus trichocarpa gene PtDHS (Potri.005G07330.1.p) and expressed in Nicotiana benthamiana leaves via Agrobacterium-mediated transformation. Two different tags, i.e., hemagglutinin (HA) and TdTomato-HA, were used for comparisons, and the P19 vector was co-transformed to prevent gene silencing.

FIG. 11 shows that the sota mutations suppress the tyra2 phenotypes to different degrees. Col-0, tyra2, and 40 M₃ sota mutants were germinated on soil and grown in a growth chamber under 12 hours photoperiod with 100 μE light exposure. The pictures show a representative phenotype of each genotype at 4-weeks old after germination. Bars=1 cm.

FIG. 12 shows that the isolated sota mutants still carry the tyra2 mutation. Genomic DNA from Col-0, tyra2, and the eight sota mutants were subjected to a PCR analysis to confirm the presence or absence of the homozygous tyra2-1 T-DNA insertion (SALK_001756). PCR was conducted using a combination of three primers, LBb1.3 (pHM0027), LP (pHM0039), and RP (pHM0038, Table 10), and amplification products were separated on 1.5% TAE-agarose gel. The WT sequence (no T-DNA insertion) was amplified as a band of 816 bp in the Col-0 sample. tyra2 T-DNA sequence was amplified as a band of ˜500 bp in the tyra2 sample as well as in all the tested metabolic sota mutant samples, demonstrating that the T-DNA insertion at tyra2 loci remained homozygous. H₂O was used instead of genomic DNA for a negative control, which showed no amplification. Band sizes were estimated using BenchTop 1 kb DNA ladder (G7541, Promega).

FIG. 13 shows a frequency analysis of single nucleotide variants (SNVs) found in the sota F₂ population. DNA from 200 F₂ bulk populations was submitted for Illumina whole genome sequencing and SNVs were identified by comparison to the TAIR10 Arabidopsis thaliana Col-0 reference genome. SNVs present in the tyra2-like population were subtracted from the ones present in the sota-like population. The remaining sota-like SNVs were scatter-plotted for their frequencies among obtained reads (y axis) and genomic position (x axis). The sotaA4 and sotaA11 mutants accumulated high frequency mutations linked to the 16 Mb region of chromosome IV, whereas sotaB4 showed a trend of high frequency mutations on the 18 Mb region of chromosome IV. These and other analyses conducted in this study revealed that sotaA4 and sotaA11 contained mutations in At4g33510 (which encodes DHS2), while sotaB4 had a mutation in At4g39980 (which encodes DHS1) (arrows). While sotaA4 and sotaA11 mutations in the DHS2 gene were found at 100% frequency in the sota-like population, the sotaB4 mutation on the DHS1 gene was found at 66.67% frequency, consistent with the complete dominance of sotaB4, which made it difficult to differentiate heterozygous and homozygous sotaB4 plants. As a result, its sota-like pool of the F₂ population most likely contained a mixture of heterozygous and homozygous seedlings leading to the observed frequency being lower than 100%.

FIG. 14 shows dCAPS genotyping of representative metabolic sota mutants. Four-week-old from F₂ populations were obtained by backcrossing sotaA4, sotaA11, sotaB4, sotaF1, sotaG1, and sotaH1 with tyra2. Representative individuals were genotyped via dCAPS. Western blots are shown for each population. In all blots, the first lane is an undigested control, and the last lane is a digested DNA from a representative tyra2-like individual plant, which serves as a control for the WT allele without any sota mutation. For each blot, the dCAPS designated restriction enzyme is shown under the gels, and − and + symbols indicate the absence or presence of the restriction enzyme, respectively. A PCR product was not incubated with the restriction enzyme was used as an undigested control. Band sizes were evaluated by using GeneRuler™ Ultra Low Range DNA ladder (Thermo Scientific). Bars=1 cm.

FIG. 15 shows that the sota mutants exhibit dominant or semidominant characteristics. Plant pictures (A) and Tyr and Phe amounts (B) of 4-weeks-old F₂ populations of the sota lines backcrossed with tyra2. dCAPS genotyping was conducted to identify individuals having homozygous DHS WT alleles (AA), as well as heterozygous (Aa) and homozygous (aa) DHS sota alleles. The tyra2 growth phenotype was recovered even in Aa, which demonstrates the dominant nature of the sota mutations. Different letters indicate statistically significant differences among genotypes (one-way ANOVA with Tukey-Kramer test, P<0.05). Data are means±SEM (n=3 to 6 independent plant samples). Bars=1 cm.

FIG. 16 shows that the growth defect in tyra2 was recovered by introducing the sota mutated DHS genes, but not WT DHS genes. (A) Plant pictures of 4-week-old complementation lines that were generated by introducing the WT DHS genes (e.g., DHS1^{WT}) or sota-mutated DHS genes (e.g., DHS1^{B4}) into the tyra2 background under the control of their own native promoters. Two independent plants for each construct were generated and shown as #1 and #2. (B) The amounts of AAAs and chlorophyll in the complementation lines were analyzed in the T₂ generation. Data are means±SEM (n=4 independent plant samples).

FIG. 17 shows the structures of PaDHS and AtDHS2. Overlaid structures of PaDHS (5uxm, white) with Trp (magenta) bound and AtDHS2 WT predicted based on PaDHS2. Residues corresponding to the sota mutations were mapped on the AtDHS2 WT structure and are highlighted in yellow. These residues are located at the opposite end of the enzyme from the catalytic site (gray circle).

FIG. 18 shows an alignment of AtDHS protein sequences, i.e., PaDHS and three AtDHS isoforms, and the locations of the eight sota mutations. The residues are colored with dark purple (>80%), medium purple (>60%), and light purple (>40%) according to the percentage of residues in each column that agree with the consensus sequence.

FIG. 19 shows that the DHS1^{B4} mutant enzyme responded to known effector molecules similarly to the DHS1^{WT} enzyme. (A) Enzymatic assay of DHS1^{WT} and DHS^{B4} enzymes in the presence of Tyr, Trp, or mixture of all AAAs at 1 mM. ns denotes no significant difference by Student's t test. (B) The IC50 curves of DHS1^{WT} and DHS^{B4} enzymes with varied concentrations of chorismate (left) and caffeate (right). Data are means±SEM (n=3 independent assays).

FIG. 20 shows that the expression of DHS genes was unaffected in the sota mutants. RT-qPCR analysis of DHS1, DHS2, and DHS3 gene expression in the mature leaves of 4-week-old Col-0 and the sota mutants. ns denotes no significant difference by one-way ANOVA with Dunnett's multiple comparisons test against the corresponding Col-0 samples. Data are means SEM (n=4 independent plant samples).

FIG. 21 shows that the DHS2^{A4} mutant enzyme responded to known effector molecules similarly to the DHS2^{WT} enzyme. (A) Enzymatic assay of DHS2^{WT} and DHS^{A4} enzymes in the presence of shikimate, arogenate, or prephenate at 1 mM. ns denotes no significant difference by Student's t test. (B) The IC₅₀ curves of DHS2^{WT} and DHS^{A4} enzymes with varied concentrations of chorismate (left) and caffeate (right). Data are means±SEM (n=3 independent assays).

FIG. 22 shows that the DHS2^{A4} enzyme is likely still able to bind to Trp and Tyr. (A) Docking simulation of AtDHS2^{WT} (pale orange) and AtDHS2^{A4} (magenta) with Trp or Tyr based on PaDHS (green). (B) Docking scores of Trp and Tyr binding to AtDHS2^{WT} and AtDHS2^{A4} (C) The differential scanning fluorimetry (DSF) protein-ligand affinity analysis of DHS2^{WT} and DHS2^{A4} proteins in the presence of individual AAA at 1 mM. Data are means±SEM (n=4). (D) The temperature that increased the fluorescence level by half was defined as melting temperature (Tₘ). ns denotes no significant difference by Student's t test. Data are means±SEM (n=4 independent measurements). Tyr or Trp at 1 mM shifted the thermal stability curves and significantly increased the Tₘ but did so similarly for both DHS2^{WT} and DHS2^{A4} mutant enzymes. Phe at 1 mM, on the other hand, had no impact on the Tₘ of DHS2^{WT} or DHS2^{A4} enzymes, consistent with the lack of DHS2 inhibition by Phe. These results suggest that both DHS2^{WT} and DHS2^{A4} enzymes can bind to Tyr or Trp with comparable affinity, but not to Phe.

FIG. 23 shows that the sota mutations relax the negative feedback inhibition mediated by Tyr- and Trp-derived metabolites. (A) Simplified pathway map of AAAs and AAA-derived metabolites used in the effector screening. HPP, 4-Hydroxyphenylpyruvate; HGA, homogentisate; PPY, phenylpyruvate; IPA, indole-3-pyruvate; ILA, indole-3-lactate; IAA, indole acetate. (B) IC50 curves of DHS WT and the sota mutant enzymes with varied concentrations of HPP, ILA or indole-3-propionate. Data are means±SEM (n=3 independent assays).

FIG. 24 shows ¹³C incorporation into various metabolites during a 6-hour time course of ¹³CO₂ labeling from the beginning of the day. (A) Three-weeks-old Col-0, sotaA4, and sotaB4 (in tyra2 background) were supplied with ¹³CO₂ under the light (150 μE) for 6 hours starting at 8 am. (B) The labeled leaf tissues were harvested after 0, 1, 3, 6 hours of the labeling and the soluble metabolites were analyzed by GC-MS. Total metabolites and percent ¹³C enrichment were used to calculate ¹³C-labeled metabolite levels. The data for Tyr, Phe, Trp, and shikimate are also available in FIG. 3A. Data are means±S.D. (n=3 biological replicates except for the 0-hour time point, which has two replicates.)

FIG. 25 shows ¹³C incorporation into various metabolites after 3 hour of CO₂ labeling towards the end of the day. Four-week-old Col and sotaA4 (in tyra2 background) were supplied with ¹³CO₂ under the light (150 μE) for 3 hours from 5 pm to 8 pm. The labeled leaf tissues were harvested at the end of the labeling in three biological replicates and the soluble metabolites were analyzed by GC-MS for total metabolites and % ¹³C enrichment, which were used to calculate unlabeled vs. ¹³C-labeled metabolite levels (top open and bottom closed bars, respectively). Data are means±S.D. (n=3 biological replicates). Significant differences in ¹³C-labeled metabolite levels are indicated by *P<0.01, **0.001, ***0.001 (Student t-test between Col-0 and sotaA4).

FIG. 26 shows a growth analysis of the sota mutants at different growth stages. (A) Representative images of 2- to 4-week-old Col-0, sotaB4, and sotaA4 (Col-0 background) plants. Bar=1 cm. (B) Growth parameters of 2- to 4-week-old Col-0, sotaB4 and sotaA4 (Col-0 background) plants. *P<0.05 and **P<0.01 denote significant differences by one-way ANOVA with Dunnett's test against the corresponding Col-0 samples (n=10 to 12 independent plant samples). (C) Representative images of 2-month-old Col-0, sotaB4 and sotaA4 (Col-0 background) plants and their seed yield per individual plants. ns denotes no significant difference by one-way ANOVA with Dunnett's multiple comparisons test against the Col-0 samples. n=6 independent plant samples.

FIG. 27 shows that the tyra2 mutation affected the ratios of tyrosine (Trp) with phenylalanine (Phe) or tryptophan (Trp) levels. The levels (A) and ratios (B) of AAAs in mature leaves of 4-week-old Col-0 (open bars) as well as in the sotaB4 and sotaA4 mutants in the presence and absence of the original tyra2 mutation (i.e., in the tyra2 or Col-0 background, respectively). Different letters indicate statistically significant differences among genotypes (one-way ANOVA with Tukey-Kramer test, P<0.05). Data are means±SEM (n=4 independent plant samples).

FIG. 28 shows that the lignin content was not affected in the sota mutants. (A) Phloroglucinol staining of the leaf and root tissues of four-week-old Col-0, sotaB4, and sotaA4 plants in the Col-0 background. Bars in the first two panels (unstained and stained) indicate 200 μm and those in the last (magnified) panel denote 100 μm. Ectopic accumulation of lignin was not observed in the sota mutants. (B) Thioglycolic acid lignin quantification of the leaves and stems of Col-0, sotaB4, and sotaA4 in the Col-0 background. The lignin levels are expressed as A₂₈₀ level per weight of cell wall residue (CWR). ns denotes no significant difference by one-way ANOVA with Dunnett's multiple comparisons test against the corresponding Col-0 samples. Data are means±SEM (n=3 independent plant samples).

FIG. 29 shows that amounts of AAA-derived compounds were still elevated in the sota mutants after high light stress. The levels of AAAs and AAA-derived metabolites in 4-weeks-old Col-0, sotaB4, and sotaA4 plants (Col-0 background) before and after a 2-day-exposure to high light (650 μE) stress. The actual values are shown in Table 4. Different letters indicate statistically significant differences among genotypes (one-way ANOVA with Tukey-Kramer test, P<0.05). Data are means±SEM (n=4 independent plant samples). (A) AAA levels remained significantly elevated in both sota mutants compared to Col-0 even after the high light stress. (B) The levels of AAA-derived metabolites were increased after the high light treatment but were similar between genotypes. I3M, indolyl-3-methyl glucosinolate.

FIG. 30 shows that AAA levels were elevated in plate-grown shoots and roots of sota mutants, except in sotaA4 roots. The levels of AAAs and AAA-derived metabolites were analyzed in 10-day-old shoots and roots of Col-0, sotaB4, and sotaA4 plants (Col-0 background) grown on 12 MS media containing 1% sucrose. The levels of AAAs and shikimate were elevated in the sota mutants compared to Col-0 in both shoot and root tissues, with the exception of sotaA4 root tissues showing AAA levels similar to Col-0. Actual values are shown in Table 5. Different letters indicate statistically significant differences among genotypes (one-way ANOVA with Tukey-Kramer test, P<0.05). Data are means±SEM (n=3 to 6 biological samples). IAA, indole acetic acid; I3M, indolyl-3-methyl glucosinolate; K3GR7R, kaempferol-3-O-(2″-O-rhamnosyl)glucoside-7-O-rhamnoside.

FIG. 31 shows that the levels of phenylpyruvate (PPY) and PPY-derived compounds are positively correlated with the Phe level in sota mutants. The correlations between the levels of Phe and PPY-derivate compounds (phenyllactate and phenylacetate), which are shown in FIG. 3B and Table 3.

FIG. 32 shows that transgenic expression of the sota-mutated DHS genes into the Col-0 wild-type background also enhanced AAA production. (A) Representative images of 5-week-old T₂ transgenic plants expressing the WT or sota DHS genes in the Col-0 background under the control of their own promoters, as well as control plants having empty vector (EV). Bar=1 cm. (B) Targeted metabolomics analysis of AAAs and AAA-derived metabolites in 5-week-old transgenic lines grown on soils. Actual values are shown in Table 7. Different letters indicate statistically significant differences among genotypes (one-way ANOVA with Tukey-Kramer test, P<0.05). Data are means±SEM (n=5 independent plant samples). (C) Correlation between the levels of AAA and their derivates shown in (B).

FIG. 33 shows that the introduction of the sota-mutated DHS genes into the Col-0 wild-type background also resulted in upregulation of CO₂ assimilation. The response curves of CO₂ assimilation rate (A) versus CO₂ concentration in intercellular air spaces (Ci) of 5-week-old T₂ transgenic plants expressing the WT or sota DHS genes in the Col-0 background under the control of their own promoters, as well as control plants having empty vector (EV, their phenotypes are shown in FIG. 32). The photosynthetic parameters calculated from the graph are listed in Table 8. Data are means±SEM (n=5 independent plant samples).

FIG. 34 shows that the sota mutations are found in amino acid residues that are well conserved among plants species including dicot and monocot crops. Amino acid sequences of DHS orthologs were obtained from Phytozome 13 for Arabidopsis, tomato (Solanum lycopersicum), tobacco (Nicotiana benthamiana), soybean (Glycine max), cotton (Gossypium raimondii), poplar (Populus trichocarpa), sorghum (Sorghum bicolor), rice (Oryza sativa), and corn (Zea mays). The residues are colored with dark purple (>80%), medium purple (>60%), and light purple (>40%) according to the percentage of residues in each column that agree with the consensus sequence. The amino acid substitutions caused by the eight sota mutations in Arabidopsis DHS enzymes (e.g., G244R DHS1^{B4}) are shown above or below the corresponding residue. The amino acid region with multiple sota mutations is indicated by a box with dotted orange lines and expanded below to indicate the most conserved sequence.

FIG. 35 is a table showing the sequence conservation among 472 DHS orthologs from 130 photosynthetic eukaryotic species at residues corresponding to the sota mutation sites.

### DETAILED DESCRIPTION

The present invention provides engineered 3-deoxy-D-arabino-heptulosonate 7-phosphate synthase (DHS) polypeptides comprising mutations that deregulate the shikimate pathway, resulting in increased production of aromatic amino acids and enhanced carbon assimilation in plants. Also provided are polynucleotides, constructs, and vectors that encode the engineered polypeptides; cells, seeds, and plants that express the engineered polypeptides; and methods for generating and using plants that express the engineered polypeptides.

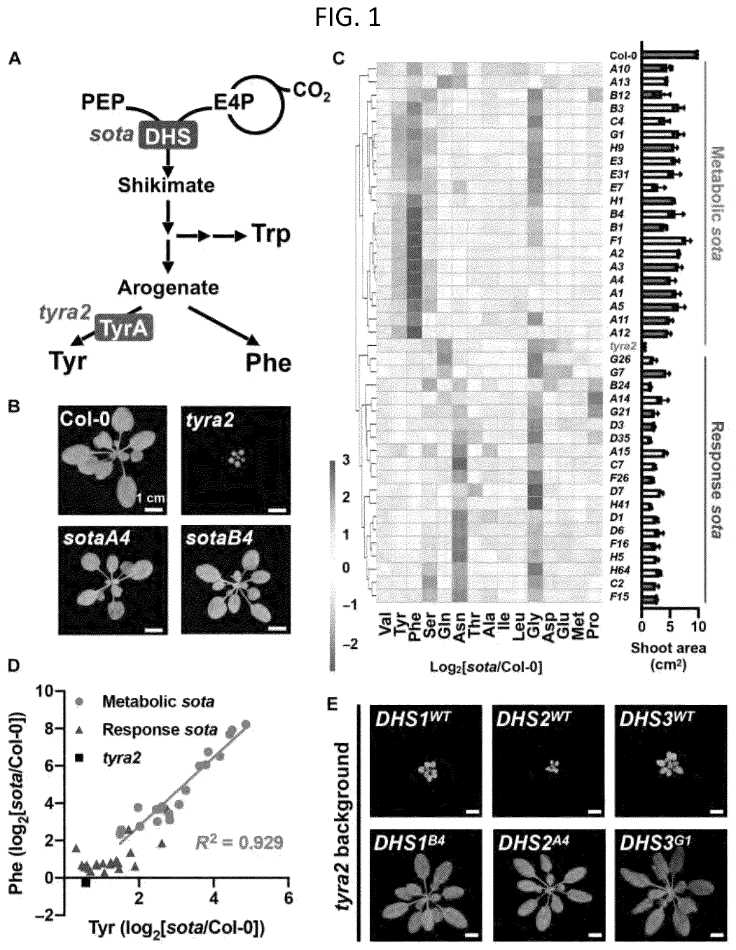

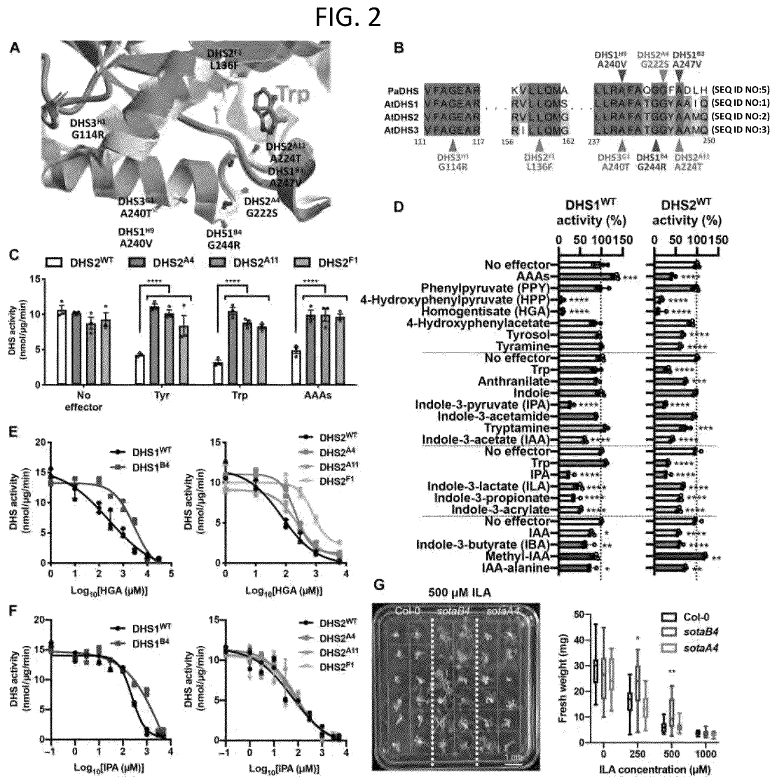

In the Examples, the inventors describe the identification of suppressor of tyra2 (sota) mutations in Arabidopsis thaliana that deregulate the first step of the shikimate pathway, i.e., a pathway that connects central carbon metabolism to the pathway for aromatic amino acid biosynthesis in plants. The sota mutations mapped to genomic loci that encode the three Arabidopsis isoforms of the enzyme 3-deoxy-D-arabino-heptulosonate 7-phosphate synthase (DHS). DHS catalyzes the first reaction of the shikimate pathway using two substrates, phosphoenolpyruvate (PEP) and erythrose-4-phosphate (E4P), which are directly supplied from glycolysis and the Calvin-Benson-Bassham (CBB) cycle, respectively (FIG. 1A) (6, 20). The inventors discovered that plants that express DHS enzymes comprising the sota mutations produce greater quantities of aromatic amino acids and assimilate greater quantities of carbon dioxide (CO₂). Plants use aromatic amino acids to produce a variety of compounds (e.g., plant hormones, nutrients, and specialized metabolites) that are widely used in our society (6). Thus, these newly discovered sota mutations can be used to increase the conversion of atmospheric CO₂ into valuable aromatic compounds.

### Engineered Polypeptides:

In a first aspect, the present invention provides engineered DHS polypeptides. The polypeptides comprise at least one mutation at a position corresponding to amino acid residue 109, 114, 159, 240, 244, 245, 247, 248, 319, 322, or 348 of the Arabidopsis DHS1 polypeptide (SEQ ID NO:1). These residues correspond to positions at which suppressor of tyra2 (sota) mutations were identified by the inventors. Identification of the mutations at residues 114, 159, 240, 244, 245, and 247 is described in Example 1, whereas identification of the mutations at residues 109, 248, 319, 322, and 348 is described in Example 2.

The terms “polypeptide,” “protein,” and “peptide” are used interchangeably herein to refer to a series of amino acid residues connected by peptide bonds between the alpha-amino and carboxy groups of adjacent residues. Polypeptides include modified amino acids. Suitable polypeptide modifications include, but are not limited to, acylation, acetylation, formylation, lipoylation, myristoylation, palmitoylation, alkylation, isoprenylation, prenylation, amidation at C-terminus, glycosylation, glycation, polysialylation, glypiation, and phosphorylation. Polypeptides may also include amino acid analogs.

The engineered DHS polypeptides described herein may be full-length polypeptides or may be fragments of a full-length polypeptide. As used herein, a “fragment” is a portion of a polypeptide that is identical in sequence to, but shorter in length than, the full-length polypeptide. For example, a fragment may comprise at least 5, 10, 15, 20, 25, 30, 40, 50, 60, 70, 80, 90, 100, 150, 250, or 500 contiguous amino acid residues of a full-length polypeptide. Fragments may be preferentially selected from certain regions of a polypeptide. A fragment may include an N-terminal truncation, a C-terminal truncation, or both an N-terminal and C-terminal truncation relative to the full-length polypeptide. Preferably, the DHS polypeptide fragments used with the present invention are functional fragments. As used herein, a “functional fragment” is a fragment that retains at least 20%, 40%, 60%, 80%, or 100% of the DHS activity of the corresponding full-length polypeptide.

The polypeptides described herein are “engineered,” meaning that they have been altered by the hand of man. Specifically, the engineered DHS polypeptides of the present invention have been altered to comprise a mutation. As used herein, the term “mutation” refers to a difference in an amino acid sequence relative to a reference sequence (e.g., the sequence of the wild-type polypeptide). Mutations include insertions, deletions, and substitutions of an amino acid relative to a reference sequence. An “insertion” refers to a change in an amino acid sequence that results in the addition of one or more amino acid residues. An insertion may add 1, 2, 3, 4, 5, 10, 20, 30, 40, 50, 60, 70, 80, 90, 100, 150, 200, or more amino acid residues to a sequence. A “deletion” refers to a change in an amino acid sequence that results in the removal of one or more amino acid residues. A deletion may remove 1, 2, 3, 4, 5, 10, 20, 50, 100, 200, or more amino acids residues from a sequence. A “substitution” refers to a change in an amino acid sequence in which one amino acid is replaced with a different amino acid. An amino acid substitution may be a conversative replacement (i.e., a replacement with an amino acid that has similar properties) or a radical replacement (i.e., a replacement with an amino acid that has different properties).

The engineered DHS polypeptides of the present invention comprise one or more mutations relative to the corresponding wild-type polypeptide (i.e., the wild-type version of the same DHS polypeptide). The term “wild-type” is used to describe the non-mutated version of a polypeptide that is most typically found in nature.

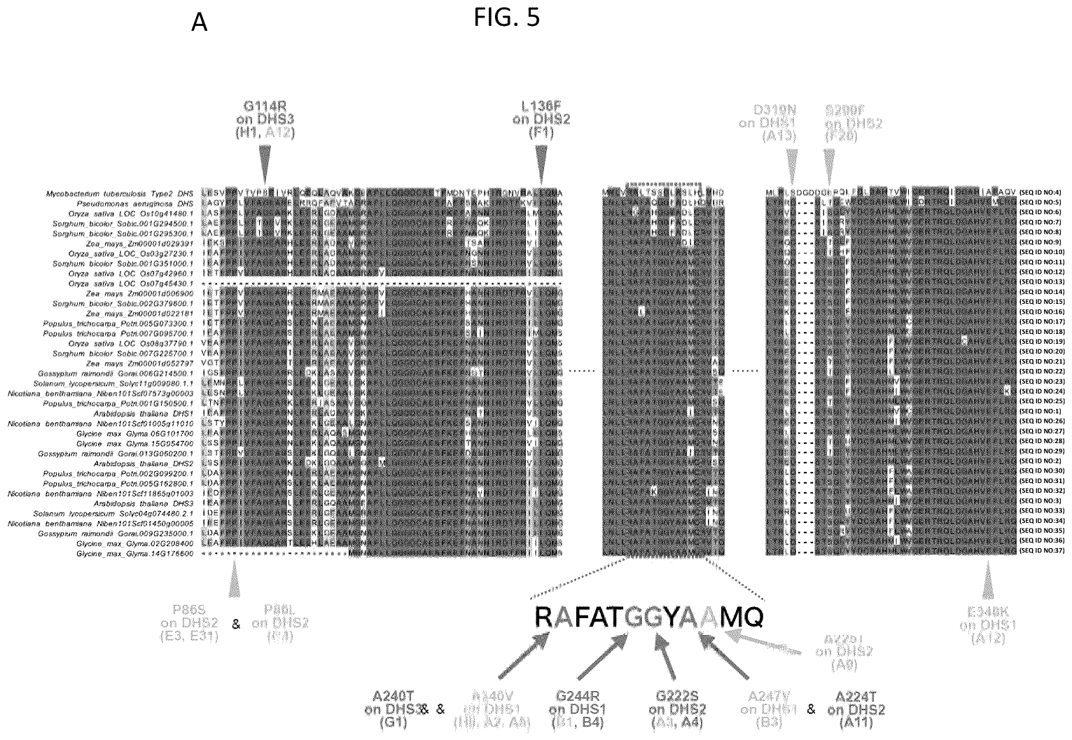

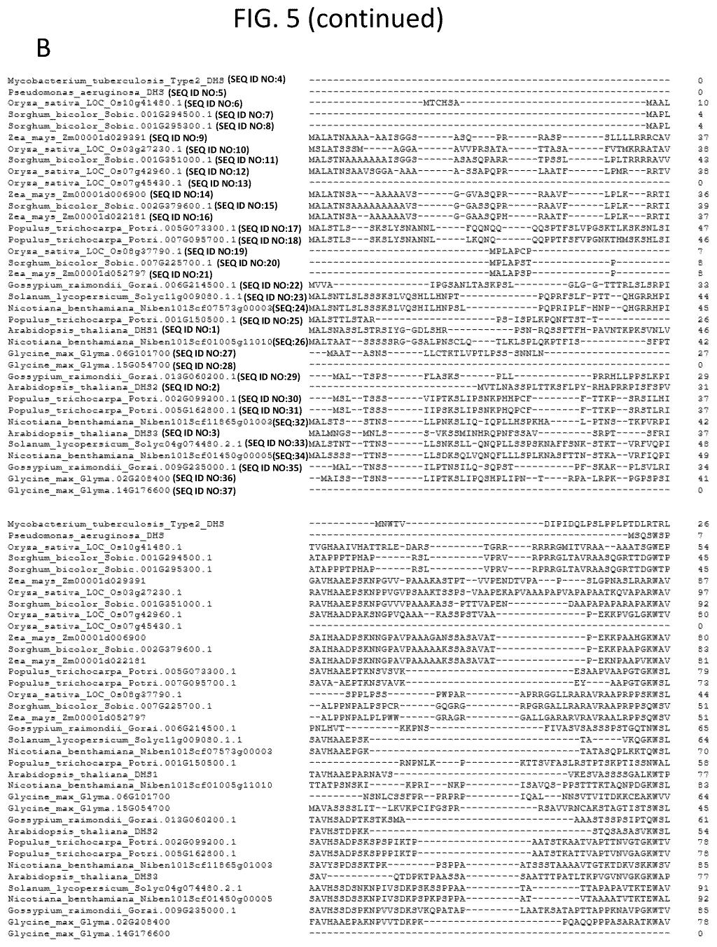

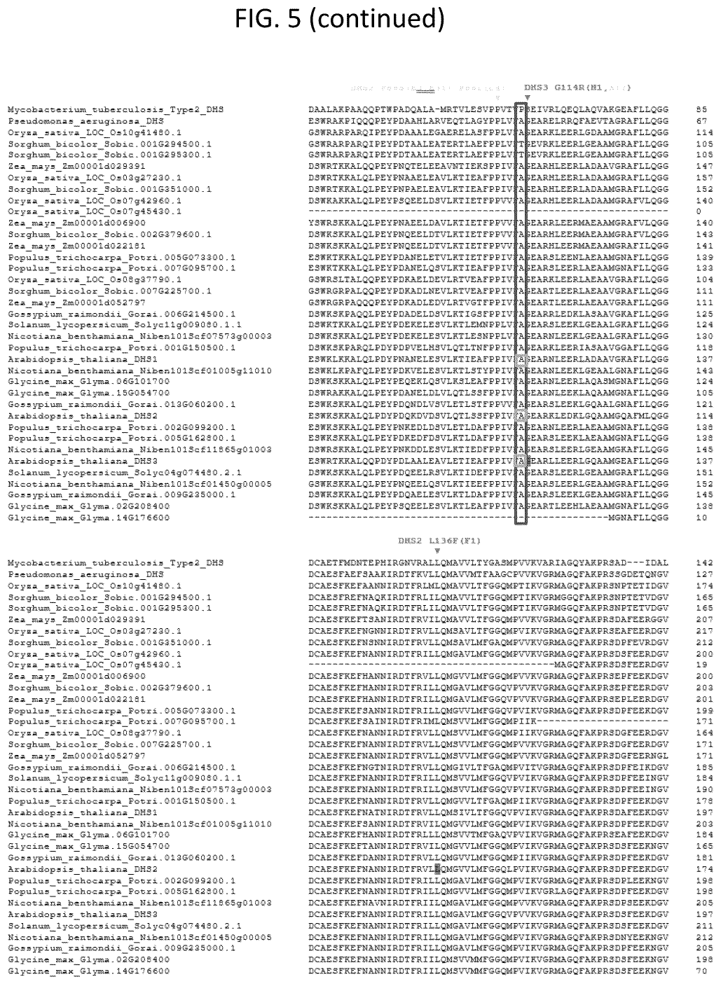

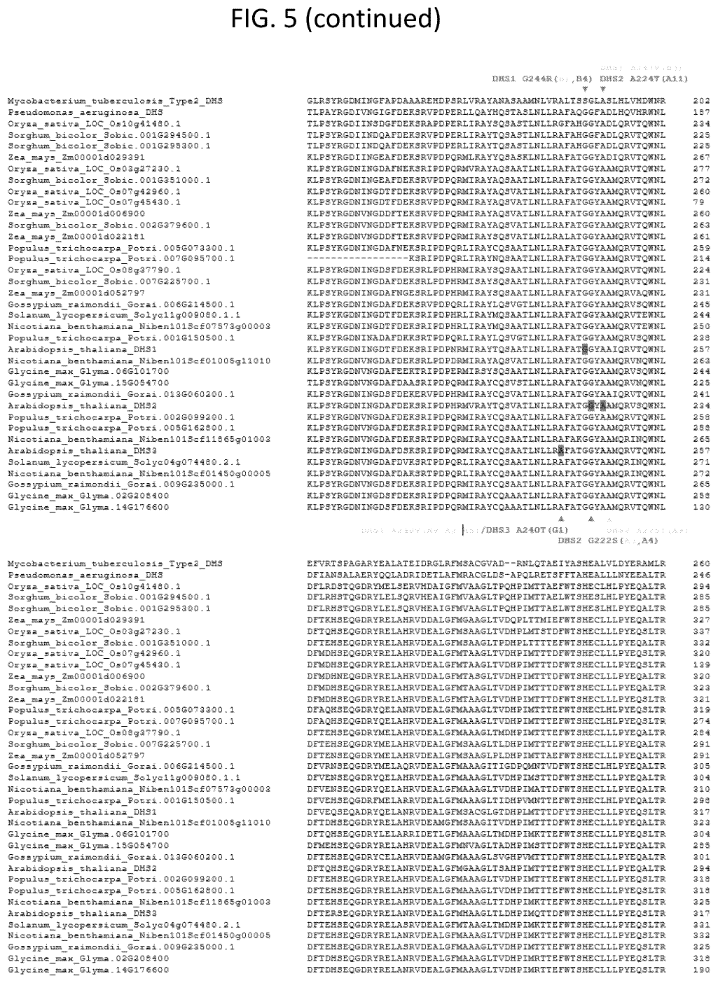

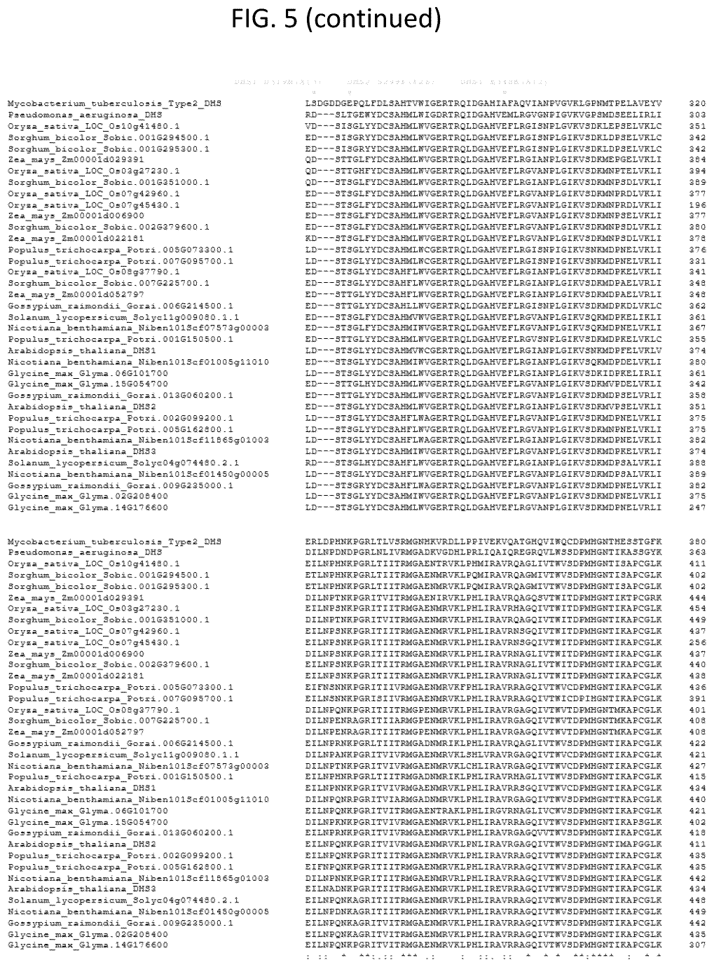

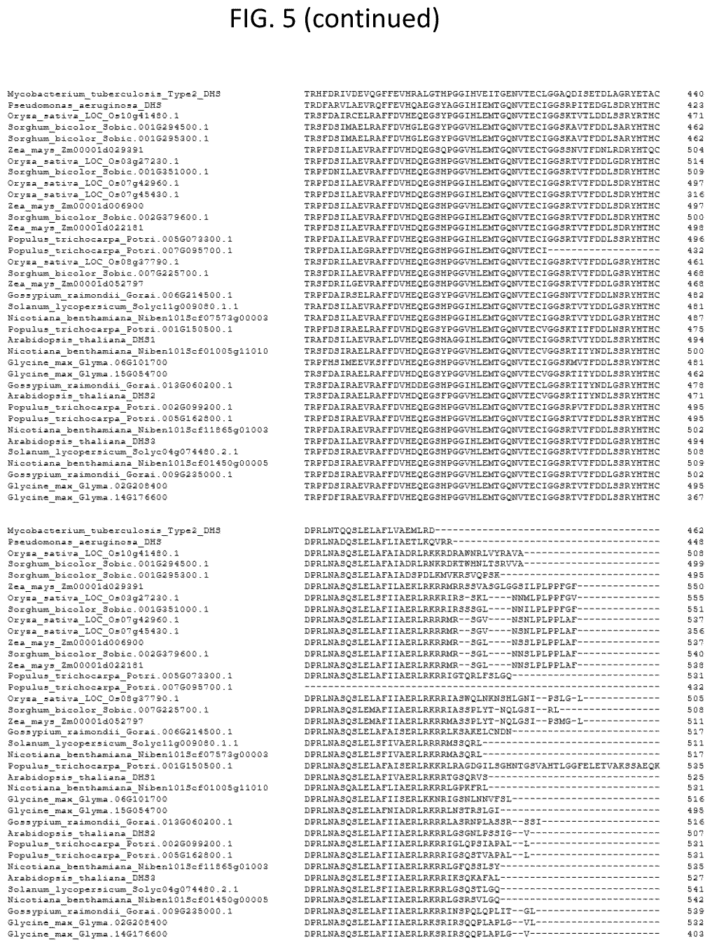

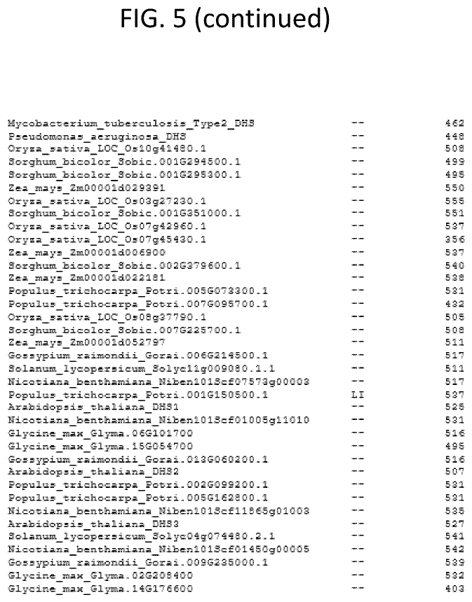

Arabidopsis thaliana expresses three isoforms of DHS, which are referred to as DHS1, DHS2, and DHS3. The sota mutations described herein were identified in one or more of these three Arabidopsis DHS isoforms. These isoforms are closely related (e.g., DHS2 has 77.58% identity to DHS1, and DHS3 has 80.53% identity to DHS1). Thus, for simplicity, we have arbitrarily used the Arabidopsis DHS1 polypeptide (SEQ ID NO:1) as a reference sequence and have specified the positions of the sota mutations using the amino acid residue numbering of this polypeptide. However, the polypeptide sequence of any related DHS polypeptide could be used instead. For example, amino acid residues 109, 114, 159, 240, 244, 245, 247, 248, 319, 322, and 348 of DHS1 (SEQ ID NO:1) correspond to residues 91, 136, 217, 218, 219, 220, 221, 222, 223, 224, and 225 of DHS2 (SEQ ID NO:2); and to residues 114, 159, 240, 241, 242, 243, 244, 245, 246, 247, and 248 of DHS3 (SEQ ID NO:3), respectively, as is demonstrated in the sequence alignment shown in FIG. 5B. Examples of other suitable reference sequences include the wild-type DHS polypeptide sequences of SEQ ID NO:1-37 (see FIG. 5B and Table 11).

In the Examples, the inventors demonstrate that expression of engineered DHS polypeptides from several plants (i.e., Arabidopsis, sorghum, and poplar) can be used to increase the aromatic amino acid production and CO₂ sequestration of a plant. DHS enzymes (which are found in bacteria and plants) are highly conserved across a wide variety of plants, as is demonstrated in FIG. 5-7. Thus, the engineered DHS enzymes used with the present invention may be from any plant species including, without limitation, a tomato plant, a tobacco plant, a soybean plant, a cotton plant, a poplar plant, a sorghum plant, a rice plant, or a corn plant. Suitable DHS polypeptides for use with the present invention include, without limitation, those having the amino acid sequences of SEQ ID NO:1-37, which may be encoded by the nucleotide sequences of SEQ ID NO:38-74, respectively (see Table 11).

In some embodiments, the engineered DHS polypeptides comprise a polypeptide or a functional fragment thereof having at least 50%, 60%, 70%, 80%, 85%, 90%, 95%, 98%, 99%, or 100% sequence identity to a polypeptide selected from SEQ ID NO:1-37. “Percentage of sequence identity” is determined by comparing two optimally aligned sequences over a comparison window. The aligned sequences may comprise additions or deletions (i.e., gaps) relative to each other for optimal alignment. The percentage is calculated by determining the number of matched positions at which an identical nucleic acid base or amino acid residue occurs in both sequences, dividing the number of matched positions by the total number of positions in the window of comparison and multiplying the result by 100 to yield the percentage of sequence identity. Protein and nucleic acid sequence identities are evaluated using the Basic Local Alignment Search Tool (“BLAST”), which is well known in the art (Proc. Natl. Acad. Sci. USA (1990) 87: 2267-2268; Nucl. Acids Res. (1997) 25: 3389-3402). The BLAST programs identify homologous sequences by identifying similar segments, which are referred to herein as “high-scoring segment pairs”, between a query amino acid or nucleic acid sequence and a test sequence which is preferably obtained from a protein or nucleic acid sequence database. Preferably, the statistical significance of a high-scoring segment pair is evaluated using the statistical significance formula Proc. Natl. Acad. Sci. USA (1990) 87: 2267-2268), the disclosure of which is incorporated by reference in its entirety. The BLAST programs can be used with the default parameters or with modified parameters provided by the user.

Regardless of their origin, the engineered DHS polypeptides of the present invention comprise at least one mutation at a position corresponding to amino acid residue 109, 114, 159, 240, 244, 245, 247, 248, 319, 322, or 348 of the Arabidopsis DHS1 polypeptide (SEQ ID NO:1). As used herein, the phrase “at a position corresponding to” refers to an amino acid position that aligns with an amino acid position in another protein in a protein sequence alignment or a protein structure alignment. For example, the phrase “at a position corresponding to amino acid residue 114 of SEQ ID NO:1” refers to an amino acid position in a polypeptide sequence that aligns with the 114^{th} amino acid residue in SEQ ID NO:1 when the two polypeptide sequences are aligned using a sequence alignment program. (Note: This position is flagged with a red arrow labeled “G114R on DHS3” above the partial sequence alignment of SEQ ID NO:1-37 shown in FIG. 5A and is labelled “DHS3 G114R” in the full-length sequence alignment shown in FIG. 5B.) To determine whether a particular polypeptide sequence has a mutation at an amino acid residue position “corresponding to” a position disclosed herein, one may align that particular polypeptide sequence with SEQ ID NO:1 using conventional alignment methods (see, e.g., Bioinformatics (2007) 23(7): 802-8) and examine the sequence alignment at the appropriate position.

FIG. 5B shows an amino acid sequence alignment of DHS polypeptides from a variety of plant species (SEQ ID NO:1-37). Based on this alignment, it is readily apparent that various amino acid residues may be mutated without substantially affecting the DHS activity of the polypeptide. For example, a person of ordinary skill in the art would appreciate that substitutions in a DHS polypeptide could be selected based on the alternative amino acid residues that occur at the corresponding position in related DHS polypeptides from other plant species. For example, the Arabidopsis DHS1 polypeptide (SEQ ID NO:1) has an alanine at position 113 while some of the other polypeptide sequences shown in FIG. 5B have a proline or threonine at this position in the alignment. Thus, exemplary modifications that could be made in the Arabidopsis DHS1 polypeptide based on this sequence alignment include A113P and A113T substitutions. Similar modifications could be made to each of SEQ ID NO:1-37 at each position of the sequence alignment shown in FIG. 5B. Additionally, a person of ordinary skill in the art could easily align other DHS polypeptide sequences with the sequences shown in FIG. 5B to identify additional mutations that could be included in the engineered DHS polypeptides.

In some embodiments, the engineered polypeptide comprises one of the specific sota mutations that were identified by the inventors in the Arabidopsis DHS enzymes in the Examples. These specific mutations include mutations corresponding to G114R, L159F, A240T, G244R, G245S, and A247T in SEQ ID NO:1 (identified in Example 1), and mutations corresponding to P109S, P109L, A240V, A247V, A248T, D319N, S322F, and E348K in SEQ ID NO:1 (identified in Example 2). Thus, in some embodiments, the at least one mutation includes at least one mutation corresponding to P109S, P109L, G114R, L159F, A240V, A240T, G244R, G245S, A247V, A247T, A248T, D319N, S322F, or E348K in SEQ ID NO:1.

In the Examples, the inventors demonstrate that the identified DHS mutations reduce inhibition by tyrosine-associated compounds and tryptophan-associated compounds (i.e., compounds consisting of or derived from tyrosine and tryptophan, respectively). Thus, in some embodiments, the engineered DHS enzymes have reduced inhibition by one or more of these compounds relative to the wild-type version of the same DHS enzyme. Exemplary tyrosine-associated compounds include, without limitation, tyrosine, tyrosol, tyramine, hydroxyphenylpyruvate (HPP), and homogentisate (HGA). Exemplary tryptophan-derived compounds include, without limitation, tryptophan, indole-3-pyruvate (IPA), indole-3-acetate (IAA; auxin), indole-3-lactate (ILA), anthranilate, and tryptamine.

Inhibition by tyrosine, tryptophan, and tyrosine/tryptophan-associated compounds may be reduced by 1.5-, 2-, 3-, 4-, 5-, 6-, 7-, 8-, 9-, 10-, 11-, 12-, 13-, 14-, 15-, 16-, 17-, 18-, 19-, 20-fold, or more as compared to the inhibition exhibited by the corresponding wild-type DHS enzyme. Inhibition by these compounds may be measured using a DHS enzyme activity assay performed in the presence of the compound. Suitable DHS enzyme activity assays include those described in Plant Cell. (2021) 33, 671-696, which is incorporated by reference in its entirety. Alternatively, DHS enzyme activity can be analyzed by measuring the loss of the substrate phosphoenolpyruvate (PEP) at absorbance 232 nm (Acta Crystallogr Sect F Struct Biol Cryst Commun (2005) 61(Pt 4): 403-6; J Biol Chem (2010) 285(40): 30567-30576). Also, the production of the product 3-deoxy-D-arabinoheptulosonate 7-phosphate (DAHP) can be directly measured via liquid chromatography-mass spectrometry (LCMS, Yokoyama R, El-Azaz J, Maeda H A, unpublished data).

### Polynucleotides:

In a second aspect, the present invention provides polynucleotides encoding the engineered polypeptides disclosed herein. The terms “polynucleotide,” “oligonucleotide,” and “nucleic acid” are used interchangeably to refer a polymer of DNA or RNA. A polynucleotide may be single-stranded or double-stranded and may represent the sense or the antisense strand. A polynucleotide may be synthesized or obtained from a natural source. A polynucleotide may contain natural, non-natural, or altered nucleotides, as well as natural, non-natural, or altered internucleotide linkages (e.g., phosphoroamidate linkages, phosphorothioate linkages). The term polynucleotide encompasses constructs, vectors, plasmids, and the like. In some embodiments, the polynucleotide is complementary DNA (cDNA; i.e., synthetic DNA that has been reverse transcribed from a messenger RNA) or genomic DNA (i.e., chromosomal DNA from an organism). Those of skill in the art understand the degeneracy of the genetic code and that a variety of polynucleotides can encode the same polypeptide.

While the polynucleotide sequences disclosed herein are derived from sequences found in plants, any polynucleotide sequence that encodes the desired engineered DHS polypeptide may be used with the present invention. For example, in some embodiments, the polynucleotides are codon-optimized for expression in a particular cell (e.g., a plant cell, bacterial cell, or fungal cell). “Codon optimization” is a process used to increase expression of a polynucleotide in a particular host cell by altering the sequence of the polynucleotide to accommodate the codon bias of the host cell. Computer programs for generating codon-optimized sequences for use in a particular host cell are known in the art.

### Constructs:

In a third aspect, the present invention provides constructs comprising a promoter operably linked to one of the polynucleotides described herein. As used herein, the term “construct” refers a to recombinant polynucleotide, i.e., a polynucleotide that was formed by combining at least two polynucleotide components from different sources, natural or synthetic. For example, a construct may comprise the coding region of one gene operably linked to a promoter that is (1) associated with another gene found within the same genome, (2) from the genome of a different species, or (3) synthetic. Constructs can be generated using conventional recombinant DNA methods.

As used herein, the term “promoter” refers to a DNA sequence defines where transcription of a polynucleotide beings. RNA polymerase and the necessary transcription factors bind to the promoter to initiate transcription. Promoters are typically located directly upstream (i.e., at the 5′ end) of the transcription start site. However, a promoter may also be located at the 3′ end, within a coding region, or within an intron of a gene that it regulates. Promoters may be derived in their entirety from a native or heterologous gene, may be composed of elements derived from multiple regulatory sequences found in nature, or may comprise synthetic DNA. A promoter is “operably linked” to a polynucleotide if the promoter is positioned such that it can affect transcription of the polynucleotide.

The promoter used in the constructs described herein may be a heterologous promoter (i.e., a promoter that is not naturally associated with the DHS polynucleotide), an endogenous promoter (i.e., a promoter that is naturally associated with the DHS polynucleotide), or a synthetic promoter that is designed to function in a desired manner in a particular host cell. Suitable promoters for use with the present invention include, but are not limited to, constitutive, inducible, temporally regulated, developmentally regulated, chemically regulated, tissue-preferred, and tissue-specific promoters. In some cases, it may be advantageous to use a tissue-specific promoter or a developmental stage-specific promoter such that the construct will drive expression of the DHS polypeptide in a particular tissue (e.g., the roots or leaves of a plant) or during a particular developmental stage (e.g., leaf maturation, seed development, senescence).

In some embodiments, the promoter is a plant promoter, i.e., a promoter that is active in plant cells. Suitable plant promoters include, without limitation, the 35S promoter of the cauliflower mosaic virus, ubiquitin, the tCUP cryptic constitutive promoter, the Rsyn7 promoter, the maize In2-2 promoter, and the tobacco PR-1a promoter.

### Vectors:

In a fourth aspect, the present invention provides vectors comprising one of the polynucleotides or constructs described herein. The term “vector” refers to a DNA molecule that is used to carry a particular DNA segment (i.e., a DNA segment included in the vector) into a host cell. Some vectors are capable of autonomous replication in a host cell (e.g., bacterial vectors that include an origin of replication and episomal mammalian vectors). Other vectors can be integrated into the genome of a host cell such that they are replicated along with the host genome (e.g., viral vectors and transposons). Vectors may include heterologous genetic elements that are necessary for propagation of the vector or for expression of an encoded gene product. Vectors may also include a reporter gene or a selectable marker gene. Suitable vectors include plasmids (i.e., circular double-stranded DNA molecules) and mini-chromosomes.

### Cells:

In a fifth aspect, the present invention provides cells comprising one of the engineered polypeptides, polynucleotides, constructs, or vectors described herein. The cells may be eukaryotic or prokaryotic. Preferably, the cell is a type of cell that can be used for large-scale production of aromatic amino acids or CO₂ sequestration. For example, in some embodiments, the cell is a plant cell, a bacterial cell, a fungal cell, or a protist cell.

In some embodiments, the cell is a plant cell. Suitable plant cells for use with the present invention include, without limitation, tomato plant cells, tobacco plant cells, soybean plant cells, cotton plant cells, poplar plant cells, sorghum plant cells, rice plant cells, corn plant cells, beet plant cells, mung bean plant cells, opium poppy plant cells, alfalfa plant cells, wheat plant cells, barley plant cells, millet plant cells, oat plant cells, rye plant cells, rapeseed plant cells, and miscanthus plant cells.

### Seeds:

In a sixth aspect, the present invention provides seeds comprising one of the engineered polypeptides, polynucleotides, constructs, vectors, or cells described herein. A “seed” is an embryonic plant enclosed in a protective outer covering. In embodiments in which the plant comprises a nucleic acid (i.e., a polynucleotide, construct, or vector) described herein, the nucleic acid may either be integrated into the genome of the seed or exist independently from the genome.

### Plants:

In a seventh aspect, the present invention provides plants grown from the seeds described herein and plants comprising one of the engineered polypeptides, polynucleotides, constructs, vectors, or cells described herein.

As used herein, the term “plant” includes both whole plants and plant parts. Examples of plant parts include, without limitation, embryos, pollen, ovules, flowers, glumes, panicles, roots, root tips, anthers, pistils, leaves, stems, seeds, pods, flowers, calli, clumps, cells, protoplasts, germplasm, asexual propagates, and tissue cultures. This term also includes chimeric plants in which only a subset of the plant's cells comprises the engineered polypeptide, polynucleotide, construct, or vector.

The plants may be of any species. In some embodiments, the plant is selected from a tomato plant, a tobacco plant, a soybean plant, a cotton plant, a poplar plant, a sorghum plant, a rice plant, and a corn plant. The protein sequences of DHS enzymes found in these plants are provided as SEQ ID NO:1-37 (see FIG. 5B and Table 11). Other suitable plants for use with the present invention include, without limitation, beet plants, mung bean plants, opium poppy plants, alfalfa plants, wheat plants, barley plants, millet plants, oat plants, rye plants, rapeseed plants, and miscanthus plants.

In the Examples, the inventors demonstrate that plants (i.e., both Arabidopsis thaliana and Nicotiana benthamiana plants) comprising sota mutant DHS enzymes (1) produce more aromatic amino acids, and (2) assimilate a greater quantity of CO₂ as compared to a control plant. As used herein, the term “control plant” refers to a comparable plant (e.g., of the same species, cultivar, and age) that was raised under the same or comparable conditions (e.g., water, sunlight, nutrients) but that does not express an engineered DHS polypeptide described herein.

In some embodiments, the plant produces a greater quantity of aromatic amino acids (i.e., tyrosine, phenylalanine, and tryptophan) or produces aromatic amino acids at a greater rate as compared to a control plant. Suitably, the plant produces at least 1.5-, 2-, 3-, 4-, 5-, 6-, 7-, 8-, 9-, 10-, 11-, 12-, 13-, 14-, 15-, 16-, 17-, 18-, 19-, or 20-fold more aromatic amino acids as compared to the control plant. Production of aromatic amino acids may be measured using ¹³CO₂ labeling followed by quantification via gas chromatography-mass spectrometry (GC-MS), liquid chromatography-mass spectrometry (LC-MS), or nuclear magnetic resonance (NMR).

In some embodiments, the plant assimilates a greater quantity of CO₂ or assimilates CO₂ at a greater rate as compared to a control plant. Suitably, the CO₂ assimilation of the plant is at least 2%, 5%, 10%, 20%, 30%, 40%, 50%, or 60% greater than that of a control plant. CO₂ assimilation may be quantified by measuring the gas exchange activity of the plant. For example, CO₂ assimilation may be measured using an LI-6400XT photosynthesis system equipped with the 6400-40 leaf chamber (LI-COR), as described in the Examples. Alternatively, labeled ¹³CO₂ can be fed to plants and the rate of ¹³C incorporation into plants can be measured over time.

### Methods for Improving Plants:

In an eighth aspect, the present invention provides methods for improving a plant by (1) increasing production of aromatic amino acids in a plant, and/or (2) increasing the amount of CO₂ sequestered by the plant. The methods comprise: introducing one of the engineered polypeptides, polynucleotides, constructs, or vectors described herein into the plant.

As used herein, “introducing” describes a process by which exogenous polypeptides or polynucleotides are introduced into a recipient cell. Suitable introduction methods include, without limitation, Agrobacterium-mediated transformation, the floral dip method, bacteriophage or viral infection, electroporation, heat shock, lipofection, microinjection, and particle bombardment. CRISPR/Cas-based gene editing systems may also be used to edit a native DHS gene in a plant to include at least one of the sota mutations described herein.

In some embodiments, the methods further comprise purifying aromatic amino acids or derivatives thereof from the plant. As used herein, the term “purifying” refers to the process of separating a desired product from other cellular components and impurities. Suitable methods for purifying aromatic amino acids and derivatives thereof include, without limitation, high performance liquid chromatography (HPLC) and other chromatographic techniques, such as affinity chromatography. A “purified” product may be at least 85% pure, at least 95% pure, or at least 99% pure.

In some embodiments, the plant to be improved is selected from a tomato plant, a tobacco plant, a soybean plant, a cotton plant, a poplar plant, a sorghum plant, a rice plant, and a corn plant.

### Methods for Using Plants:

In a ninth aspect, the present invention provides methods for using the plants described herein to (1) produce aromatic amino acids or derivatives thereof, or (2) sequester CO₂. Both sets of methods comprise growing the plants described herein. The methods for producing aromatic amino acids or derivatives thereof further comprise purifying the aromatic amino acids or derivatives thereof produced by the plant.

Exemplary aromatic amino acid derivatives that could be produced using the methods of the present invention include the tyrosine derivatives homogentisate (HGA), α-tocopherols, and γ-tocopherols, which were found to be produced at increased levels in plants comprising engineered DHS polynucleotides.

“Carbon sequestration” is a process in which atmospheric CO₂ is captured and stored. It is one method for reducing the amount of CO₂ in the atmosphere (i.e., to reduce global climate change). In some embodiments, the methods further comprise harvesting part of the plant while leaving the roots of the plant in the soil such that the carbon contained in the roots is sequestered therein. Harvestable parts of plants include, without limitation, flowers, pollen, seedlings, tubers, leaves, stems, fruit, seeds, roots, cuttings, and the like. Above ground tissues that are enriched for aromatic compounds will be decomposed slowly by soil microbes, which also enhances carbon sequestration.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Example 1

Terrestrial plants can convert atmospheric CO₂ into diverse and abundant aromatic compounds, which have unusual stability due to their aromaticity (i.e., electron delocalization) and hence are promising sinks for carbon storage of atmospheric CO₂. However, it is unclear how plants control the shikimate pathway, which connects the photosynthetic carbon fixation pathway (i.e., the Calvin-Benson-Bassham (CBB) cycle) to the pathways responsible for the biosynthesis of aromatic amino acids (AAs) and aromatic phytochemicals (FIG. 1A). Many studies have shown that the branch point enzymes involved in AAA biosynthesis in plants are differently regulated than their counterparts in microbes and that these differences may stem from the diverse biosynthetic uses of AAAs in plants (7-9). In prior studies, genetic screens have been performed to identify plants that are resistant to the shikimate pathway inhibitor glyphosate and toxic AAA analogs have been conducted. However, these studies were either unsuccessful or identified mutations in genes encoding 5-enolpyruvylshikimate-3-phosphate synthase, the glyphosate target, or branch point enzymes specific to AAA biosynthesis (10-14) rather than enzymes that regulate carbon flux through the entire shikimate pathway.

In the following example, we identify suppressor of tyra2 (sota) mutations in Arabidopsis thaliana that deregulate the first step of the plant shikimate pathway by alleviating effector-mediated feedback regulation. Plants with these sota mutations showed hyperaccumulation of aromatic amino acids accompanied by up to a 30% increase in net CO₂ assimilation. Thus, the identified mutations could be used to enhance plant-based conversion of atmospheric CO₂ into high-energy and high-value aromatic compounds.

### Results:

Suppressor of Tyra2 (sota) Identified Dominant Mutations Targeting the Entry Step of the Shikimate Pathway

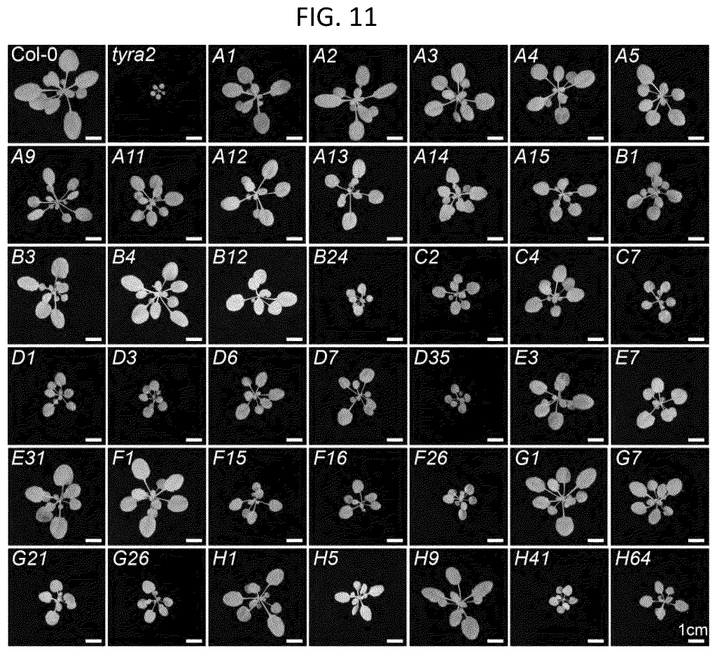

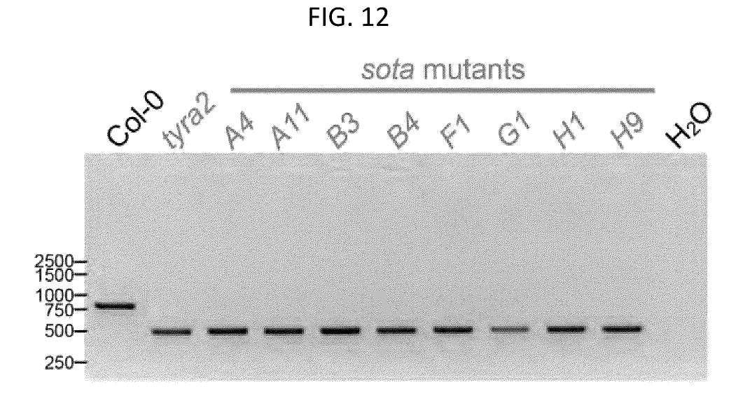

We conducted genetic screening to isolate suppressors of the Arabidopsis thaliana tyra2 knockout mutant, which lacks one of two TyrA genes of tyrosine biosynthesis (FIG. 1A) and exhibits compromised growth and reticulated leaf phenotypes (FIG. 1B) (15). Roughly 10,000 tyra2 seeds were mutagenized using ethyl methanesulfonate (EMS) and grown in eight separate pools (A to H). More than 10,000 M₂ seeds were harvested from each pool and screened for recovery of growth and/or the reticulated leaf phenotypes of tyra2. From this screen, we isolated a total of 351 suppressor of tyra2 (sota) mutants. Some lines (e.g., sotaA4 and sotaH1) recovered both growth and reticulate phenotypes, whereas other lines (e.g., sotaB4 and sotaG1) recovered growth but remained reticulate (FIG. 1B and FIG. 11) despite maintaining tyra2 deficiency (FIG. 12). When the overall profile of soluble metabolites was analyzed by gas chromatography-mass spectrometry (GC-MS) in 40 representative sota mutants, which were selected from different pools based on a range of visible phenotypes, 21 lines showed elevated tyrosine (Tyr) and phenylalanine (Phe) levels (FIG. 1C). Because these Phe and Tyr levels were positively correlated with each other (R²=0.929) (FIG. 1D), these sota mutants likely affected the upstream synthesis of arogenate, the common substrate of Phe and Tyr, from the shikimate pathway (FIG. 1A). We designated these mutants as “metabolic” sota mutants and focused our further study on them. We designated them as “metabolic” sota mutants and focused our further study on them.

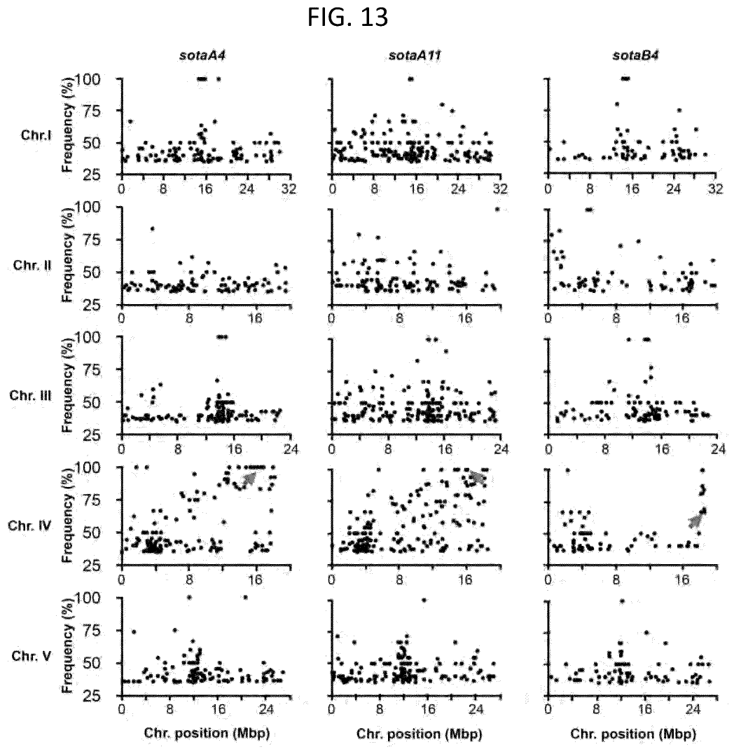

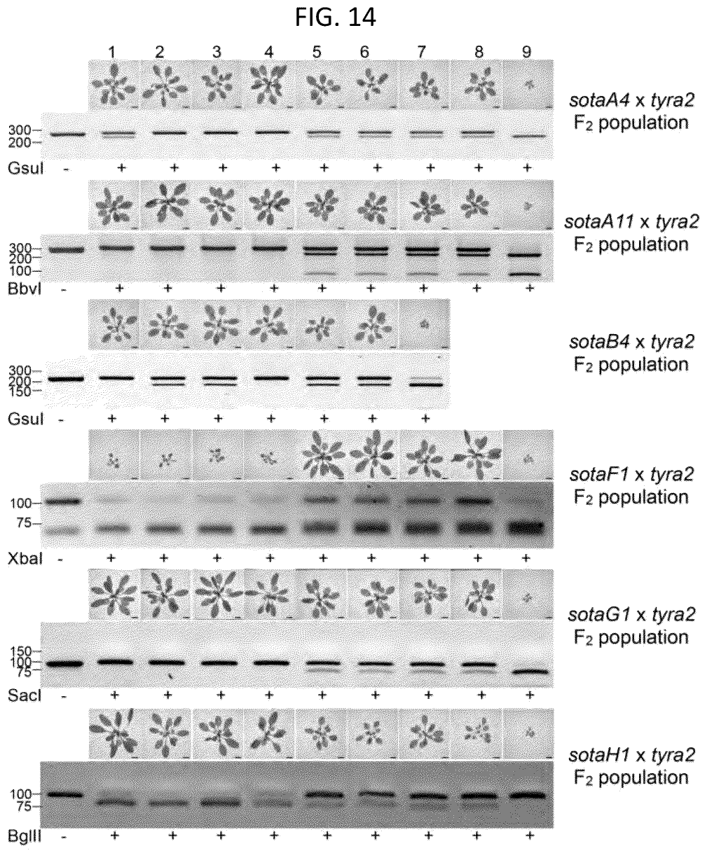

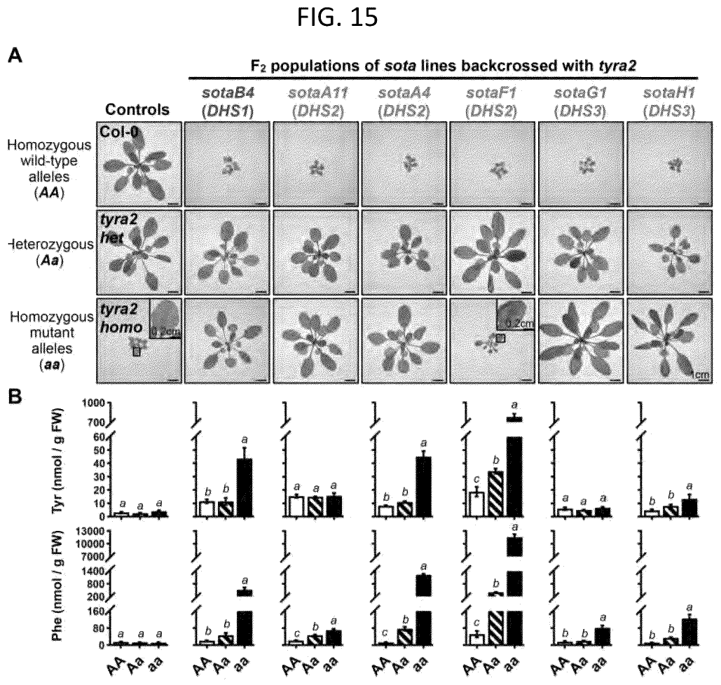

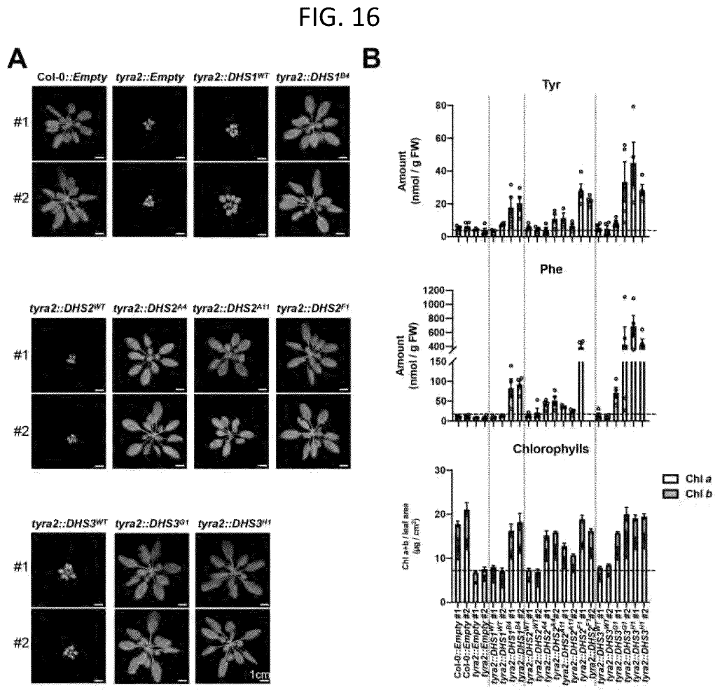

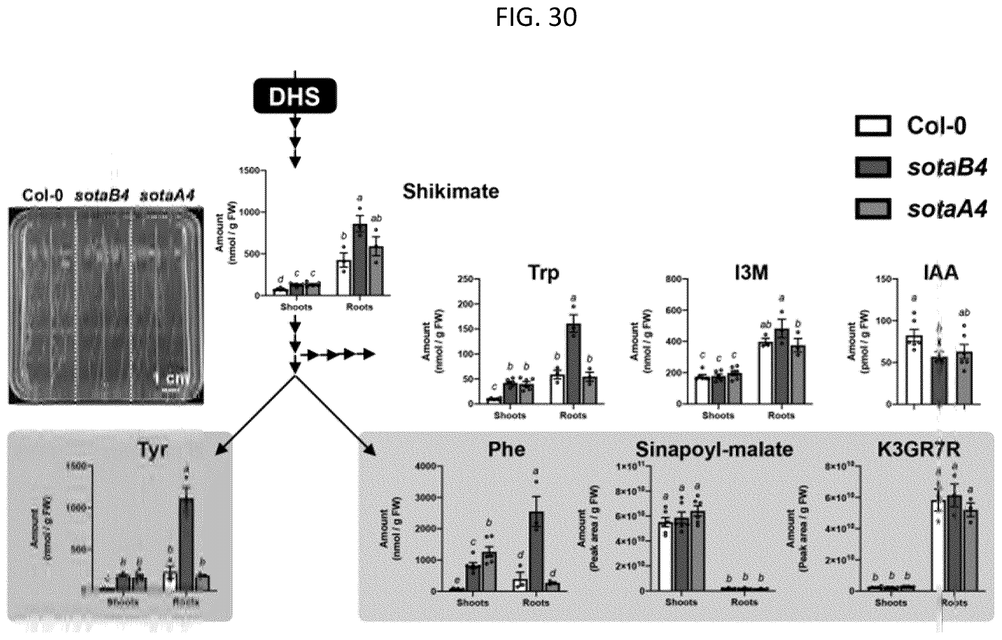

For genetic mapping, eight representative lines (i.e., sotaA4, sotaA11, sotaB3, sotaB4, sotaF1, sotaG1, sotaH1, and sotaH9) were backcrossed with the original Arabidopsis tyra2 mutant. Illumina whole-genome sequencing of tyra2-like and/or sota-like F₂ progenies identified high-frequency missense mutations from all eight lines in At4g39980, At4g33510, or At5g05920, which are the three loci encoding 3-deoxy-D-arabino-heptulosonate 7-phosphate synthase (DAHP synthase or DHS) isoforms (FIG. 13 and Table 1). In FIG. 30, the eight mapped mutations are marked on a sequence alignment of DHS orthologs from several crop species. DHS is an enzyme that catalyzes the first reaction of the shikimate pathway (FIG. 1A). The DHS sota mutations segregated with the tyra2 suppression phenotypes, as confirmed by derived cleaved amplified polymorphic sequences (dCAPS) marker genotyping in representative F₂ (FIG. 14). Their F₂ populations showed dominant or semidominant characteristics in terms of their growth recovery and Tyr and Phe accumulation phenotypes (FIG. 15), with the exception of the sotaF1 homozygous (but not heterozygous) line, which showed dwarfism that was likely due to its extreme accumulation of AAAs (FIG. 15A). Transgenic expression of DHS genes with a sota mutation (e.g., DHS1^{B4}), but not the corresponding wild-type (WT) DHS genes (e.g., DHS1^{WT}), driven by their respective endogenous promoters, in the tyra2 mutant recovered its dwarf plant and reticulated leaf phenotypes (FIG. 1E and FIG. 16A) and also led to elevated Phe and Tyr levels (FIG. 16B), phenocopying the metabolic sota lines (FIG. 1). These results provide genetic evidence that these DHS sota mutations suppress the tyra2 phenotype and enhance Tyr and Phe levels in a dominant fashion.

sota Mutations Alleviate the Complex Regulation of Plant DHS Enzymes

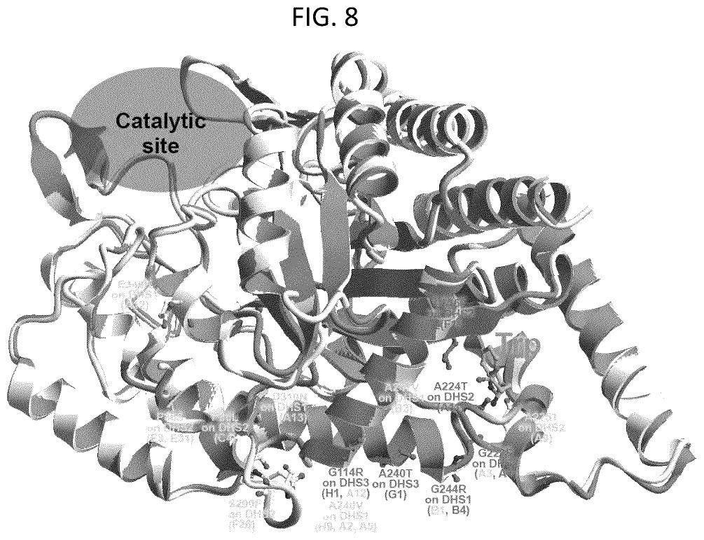

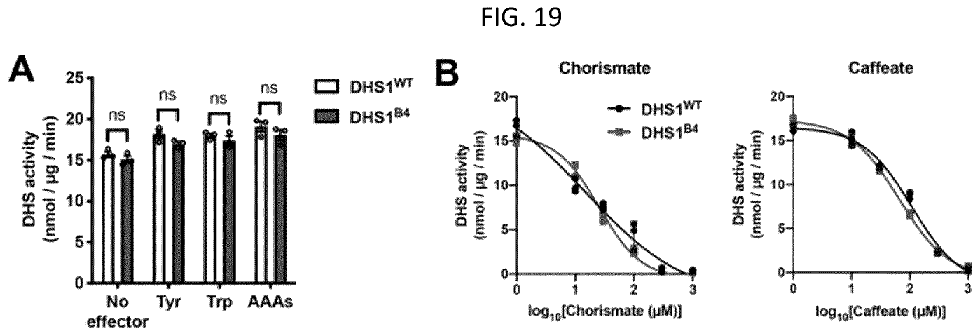

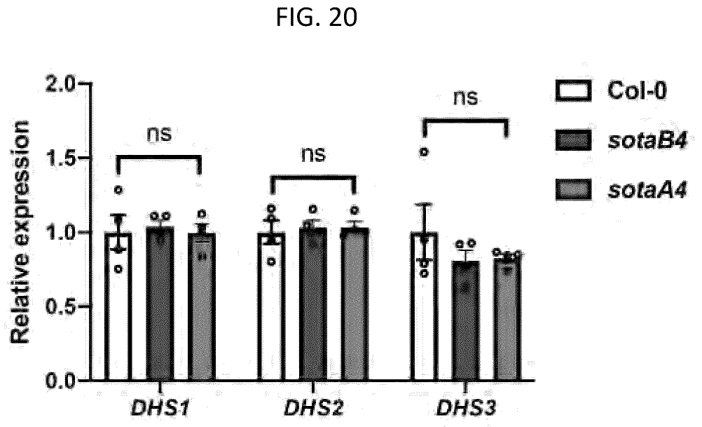

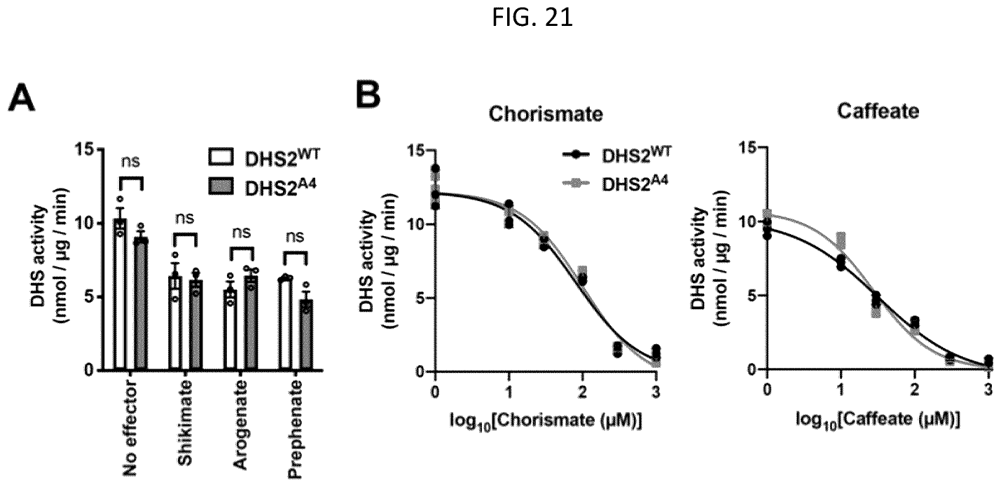

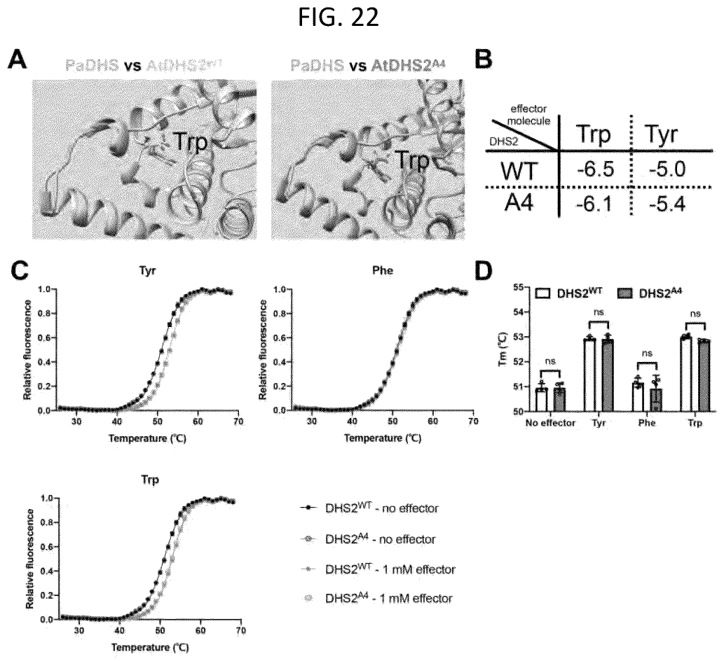

Within the DHS proteins, the identified DHS sota mutations were located near a predicted effector binding site away from the active site (FIG. 2A,B and FIG. 17, FIG. 8) as predicted from a model of Arabidopsis DHS2 generated from the Pseudomonas aeruginosa type II DHS protein structure (16). Introduction of sota mutations into recombinant DHS enzymes did not alter overall catalytic activity (FIG. 2C and FIG. 19A). Also, DHS transcript levels were unchanged in the sota mutants (FIG. 20). Thus, we hypothesized that the sota mutations might affect DHS enzyme regulation. We previously showed that tyrosine (Tyr) and tryptophan (Trp) inhibit Arabidopsis DHS2, but not the DHS1 or DHS3 isoforms (9). Chorismate and caffeate strongly inhibit all Arabidopsis DHS isoforms, while shikimate, prephenate, and arogenate slightly inhibit DHS2 (9). Here, we found that the DHS2 enzyme with the sotaA4 mutation (DHS2^{A4}) is still inhibited by shikimate, prephenate, and arogenate as well as chorismate and caffeate, with similar median inhibitory concentration (IC₅₀) values to the corresponding DHS2 WT enzyme (DHS2^{WT}) (FIG. 21 and Table 2). Unlike DHS2^{WT}, the activity of the DHS2^{A4} DHS2^{A}11, and DHS2^{F1} mutants was not inhibited by Tyr, Trp, or AAA mixtures at a concentration of up to 1 mM (FIG. 2C). Both structural docking stimulation and differential scanning fluorimetry suggested that the DHS2^{A4} mutant enzyme still binds Tyr and Trp (FIG. 22). Thus, these sota mutations completely eliminate the sensitivity of DHS2 to Tyr and Trp without altering their binding to the protein.

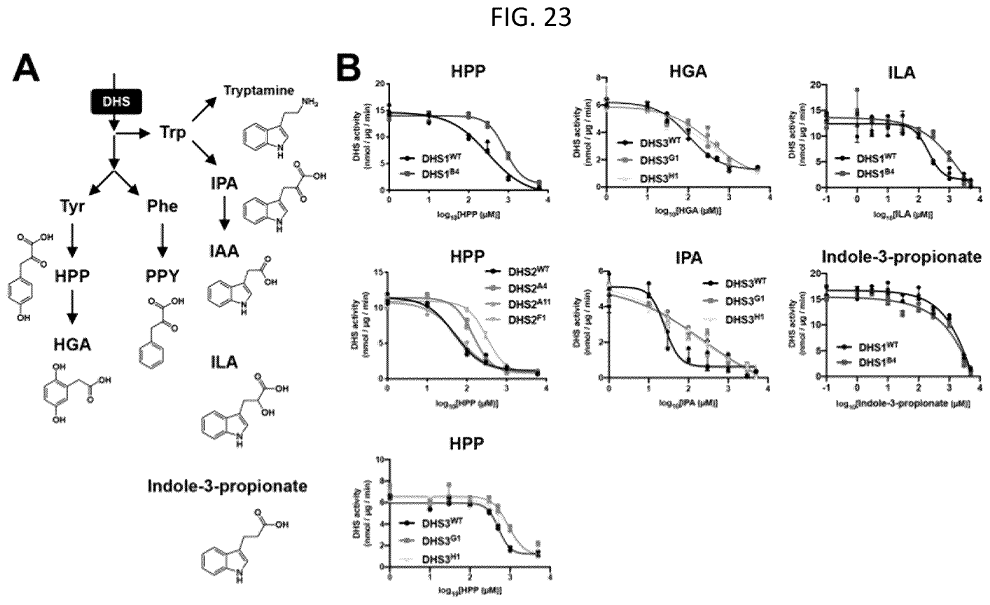

Unlike DHS2, DHS1 is not inhibited by AAAs (9), and this was also the case for the DHS1^{B4} mutant enzyme (FIG. 19A). We, therefore, hypothesized that the sotaB4 mutation might eliminate inhibition DHS1 by chorismate and caffeate. DHS1^{B4} was, however, still strongly inhibited by these effectors with comparable IC₅₀ values to that of DHS1^{WT} (FIG. 19B and Table 2). To explore how the sotaB4 mutation affects DHS1 functionality, we further screened for additional aromatic compounds downstream of AAAs that might inhibit DHS1 and DHS2 (FIG. 2D and FIG. 23A). Tyrosol and tyramine modestly inhibited DHS2 by ˜30% at 1 mM (FIG. 2D). 4-Hydroxyphenylpyruvate (HPP) and homogentisate (HGA), but neither phenylpyruvate (PPY) or 4-hydroxylphenylacetate, effectively inhibited all DHS^{WT} isoforms (FIG. 2D) with IC₅₀ of 75-250 μM for HGA (FIG. 2E, FIG. 23B, and Table 2). Notably, nearly all of the sota mutants showed significantly higher IC₅₀ for HPP and HGA (up to 7- and 12-fold increase, respectively) than corresponding WT enzymes. The one exception was DHS2^{A11} (the weakest DHS2 sota allele), which showed no significant change in IC₅₀ with HPP and HGA (FIG. 2E, FIG. 23B, and Table 2). Thus, Tyr-derived compounds can also effectively inhibit the three DHS isoforms of Arabidopsis, and the sota mutations weaken this regulation.

Further screening demonstrated that Trp-derived indole-3-pyruvate (IPA), the immediate precursor of the plant hormone indole-3-acetate (IAA; auxin), and, to a lesser extent, IAA itself inhibit both DHS1 and DHS2 (FIG. 2D) with IC₅₀ of 241 and 58 μM, respectively, for IPA (FIG. 2F and Table 2). Indole-3-lactate (ILA), but not indole-3-acetamide, also reduced the activity of both DHS1 and DHS2, with ILA having a similar inhibitory effect as IPA (FIG. 2D, FIG. 23B, and Table 2). Anthranilate and indole, intermediates of Trp biosynthesis, and tryptamine did not affect the activity of DHS1, but DHS2 showed a slight reduction in the presence of anthranilate and tryptamine (FIG. 2D). Importantly, the IPA-mediated inhibition was attenuated in the sota mutants of DHS1 and DHS3 (e.g., DHS1^{B4} and DHS3^{G1}), but not DHS2 (e.g., DHS2^{A4}), having 4 to 20-times higher IC₅₀ than corresponding WT (FIG. 2F, FIG. 23B, and Table 2). Similarly, DHS1^{B4} had a higher IC₅₀ than DHS1^{WT} for ILA but not for indole-3-propionate (FIG. 23B and Table 2). Although IPA impaired plant growth, independent of genotype, even at very low concentrations (<10 μM) likely due to its conversion to the plant hormone auxin, ILA feeding led to growth inhibition of Arabidopsis Col-0 WT plants, and this inhibition was significantly weakened in the DHS1 sotaB4 mutant plants (FIG. 2G). These in vitro and in vivo data together indicate that Arabidopsis DHS enzymes are inhibited by Trp-derived indolic compounds, and that this inhibition that is attenuated by the sota mutations of DHS1 and DHS3.

sota Mutations Deregulate the Shikimate Pathway and Elevate AAAs

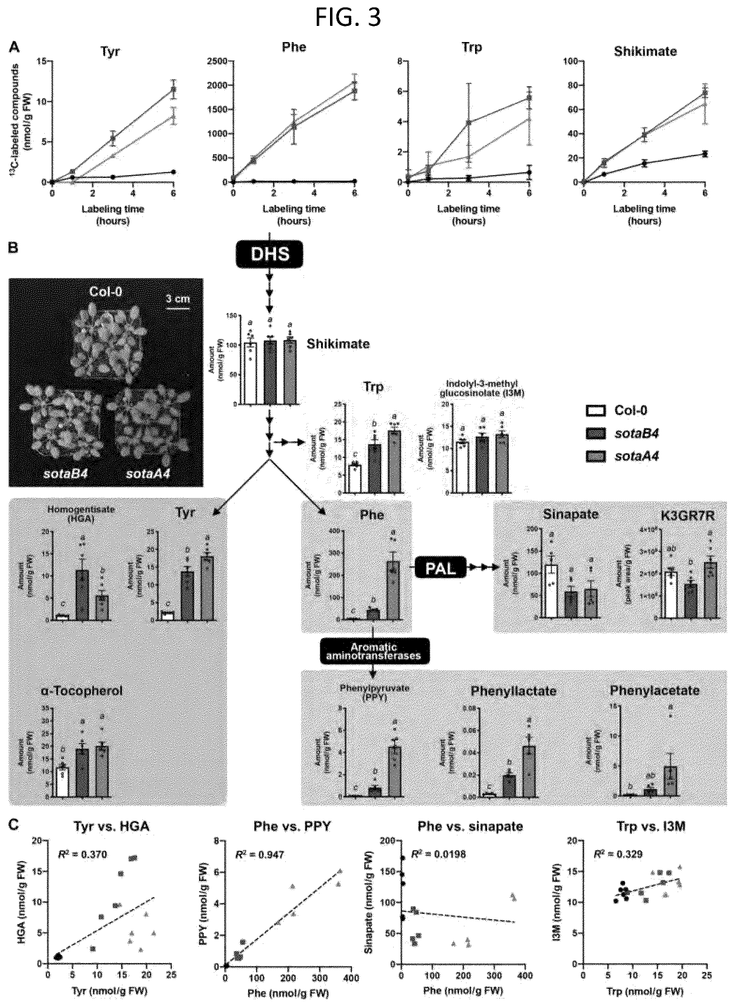

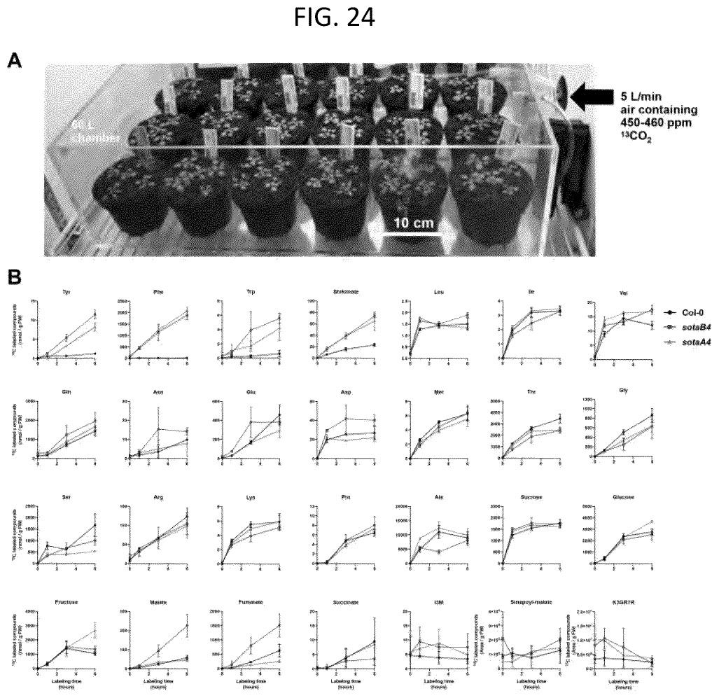

To directly test whether the relaxed feedback regulation of DHS enzymes with sota mutations increases the shikimate pathway activity in plants, Arabidopsis Col-0 (WT) and the sotaB4 and sotaA4 mutant plants were fed with stable isotope-labeled ¹³CO₂ in the light for 6 hours from the beginning of the day. The following time course metabolite analyses showed that the ¹³C label was gradually incorporated into various metabolites (FIG. 24). Compared to WT, the sotaB4 and sotaA4 mutants accumulated much higher levels of ¹³C-labeled shikimate and AAAs (FIG. 3A), but not of other amino acids, with the exception of glycine (Gly), which displayed slightly lower ¹³C incorporation in both sota lines than WT. Similar results were obtained for 3-hour ¹³CO₂ labeling also at the end of the day (FIG. 25). These labeling studies are consistent with the GC-MS profiling of the overall metabolite pools of 21 metabolic sota mutants, which revealed a large increase in AAAs, a slight reduction in Gly, but little change in other amino acids (FIG. 1C). These results indicate that the sota mutations specifically increased carbon flux through the shikimate pathway towards the biosynthesis of all three AAAs in planta.

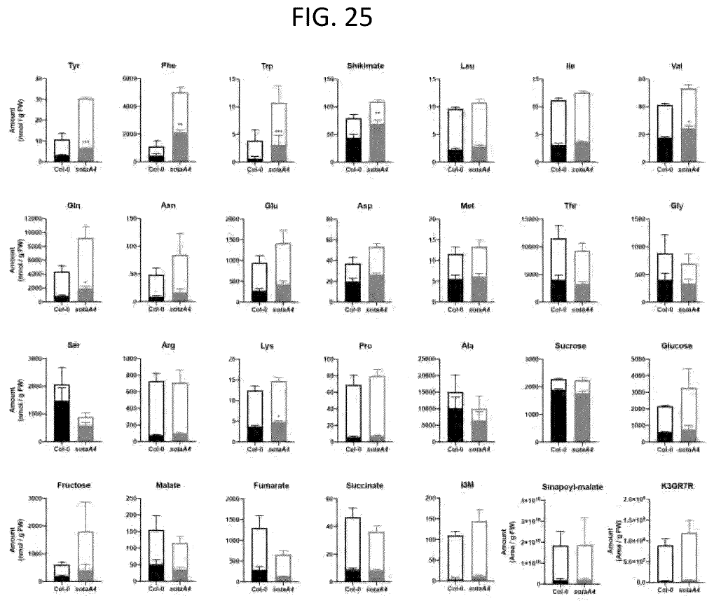

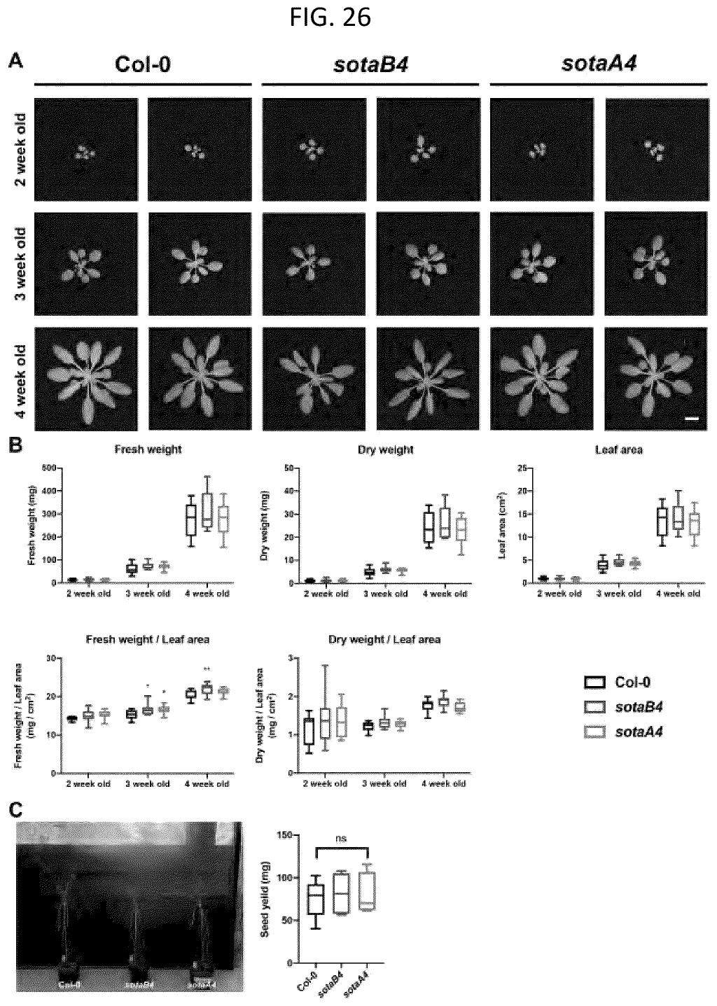

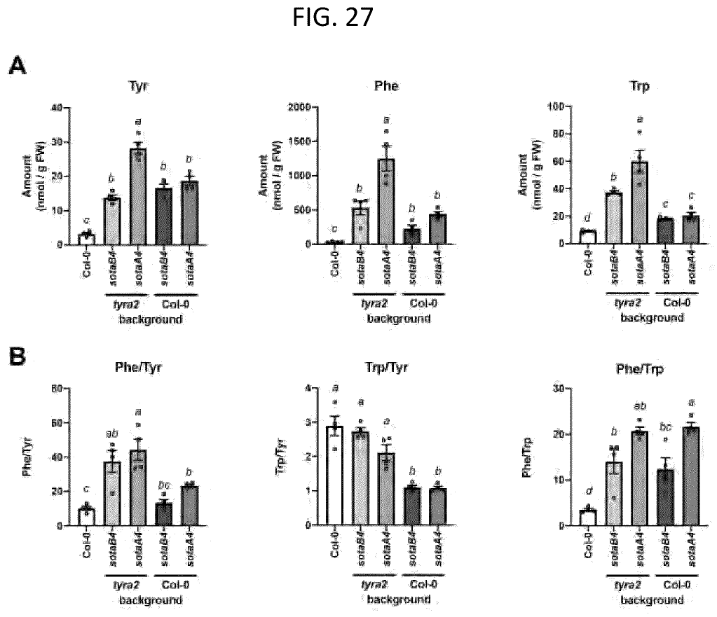

To further assess the impacts of the sota mutations on AAA and AAA-derived metabolites, we conducted targeted metabolite profiling using GC-MS and liquid chromatography (LC)-MS. First, we generated the sotaB4 and sotaA4 mutants in the Arabidopsis Col-0 background by outcrossing to Col-0. Overall, these plants were indistinguishable from Col-0 in terms of their growth and seed yield (Table 6 and FIG. 26). Comparison of AAA profiles of the sota mutants in the Col versus tyra2 backgrounds revealed that the presence of the tyra2 mutation increased Phe and Trp levels, resulting in elevated Phe/Tyr and Trp/Tyr ratios without altering the Phe/Trp ratio (FIG. 27). However, the levels of all three AAAs remained elevated in the sota mutants in the Col-0 background (FIG. 3B), which was therefore used in following analyses to eliminate effects of the original tyra2 mutation.

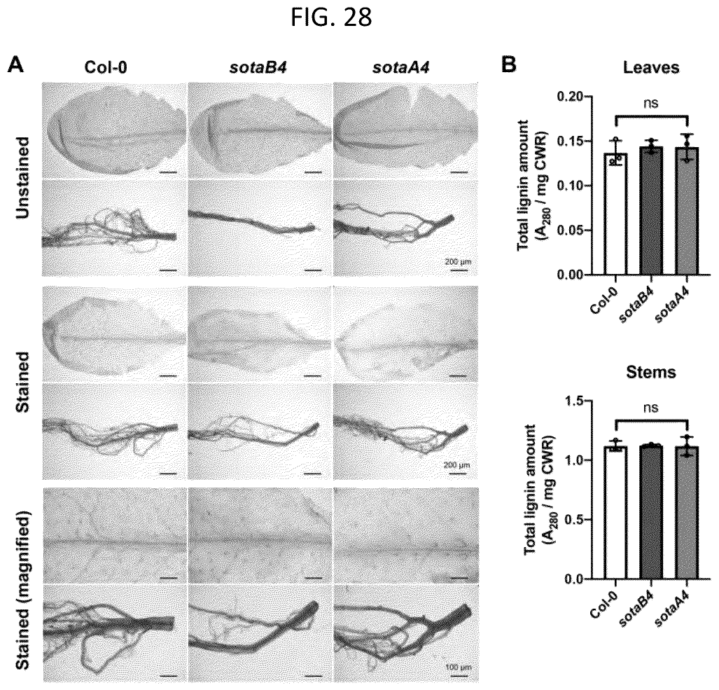

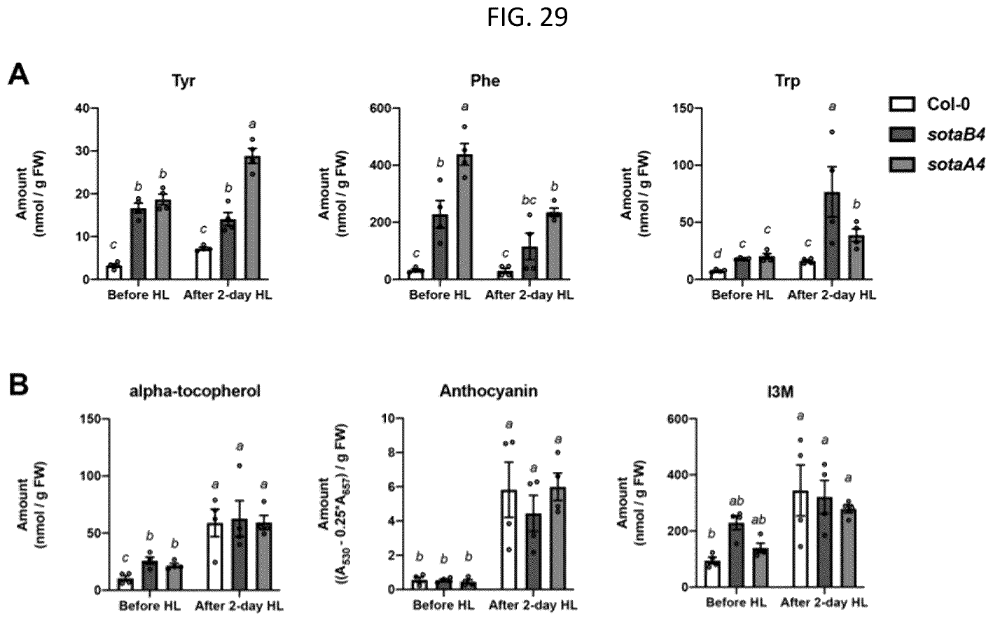

The levels of HGA and α- and γ-tocopherols derived from Tyr were, like Tyr, also elevated in both sotaB4 and sotaA4 mutants (FIG. 3B and Table 3). In contrast, the levels of Trp-derived indole glucosinolates, such as indolyl-3-methyl glucosinolate (I3M), were not elevated in the sota lines (FIG. 3B and Table 3). Similarly, the sota mutants and Col-0 had comparable levels of sinapate, sinapoylmalate, and flavonoids, including kaempferol-3-O-(2″-O-rhamnosyl)glucoside-7-O-rhamnoside (K3GR7R), which are phenylpropanoid compounds produced via Phe deamination catalyzed by Phe ammonia lyase (PAL) (FIG. 3B and Table 3) (17, 18). ¹³C-labelling of I3M, sinapoylmalate, and K3GR7R was also not increased within 6 hours of ¹³CO₂ labeling in the sota mutants compared to Col-0 (FIG. 24). The overall lignin deposition, based on phloroglucinol staining and thioglycolic acid analyses, was unaltered in sotaB4 and sotaA4 mutants (FIG. 28), unlike the ectopic lignin accumulation previously observed in some Arabidopsis transgenics (19). After high light stress, which promotes the production of numerous AAA-derived compounds, Phe-derived phenylpropanoids, such as anthocyanins and K3GR7R, and Trp-derived compounds were elevated similarly between genotypes, despite all AAA levels being always higher in sotaB4 and sotaA4 than Col-0 (FIG. 29 and Table 4). AAA and shikimate levels were also elevated in shoots and roots of plate-grown sotaB4 and sotaA4 mutants, with the one exception of sotaA4 roots (FIG. 30 and Table 5), possibly due to their isoform-specific functions (9). Again, the levels of these phenylpropanoids and Trp-derived metabolites were not significantly different between genotypes (FIG. 30 and Table 5). Thus, all three AAAs are consistently and significantly accumulated in the sota lines, but many of the downstream metabolites, particularly those derived from Phe and Trp, were not elevated. These results are consistent with the presence of multiple layers of regulations in the plant phenylpropanoid and indole metabolic network, which include both transcriptional and posttranscriptional regulations (18, 20).

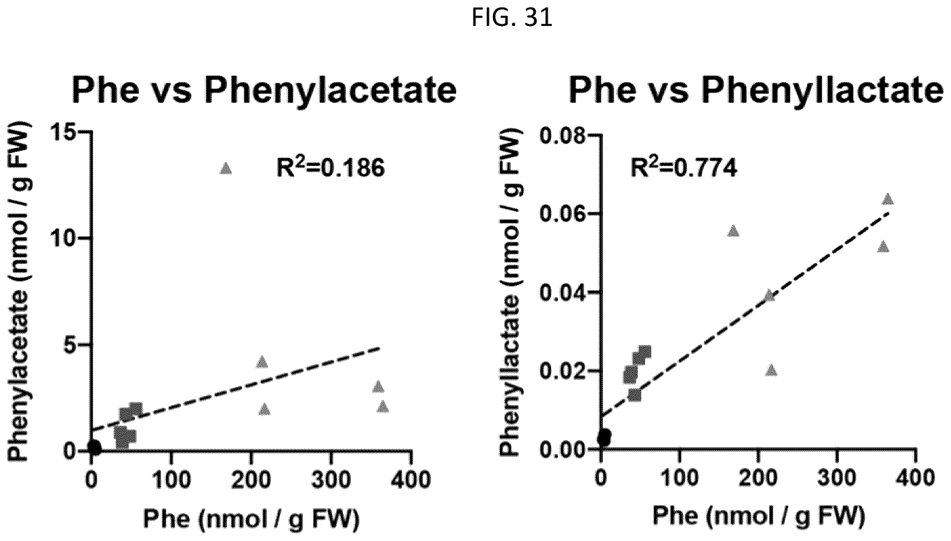

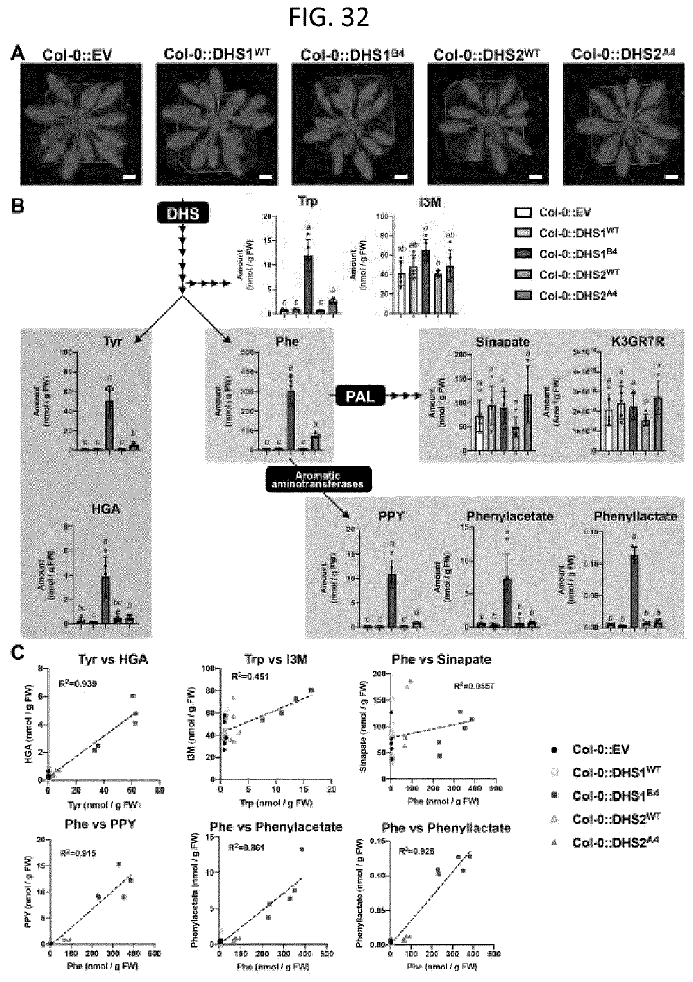

Further careful comparisons of GC-MS traces between genotypes revealed that a few previously unidentified peaks appeared in both sotaB4 and sotaA4 mutants but not in Col-0 samples. On the basis of the National Institute of Standards and Technology library search and subsequent comparison to respective authentic standards, these peaks were identified as PPY, the keto acid of Phe produced by aromatic aminotransferases (21-23), as well as phenylacetate and phenyllactate, which are both likely derived from PPY (FIG. 3B) (24, 25). Notably, the levels of PPY and PPY derivatives detected in sota mutants were positively correlated with the Phe level (FIG. 3C, FIG. 31, and Table 3). When the Col-0 WT plant was transformed with the DHS1^{B4} or DHS2^{A4} genes, but not their WT counterparts or an empty vector control, the levels of AAAs were also elevated (FIG. 32), as seen in sota mutants. Moreover, in these transgenic plants, the levels of Phe positively correlated with those of PPY and PPY-derived compounds without significant changes in the levels of phenylpropanoids, such as sinapate and K3GR7R (FIG. 32 and Table 7). Thus, the transgenic expression of a sota-mutated DHS gene, even in the presence of endogenous WT DHS genes, leads to elevated accumulation of all three AAAs and specific downstream products (e.g., HGA and PPY) inplanta.

### Deregulating the Shikimate Pathway Enhances CO₂ Assimilation

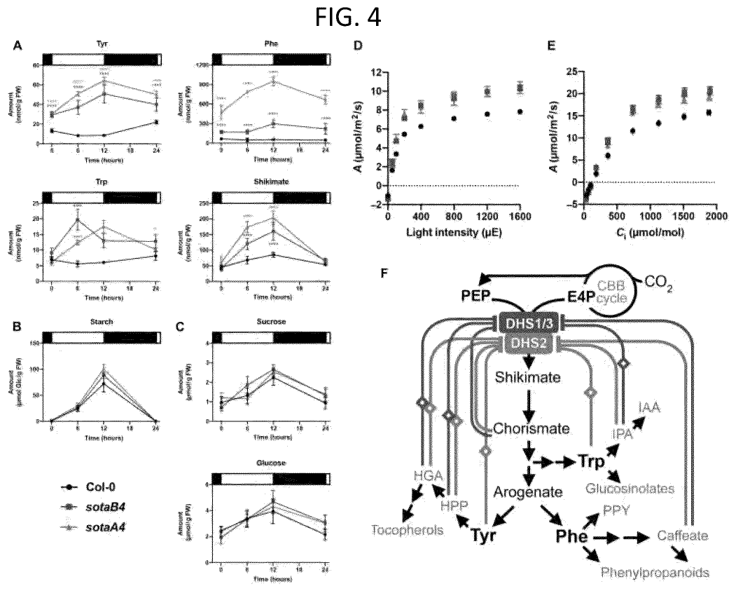

DHS uses two substrates, phosphoenolpyruvate (PEP) and erythrose-4-phosphate (E4P) that are directly supplied from glycolysis and the CBB cycle, respectively (FIG. 1A) (6, 26). We tested whether the markedly elevated AAA production in the sota mutants is supported by either starch or sugar storage pools by analyzing their levels during a day and night cycle. Compared to Col-0 WT, the sotaB4 and sotaA4 mutants had larger pools of Tyr and Phe and similar pools of Trp and shikimate at dawn. By the end of the day, the mutants had increased their levels of Tyr, Phe, Trp, and shikimate up to 7.6-, 18-, 2.9-, and 2.4-fold higher levels than Col-0, respectively. These large metabolite pools were then decreased during the night (FIG. 4A). Amounts of starch and soluble sugars, including sucrose and glucose, rose and declined during the day and night, respectively. However, despite a trend to higher dusk starch levels, carbohydrates were not significantly different in sotaB4 and sotaA4 compared to Col-0 at all timepoints (FIG. 4B,C).

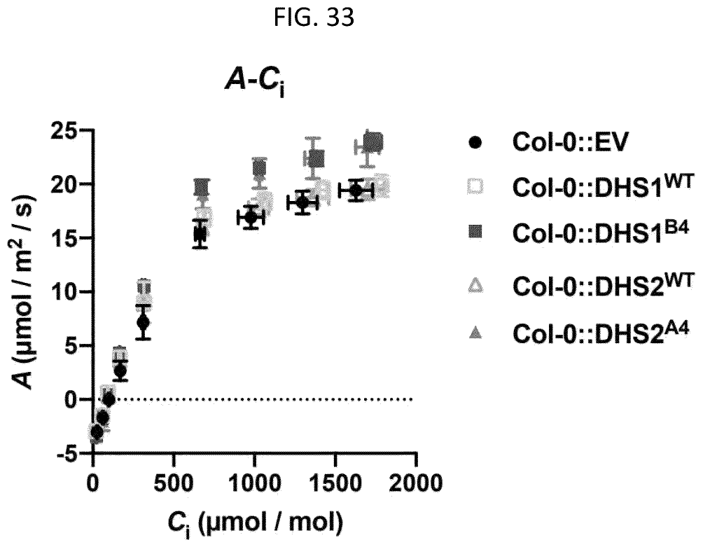

To further test potential impacts of the sota mutations on photosynthetic carbon fixation, net CO₂ assimilation rates (A) in response to different light intensity were analyzed by measuring the gas exchange activity of Col-0, sotaB4, and sotaA4 plants. Both sota mutant plants exhibited significantly higher A levels at all light intensities at and above 100 microeinstein (μE), the growth light condition used in this study, and eventually reached a plateau to an approximately 30% higher assimilation than Col-0 (FIG. 4D). When A was analyzed under different intercellular CO₂ concentrations (Cᵢ), the sota mutants exhibited up to 30% higher A than Col-0, especially at increased Cᵢ(FIG. 4E). Although total protein and Rubisco contents were unaltered in the sota mutants (Table 6), the V_{cmax} values of both sota mutants were 50% higher than that of Col-0, suggesting that the carboxylation activity of Rubisco was elevated in the sota mutants. The CO₂ compensation point (CCP) was comparable between genotypes, but R_{d} values, which represent dark respiration, were elevated in the sota mutants, which may further support production of energy-intensive AAA biosynthesis (Table 6) (27). The enhanced CO₂ assimilation was similarly observed in the transgenic lines of the Col-0 background expressing the mutated DHS1^{B4} or DHS2^{A4} genes but not the WT DHS genes (FIG. 33 and Table 8). These results revealed that deregulation of the shikimate pathway by the sota mutations is accompanied by increased activity of carbon fixation.

### Discussion:

The DHS-catalyzed reaction has been assumed to be important for the regulation of the plant shikimate pathway based on prior microbial studies (26, 28) and expression of deregulated microbial DHS in plants (29-31). Our study provides strong genetic evidence to support this notion, as all eight studied metabolic sota mutations mapped to the loci encoding DHSs, but not other shikimate pathway enzymes. Unlike microbial DHSs that are directly by inhibited by the pathway product (i.e., AAAs), this study found that plant DHSs are subjected to highly complex feedback regulation mediated by not only AAAs but also by many AAA-derived compounds (FIG. 4F). The identified sota mutations relax DHS feedback inhibition without affecting effector binding per se (FIG. 22), similar to a recently reported analogous mutation in Mycobacterium tuberculosis DHS (32). As no significant conformational change was observed in the protein structure (32), the molecular basis of how these mutations deregulate the feedback inhibition in microbial and plant DHS enzymes remains unknown. Although the sota mutations either abolished or attenuated DHS regulation by multiple effectors and we cannot pinpoint a specific molecule, the degree of HGA and HPP inhibition (FIG. 2E and FIG. 23B) inversely correlated with that of AAA accumulation among different DHS2 sota lines (FIG. 15 and FIG. 16). Thus, plant DHS monitors the levels of multiple downstream AAA-derived compounds and plays crucial roles in controlling the shikimate pathway and AAA production in plants. Importantly, the dominant nature of the sota mutations (FIG. 1 and FIG. 14-16 and FIG. 32) provides us ways to overcome the negative regulation of endogenous DHS enzymes in other plants, e.g., in a specific tissue or developmental stage.

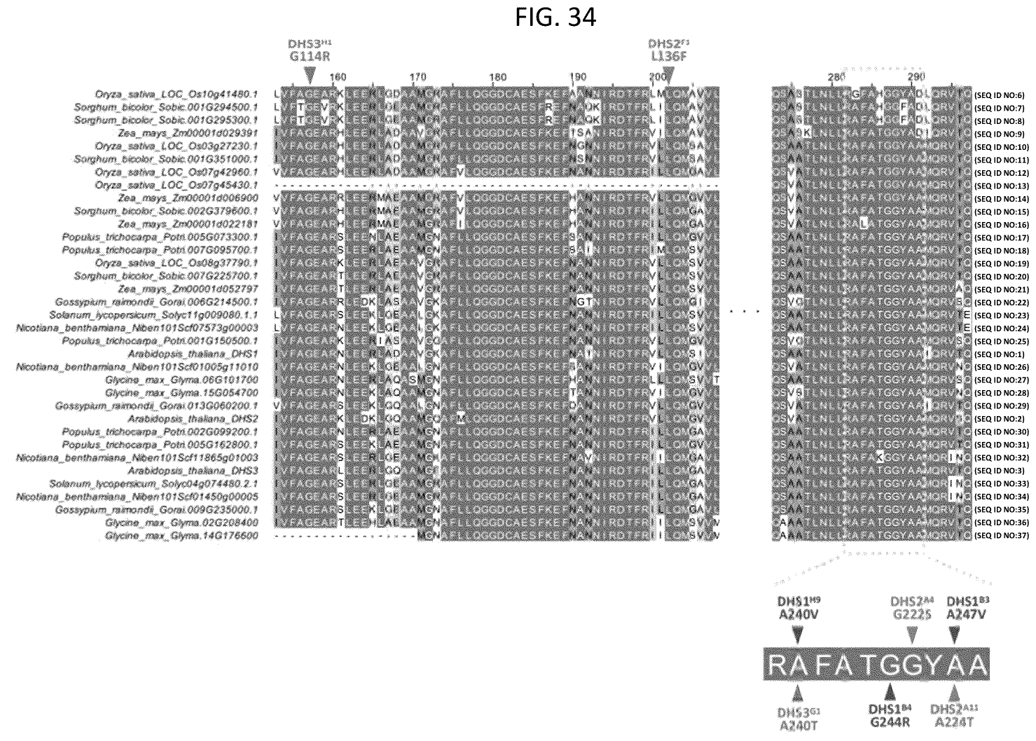

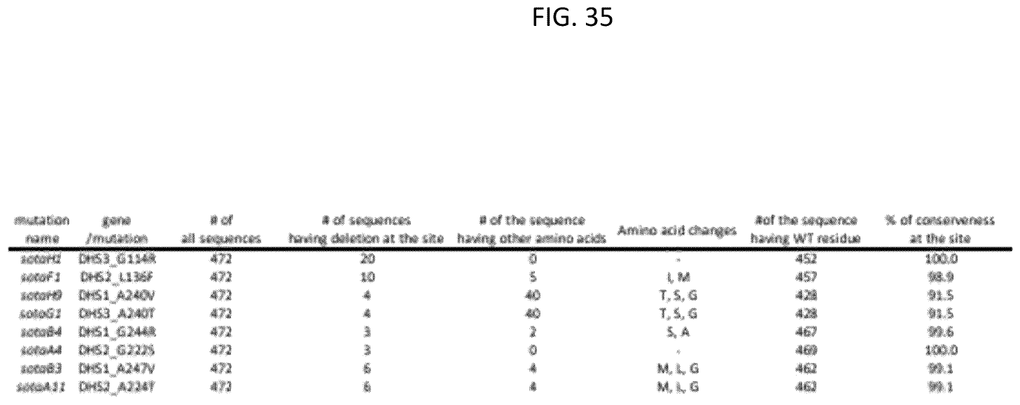

The elevated CO₂ assimilation observed in the sota mutants was striking and is likely important for efficient supply of E4P (FIG. 4F). This also agrees with prior reports that plant DHSs have high Kₘ for E4P (9) and that transketolase activity, which produce E4P in the CBB cycle, is important for AAA production in plants (33). Unlike the Arabidopsis transgenics overexpressing CBB pathway enzymes that had elevated CO₂ assimilation and increased biomass (34), the sota mutations did not alter plant biomass (Table 6 and FIG. 26). Instead, the increased photosynthesis observed in the sota mutant plants (FIG. 4E, Table 6, and FIG. 33) would provide additional energy to support the elevated activity of the highly energy-intensive shikimate pathway and AAA biosynthesis (27). Although the exact mechanism of the elevated CO₂ assimilation is currently unknown, a rapid use of E4P might alleviate negative regulations of the CBB cycle in the sota lines (35, 36). Notably, unlike the AAA imbalances and compromised growth caused by deregulation of a specific AAA biosynthetic branch (13, 15), the sota mutations had limited impacts on overall DHS activity in the absence of effectors (FIG. 2C, FIG. 19, and FIG. 21) and overall plant growth (Table 6, FIG. 26, and FIG. 32). In addition, these sota mutations occur in amino acid residues of DHSs that are well-conserved among different plants, including important agricultural and bioenergy crops (e.g., maize and sorghum; FIG. 34 and FIG. 35), and hence can be directly introduced into crops via gene editing (37). Thus, the series of the DHS point mutations identified in this study provides useful genetic tools to enhance the conversion of CO₂ into aromatic compounds in plants for sustainable production of high-value compounds while concomitantly reducing atmospheric CO₂.

### Materials and Methods:

### Plant Materials

Arabidopsis thaliana plants used in this study were grown under a 12-hour/12-hour 100-μE light/dark cycle with 85% air humidity in soil supplied with Hoagland solution or on the agarose-containing 0.5-strength Murashige and Skoog (MS) medium with 1% sucrose, unless stated otherwise.

Screen for Suppressor of Tyra2 (sota) Mutations

The seeds of the tyra2-1 transfer DNA insertion mutant (SALK_001756), which were previously characterized determined to be null homozygous with a dwarf and reticulate phenotype (15), were used to conduct a forward genetic suppressor screen using ethyl methanesulfonate (EMS), following a method by Weigel and Glazebrook (38) with a few modifications. Briefly, ˜10,000 tyra2 homozygous seeds were mutagenized with 0.2% EMS (M0880, Sigma-Aldrich) for 15 hours in a 50-mL Falcon tube on a rocking platform. Seeds were rinsed with ultrapure water 10 times and soaked in the last rinse for 1 hour. Subsequently, seeds were suspended in 400 mL 0.1% agarose and spread on eight different trays (˜50 mL on each tray, the 1020 tray; CN-FLXHD, Greenhouse Megastore, Danville) containing germination soil mix (8269028, Sungro). Eight M₁ pools from different trays were named with alphabet letters (A to H). Each pool contained approximately 1000 M₁ plants. Mutagenesis efficiency was calculated by applying the Poisson distribution, as described previously (38). Observation of siliques from 50 M₁ plants identified 15 plants without aborted seeds, indicating that the mutagenesis was successful. M₂ screening was performed by germinating ˜10,000 seeds from each M₁ pool on 10 trays containing the germination mix. A total of ˜80,000 M₂ seeds were germinated on 80 trays. Phenotypes were evaluated at 4 to 5 weeks after germination. Col-0 and tyra2-1 were germinated side by side with EMS mutants in each tray for comparison. Plants showing the tyra2-like dwarf and reticulate leaf phenotypes were removed, while ones showing any recovery of either one or both of the tyra2 phenotypes were kept and deemed to be suppressor of tyra2 (sota) lines. Each sota line was named based on the pool (i.e., A to H) from which it originated followed by a number. For example, the line sotaB4 is the fourth sota line recovered from pool B. Each M₂ sota line was allowed to self-fertilize, and the resulting M₃ seeds were collected for further experiments.

Whole-Genome Sequencing-Based Mapping of sota Mutations

To identify the causal mutations leading to the suppression of the tyra2 phenotypes and the accumulation of aromatic amino acids (AAA) in the metabolic sota lines, the M₃ plants of a first subset of the sota lines (i.e., sotaA4, sotaA11, and sotaB4) were backcrossed with tyra2. Note: The remaining sota lines were analyzed later, see below. The F₁ population also showed the tyra2 recovery phenotype, indicating that all three of the tested sota mutations had semidominant or dominant characteristics, with the F₁ plants of sotaB4 being almost indistinguishable from its M₃ parent. As expected, roughly one quarter of F₂ segregating populations showed the tyra2-like phenotypes (FIG. 14). For genetic mapping, roughly 200 seedlings showing tyra2-like and sota-like phenotypes were separately harvested and pooled into six samples. The genomic DNA of the pooled seedlings was isolated using the DNeasy Plant Mini Kit (Qiagen) according to the manufacturer's protocol, and the DNA samples were submitted to a sequencing facility for DNA library preparation, barcoding, and whole genome sequencing (100 bp single-end reads) using the Illumina HiSeq 2500 sequencer. To look for causal mutations that are present in the sota-like F₂ population but not in the tyra-like F₂ population, the sequencing data for both populations were analyzed using CLC Genomics Workbench 11.0.1 (QIAGEN). Single nucleotide variants (SNVs) were obtained by comparing each sequencing result to the TAIR10 reference genome. SNVs that were also identified in the tyra2-like population were then subtracted from the list of SNVs identified in the sota-like population. The remaining SNVs identified in the sota-like population were plotted based on their frequency among obtained reads (y axis) and their genomic position (x axis) (FIG. 13 and Table 1). This frequency calculation allowed us to identify candidate causal mutations within genetic loci encoding DHSs (Table 1). Subsequently, five additional metabolic sota lines, namely sotaB3, sotaF1, sotaG1, sotaH1, and sotaH9, were also backcrossed to the tyra2 mutant and subjected to genetic mapping using next-generation sequencing, as described above, but this time only the sota-like population of their F₂ population was sequenced. Some of the identified mutations were further confirmed by dCAPS analysis (FIG. 14) and complementation experiments (FIG. 1E and FIG. 16), as described below.

dCAPS-Based Genotyping of the sota Mutants

To determine if the DHS sota mutations identified by the whole genome sequencing segregated with the sota-like phenotype (i.e., suppression of tyra2 phenotypes), the presence and absence of each DHS sota mutation was examined in F₂ populations via a cleaved amplified polymorphic sequence (dCAPS) analysis. Primers for each sota SNV were designed using the bioinformatic tool dCAPS Finder 2.0 (39), while complementary primers for each dCAPS primer were designed using primer3 v.0.4.0 (40). The sequences of these primers are listed in Table 9 and Table 10. Polymerase chain reaction (PCR) was performed using EconoTaq PLUS green 2× master mix (Lucigen) in a 20-μL reaction containing ˜10 ng genomic DNA and 0.5 μM of each primer. After amplification, the PCR product was visualized on a 4% 1× tris-borate EDTA (TBE)-agarose gel via electrophoresis and 5 μL of the PCR product was digested using the restriction enzyme indicated in Table 9 (Thermo Scientific) in a 20-μL reaction. Digested fragments were separated by electrophoresis in a 4-5% 1×TBE-agarose gel containing ethidium bromide. The GeneRuler Ultra Low Range DNA Ladder (Thermo Scientific) was used to verify the sizes of the digested fragments. In all eight sota lines, the corresponding DHS sota mutation was found only in F₂ individuals exhibiting the tyra2 suppression phenotypes (FIG. 14).

### Generation of Transgenic Plants

We next determined whether the identified sota mutations were responsible for the observed phenotypes, including the tyra2 suppression phenotypes and the elevated levels of Tyr and Phe (FIG. 1B,C). In view of the dominant (or semidominant) nature of the sota mutations, we transformed the original tyra2-1 mutant line with a DHS gene, either with or without an identified sota mutation, to see if we could recapitulate the sota-like phenotypes. Site-directed mutagenesis was used introduce different sota mutations into binary vectors containing the wild-type (WT) versions of the DHS1, DHS2, and DHS3 cDNA, which we previously used to rescue the corresponding dhs knockout mutants (9). These vectors also contain a hygromycin resistance gene and the pFAST-R construct, a C-terminal red fluorescence protein (RFP) fusion protein driven by a seed-specific Oleosin1 (At4g25140) native promoter (41, 42).

Mutagenesis PCR was carried out by mixing 1 ng ribonuclease-treated plasmid as template, 2× PrimeSTAR® MAX DNA polymerase mix (R045A, Takara Bio USA), and 0.5 μM oligonucleotide primers (Table 10), which were designed using the Takara Web tool for mutagenesis (www.takarabio.com/learning-centers/cloning/primer-design-and-other-tools). After 20 cycles of PCR (98° C. for 15 seconds, 58° C. for 10 seconds, 72° C. for 2 minutes, and final extension at 72° C. for 5 minutes), the PCR product was treated with FastDigest DpnI (Thermo Scientific), purified using QIAquick PCR Purification Kit (QIAGEN), and introduced into ultracompetent E. coli MC1061 cells (Lucigen). The final binary vector sequence was confirmed by whole-plasmid sequencing (MGH DNA Core).

To generate transgenic Arabidopsis plants in the tyra2-1 mutant background, tyra2-1 seeds were germinated on the germination mix and grown until flowering before being transformed with each construct using the floral dip method (43). The transformed T₀ plants were allowed to complete their life cycle in the growth chamber, and dried T₁ seeds were harvested. The positive T₁ transformants were then selected based on RFP fluorescent marker expression, i.e., by observing the seeds under the AxioZoom V16 (Zeiss) stereo fluorescent microscope with RFP settings (EX 572/25, BA590, EM 629/62). T₂ seeds were used to select lines that contain a single insertion of the transgene. Overall, eight individual T₂ plants from each single insertion line were allowed to complete their life cycle and their seeds were observed under a stereo RFP fluorescent microscope to identify homozygous T₃ seeds, which were used for further analyses. Due to positional effects, some T₂ homozygous plants could not complete their life cycle because of high accumulation of AAAs, similar to the sotaF1 homozygous line. For these specific lines, T₂ heterogeneous plant populations were used for further analysis. Notably, although the hygromycin resistance gene was also present, seed selection based on RFP expression was more efficient and less aggressive, allowing for the germination of positive transformants directly on soil.

To generate transgenic lines expressing the WT or sota-mutated DHS genes in the Col-0 background, the same constructs that were used for the complementation test were transformed into Col-0 plants. One leaf of each 5-week-old T₂ plant of each line was first analyzed for photosynthetic measurement, and then other leaves were harvested from the same plant for metabolite analysis.

### Enzyme Preparation and Enzymatic Assay

To generate recombinant DHS proteins the pET28a vectors carrying the A. thaliana DHS1 (AtDHS1), AtDHS2, or AtDHS3 WT sequence without the predicted plastid transit peptide (amino acid residues; 49-525, 34-507, and 52-527, respectively) were expressed in E. coli Rosetta-2 cells and purified using Ni-affinity chromatography, exactly as was conducted previously (9). To generate DHS proteins with individual sota mutations via site-directed mutagenesis, these pET28a plasmid templates were diluted by 500-fold, mixed with 0.04 U/μL Phusion DNA polymerase (Thermo Scientific), 0.2 mM deoxynucleoside triphosphates (dNTPs), 1× Phusion reaction buffer (Thermo Scientific), and 0.5 μM forward and reverse mutagenesis primers (Table 10). The PCR reaction was run using the following protocol: 98° C. for 30 s followed by 20 cycles of 10 s at 98° C., 20 s at 70° C., 4.5 min at 72° C. with a final extension at 72° C. for 10 min. The PCR products were purified using a QIAquick Gel Extraction Kit (QIAGEN), treated with FastDigest DpnI (Thermo Scientific) to digest methylated plasmid template DNA for 20 min at 37° C., and transformed into E. coli cells. The mutagenized pET28a plasmids were sequenced to confirm that no errors were introduced during the mutagenesis process.

The DHS enzyme assays were conducted using the colorimetric method that we recently described (9). Briefly, the enzyme solution (7.7 μl) containing 50 mM Hepes (pH 7.4) was preincubated with an effector molecule(s) at room temperature for 15 min. For assays using recombinant protein and enzyme fractions isolated from plant leaves, 0.01 to 0.1 μg and approximately 50 μg of proteins were used, respectively. After adding 0.5 μl of 0.1 M dithiothreitol, the samples were further incubated at room temperature for 15 min. During these incubations, the substrate solution containing 50 mM Hepes (pH 7.4), 2 mM MnCl₂, 4 mM E4P, and 4 mM PEP at final concentration was preheated at 37° C. The enzyme reaction was started by adding 6.8 μl of the substrate solution, then incubated at 37° C. for 30 min, and terminated by adding 30 μl of 0.6 M trichloroacetic acid. After a brief centrifugation, 5 μl of 200 mM NaIO₄ (sodium meta-periodate) in 9 N H₃PO₄ was added to oxidize the enzymatic product and to incubate at 25° C. for 20 min. To stop the oxidation reaction, 20 μl of 0.75 M NaAsO₂ (sodium arsenite), which was dissolved in 0.5 M Na₂SO₄ and 0.05 M H₂SO₄, was added and immediately mixed. After 5 min of incubation at room temperature, one-third of the sample solution was transferred to a new tube to be mixed with 50 μl of 40 mM thiobarbituric acid and incubated at 99° C. for 15 min in a thermal cycler. The mixture was added to 600 μl of cyclohexanone in eight-strip solvent-resistant plastic tubes, mixed vigorously, and centrifuged at 4500 g for 3 min to separate water- and cyclohexanone-based layers for the extraction off the developed pink chromophore. The absorbance of the pink supernatant was read at 549 nm with the microplate reader (Infinite 200 PRO, TECAN) to calculate DAHP production with the molar extinction coefficient at 549 nm (ε=549 nm) of 4.5×10⁴ M⁻¹ cm⁻¹. Reaction mixtures with boiled enzymes were run in parallel and used as negative controls to estimate the background signal.

### Structural Modeling and Differential Scanning Fluorimetry Analysis

The three-dimensional structure of DHS2 WT was generated by homology modeling using the high resolution structure 5uxm.pdb of type II DHS from Pseudomonas aeruginosa as a template structure (16). DHS2 WT has more than 60% sequence identity with the template. Homology modeling was performed using Modeller 9.24 (44). The model with the lowest discrete optimized protein energy (DOPE) value was chosen for further validation. The modelled structure was validated by inspection of phi/psi distributions of a Ramachandran plot obtained through PROCHECK (45) and the significance of consistency between template and models was evaluated using the ProSA server (46). In addition, the root mean square deviation (RMSD) was analyzed by Chimera (match-maker) (47) on superimposition of template (5uxm.pdb) with predicted structures to check the reliability of models. The model shows RMSD of 0.207 Å to 5uxm.pdb Trp for 441 atom pairs. The Trp binding site was mapped in the model on Chimera by superposition of Trp-bound 5uxm.pdb.

To examine the impact of the sota mutations on the interaction between DHS and AAA effectors (FIG. 22), a differential scanning fluorimetry (DSF) analysis (48) was conducted using the recombinant DHS2^{WT} and DHS2^{A4} proteins. After diluting each recombinant protein solution to 0.1 μg/μL, 15 μL of the protein solution was mixed with 4 μL of 25-times SYPRO orange fluorescence dye (Sigma-Aldrich) and 1 μL of 20 μM AAA ligand solution dissolved in 40% ethanol. The fluorescence signal was monitored during the stepwise increase in temperature (1° C. per minute from 25° C. to 95° C.). The Tₘ was calculated by nonlinear regression analysis using the Boltzmann sigmoidal equation (48).

### Soluble Metabolite Analyses

Approximately 50 to 80 mg of fully expanded mature leaves were pooled from multiple plants at the same developmental stages. For seedling analyses, approximately 50 mg of shoots and 10 to 20 mg of roots were pooled from more than five 10-day-old seedlings. After quickly measuring their fresh weight, obtained tissues were immediately frozen in liquid nitrogen and kept at −80° C. until use. The frozen tissues were mixed in 800 μl of extraction buffer containing (v/v) 2:1 of methanol and chloroform with isovitexin (0.5 μg/ml) (MilliporeSigma), 100 μM norvaline (Thermo Fisher Scientific), and Tocol (1.25 μg/ml) (Matreya LLC), as internal standards for soluble metabolite analysis by LC-MS and GC-MS and tocopherol analysis by GC-MS, respectively. The mixtures were immediately homogenized for at least 3 min using the 1600 MiniG Tissue Homogenizer (SPEX SamplePrep) and 3-mm glass beads. After adding 600 μl of H₂O and then 250 μl of chloroform, polar phase containing amino acids and nonpolar phase containing tocopherols were separated by centrifugation and dried in new tubes for further analysis.

Metabolite analyses of amino acids and tocopherols using GC-MS were carried out after derivatization of the polar and nonpolar metabolites with N-tert-butyldimethylsilyl-N-methyltrifluoroacetamide with 1% tert-butyldimethylchlorosilane (Cerilliant) and N-methyl-N-(trimethylsilyl)trifluoroacetamide (MSTFA) with 1% trimethylchlorosilane (Restek), respectively, exactly as we previously described (15, 49).

For targeted metabolite analysis of Trp and AAA-derived compounds, reverse-phase LC-MS analysis with the Vanquish UHPLC system coupled with the Q Exactive Quadrupole-Orbitrap MS (Thermo Fisher Scientific) was conducted as previously described (9), with some modifications. The metabolites were dissolved in 70 μl of LC-MS-grade 80% methanol and separated using the mobile phases of 0.1% formic acid in LC-MS-grade water (solvent A) and 0.1% formic acid in LC-MS-grade acetonitrile (solvent B) at a flow rate of 0.4 ml/min and a column temperature of 40° C. The binary 25-min linear gradient with the following ratios of solvent B was used: 0 to 1 min, 1%; 1 to 10 min, 1 to 10%; 10 to 13 min, 10 to 30%; 13 to 14.5 min, 30 to 70%; 14.5 to 15.5 min, 70 to 99%; 15.5 to 21 min, 99%; 21 to 22.5 min, 99 to 10%; 22.5 to 23 min, 10 to 1%; and 23 to 25 min, 1%. The spectra were recorded using the full scan mode of negative ion detection, covering a mass range from mass/charge ratio (m/z) 100 to 1500. The resolution was set to 25,000, and the maximum scan time was set to 250 ms. The sheath gas was set to a value of 60, while the auxiliary gas was set to 35. The transfer capillary temperature was set to 150° C., while the heater temperature was adjusted to 300° C. The spray voltage was fixed at 3 kV, with a capillary voltage and a skimmer voltage of 25 and 15 V, respectively. The identity of amino acids and I3M peaks was confirmed by comparing their accurate masses and retention times with those of the corresponding authentic standards. The identity of the other compounds was confirmed by LC-tandem MS analysis as previously performed (9). Quantification was based on the standard curves generated by injecting different concentrations of authentic chemical standards. The isovitexin peak of each sample was detected to normalize the sample-to-sample variation and to calculate the recovery rate by comparing with a blank sample corresponding to 800 μl of the extraction buffer.

For quantification of some highly polar metabolites such as shikimate, we used hydrophilic interaction chromatography (HILIC) followed by compound detection with a Vanquish UHPLC (ultrahigh-performance LC) system coupled with the Q Exactive MS (Thermo Fisher Scientific). The same samples used for reverse-phase LC-MS analysis was injected onto a HPLC Poroshell 120 HILIC-Z column (150-mm by 2.1-mm inner diameter, 2.7-μm particle size; Agilent) and eluted using mobile phases of 0.2% acetic acid in LC-MS-grade water containing 5 mM ammonium acetate (solvent A) and 0.2% acetic acid in LC-MS-grade acetonitrile containing 5 mM ammonium acetate (solvent B) with the following 22.5-min gradient at a flow rate of 0.45 ml/min and column temperature of 40° C. The binary linear gradient with the following ratios of solvent B was used: 0 to 1 min, 100%; 1 to 11 min, 100 to 89%; 11 to 15.75 min, 89 to 70%; 15.75 to 16.25 min, 70 to 20%; 16.25 to 18.5 min, 20%; 18.5 to 18.6 min, 20 to 100%; and 18.6 to 22.5 min, 100%. The spectra were recorded using the full-scan negative-ion mode, covering a mass range from m/z 70 to 1050. The resolution was set to 70,000, and the maximum scan time was set to 100 ms. The sheath gas was set to a value of 60, while the auxiliary gas was set to 35. The transfer capillary temperature was set to 150° C., while the heater temperature was adjusted to 300° C. The spray voltage was fixed at 3 kV, with a capillary voltage and a skimmer voltage of 25 and 15 V, respectively. Retention times, MS spectra, and associated peak intensities were extracted from the raw files using the Xcalibur software (Thermo Fisher Scientific). The identities of metabolite peaks were confirmed by comparing their accurate masses and retention times with those of the corresponding authentic standards. Quantification was based on the standard curves generated by injecting different concentrations of authentic chemical standards. The isovitexin peak was also detected as an internal standard for the normalization and the recovery rate calculation as used in the reverse-phase LC-MS analysis above.

The IAA level was quantified as previously reported (50), with some modifications. Approximately 150 mg of 10-day-old Arabidopsis WT and the sota mutant seedlings grown on the agar plates were pooled and quickly frozen in a tube with three 3-mm glass beads. After grounding frozen tissues with the 1600 MiniG Tissue Homogenizer (SPEX SamplePrep), the sample was dissolved in 1 ml of ice-cold sodium phosphate buffer (100 mM; pH 7.0) containing 1% (w/v) diethyldithiocarbamic acid and 1 μM isovitexin and shaken on an orbital shaker for 20 min at 4° C. After the centrifugation at 23,000 g, 4° C. for 20 min, the pH of the supernatant was adjusted to below 3.0 with 1 N hydrochloric acid. The IAA metabolite was obtained by solid-phase extraction using Oasis HLB columns (1 ml/30 mg; Waters), which were conditioned with 1 ml of methanol and then 1 ml of water and equilibrated with 0.5 ml of sodium phosphate buffer (acidified with 1 N hydrochloric acid below 3). After the sample application, the column was washed with 2 ml of 5% methanol and then eluted with 2 ml of 80% methanol. The eluate was evaporated and stored at −20° C. until LC-MS analysis. IAA was detected by the same reverse-phase LC-MS method as described above, with the following modifications. The metabolites were separated using the mobile phases of 0.1% formic acid in LC-MS-grade water (solvent A) and 0.1% formic acid in LC-MS-grade acetonitrile (solvent B) at a flow rate of 0.2 ml/min. The binary 25-min linear gradient with the following ratios of solvent B was used: 0 to 0.5 min, 10%; 0.5 to 10 min, 10 to 50%; 10 to 12.5 min, 50 to 60%; 12.5 to 14.5 min, 60 to 70%; 14.5 to 16 min, 70 to 99%; 16 to 21 min, 99%; 21 to 22.5 min, 99 to 10%; and 22.5 to 25 min, 10%. The separated metabolites were detected as described above in the reverse-phase LC-MS analysis, with a selective ion monitoring (SIM) mode. The identity of the IAA peak was confirmed by comparing its accurate mass and retention times with those of the corresponding authentic standards. Quantification was based on the standard curves generated by injecting different concentrations of authentic chemical standards. The isovitexin peak was also detected as an internal standard for the normalization and the recovery rate calculation.

For anthocyanin quantification, the polar phase isolated for amino acid analysis was diluted 10 times with water in a new tube. After adding 5 μl of 5 N HCl for acidification, the absorption was measured at 530 and 657 nm with a microplate reader (Infinite 200 PRO, TECAN) to calculate anthocyanin contents with the formula A₅₃₀−0.25×A₆₅₇ (51). For chlorophyll quantification, the nonpolar phase was dried down and then resuspended in 1 ml of 90% methanol. Several serial dilutions were prepared, and absorbance at 652 and 665 nm was measured using a microplate reader (Infinite 200 PRO, TECAN). The quantities of chlorophylls in each dilution were estimated by the following equations: Chl a=16.72×A₆₆₅−9.16×A₆₅₂ and Chl b=34.09×A₆₅₂−15.28×A₆₆₅ (52).

¹³CO₂ Labeling Experiments

The ¹³CO₂ labeling experiments were conducted following the previously published protocol (53, 54). Briefly, for the time course labeling experiment, Col-0 WT and the sotaB4 and sotaA4 mutants (in the tyra2 background) were grown for 3 weeks under 12 hours of 150-μE light and 12 hours of darkness. These plants were transferred to a 60-liter labeling chamber (75 cm in width, 40 cm in depth, and 20 cm in height; FIG. 25A), 1 hour before the beginning of the light period (˜7 a.m.), to which the air containing 450 to 460 parts per million of ¹³CO₂ was provided at approximately 5 liter/min. After the light was turned on at 8 a.m., the 1-, 3-, and 6-hour samples were harvested at 9 a.m., 11 a.m., and 2 p.m. At each time point, entire shoots (above ground tissues) were harvested and immediately frozen with liquid nitrogen, in three biological replicates per genotype, where three or four individual plants were pooled together to make one replicate. As a nonlabeled control, the samples for the 0-hour time point were harvested in duplicate right before the light period without any ¹³CO₂ labeling. Separately from the 6-hour time course experiment at the beginning of the day, Col-0 WT and the sotaA4 mutant were also labeled with ¹³CO₂ for 3 hours toward the end of the day. Again, 3-week-old plants were placed in the labeling chamber at 4:45 p.m. After 3 hours of ¹³CO₂ labeling, plants were harvested as above at 7:45 p.m., just before the light was turned off.

The harvested shoot samples were ground-frozen to fine powders using the Retsch Ball Mill MM400, and soluble metabolites were extracted as described above, except ribitol, in addition to isovitexin, which was added as an internal standard for GC-MS analysis. Soluble metabolites were dried and derivatized by MSTFA and analyzed by GC-time-of-flight-MS as described previously (55). For quantification of shikimate and Trp, the dried samples were dissolved in 100 μl of 80% MeOH and analyzed by the HILIC LC-MS and the reverse-phase LC-MS methods, respectively, as described above, with the following modified HILIC mobile phase gradient: 0 to 1 min, 100%; 1 to 1.5 min, 100 to 89%; 1.5 to 15.75 min, 89 to 70%; 15.75 to 16.25 min, 70 to 20%; 16.25 to 18.5 min, 20%; 18.5 to 18.6 min, 20 to 100%; and 18.6 to 22.5 min, 100%. To increase the sensitivity of peak detections, especially for ¹³C-labeled fragments, the MS compound detection was performed by a SIM mode.

The peak integration and labeling calculation were carried out as described previously (54). Briefly, the peak areas of nonlabeled and labeled ions (isotopomers) in different samples were integrated using the Xcalibur software (Thermo Fisher Scientific). The obtained data were corrected for natural abundance by comparing to unlabeled control samples using the CORRECTOR software as described previously (54). The amounts of ¹³C-labeled metabolites (nmol/mg of fresh weight) were calculated by multiplying the total metabolite pool sizes (nmol/mg of fresh weight) with the percent of ¹³C-labeled over total metabolite (the sum of both ¹²C- and ¹³C-labeled metabolites).

### Quantification of Starch, Sugar, Protein, and Lignin Contents

Quantification of starch and sugar contents was conducted as previously described (56), with some modifications. Thirty to 50 mg of 4-week-old fully mature leaves were harvested for each biological sample at indicated time points and frozen in a tube with three 3-mm glass beads. Soluble sugars were extracted twice by boiling the sample in 700 μl of 80% ethanol at 80° C. for 45 min until the leaves became bleached. The ethanol extract was evaporated and dissolved in 200 μl of distilled water. The sucrose and glucose levels were determined using the Total Sugar Assay Kit (Megazyme) according to the manufacturer's instruction. For starch analysis, the bleached leaves' tissues were air-dried and then ground in 1 ml of 100 mM sodium acetate buffer (pH 5.0) containing 5 mM CaCl₂. The solubilized starch was enzymatically hydrolyzed into glucose by incubating with 10 μl of α-amylase (3 U/μl; Megazyme) at 100° C. for 15 min. After cooling to room temperature, the mixture was further incubated with 10 μl of amyloglucosidase (3 U/μl; Megazyme) at 50° C. for 50 min. The glucose concentration was determined using the Total Starch Assay Kit (Megazyme) according to the manufacturer's instruction and expressed as micromole glucose equivalent/g fresh weight (FW).

For determination of total protein content, frozen leaf tissues harvested from 4-week-old Arabidopsis plants were ground in liquid nitrogen and dissolved in 500 μl of ice-cold isolation buffer containing 20 mM Hepes (pH 7.4) and 2.5 mM EDTA to determine the protein concentration via a Bradford assay (57). For analyzing the protein amount of Rubisco large subunit (RbcL), the same samples were applied to 4 to 20% Mini-PROTEAN TGX Stain-Free Protein Gels (Bio-Rad) to visualize and quantify the RbcL bands.

To determine the lignin deposition, 4-week-old leaves and roots were first fixed in formaldehyde/acetic acid/ethanol/water at a ratio of 5:5:45:45 (v/v) and decolorized with ethanol/acetic acid at a ratio of 6:1 (v/v). Phloroglucinol staining was conducted as previously described (58). Briefly, tissues were incubated in a mixture of one volume of 37% HCl (v/v) and two volumes of 3% phloroglucinol in ethanol (w/v) for 10 min and observed under bright-field lighting with an Olympus SZX12 stereoscope. For quantifying lignin content, 4-week-old leaves (whole aerial parts) and matured inflorescence stems were harvested and freeze-dried. Three individual plant samples were obtained for each genotype. The tissues were homogeneously pulverized with a tissue homogenizer (1600 MiniG, Spex SamplePrep). The homogenate was then extracted sequentially with distilled water, methanol, and hexane and then freeze-dried to give cell wall residues (CWRs). Thioglycolic acid lignin analysis was performed as described previously (59). The relative lignin content was expressed as absorbance of thioglycolic acid lignin at 280 nm (A₂₈₀) per weight of CWRs (mg).

### Gas Exchange Measurement

The rate of net CO₂ assimilation was measured using an LI-6400XT photosynthesis system equipped with the 6400-40 leaf chamber (LI-COR). Arabidopsis plants were grown in the growth chamber under the condition of a 12-hour/12-hour 100-μE light/dark cycle with 85% air humidity for 4 weeks after germination, and fully expanded nonshaded leaves were used for the measurement. Because leaves did not fully fill the cuvette area, the leaf area inside the cuvette was photographed and quantified by ImageJ to normalize each assimilation rate. The temperature was kept at 25° C. for all measurements. For analysis of the light response curve, the CO₂ concentration in the airstream was maintained at 400 mol/mol. For analysis of the A-Cᵢ curve, the light intensity was saturated at 1500 E. After acclimating the leaves at the Cᵢ level of 400 μmol/mol to achieve a steady-state rate of assimilation, the Cᵢ level of the response curve was set at 400, 185, 70, 35, 740, 1100, 1500, and 1900 μmol/mol, and measurements were taken when assimilation reached a steady-state rate. To determine the V_{cmax}, Jₘₐₓ, and R_{d} values, each A-Cᵢ curve was fitted to the Farquhar-von Caemmerer-Berry model by the “plantecophys” R package (60, 61). The initial slope and CO₂ compensation point of the light response curves and A-Cᵢ curves were determined using the first three and five points at low light and low Cᵢ points, respectively, as previously calculated (62).

### Quantitative PCR Expression Analysis

To test the effects of the sota mutations on the DHS gene expression, the transcript levels of DHS1, DHS2, and DHS3 were analyzed by reverse transcription quantitative PCR (RT-qPCR). Approximately 20 to 30 mg of fully expanded mature leaves were pooled from multiple 4-week-old plants grown on soils, immediately frozen in liquid nitrogen in a tube with three 3-mm glass beads, and ground using the 1600 MiniG Tissue Homogenizer (SPEX SamplePrep). Total RNA was isolated as previously described (63), treated with deoxyribonuclease I (Thermo Fisher Scientific), and reverse-transcribed to synthesize cDNA with M-MuLV reverse transcriptase and random hexamer primers (Promega) according to the manufacturer's protocol. RT-qPCR was conducted by the Stratagene Mx3000P (Agilent Technologies) using the GoTaq qPCR Master Mix (Promega), and target gene-specific primers listed in Table 10. Four biological replicates with two technical RT-qPCR replicates were conducted. Expression of the UBQ9 gene was used to normalize the sample-to-sample variations between different cDNA preparations. Relative expression levels among different genotypes were analyzed for each DHS gene using the 2^{-ΔΔCt} method.

### Amino Acid Sequence Alignment

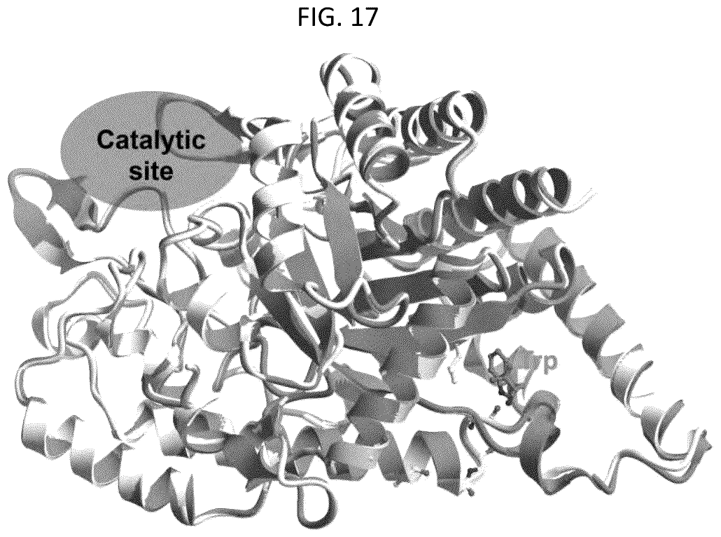

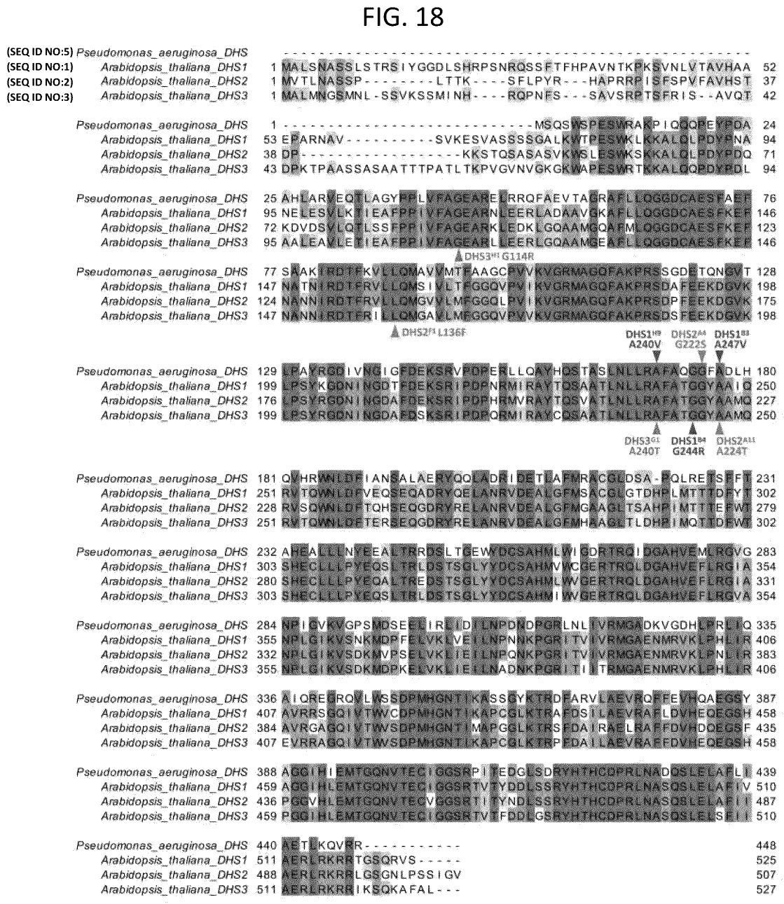

DHS orthologs were first identified by BlastP searches using the amino acid sequence of AtDHS1 as a query against Phytozome 13 (64). Nicotiana benthamiana DHSs were searched from the N. benthamiana draft genome sequence v1.0.1 (65). The sequence alignment of FIG. 18 and FIG. 34 was conducted with the MUSCLE algorithm and then visualized with Jalview (66), highlighting the residues by different depths of purple according to the percentage of the residues in each column that agree with the consensus sequence (>80, >60, >40, and <40%). The sequence alignment information provided in the table in FIG. 35 was generated with the MUSCLE algorithm and then visualized with Excel.

### Tables:

TABLE 1
sota mutations identified in this study. The mutation frequencies were calculated based on a single nucleotide variant (SNV)
analysis on bulk sota F₂ populations as shown for sotaA4, sotaA11, and sotaB4 in FIG. 13. The sotaB3, sotaG1, sotaH1 and sotaH9
mutant lines were also analyzed in the same way and showed semidominant characteristics with 100% frequency for sotaB3, sotaG1
and sotaH1, and 80% for sotaH9 due to near complete dominant characteristics like sotaB4 (see FIG. 13 legend for detailed
explanations). Although the sotaF1 F₂ population also showed a dominant characteristic, only 50% of its F₂ population were
suppressor-like plants with the remaining non-tyra2-like plants exhibiting a pleiotropic dwarf phenotype (FIG. 15). dCAPS
genotyping later confirmed that these pleiotropic dwarf plants are sotaF1 homozygous plants (see FIG. 14).

| sota ID | Gene ID | Protein | Amino acid alteration | Mutation frequency (%) |
|---|---|---|---|---|
| A4 | At4g33510 | DHS2 | G222S | 100 |
| A11 | At4g33510 | DHS2 | A224T | 100 |
| B3 | At4g39980 | DHS1 | A247V | 100 |
| B4 | At4g39980 | DHS1 | G244R | 66.7 |
| F1 | At4g33510 | DHS2 | L136F | 50 |
| G1 | At1g22410 | DHS3 | A240T | 100 |
| H1 | At1g22410 | DHS3 | G114R | 100 |
| H9 | At4g39980 | DHS1 | A240V | 80 |

TABLE 2
IC50 values (μM) of the sota mutant enzymes for various effector molecules. The IC50
values for the AAAs, chorismate, and caffeate were obtained from Yokoyama et al., Plant
Cell, 2021. HPP, 4-Hydroxyphenylpyruvate; HGA, homogentisate; ILA, Indole-3-lactate.

| | AAAs | | Tyr-derived | | Trp-derived | | | Others | |
|---|---|---|---|---|---|---|---|---|---|
| | Tyr | Trp | HPP | HGA | IPA | Indole-3-propionic acid | ILA | Chorismate | Caffeate |
| DHS1^{WT} | Not inhibited | Not inhibited | 305.1 | 253 | 241.3 | 2059 | 209.5 | 14.57 | 107.8 |
| DHS1^{B4} | Not inhibited | Not inhibited | 834.8 | 2992 | 4995 | 1997 | 2880 | 25.26 | 65.18 |
| DHS2^{WT} | 230.4 | 225.1 | 48.4 | 73.9 | 58.34 | — | — | 80.97 | 32.03 |
| DHS2^{A4} | Not inhibited | Not inhibited | 143 | 212.2 | 73.43 | — | — | 101.5 | 27.47 |
| DHS2^{A11} | Not inhibited | Not inhibited | 58.6 | 241.9 | 90.64 | — | — | — | — |
| DHS2^{F1} | Not inhibited | Not inhibited | 321.3 | 788.4 | 73.99 | — | — | — | — |
| DHS3^{WT} | Not inhibited | Not inhibited | 515.9 | 101.8 | 25.33 | — | — | — | — |
| DHS3^{G1} | Not inhibited | Not inhibited | 915 | 375 | 272.7 | — | — | — | — |
| DHS3^{H1} | Not inhibited | Not inhibited | 902.7 | 404.6 | 108.1 | — | — | — | — |

TABLE 3
Metabolite levels in 4-week-old mature leaves grown on soils. Levels of amino acids and AAA-derived metabolites
were measured in mature leaves of 4-week-old Col-0, sotaB4, and sotaA4 plants (Col-0 background) grown on soils
under standard growth conditions, as shown in graphs in FIG. 3B. Different letters indicate statistically significant
differences among genotypes (one-way ANOVA with Tukey-Kramer test, P < 0.05). Data are means ± SEM
(n = 5 to 6 replicated samples). HGA, homogentisate; PPY, phenylpyruvate; I3M, indolyl-3-methyl glucosinolate;
4MOI3M, 4-methoxy-indol-3-ylmethyl glucosinolate; 1MOI3M, 1-methoxy-3-indolylmethyl glucosinolate; 4MSOB, 4-
methylsulfinylbutyl glucosinolate; 5MSOP, 5-methylsulfinylpentyl glucosinolate; 4MTB, 4-methylthiobutyl glucosinolate;
8MSOO, 8-methylsulfinyloctyl glucosinolate; 7MTH, 7-methylthioheptyl glucosinolate; Q3GR7R, quercetin-3-O-(2″-O-
rhamnosyl)glucoside-7-O-rhamnoside; K3GR7R, kaempferol-3-O-(2″-O-rhamnosyl)glucoside-7-O-rhamnoside; Q3G7R,
quercetin-3-O-glucoside-7-O-rhamnoside; K3G7R, kaempferol-3-O-glucoside-7-O-rhamnoside; Q3R7R, quercetin-3-O-
rhamoside-7-O-rhamnoside; K3R7R, keampferol-3-O-rhamnoside-7-O-rhamnoside.

| | | Col-0 | | sotaB4 | | sotaA4 | |
|---|---|---|---|---|---|---|---|
| Metabolite | Unit | Amount ± SEM | | Amount ± SEM | | Amount ± SEM | |
| Tyr | nmol/g FW | 2.16 ± 0.11 | c | 13.81 ± 1.36 | b | 18.07 ± 1.02 | a |
| Phe | nmol/g FW | 10.63 ± 0.35 | c | 73.72 ± 8.71 | b | 172.38 ± 6.70 | a |
| Trp | nmol/g FW | 7.93 ± 0.32 | c | 13.72 ± 1.29 | b | 17.62 ± 0.91 | a |
| Shikimate | nmol/g FW | 104.39 ± 7.51 | a | 107.81 ± 6.55 | a | 108.60 ± 5.15 | a |
| Leu | nmol/g FW | 0.36 ± 0.02 | b | 0.43 ± 0.02 | ab | 0.47 ± 0.03 | a |
| Ile | nmol/g FW | 4.47 ± 0.17 | a | 4.35 ± 0.22 | a | 5.04 ± 0.37 | a |
| Val | nmol/g FW | 130.21 ± 1.78 | a | 134.61 ± 1.94 | a | 119.37 ± 1.86 | a |
| Met | nmol/g FW | 19.44 ± 1.35 | a | 19.80 ± 1.88 | a | 17.61 ± 1.60 | a |
| Ala | nmol/g FW | 346.08 ± 7.04 | a | 238.49 ± 14.21 | b | 180.02 ± 7.80 | c |
| Thr | nmol/g FW | 1603.75 ± 55.97 | a | 1373.71 ± 77.45 | b | 1367.36 ± 34.95 | b |
| Ser | nmol/g FW | 157.01 ± 4.09 | a | 146.82 ± 5.21 | a | 145.08 ± 13.78 | a |
| Pro | nmol/g FW | 40.73 ± 0.97 | a | 31.07 ± 1.15 | a | 29.15 ± 0.96 | a |
| Glu | nmol/g FW | 718.82 ± 38.05 | a | 768.35 ± 58.29 | a | 776.07 ± 53.87 | a |
| Gly | nmol/g FW | 42.05 ± 2.48 | a | 40.84 ± 2.66 | a | 36.98 ± 2.19 | a |
| Asp | nmol/g FW | 7.73 ± 0.60 | a | 8.39 ± 0.81 | a | 9.36 ± 1.14 | a |
| Lys | nmol/g FW | 4.07 ± 0.20 | b | 4.52 ± 0.33 | ab | 5.47 ± 0.33 | a |
| HGA | nmol/g FW | 1.07 ± 0.05 | c | 11.38 ± 2.41 | a | 5.62 ± 1.11 | b |
| alpha-tocopherol | nmol/g FW | 11.73 ± 1.11 | b | 19.08 ± 2.02 | a | 20.15 ± 1.49 | a |
| gamma-tocopherol | nmol/g FW | 0.082 ± 0.0054 | b | 0.37 ± 0.063 | a | 0.40 ± 0.046 | a |
| PPY | nmol/g FW | 0.066 ± 0.0014 | c | 0.83 ± 0.0860 | b | 4.54 ± 0.28 | a |
| Phenylacetate | nmol/g FW | 0.18 ± 0.016 | b | 1.15 ± 0.14 | ab | 4.96 ± 0.95 | a |
| Phenyllactate | nmol/g FW | 0.0031 ± 0.00014 | c | 0.020 ± 0.00086 | b | 0.046 ± 0.0034 | a |
| I3M | nmol/g FW | 11.54 ± 0.44 | a | 12.69 ± 0.76 | a | 13.24 ± 0.76 | a |
| 4MOI3M | Area/g FW | 841170 ± 27453 | a | 641097.90 ± 21102.42 | b | 689467.52 ± 22268.27 | b |
| 1MOI3M | Area/g FW | 41150 ± 3832 | a | 41390.48 ± 4737.36 | a | 48379.56 ± 5759.12 | a |
| 4MSOB | Area/g FW | 1347575 ± 187637 | a | 1377755.83 ± 151837.46 | a | 1680271.67 ± 151668.54 | a |
| 5MSOP | Area/g FW | 36927 ± 6513 | a | 34884.56 ± 4029.31 | a | 37431.95 ± 4487.09 | a |
| 7MTH | Area/g FW | 587653 ± 21913 | b | 754399.90 ± 47340.40 | a | 524271.55 ± 16968.40 | b |
| 8MTO | Area/g FW | 2917012 ± 143650 | a | 2611425.83 ± 140474.39 | a | 2003281.17 ± 88988.67 | b |
| Sinapate | μmol/g FW | 2.79 ± 0.083 | a | 2.99 ± 0.29 | a | 2.73 ± 0.20 | a |
| Sinapoyl-O- | Area/g FW | 155954 ± 19772 | a | 176446 ± 40637 | a | 173682 ± 37249 | a |
| glucoside | | | | | | | |
| Sinapoyl- | Area/g FW | 4698709411 ± 223026261 | a | 4961057952 ± 154000394 | a | 4910991591 ± 100630122 | a |
| malate | | | | | | | |
| Q3GR7R | Area/g FW | 6075 ± 445 | a | 3132 ± 265 | b | 7055 ± 688 | a |
| K3GR7R | Area/g FW | 210204863 ± 17365460 | ab | 154889923 ± 15290551 | b | 252872967 ± 27606422 | a |
| Q3G7R | Area/g FW | 8135 ± 271 | ab | 5618 ± 458 | b | 10200 ± 919 | a |
| K3G7R | Area/g FW | 161782 ± 15783 | b | 145374 ± 21436 | b | 238153 ± 25544 | a |
| Q3R7R | Area/g FW | 161791 ± 15783 | b | 145372 ± 21437 | b | 238151 ± 25545 | a |
| K3R7R | Area/g FW | 499879 ± 50166 | ab | 380400 ± 47162 | b | 706282 ± 89459 | a |

TABLE 4
Metabolite levels in mature leaves before and after high light treatment. Levels of amino acids and AAA-derived metabolites
were measured in mature leaves of 4-week-old Col-0, sotaB4, and sotaA4 plants (Col-0 background) before and after a 2-
day high light (HL) treatment (650 μE), as shown in graphs in FIG. 29. Different letters indicate statistically significant
differences between the samples before and after HL stress (one-way ANOVA with Tukey-Kramer test, P < 0.05). Data
are means ± SEM (n = 4 replicated samples). I3M; K3GR7R, kaempferol-3-O-(2″-O-rhamnosyl)glucoside-7-O-rhamnoside.

| | | Col-0 | sotaB4 | sotaA4 |
|---|---|---|---|---|
| Metabolite | Unit | Amount ± SEM | Amount ± SEM | Amount ± SEM |

Before HL

| | | | | | | | |
|---|---|---|---|---|---|---|---|
| Tyr | nmol/g FW | 3.24 ± 0.39 | c | 16.66 ± 1.15 | b | 18.68 ± 1.24 | b |
| Phe | nmol/g FW | 32.22 ± 4.27 | c | 228.12 ± 47.93 | b | 438.69 ± 37.78 | a |
| Trp | nmol/g FW | 9.05 ± 0.51 | d | 18.19 ± 0.46 | c | 20.41 ± 2.31 | c |
| α-tocopherol | nmol/g FW | 10.42 ± 2.30 | c | 25.76 ± 3.12 | b | 21.63 ± 1.78 | b |
| γ-tocopherol | nmol/g FW | 0.11 ± 0.018 | d | 0.40 ± 0.011 | c | 0.33 ± 0.035 | c |
| I3M | nmol/g FW | 164.76 ± 11.77 | b | 189.39 ± 24.80 | ab | 139.53 ± 17.16 | ab |
| Anthocyanin | (A530 − | 0.57 ± 0.14 | b | 0.55 ± 0.07 | b | 0.45 ± 0.11 | b |
| | 0.25*A657)/g | | | | | | |
| | FW | | | | | | |
| Sinapoyl- | Area/g FW | 26590277460 ± 1933244586 | a | 38180008294 ± 1688446771 | ab | 34520444809 ± 746265694 | ab |
| malate | | | | | | | |
| K3GR7R | Area/g FW | 677527832 ± 132095379 | b | 1134071832 ± 144697558 | b | 897383224 ± 169834558 | b |

After 2-day HL

| | | | | | | | |
|---|---|---|---|---|---|---|---|
| Tyr | nmol/g FW | 7.22 ± 0.31 | c | 29.89 ± 2.46 | b | 22.26 ± 2.92 | a |
| Phe | nmol/g FW | 30.48 ± 8.90 | c | 308.58 ± 31.29 | bc | 938.01 ± 49.46 | b |
| Trp | nmol/g FW | 18.63 ± 3.17 | c | 84.47 ± 19.83 | a | 101.11 ± 18.19 | b |
| α-tocopherol | nmol/g FW | 58.93 ± 11.98 | a | 62.51 ± 15.76 | a | 59.26 ± 6.28 | a |
| γ-tocopherol | nmol/g FW | 2.64 ± 0.41 | a | 1.65 ± 0.23 | ab | 1.36 ± 0.14 | b |
| I3M | nmol/g FW | 344.40 ± 90.80 | a | 321.16 ± 59.26 | a | 278.89 ± 14.36 | a |
| Anthocyanin | (A530 − | 5.83 ± 1.61 | a | 4.45 ± 1.05 | | 6.00 ± 0.81 | a |
| | 0.25*A657)/g | | | | | | |
| | FW | | | | | | |
| Sinapoyl- | Area/g FW | 27131767862 ± 546939564 | a | 32609318335 ± 930394501 | a | 36824547556 ± 265842619 | a |
| malate | | | | | | | |
| K3GR7R | Area/g FW | 4624478653 ± 945771292 | a | 3275263526 ± 689297905 | a | 4133185978 ± 254902672 | a |

TABLE 5
Metabolite levels of 10-day-old shoots and roots grown on agar plates. Levels of amino acids and AAA-derived metabolites of shoots
and roots of 10-day-old Col-0, sotaB4, and sotaA4 plants (Col-0 background) grown on ½ MS medium agar containing
1% sucrose, as shown in graphs in FIG. 30. Different letters indicate statistically significant differences between the shoot
and root samples (one-way ANOVA with Tukey-Kramer test, P < 0.05). Data are means ± SEM (n = 3-6 replicated samples).
I3M, indolyl-3-methyl glucosinolate; 4MOI3M, 4-methoxy-indol-3-ylmethyl glucosinolate; 1MOI3M, 1-methoxy-3-indolylmethyl glucosinolate;
Q3GR7R, quercetin-3-O-(2″-O-rhamnosyl)glucoside-7-O-rhamnoside; K3GR7R, kaempferol-3-O-(2″-O-rhamnosyl)glucoside-7-O-rhamnoside;
Q3G7R, quercetin-3-O-glucoside-7-O-rhamnoside; K3G7R, kaempferol-3-O-glucoside-7-O-rhamnoside; Q3R7R, quercetin-3-O-rhamoside-
7-O-rhamnoside; K3R7R, keampferol-3-O-rhamnoside-7-O-rhamnoside, IAA, indole-3-acetate.

| | | Col-0 | sotaB4 | sotaA4 |
|---|---|---|---|---|
| Metabolite | Unit | Amount ± SEM | Amount ± SEM | Amount ± SEM |

Shoots

| | | | | | | | |
|---|---|---|---|---|---|---|---|
| Tyr | nmol/g FW | 16.69 ± 1.61 | c | 179.96 ± 7.24 | b | 175.38 ± 22.88 | b |
| Phe | nmol/g FW | 58.94 ± 10.60 | e | 826.41 ± 90.53 | c | 1262.92 ± 160.61 | b |
| Trp | nmol/g FW | 9.64 ± 0.90 | c | 42.28 ± 2.94 | b | 39.93 ± 4.68 | b |
| shikimate | nmol/g FW | 71.26 ± 6.01 | d | 126.85 ± 7.37 | c | 130.91 ± 10.55 | c |
| Ala | nmol/g FW | 688.62 ± 66.91 | c | 727.02 ± 35.05 | c | 779.33 ± 56.42 | c |
| Ser | nmol/g FW | 1699.49 ± 200.24 | c | 1640.26 ± 63.92 | c | 1509.05 ± 100.50 | c |
| Leu | nmol/g FW | 15.63 ± 3.13 | b | 10.05 ± 0.92 | b | 19.24 ± 3.74 | b |
| Ile | nmol/g FW | 23.10 ± 3.60 | b | 17.26 ± 1.07 | b | 27.54 ± 3.42 | b |
| I3M | nmol/g FW | 175.76 ± 11.68 | c | 178.37 ± 12.30 | c | 197.76 ± 15.92 | c |
| 4MOI3M | Area/g FW | 4419053145 ± 514502100 | b | 6330537147 ± 399261351 | b | 5664608157 ± 401692958 | b |
| 1MOI3M | Area/g FW | 4105720837 ± 325673063 | b | 4383166709 ± 632588904 | b | 8028160366 ± 896698681 | b |
| Sinapoyl- | Area/g FW | 55108222481 ± 3701841572 | a | 58676527559 ± 4580500716 | a | 64111228999 ± 4069835976 | a |
| malate | | | | | | | |
| Q3GR7R | Area/g FW | 155656502 ± 15234296 | b | 116363760 ± 6980472 | b | 234032171 ± 30122588 | b |
| K3GR7R | Area/g FW | 2226333408 ± 249160589 | b | 2182860264 ± 88186447 | b | 2703275892 ± 83388540 | b |
| Q3G7R | Area/g FW | 1156676702 ± 116547306 | b | 882592198 ± 78394537 | b | 1794513400 ± 177483966 | b |
| K3G7R | Area/g FW | 5090332920 ± 474541444 | b | 5881524146 ± 230110844 | b | 7446528385 ± 523178381 | b |
| Q3R7R | Area/g FW | 11939990 ± 2793547 | b | 23600282 ± 3778760 | b | 33888209 ± 3375845 | b |
| K3R7R | Area/g FW | 3921 ± 115 | a | 4479 ± 75 | a | 3756 ± 144 | a |

Roots

| | | | | | | | |
|---|---|---|---|---|---|---|---|
| Tyr | nmol/g FW | 223.10 ± 76.65 | b | 1113.94 ± 131.10 | a | 183.62 ± 11.29 | b |
| Phe | nmol/g FW | 390.35 ± 218.26 | d | 2551.59 ± 478.96 | a | 269.06 ± 29.87 | d |
| Trp | nmol/g FW | 58.75 ± 8.63 | b | 160.95 ± 17.54 | a | 54.66 ± 8.86 | b |
| shikimate | nmol/g FW | 426.12 ± 85.89 | b | 861.10 ± 97.15 | a | 590.51 ± 113.72 | ab |
| Ala | nmol/g FW | 3302.62 ± 810.61 | b | 5467.75 ± 1240.36 | a | 4550.48 ± 773.43 | ab |
| Ser | nmol/g FW | 3232.15 ± 517.12 | b | 5921.23 ± 1188.79 | a | 4864.82 ± 450.64 | ab |
| Leu | nmol/g FW | 215.11 ± 75.24 | a | 424.79 ± 71.42 | a | 355.25 ± 48.03 | a |
| Ile | nmol/g FW | 255.24 ± 82.83 | a | 495.50 ± 82.75 | a | 405.93 ± 59.31 | a |
| I3M | nmol/g FW | 397.65 ± 22.97 | ab | 482.79 ± 58.81 | a | 375.84 ± 43.32 | b |
| 4MOI3M | Area/g FW | 126509085878 ± 5616973267 | a | 129284376757 ± 7949555673 | a | 130844865926 ± 6749068987 | a |
| 1MOI3M | Area/g FW | 874514254080 ± 77784351075 | a | 1106723852949 ± 79092142361 | a | 870377058471 ± 31296309998 | a |
| Sinapoyl- | Area/g FW | 1944366533 ± 249041372 | b | 1814232976 ± 360324869 | b | 1576400680 ± 271631595 | b |
| malate | | | | | | | |
| Q3GR7R | Area/g FW | 7114380273 ± 839937502 | a | 7092432320 ± 534579594 | a | 5914111389 ± 670970848 | a |
| K3GR7R | Area/g FW | 58289162889 ± 7005621267 | a | 61338881688 ± 7323055806 | a | 52060420902 ± 4373332228 | a |
| Q3G7R | Area/g FW | 86548909460 ± 3506127444 | a | 93637900020 ± 6226730021 | a | 100056430285 ± 7922803435 | a |
| K3G7R | Area/g FW | 218410952687 ± 15863013459 | a | 235113773241 ± 16087886928 | a | 218456644133 ± 9362376881 | a |
| Q3R7R | Area/g FW | 7510022595 ± 1197394294 | a | 8985394599 ± 607675168 | a | 6306912256 ± 978366039 | a |
| K3R7R | Area/g FW | 260 ± 7 | b | 216 ± 4 | b | 231 ± 13 | b |

Shoots + Roots

| | | | | | | | |
|---|---|---|---|---|---|---|---|
| IAA | pmol/g FW | 82.437 ± 7.28 | a | 56.95 ± 5.83 | ab | 63.07 ± 8.56 | b |

TABLE 6
Growth parameters, contents of total protein, and photosynthetic parameters determined from the A-Cᵢ curves shown in FIG.
4E. V_{cmax}, Jₘₐₓ, and R_{d} values represent the maximum rate of Rubisco carboxylation activity, the potential rate of electron transport,
and the rate of mitochondrial dark respiration, respectively. The initial slope and CO₂ compensation point (CCP) of the light response
curves and A-Cᵢ curves were determined using the first three and five points at low light and low Cᵢ points, respectively (FIG. 4D,
E). Different letters (a and b) indicate statistically significant differences among genotypes (one-way ANOVA with Tukey-Kramer
test, P < 0.05). Data are means ± SEM (n = 8 independent plant samples for the growth and protein data and n = 5 to 6 for the
photosynthetic parameters). FW, fresh weight; RbcL, Rubisco large subunit.

| | Col-0 | sotaB4 | sotaA4 |
|---|---|---|---|
| Individual shoot FW (g FW) | 0.23 ± 0.0051 ^{a} | 0.20 ± 0.010 ^{a} | 0.24 ± 0.0090 ^{a} |
| Shoot area/shoot FW | 0.018 ± 0.000076 ^{a} | 0.018 ± 0.00022 ^{a} | 0.018 ± 0.00015 ^{a} |
| (cm²/g FW) | | | |
| Total protein amount | 9.11 ± 0.22 ^{a} | 9.45 ± 0.19 ^{a} | 8.86 ± 0.21 ^{a} |
| (mg/g FW) | | | |
| Relative RbcL amount | 1.00 ± 0.018 ^{a} | 0.97 ± 0.018 ^{a} | 0.92 ± 0.027 ^{a} |
| V_{cmax} (μmol/m² per second) | 18.57 ± 0.34 ^{b} | 29.06 ± 0.89 ^{a} | 27.46 ± 0.80 ^{a} |
| Jₘₐₓ (μmol/m² per second) | 61.14 ± 0.61 ^{b} | 83.51 ± 1.86 ^{a} | 79.82 ± 1.71 ^{a} |
| R_{d} (μmol/m² per second) | 1.80 ± 0.10 ^{b} | 2.37 ± 0.10 ^{a} | 2.67 ± 0.081 ^{a} |
| Initial slope | 0.04 ± 0.0010 ^{b} | 0.06 ± 0.0013 ^{a} | 0.06 ± 0.0023 ^{a} |
| CCP (μmol/m² per second) | 133.00 ± 3.81 ^{a} | 121.96 ± 1.85 ^{a} | 133.48 ± 1.18 ^{a} |

TABLE 7
Metabolite levels of transgenic lines expressing mutated DHS genes in the Col-0 wild-type background. Levels of amino acids
and AAA-derived metabolites were measured in mature leaves of 5-week-old T₂ transgenic plants expressing the WT or sota DHS
genes in the Col-0 background under the control of their own promoters, as well as control plants having empty vector (EV).
Plants were grown on soils under standard growth condition, as shown in FIG. 32A. Different letters indicate statistically
significant differences among genotypes (one-way ANOVA with Tukey-Kramer test, P < 0.05). Data are means ± SEM (n
= 5 independent plant samples). HGA, homogentisate; PPY, phenylpyruvate; I3M, indolyl-3-methyl glucosinolate; 4MOI3M,
4-methoxy-indol-3-ylmethyl glucosinolate; 1MOI3M, 1-methoxy-3-indolylmethyl glucosinolate; Q3GR7R, quercetin-3-O-(2″-O-
rhamnosyl)glucoside-7-O-rhamnoside; K3GR7R, kaempferol-3-O-(2″-O-rhamnosyl)glucoside-7-O-rhamnoside; Q3G7R, quercetin-3-
O-glucoside-7-O-rhamnoside; K3G7R, kaempferol-3-O-glucoside-7-O-rhamnoside; K3R7R, keampferol-3-O-rhamnoside-7-O-rhamnoside.

| | | Col-0::EV | | Col-0::DHS1^{WT} | | Col-0::DHS1^{B4} | |
|---|---|---|---|---|---|---|---|
| Metabolite | Unit | Amount SEM | | Amount SEM | | Amount SEM | |
| Tyr | nmol/g FW | 0.52 ± 0.022 | c | 0.58 ± 0.020 | c | 51.03 ± 3.01 | a |
| Phe | nmol/g FW | 3.78 ± 0.16 | c | 4.76 ± 0.29 | c | 305.63 ± 14.25 | a |
| Trp | nmol/g FW | 0.79 ± 0.03 | c | 0.85 ± 0.02 | c | 11.93 ± 0.65 | a |
| Leu | nmol/g FW | 1.92 ± 0.053 | a | 1.78 ± 0.080 | a | 1.95 ± 0.071 | a |
| Ile | nmol/g FW | 2.64 ± 0.024 | a | 2.53 ± 0.061 | a | 2.54 ± 0.044 | a |
| Val | nmol/g FW | 10.44 ± 0.20 | b | 10.39 ± 0.19 | b | 12.84 ± 0.30 | a |
| Met | nmol/g FW | 1.92 ± 0.015 | a | 1.75 ± 0.059 | a | 2.09 ± 0.033 | a |
| Ala | nmol/g FW | 53.51 ± 2.86 | a | 53.10 ± 2.27 | a | 46.09 ± 1.53 | a |
| Thr | nmol/g FW | 122.36 ± 1.86 | a | 132.75 ± 5.86 | ab | 101.79 ± 3.52 | b |
| Ser | nmol/g FW | 192.46 ± 4.81 | a | 155.98 ± 3.12 | a | 136.17 ± 6.35 | a |
| Pro | nmol/g FW | 15.68 ± 0.48 | a | 18.88 ± 2.26 | a | 16.45 ± 0.40 | a |
| Gln | nmol/g FW | 38.26 ± 1.54 | a | 44.05 ± 2.98 | a | 45.55 ± 2.23 | a |
| Glu | nmol/g FW | 187.64 ± 3.55 | a | 214.40 ± 10.33 | a | 218.74 ± 9.05 | a |
| Gly | nmol/g FW | 6.84 ± 0.16 | ab | 6.25 ± 0.11 | ab | 6.61 ± 0.22 | ab |
| Asn | nmol/g FW | 15.68 ± 0.40 | a | 17.14 ± 0.76 | a | 16.55 ± 0.57 | a |
| Asp | nmol/g FW | 38.86 ± 1.19 | a | 46.81 ± 2.04 | a | 43.49 ± 1.73 | a |
| Lys | nmol/g FW | 0.93 ± 0.041 | a | 1.02 ± 0.044 | a | 1.01 ± 0.037 | a |
| HGA | nmol/g FW | 0.52 ± 0.022 | c | 0.58 ± 0.020 | c | 51.03 ± 3.01 | a |
| PPY | nmol/g FW | 0.076 ± 0.0030 | c | 0.068 ± 0.0032 | cc | 10.96 ± 0.56 | a |
| Phenylacetate | nmol/g FW | 0.40 ± 0.029 | b | 0.28 ± 0.031 | b | 7.30 ± 0.72 | a |
| Phenyllactate | nmol/g FW | 0.0044 ± 0.00034 | b | 0.0024 ± 0.00015 | b | 0.11 ± 0.0024 | a |
| I3M | nmol/g FW | 41.35 ± 2.57 | b | 48.22 ± 2.37 | ab | 65.33 ± 2.20 | a |
| 4MOI3M | Area/g FW | 114693978147 ± 3383144678 | a | 110226070966 ± 3611123824 | a | 168677816972 ± 5829244353 | a |
| 1MOI3M | Area/g FW | 6676364594 ± 304970404.7 | a | 6213761532 ± 401927102.2 | a | 7988064911 ± 309041330.4 | a |
| Sinapate | nmol/g FW | 73.47 ± 6.62 | a | 95.22 ± 8.15 | a | 90.62 ± 6.73 | a |
| Sinapoyl- | Area/g FW | 580918423107 ± 18162042570 | a | 685813081665 ± 24672758977 | a | 968872392975 ± 32080969515 | a |
| malate | | | | | | | |
| Q3GR7R | Area/g FW | 496000997 ± 37122597 | a | 567926950 ± 34438882 | a | 455902382 ± 19403490 | a |
| K3GR7R | Area/g FW | 20977721097 ± 1584974145 | a | 24492803670 ± 1654049230 | a | 22675989870 ± 1317811311 | a |
| Q3G7R | Area/g FW | 714605077 ± 56984745 | a | 796658462 ± 47347042 | a | 768388719 ± 38109592 | a |
| K3G7R | Area/g FW | 24817622426 ± 2160421506 | a | 29131340653 ± 2437529159 | a | 30739765370 ± 1933091667 | a |
| K3R7R | Area/g FW | 69261475040 ± 5411365182 | a | 76535126884 ± 6117592147 | a | 75729366810 ± 4586731946 | a |

| | | Col-0::DHS2^{WT} | | Col-0::DHS2^{A4} | |
|---|---|---|---|---|---|
| Metabolite | Unit | Amount SEM | | Amount SEM | |
| Tyr | nmol/g FW | 0.67 ± 0.040 | c | 5.09 ± 0.35 | b |
| Phe | nmol/g FW | 3.62 ± 0.079 | c | 74.18 ± 2.40 | b |
| Trp | nmol/g FW | 0.65 ± 0.0092 | c | 1.74 ± 0.11 | a |
| Leu | nmol/g FW | 1.99 ± 0.072 | a | 1.72 ± 0.076 | a |
| Ile | nmol/g FW | 2.64 ± 0.049 | a | 2.34 ± 0.064 | a |
| Val | nmol/g FW | 10.06 ± 0.12 | b | 10.18 ± 0.24 | b |
| Met | nmol/g FW | 2.11 ± 0.044 | a | 1.87 ± 0.021 | a |
| Ala | nmol/g FW | 48.17 ± 2.28 | a | 44.54 ± 1.14 | a |
| Thr | nmol/g FW | 120.88 ± 3.85 | a | 98.74 ± 5.19 | ab |
| Ser | nmol/g FW | 184.35 ± 5.37 | a | 161.46 ± 5.00 | a |
| Pro | nmol/g FW | 21.68 ± 1.60 | a | 11.17 ± 0.40 | a |
| Gln | nmol/g FW | 42.79 ± 3.76 | a | 30.76 ± 1.20 | a |
| Glu | nmol/g FW | 204.74 ± 10.85 | a | 206.83 ± 12.84 | a |
| Gly | nmol/g FW | 7.85 ± 0.21 | a | 6.15 ± 0.10 | b |
| Asn | nmol/g FW | 16.16 ± 0.97 | a | 13.11 ± 0.34 | a |
| Asp | nmol/g FW | 43.45 ± 3.04 | a | 37.92 ± 3.03 | a |
| Lys | nmol/g FW | 0.86 ± 0.032 | a | 0.88 ± 0.037 | a |
| HGA | nmol/g FW | 0.67 ± 0.040 | c | 5.09 ± 0.35 | b |
| PPY | nmol/g FW | 0.076 ± 0.0050 | c | 0.87 ± 0.009702424 | b |
| Phenylacetate | nmol/g FW | 0.58 ± 0.16 | b | 0.64 ± 0.043 | b |
| Phenyllactate | nmol/g FW | 0.0070 ± 0.00062 | b | 0.0089 ± 0.00064 | b |
| I3M | nmol/g FW | 40.83 ± 0.59 | b | 49.00 ± 3.29 | ab |
| 4MOI3M | Area/g FW | 103491442102 ± 1369780813 | a | 127855967403 ± 3634274416 | a |
| 1MOI3M | Area/g FW | 5778255771 ± 96285423.95 | a | 7997350523 ± 422229781.2 | a |
| Sinapate | nmol/g FW | 49.62 ± 4.22 | a | 117.65 ± 11.78 | a |
| Sinapoyl- | Area/g FW | 539506171248 ± 7967141596 | a | 888307788562 ± 28066394150 | a |
| malate | | | | | |
| Q3GR7R | Area/g FW | 386980558 ± 14279340 | a | 654565897 ± 43125208 | a |
| K3GR7R | Area/g FW | 15813490748 ± 595582833 | a | 27213799599 ± 1720364851 | a |
| Q3G7R | Area/g FW | 514914749 ± 16195350 | a | 945763316 ± 66555874 | a |
| K3G7R | Area/g FW | 17562002981 ± 898027097 | a | 34838063767 ± 2562855693 | a |
| K3R7R | Area/g FW | 51097237591 ± 2401621928 | a | 79058054585 ± 5442748230 | a |

TABLE 8
Photosynthetic parameters of transgenic lines expressing mutated DHS genes in the Col-0
wild-type background. V_{cmax}, Jₘₐₓ, and R_{d} values represent the maximum rate of Rubisco
carboxylation activity, the potential rate of electron transport, and the rate of
mitochondrial dark respiration, respectively. These values are derived from the A-Ci
curves in FIG. 33. Different letters indicate statistically significant differences
among genotypes (one-way ANOVA with Tukey-Kramer test, P < 0.05). Data are means
± SEM (n = 5 independent plant samples for the photosynthetic parameters).

| | | Col- | | | | Col- | | | | Col | | |
|---|---|---|---|---|---|---|---|---|---|---|---|---|
| | Unit | 0::EV | | SEM | | 0::DHS1^{WT} | | SEM | | 0::DHS1^{B4} | | SEM |
| V_{cmax} | μmol/ | 26.54 | ± | 1.59 | c | 29.06 | ± | 2.58 | bc | 33.88 | ± | 1.47 |
| | m²/s | | | | | | | | | | | |
| Jₘₐₓ | μmol/ | 75.26 | ± | 3.94 | b | 76.15 | ± | 3.69 | b | 91.33 | ± | 2.47 |
| | m²/s | | | | | | | | | | | |
| R_{d} | μmol/ | 1.16 | ± | 0.33 | a | 0.84 | ± | 0.19 | a | 1.13 | ± | 0.267 |
| | m²/s | | | | | | | | | | | |

| | | Col- | | | | Col- | | | |
|---|---|---|---|---|---|---|---|---|---|
| | | 0::DHS2^{WT} | | SEM | | 0::DHS2^{A4} | | SEM | |
| V_{cmax} | a | 28.64 | ± | 1.35 | bc | 32.02 | ± | 0.92 | b |
| Jₘₐₓ | a | 74.54 | ± | 3.74 | b | 90.96 | ± | 9.01 | a |
| R_{d} | a | 0.94 | ± | 0.37 | a | 1.35 | ± | 0.55 | a |

TABLE 9
dCAPS markers developed in this study.

| | Amino acid | | | | | |
|---|---|---|---|---|---|---|
| Mutant | alteration | Wild-type sequence | Mutated sequence | | | |
| sotaA4 | DHS2 G222S | GAGCATTTGCTACTGG | GAGCATTTGCTACTG | | | |
| | | AGGTTATGCAGCTAT | GAAGTTATGCAGCTAT | | | |
| | | (SEQ ID NO: 76) | (SEQ ID NO: 82) | | | |
| sotaA11 | DHS2 A224T | ATTTGCTACTGGAGGTT | ATTTGCTACTGGAGGT | | | |
| | | ATGCAGCTATGCAGAG | TATACAGCTATGCAGAG | | | |
| | | (SEQ ID NO: 77) | (SEQ ID NO: 83) | | | |
| sotaB4 | DHS1 G244R | TTAGAGCCTTTGCCAC | TTAGAGCCTTTGCCA | | | |
| | | TGGAGGTTACGCTGC | CTAGAGGTTACGCTGC | | | |
| | | (SEQ ID NO: 78) | (SEQ ID NO: 84) | | | |
| sotaF1 | DHS2 L136F | GACACCTTTAGGGTTC | GACACCTTTAGGGTTC | | | |
| | | TTCTTCAGATGGGT | TTTTTCAGATGGGT | | | |
| | | (SEQ ID NO: 79) | (SEQ ID NO: 85) | | | |
| sotaG1 | DHS3 A240T | TTTGAATCTTTTGAG | TTTGAATCTTTTGAG | | | |
| | | AGCTTTTGCTACTG | AACTTTTGCTACTG | | | |
| | | (SEQ ID NO: 80) | (SEQ ID NO: 86) | | | |
| sotaH1 | DHS3 G114R | ATCGTGTTCGCCGGAG | ATCGTGTTCGCCAGAG | | | |
| | | AAGCTAGATTGCTTG | AAGCTAGATTGCTTG | | | |
| | | (SEQ ID NO: 81) | (SEQ ID NO: 87) | | | |
| | | | Product | | | |
| | | | size | size | | type or |
| Mutant | Forward primer | Reverse primer | (bp) | (bp) | name | mutants? |
| sotaA4 | TTTTCAGGTGGGAAGAATGG | TGTTGCGTGAAATCAAGGTT | 267 | 209 + 58 | GsuI | Wild type |
| | (SEQ ID NO: 88) | (SEQ ID NO: 89) | | | | |
| sotaA11 | GAGTTACAGAGGAGATAAC | ACCCATGAATCCCAAAGCC | 372 | 233 + 139 | BbvI | Wild type |
| | (SEQ ID NO: 90) | (SEQ ID NO: 91) | | | | |
| sotaB4 | TTGAGGAGAAGG | TCAGCTTGCTCA | 229 | 156 + 73 | GsuI | Wild type |
| | ATGGAGTGA | CTTTGTTCA | | | | |
| | (SEQ ID NO: 92) | (SEQ ID NO: 93) | | | | |
| sotaF1 | GGTGGTGATTGTGCT | TGGCCACCGAACATGAGA | 104 | 68 + 36 | XbaI | Wild type |
| | GAGAGTTTC | ACAACACCCATCTCTA | | | | |
| | (SEQ ID NO: 94) | (SEQ ID NO: 95) | | | | |
| sotaG1 | CCTGATCCACAGAGGA | CATAGCAGCATAACC | 93 | 61 + 32 | SacI | Wild type |
| | TGATTAGAGC | ACCAGTAGCAAGAG | | | | |
| | (SEQ ID NO: 96) | (SEQ ID NO: 97) | | | | |
| sotaH1 | GGACTATCCAGATTTA | CGTTCCTCAAGCAATCT | 99 | 73 + 26 | BgIII | Mutant |
| | GCTGCGC | AGCAGATC | | | | |
| | (SEQ ID NO: 98) | (SEQ ID NO: 99) | | | | |

TABLE 10
Primers used in this study. Lowercase letters denote nucleotides
that were mutated via site-directed mutagenesis.

| Sequence | | Target | Laboratory |
|---|---|---|---|
| (5′ to 3′) | Use | gene | ID |
| GCGGAGAGCG | qPCR | AtDHS1 | pHM1475 |
| TACCAGAC | | | |
| (SEQ ID NO: 100) | | | |
| GATCCATTTT | qPCR | AtDHS1 | pHM1476 |
| GTTGCTCACC | | | |
| TT | | | |
| (SEQ ID NO: 101) | | | |
| CAATGCACGG | qPCR | AtDHS2 | pHM1477 |
| AAACACAATC | | | |
| (SEQ ID NO: 102) | | | |
| ACGTCGAAGA | qPCR | AtDHS2 | pHM1478 |
| ACGCTCTCA | | | |
| (SEQ ID NO: 103) | | | |
| CAAGACTCGT | qPCR | AtDHS3 | pHM1479 |
| CCCTTTGACG | | | |
| (SEQ ID NO: 104) | | | |
| GGGTGGCTAC | qPCR | AtDHS3 | pHM1480 |
| CTTCTTGCT | | | |
| (SEQ ID NO: 105) | | | |
| TCCTACTTCA | qPCR | UBQ9 | pHM0111 |
| TGTAGCGCAG | | | |
| GAC | | | |
| (SEQ ID NO: 106) | | | |
| TCCTCCAGAA | qPCR | UBQ9 | pHM0112 |
| TAAGGGCTAT | | | |
| CCG | | | |
| (SEQ ID NO: 107) | | | |
| CTTTGCCACT | Mutagenesis of | AtDHS1 | pHM1186 |
| aGAGGTTACGC | sotaB4 mutation | | |
| TGCTATTCAA | | | |
| AG | | | |
| (SEQ ID NO: 108) | | | |
| GCGTAACCTC | Mutagenesis of | AtDHS1 | pHM1187 |
| tAGTGGCAAAG | sotaB4 mutation | | |
| GCTCTAAGAA | | | |
| G | | | |
| (SEQ ID NO: 109) | | | |
| GCTACTGGAaG | Mutagenesis of | AtDHS2 | pHM1188 |
| TTATGCAGC | sotaA4 mutation | | |
| TATGCAGAGA | | | |
| GTTAG | | | |
| (SEQ ID NO: 110) | | | |
| CTGCATAACI | Mutagenesis of | AtDHS2 | pHM1189 |
| TCCAGTAGCA | sotaA4 mutation | | |
| AATGCTCTCA | | | |
| AGAG | | | |
| (SEQ ID NO: 111) | | | |
| GGAGGTTATaC | Mutagenesis of | AtDHS2 | pHM1190 |
| AGCTATGCA | sotaAll mutation | | |
| GAGAGTTAGC | | | |
| CAG | | | |
| (SEQ ID NO: 112) | | | |
| GCATAGCTGtAT | Mutagenesis of | AtDHS2 | pHM1191 |
| AACCTCCA | sotaAll mutation | | |
| GTAGCAAATG | | | |
| CTC | | | |
| (SEQ ID NO: 113) | | | |
| TAGGGTTCTT | Mutagenesis of | AtDHS2 | pHM1481 |
| (TTCAGATGGG | sotaF1 mutation | | |
| TGTTGTTCTC | | | |
| ATG | | | |
| (SEQ ID NO: 114) | | | |
| CCCATCTGAA | Mutagenesis of | AtDHS2 | pHM1482 |
| aAAGAACCCTA | sotaF1 mutation | | |
| AAGGTGTCTC | | | |
| TAATG | | | |
| (SEQ ID NO: 115) | | | |
| CTTTGAATCT | Mutagenesis of | AtDHS3 | pHM1770 |
| TTTGAGAaCTTTTGCTAC | sotaG1 mutation | | |
| TGGTGG | | | |
| (SEQ ID NO: 116) | | | |
| CCACCAGTAG | Mutagenesis of | AtDHS3 | pHM1771 |
| CAAAAGITCT | sotaG1 mutation | | |
| CAAAAGATTC | | | |
| AAAG | | | |
| (SEQ ID NO: 117) | | | |
| CGTGTTCGCC | Mutagenesis of | AtDHS3 | pHM1379 |
| aGAGAAGCTAG | sotaH1 mutation | | |
| ATTGCTTGAG | | | |
| GAAC | | | |
| (SEQ ID NO: 118) | | | |
| CTAGCTTCTC | Mutagenesis of | AtDHS3 | pHM1380 |
| IGGCGAACAC | sotaH1 mutation | | |
| GATCGGAGGA | | | |
| AAAGC | | | |
| (SEQ ID NO: 119) | | | |
| ATTTTGCCGA | Genotyping of | SALK T-DNA | pHM0027 |
| TTTCGGAAC | SALK lines | left boarder | |
| (SEQ ID NO: 120) | | | |
| TCCTCCTTTG | Genotyping of | tyra2 right | pHM0038 |
| GCGCAACCGC | tyra2 | boarder | |
| (SEQ ID NO: 121) | | | |
| GCGTCACGAA | Genotyping of | tyra2 left | pHM0039 |
| TTGCGATCCA | tyra2 | boarder | |
| GC | | | |
| (SEQ ID NO: 122) | | | |
| TTTTCAGGTG | dCAPS for sotaA4 | AtDHS2 | pHM1166 |
| GGAAGAATGG | | | |
| (SEQ ID NO: 88) | | | |
| TGTTGCGTGA | dCAPS for sotaA4 | AtDHS2 | pHM1167 |
| AATCAAGGTT | | | |
| (SEQ ID NO: 89) | | | |
| GAGTTACAGA | dCAPS for sotaA11 | AtDHS2 | pHM1383 |
| GGAGATAAC | | | |
| (SEQ ID NO: 90) | | | |
| ACCCATGAAT | dCAPS for sotaA11 | AtDHS2 | pHM1384 |
| CCCAAAGCC | | | |
| (SEQ ID NO: 91) | | | |
| TTGAGGAGAA | dCAPS for sotaB4 | AtDHS1 | pHM1168 |
| GGATGGAGTG | | | |
| A | | | |
| (SEQ ID NO: 92) | | | |
| TCAGCTTGCT | dCAPS for sotaB4 | AtDHS1 | pHM1169 |
| CACTTTGTTC | | | |
| A | | | |
| (SEQ ID NO: 93) | | | |
| GGTGGTGATT | dCAPS for sotaF1 | AtDHS2 | pHM1416 |
| GTGCTGAGAG | | | |
| TTTC | | | |
| (SEQ ID NO: 94) | | | |
| TGGCCACCGA | dCAPS for sotaF1 | AtDHS2 | pHM1417 |
| ACATGAGAAC | | | |
| AACACCCATC | | | |
| TCTA | | | |
| (SEQ ID NO: 95) | | | |
| CCTGATCCAC | dCAPS for sotaG1 | AtDHS3 | pHM1602 |
| AGAGGATGAT | | | |
| TAGAGC | | | |
| (SEQ ID NO: 96) | | | |
| CATAGCAGCA | dCAPS for sotaG1 | AtDHS3 | pHM1603 |
| TAACCACCAG | | | |
| TAGCAAGAG | | | |
| (SEQ ID NO: 97) | | | |
| GGACTATCCA | dCAPS for sotaH1 | AtDHS3 | pHM1523 |
| GATTTAGCTG | | | |
| CGC | | | |
| (SEQ ID NO: 98) | | | |
| CGTTCCTCAA | dCAPS for sotaH1 | AtDHS3 | pHM1525 |
| GCAATCTAGC | | | |
| AGATC | | | |
| (SEQ ID NO: 99) | | | |

### REFERENCES

1. U.S. Department of Energy, Accelerating breakthrough innovation in carbon capture, utilization, and storage (2017); www.energy.gov/fe/downloads/accelerating-breakthrough-innovation-carbon-capture-utilization-and-storage.
2. Global Aromatic Market: Information by type (benzene, toluene, O-xylene, P-xylene and others), by application (solvent, additive), by end-use industry (paint & coating, adhesive, pharmaceuticals, chemicals and others), region (North America, Europe, Asia Pacific, Latin America and Middle East & Africa)—Forecast till 2025 (Market Research Future, 2020); www.marketresearchfuture.com/reports/aromatics-market-930.
3. Li T., Shoinkhorova T., Gascon J., Ruiz-Martinez., Aromatics production via methanol-mediated transformation routes. ACS Catal. 11, 7780-7819 (2021).
4. Boerjan W., Ralph J., Baucher M., Lignin biosynthesis. Anm. Rev. Plant Biol. 54, 519-546 (2003).
5. Ragauskas A. J., Beckharn G. T., Biddy M. J., Chandra R., Chen F., Davis M. F., Davison B. H., Dixon R. A., Gilna P., Keller M., Langan P., Naskar A. K., Saddler J. N., Tschaplinski T. J., Tuskan G. A., Wyman C. E., Lignin valorization: Improving lignin processing in the biorefinery. Science 344, 1246843 (2014).
6. Maeda H., Dudareva N., The shikimate pathway and aromatic amino acid biosynthesis in plants. Annu. Rev. Plant Biol. 63, 73-105 (2012).
7. Westfall C. S., Xu A., Jez J. M., Structural evolution of differential amino acid effector regulation in plant chorismate rnutases. J. Biol. Chem. 289, 28619-28628 (2014).
8. Schenck C. A., Chen S., Siehl D. L., Maeda H. A., Non-plastic, tyrosine-insensitive prephenate dehydrogenases from legumes. Nat. Chem. Biol. i1, 52-57 (2015).
9. Yokoyama R., de Oliveira M. V. V., Kleven B., Maeda H. A., The entry reaction of the plant shikimate pathway is subjected to highly complex metabolite-mediated regulation. Plant Cell 33, 671-696 (2021).
10. Jander G., Baerson S. R., Hudak J. A., Gonzalez K. A., Gruys K. J., Last R.

L., Ethylmethanesulfonate saturation mutagenesis in Arabidopsis to determine frequency of herbicide resistance. Plant Physiol. 131, 139-146 (2003).
11. Brotherton J. E., Jeschke M. R., Tranel P. J., Widholm J. M., Identification of Arabidopsis thaliana variants with differential glyphosate responses. J. Plant Physiol. 164, 1337-1345 (2007).
12. Li J., Last R. L., The Arabidopsis thaliana trp5 mutant has a feedback-resistant anthranilate synthase and elevated soluble tryptophan. Plant Physiol. 110, 51-59 (1996).
13. Huang T., Tohge T., Lytovchenko A., Fernie A. R., Jander G., Pleiotropic physiological consequences of feedback-insensitive phenylalanine biosynthesis in Arabidopsis thaliana. Plant J. 63, 823-835 (2010).
14. Pollegioni L., Schonbrunn E., Siehl D., Molecular basis of glyphosate resistance: Different approaches through protein engineering. FEBS J. 278, 2753-2766 (2011).
15. de Oliveira M. V. V., Jin X., Chen X., Griffith D., Batchu S., Maeda H. A., Imbalance of tyrosine by modulating TyrA arogenate dehydrogenases impacts growth and development of Arabidopsis thaliana. Plant J. 97, 901-922 (2019).
16. Sterritt O. W., Kessans S. A., Jameson G. B., Parker E. J., A pseudoisostructural type II DAH7PS enzyme from Pseudomonas aeruginosa: Alternative evolutionary strategies to control shikimate pathway flux. Biochemistry 57, 2667-2678 (2018).
17 Vogt T., Phenylpropanoid biosynthesis. Mol. Plant 3, 2-20 (2010).
18. Zhang X., Liu C.-J., Multifaceted regulations of gateway enzyme phenylalanine ammonia-lyase in the biosynthesis of phenylpropanoids. Mol. Plant 8, 17-27 (2015).
19. Newman L. J., Perazza D. E., Juda L., Campbell M. M., Involvement of the R2R3-MYB, AtMYB61, in the ectopic lignification and dark-photomorphogenic components of the det3 mutant phenotype. Plant J. 37, 239-250 (2004).
20. Dubos C., Stracke R., Grotewold E., Weisshaar B., Martin C., Lepiniec L., MYB transcription factors in Arabidopsis. Trends Plant Sci. 15, 573-581 (2010).
21. Yoo H., Widhalm J. R., Qian Y., Maeda H., Cooper B. R., Jannasch A. S., Gonda I., Lewinsohn E., Rhodes D., Dudareva N, An alternative pathway contributes to phenylalanine biosynthesis in plants via a cytosolic tyrosine:phenylpyruvate aninotransferase. Nat. Commun. 4, 2833 (2013).
22. Wang M., Toda K., Maeda H. A., Biochemical properties and subcellular localization of tyrosine aminotransferases in Arabidopsis thaliana. Phytochemistry 132, 16-25 (2016).
23. Wang M., Toda K., Block A., Maeda H. A., TAT1 and TAT2 tyrosine aminotransferases have both distinct and shared functions in tyrosine metabolism and degradation in Arabidopsis thaliana. J Biol Chem. 294, 3563-3576 (2019).
24. Wang X., Hou Y., Liu L., Li J., Du G., Chen J., Wang M., A new approach for efficient synthesis of phenyllactic acid from L-phenylalanine: Pathway design and cofactor engineering. J. Food Biochem. 42, e12584 (2018).
25. Valera M. J., Boido E., Rarnos J C., Manta E., Radi R., Dellacassa E., Carrau F, The mandelate pathway, an alternative to the phenylalanine ammonia lyase pathway for the synthesis of benzenoids in Ascomycete yeasts. Appl Environ. Microbiol. 86, e00701-20 (2020).
26. Bentley R., The shikimate pathway—A metabolic tree with many branches. Crit. Rev. Biochem. Mol. Biol 25, 307-384 (1990).
27. Arnold A., Nikoloski Z., Bottom-up metabolic reconstruction of Arabidopsis and its application to determining the metabolic costs of enzyme production. Plant Physiol 165, 1380-1391 (2014).
28. Jiao W., Lang E. J., Bai Y., Fan Y., Parker E. J., Diverse allosteric componentry and mechanisms control entry into aromatic metabolite biosynthesis. Curr. Opin. Struct. Biol 65, 159-167 (2020).
29. Tzin V., Malitsky S., Zvi M. M. B., Bedair M., Sumner L., Aharoni A., Galili G., Expression of a bacterial feedback-insensitive 3-deoxy-D-arabino-heptulosonate 7-phosphate synthase of the shikimate pathway in Arabidopsis elucidates potential metabolic bottlenecks between primary and secondary metabolism. New Phytol 194, 430-439 (2012).
30. Tzin V., Rogachev I., Meir S., Moyal Ben Zvi M., Masci T., Vainstein A., Aharoni A., Galili G., Tomato fruits expressing a bacterial feedback-insensitive 3-deoxv-d-arabino-heptulosonate 7-phosphate synthase of the shikimate pathway possess enhanced levels of multiple specialized metabolites and upgraded aroma. J. Exp. Bot. 64, 4441-4452 (2013).
31. Oliva M., Guy A., Galili G., Dor E., Schweitzer R., Amir R., Hacham Y., Enhanced production of aromatic amino acids in tobacco plants leads to increased phenylpropanoid metabolites and tolerance to stresses. Front. Plant Sci. 11, 604349 (2020).
32. Jiao W., Fan Y., Blackmore N. J., Parker E. J., A single amino acid substitution uncouples catalysis and allostery in an essential biosynthetic enzyme in Mycobacterium tuberculosis. J Biol. Chem. 295, 6252-6262 (2020).
33. Henkes S., Sonnewald U., Badur R., Flachmann R., Stitt M., A small decrease of plastid transketolase activity in antisense tobacco transformants has dramatic effects on photosynthesis and phenylpropanoid metabolism. Plant Cell 13, 535-551 (2001).
34. Sinkin A. J., Lopez-Calcagno P E, Davey P. A., Headland L. R., Lawson T., Timm S., Bauwe H., Raines C. A., Simultaneous stimulation of sedoheptulose 1,7-bisphosphatase, fructose 1,6-bisphosphate aldolase and the photorespiratory glycine decarboxylase-H protein increases CO₂ assimilation, vegetative biomass and seed yield in Arabidopsis. Plant Biotechinol. J. 15, 805-816 (2017).
35 Gardemnann A., Schimkat D., Heldt H. W., Control of CO₂ fixation regulation of stromal fructose-1,6-bisphosphatase in spinach by pH and Mg²⁺ concentration. Planta 168, 536-545 (1986).
36. Parrv M. A. J., Keys A. J., Madgwick P. J., Carmo-Silva A. E., Andralojc P. J., Rubisco regulation: A role for inhibitors. J. Exp. Bot. 59, 1569-1580 (2008).
37. Molla K. A., Sretenovic S., Bansal K. C., Qi Y., Precise plant genome editing using base editors and prime editors. Nat. Plants 7, 1166-1187 (2021).
38. Weigel D., Glazebrook J., EMS mutagenesis of Arabidopsis seed. Cold Spring Harb. Protoc. 2006, pdb.prot4621 (2006).
39. Neff M. M., Turk E., Kalishman M., Web-based primer design for single nucleotide polymorphism analysis. Trends Genet. 18, 613-615 (2002).
40. Untergasser A., Cutcutache I, Koressaar T, Ye J., Faircloth B. C., Remm M., Rozen S. G., Primer3—New capabilities and interfaces. Nucleic Acids Res. 40, e115 (2012).
41. Shimada T. L., Shimada T., Hara-Nishimura I., A rapid and non-destructive screenable marker, FAST, for identifying transformed seeds of Arabidopsis thaliana. Plant J. 61, 519-528 (2010).
42. Engler C., Youles M., Gruetzner R., Ehnert T.-M., Werner S., Jones J. D. G., Patron N. J., Marillonnet S., A golden gate modular cloning toolbox for plants. ACS Synth. Biol. 3, 839-843 (2014).
43. Clough S. J., Bent A. F., Floral dip: A simplified method for Agrobacterium-mediated transformation of Arabidopsis thaliana. Plant J. 16, 735-743 (1998).
44. Webb B., Sali A., Comparative protein structure modeling using MODELLER. Curr. Protoc. Bioinformatics 54, 5.6.1-5.6.37 (2016).
45. Laskowski R. A., MacArthur M. W., Moss D. S., Thornton J. M., PROCHECK: A program to check the stereochemical quality of protein structures. J. Appl. Cryst. 26, 283-291 (1993).
46. Wiederstein M., Sippl M. J., ProSA-web: Interactive web service for the recognition of errors in three-dimensional structures of proteins. Nucleic Acids Res. 35, W407-W410 (2007).
47. Pettersen E. F., Goddard T. D., Huang C. C., Couch G. S., Greenblatt D. M., Meng E. C., Ferrin T. E., UCSF Chimera—A visualization system for exploratory research and analysis. J. Comput. Chem. 25, 1605-1612 (2004).
48. Niesen F. T., Berglund H., Vedadi M., The use of differential scanning fluorimetry to detect ligand interactions that promote protein stability. Nat. Protoc. 2, 2212-2221 (2007).
49. Wang M., Lopez-Nieves S., Goldman I. L., Maeda H. A., Limited tyrosine utilization explains lower betalain contents in yellow than in red table beet genotypes. J. Agric. Food Chem. 65, 4305-4313 (2017).
50. Novák O., Hényková E., Sairanen I., Kowalczyk M., Pospisil T., Ljung K., Tissue-specific profiling of the Arabidopsis thaliana auxin metabolome. Plant J. 72, 523-536 (2012).
51. Mancinelli A. L., Iinteraction between light quality and light quantity in the photoregulation of anthocyanin production. Plant Physiol. 92, 1191-1195 (1990).
52. Wellburn A. R., The spectral determination of chlorophylls a and b, as well as total carotenoids, using various solvents with spectrophotometers of different resolution. J. Plant Physiol. 144, 307-313 (1994).
53. Szecowka M., Heise R., Tohge T., Nunes-Nesi A., Vosloh D., Fluege J., Feil R., Lunn J., Nikoloski Z., Stitt M., Fernie A. R., Arrivault S., Metabolic fluxes in an illuminated Arabidopsis rosette. Plant Cell 25, 694-714 (2013).
54. Heise R., Arrivault S., Szecowka M., Tohge T., Nunes-Nesi A., Stitt M., Nikoloski Z., Fernie A. R., Flux profiling of photosynthetic carbon metabolism in intact plants. Nat. Protoc. 9, 1803-1824 (2014).
55. Lisec J., Schauer N., Kopka J., Willmitzer L., Fernie A. R., Gas chromatography mass spectrometry-based metabolite profiling in plants. Nat. Protoc. 1, 387-396 (2006).
56. Maeda H., Song W., Sage T. L., DellaPenna D., Tocopherols play a crucial role in low-temperature adaptation and phloem loading in Arabidopsis. Plant Cell 18, 2710-2732 (2006).
57. Bradford M. M., A rapid and sensitive method for the quantitation of microgram quantities of protein utilizing the principle of protein-dye binding. Anal Biochem. 72, 248-254 (1976).
58. Pradhan Mitra P., Loqué D., Histochemical staining of Arabidopsis thaliana secondary cell wall elements. J. Vis. Exp. , (2014).
59. Suzuki S., Suzuki Y., Yamamoto N., Hattori T., Sakamoto M., Umezawa T., High-throughput determination of thioglycolic acid lignin from rice. Plant Biotechnol. 26, 337-340 (2009).
60. Farquhar G. D., von Caemmerer S., Berry J. A., A biochemical model of photosynthetic CO₂ assimilation in leaves of C3 species. Planta 149, 78-90 (1980).
61. Duursma R. A., Plantecophys—An R package for analysing and modelling leaf gas exchange data. PLOS ONE 10, e0143346 (2015).
62. Kromdijk J., Glowacka K., Long S. P., Photosynthetic efficiency and mesophyll conductance are unaffected in Arabidopsis thaliana aquaporin knock-out lines. J. Exp. Bot. 71, 318-329 (2020).
63. Oñate-Sánchez L., Vicente-Carbajosa J., DNA-free RNA isolation protocols for Arabidopsis thaliana, including seeds and siliques. BMC. Res. Notes 1, 93 (2008).
64. Goodstein D. M., Shu S., Howson R., Neupane R., Hayes R. D., Fazo J., Mitros T., Dirks W., Hellsten U., Putnam N., Rokhsar D. S., Phytozome: A comparative platform for green plant genomics. Nucleic Acids Res. 40, D1178-D1186 (2012).
65. Bombarely A., Rosli H. G., Vrebalov J., Moffett P., Mueller L. A., Martin G. B., A draft genome sequence of Nicotiana benthamiana to enhance molecular plant-microbe biology research. Mol. Plant Microbe Interact. 25, 1523-1530 (2012).
66. Waterhouse A. M., Procter J. B., Martin D. M. A., Clamp M., Barton G. J., Jalview Version 2—A multiple sequence alignment editor and analysis workbench. Bioinformatics 25, 1189-1191 (2009).
67. Kopka J., Schauer N., Krueger S., Birkemeyer C., Usadel B., Bergmuller E., Dörmann P., Weckwerth W., Gibon Y., Stitt M., Willnitzer L., Fernie A. R., Steinhauser D., GMD@CSB.DB: The Golm Metabolome Database. Bioinformatics 21, 1635-1638 (2005).
68. Schauer N., Steinhauser D., Strelkov S., Schoniburg D., Allison G., Moritz T., Lundgren K., Roessner-Tunali U., Forbes M. G., Willmitzer L., Fernie A. R., Kopka J., GC-MS libraries for the rapid identification of metabolites in complex biological samples. FEBS Lett. 579, 1332-1337 (2005).

### Example 2

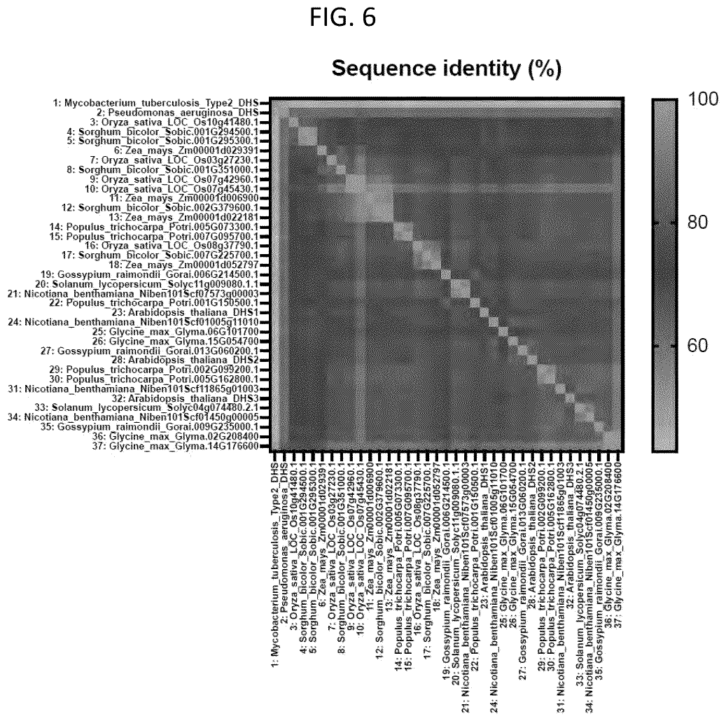

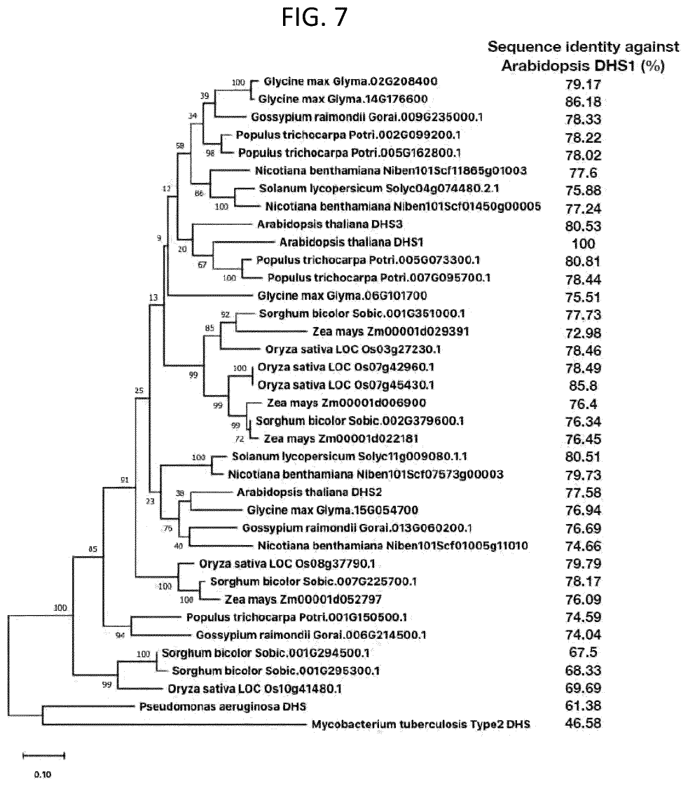

We also conducted Illumina whole-genome sequencing on 12 additional sota lines (sotaA12, sotaE3, sotaE31, sotaC4, sotaA2, sotaA5, sotaB1, sotaA3, sotaA9, sotaA13, sotaF26, and sotaA12) using the methods described in Example 1. However, here it was the mutants themselves that were sequenced rather than backcrossing them with Arabidopsis tyra2 to generate a population. These additional sota lines were selected based on the data presented in FIG. 1, which shows that each of these mutants produce high levels of tyrosine and phenylalanine as compared to wild-type plants. The sequencing result revealed that these additional sota lines comprise mutations at several of the same positions as well as several new positions within the three Arabidopsis DHS isoforms (i.e., DHS1, DHS2, and DHS3). In FIG. 5, the positions of all 20 mapped mutations are indicated on a sequence alignment of DHS orthologs from several bacterial and crop species (SEQ ID NO:1-37). The sequences of these DHS orthologs are outlined in Table 11. The DHS orthologs are highly conserved and share a substantial degree of sequence identity (FIG. 6, FIG. 7). In FIG. 8, the locations of the 20 mapped mutations are shown on an Arabidopsis DHS2 protein model.

TABLE 11
DHS sequences disclosed herein.

| | Amino acid | Exemplary DNA |
|---|---|---|
| DHS polypeptide | sequence | sequence |
| Arabidopsis_thaliana_DHS1 | SEQ ID NO: 1 | SEQ ID NO: 38 |
| Arabidopsis_thaliana DHS2 | SEQ ID NO: 2 | SEQ ID NO: 39 |
| Arabidopsis_thaliana DHS3 | SEQ ID NO: 3 | SEQ ID NO: 40 |
| Mycobacterium_tuberculosis_Type2_DHS | SEQ ID NO: 4 | SEQ ID NO: 41 |
| Pseudomonas_aeruginosa_DHS | SEQ ID NO: 5 | SEQ ID NO: 42 |
| Oryza_sativa_LOC_Os10g41480.1 | SEQ ID NO: 6 | SEQ ID NO: 43 |
| Sorghum_bicolor_Sobic.001G294500.1 | SEQ ID NO: 7 | SEQ ID NO: 44 |
| Sorghum_bicolor_Sobic.001G295300.1 | SEQ ID NO: 8 | SEQ ID NO: 45 |
| Zea_mays_Zm00001d029391 | SEQ ID NO: 9 | SEQ ID NO: 46 |
| Oryza_sativa_LOC_Os03g27230.1 | SEQ ID NO: 10 | SEQ ID NO: 47 |
| Sorghum_bicolor_Sobic.001G351000.1 | SEQ ID NO: 11 | SEQ ID NO: 48 |
| Oryza sativa_LOC_Os07g42960.1 | SEQ ID NO: 12 | SEQ ID NO: 49 |
| Oryza_sativa_LOC_Os07g45430.1 | SEQ ID NO: 13 | SEQ ID NO: 50 |
| Zea_mays_Zm00001d006900 | SEQ ID NO: 14 | SEQ ID NO: 51 |
| Sorghum_bicolor_Sobic.002G379600.1 | SEQ ID NO: 15 | SEQ ID NO: 52 |
| Zea_mays_Zm00001d022181 | SEQ ID NO: 16 | SEQ ID NO: 53 |
| Populus_trichocarpa_Potri.005G073300.1 | SEQ ID NO: 17 | SEQ ID NO: 54 |
| Populus_trichocarpa_Potri.007G095700.2 | SEQ ID NO: 18 | SEQ ID NO: 55 |
| Oryza_sativa_LOC_Os08g37790.1 | SEQ ID NO: 19 | SEQ ID NO: 56 |
| Sorghum_bicolor_Sobic.007G225700.1 | SEQ ID NO: 20 | SEQ ID NO: 57 |
| Zea_mays_Zm00001d052797 | SEQ ID NO: 21 | SEQ ID NO: 58 |
| Gossypium_raimondii_Gorai.006G214500.1 | SEQ ID NO: 22 | SEQ ID NO: 59 |
| Solanum_lycopersicum_Solyc11g009080.1.1 | SEQ ID NO: 23 | SEQ ID NO: 60 |
| Nicotiana_benthamiana_Niben101Scf07573g00003 | SEQ ID NO: 24 | SEQ ID NO: 61 |
| Populus_trichocarpa_Potri.001G150500.2 | SEQ ID NO: 25 | SEQ ID NO: 62 |
| Nicotiana_benthamiana_Niben101Scf01005g11010 | SEQ ID NO: 26 | SEQ ID NO: 63 |
| Glycine_max_Glyma.06G101700 | SEQ ID NO: 27 | SEQ ID NO: 64 |
| Glycine_max_Glyma.15G054700 | SEQ ID NO: 28 | SEQ ID NO: 65 |
| Gossypium_raimondii_Gorai.013G060200.1 | SEQ ID NO: 29 | SEQ ID NO: 66 |
| Populus_trichocarpa_Potri.002G099200.1 | SEQ ID NO: 30 | SEQ ID NO: 67 |
| Populus_trichocarpa_Potri.005G162800.1 | SEQ ID NO: 31 | SEQ ID NO: 68 |
| Nicotiana_benthamiana_Niben101Scf11865g01003 | SEQ ID NO: 32 | SEQ ID NO: 69 |
| Solanum_lycopersicum_Solyc04g074480.2.1 | SEQ ID NO: 33 | SEQ ID NO: 70 |
| Nicotiana_benthamiana_Niben101Scf01450g00005 | SEQ ID NO: 34 | SEQ ID NO: 71 |
| Gossypium_raimondii_Gorai.009G235000.1 | SEQ ID NO: 35 | SEQ ID NO: 72 |
| Glycine_max_Glyma.02G208400 | SEQ ID NO: 36 | SEQ ID NO: 73 |
| Glycine_max_Glyma.14G176600 | SEQ ID NO: 37 | SEQ ID NO: 74 |

### Example 3

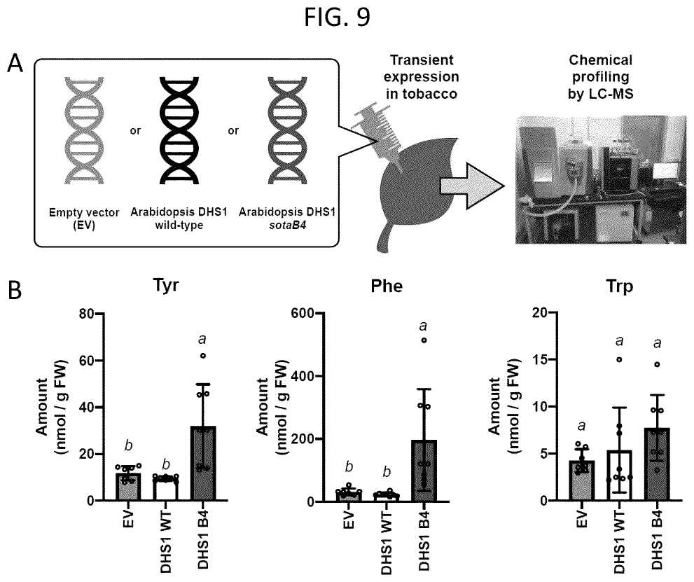

In the following example, we demonstrate that the identified mutant Arabidopsis DHS proteins can be used to increase production of AAAs in other plant species. The Arabidopsis DHS1 sotaB4 mutant was transiently expressed in tobacco, and the levels of the three AAAs were measured using LC-MS. As is shown in FIG. 9, expression of the mutant DHS1 protein resulted in significantly elevated production of tyrosine and phenylalanine in the tobacco plant.

### Vector Construction

Vectors for plant expression were made as previously described using MoClo modular cloning technology. For transient expression in Nicotiana benthamiana, gene expression of the protein coding sequence (CDS) of DHS1 WT and B4 were driven by a 1987-bp sequence obtained from the upstream region of the ubiquitin 10 gene (At4g05320) from Arabidopsis. In addition, a synthetic hemagglutinin (HA) tag comprising 6 repeats of the sequence YPYDVPDYA (SEQ ID NO:75) was added to the C-terminus of the protein for quantification of protein expression using an anti-HA antibody. Vectors containing the promoter, CDS, epitope HA-tag, and a terminator were transformed into Agrobacterium tumefaciens via electroporation.

Transient Expression in Nicotiana benthamiana

Positive transformants were used to perform transient expression in Nicotiana benthamiana. Single colony bacteria were grown in LB media supplemented with kanamycin (100 mg/L) and gentamycin (100 mg/L) at 28° C. with constant agitation at 200 rpm. 10 mL of initial culture was expanded to 50 mL by inoculating 50 mL of fresh LB media supplemented with same antibiotics plus 10 mM MES and 200 uM acetosyringone with 3 mL of overnight culture. Bacteria cultures were allowed to grow at 28° C. and 200 rpm agitation for 16 hours. Bacteria cultures were sedimented in 50 mL Falcon tubes via centrifugation at room temperature and 4000 g for 20 min. After centrifugation, growth media was decanted and bacteria were resuspended in 5 mL of inoculation solution (10 mM MES, 10 mM MgCl₂ 200 uM acetosyringone). After complete resuspension, bacteria concentration was evaluated by spectrometry using optical density (OD) at 600 nm. Bacteria solution was diluted to final OD₆₀₀=1.0 using inoculation solution. Diluted bacteria were incubated at room temperature without agitation for 3 hours and used to inoculate Nicotiana leaves via needle-less 1 mL syringes.

Each inoculated leaf was separated into 4 quadrants, and each quadrant received a different bacteria solution containing a different vector. The experiment was completely randomized to allow the production of aromatic amino acids by DHS1 WT and DHS1 B4 to be compared. After inoculation, the inoculated area was marked by black sharpie, the excess of bacterial solution on the leaves were gently removed using tissue papers, and plants were returned to growth chambers. Samples for metabolite analysis were collected two days after inoculation and processed as previously described for quantification of aromatic amino acids.

### Example 4

In the following example, we demonstrate that introducing sota mutations into DHS genes from sorghum and poplar also dramatically enhances AAA production in plants. The sota mutations sotaB4 and sotaF1 were introduced into the Sorghum bicolor gene SbDHS (Sobic.007G225700.1.p) and the Populus trichocarpa gene PtDHS (Potri.005G073300.1.p) and expressed in Nicotiana benthamiana leaves via Agrobacterium-mediated transformation.

To generate these mutant genes, DNA sequences encoding SbDHS (SEQ ID NO:20; which was cloned from Sorghum cDNA) and PtDHS (SEQ ID NO:17; which was synthesized) were cloned into E. coli expression vectors. Notably, the portions of these sequences that encode plastid transit peptides were omitted from the cloned sequences to aid in the production of recombinant protein. The sequences were modified to include the sotaB4 and sotaF1 mutations via site-directed mutagenesis. Then, both wild-type and sota mutant versions of the CDS were cloned into the modular cloning (MoClo) vector pAGM1287, wherein each sequence was flanked by the quadruplets AATG and TTCG for future cloning purposes. In this vector, expression of the DHS proteins was driven by a 739-bp sequence containing the promoter and 5′-UTR from the upstream region of the rbcS2 (ribulose bisphosphate carboxylase small subunit, chloroplastic 2) gene from Solanum lycopersicum (SEQ ID NO:123). This regulatory sequence was obtained from the MoClo plasmid pICH71301 and was modified to be flanked by the quadruplets GGAG and CCAT in the vector. Additionally, to allow the DHS proteins to be expressed in the plastids, a 176-bp synthetic DNA fragment encoding the rubisco complex (RbcS) plastid transit peptide (SEQ ID NO:124; obtained from the MoClo plasmid pICH78133) was included in the vector and was modified to be flanked by the quadruples CCAT and AATG. Two different tags, i.e., hemagglutinin (HA) and TdTomato-HA, were used to monitor protein expression, and the P19 vector was co-transformed to prevent gene silencing. A dipeptide (glycine-serine) linker was included between the C-terminus of the DHS proteins and the HA/TdTomato-HA tags. Additionally, the PtDHS protein contained a 6×-His-Tag at its N-terminus (introduced during sequence synthesis) for purification using Ni⁺-affinity chromatography. The sequences of the components used in these expression vectors are outlined in Table 12, and the sequences of the proteins expressed from these vectors are outlined in Table 13.

TABLE 12
Components of vectors used to express DHS proteins in Nicotiana benthamiana

| | | Amino acid |
|---|---|---|
| Vector component | DNA sequence | sequence |
| Promoter and 5′UTR | SEQ ID NO: 123 | Not translated |
| Plastid transit peptide | SEQ ID NO: 124 | SEQ ID NO: 131 |
| WT Sorghum bicolor DHS CDS | SEQ ID NO: 125 | SEQ ID NO: 132 |
| Sorghum bicolor DHS CDS with sotaB4 mutation | SEQ ID NO: 126 | SEQ ID NO: 133 |
| Sorghum bicolor DHS CDS with sotaF1 mutation | SEQ ID NO: 127 | SEQ ID NO: 134 |
| WT Populus trichocarpa DHS CDS | SEQ ID NO: 128 | SEQ ID NO: 135 |
| Populus trichocarpa DHS CDS with sotaB4 | SEQ ID NO: 129 | SEQ ID NO: 136 |
| mutation | | |
| Populus trichocarpa DHS CDS with sotaF1 | SEQ ID NO: 130 | SEQ ID NO: 137 |
| mutation | | |

TABLE 13
Sequences of tagged DHS proteins expressed in Nicotiana benthamiana

| DHS protein | Amino acid sequence |
|---|---|
| WT Sorghum bicolor DHS | SEQ ID NO: 138 |
| Sorghum bicolor DHS with sotaB4 mutation | SEQ ID NO: 139 |
| Sorghum bicolor DHS with sotaF1 mutation | SEQ ID NO: 140 |
| WT Populus trichocarpa DHS | SEQ ID NO: 141 |
| Populus trichocarpa DHS with sotaB4 mutation | SEQ ID NO: 142 |
| Populus trichocarpa DHS with sotaF1 mutation | SEQ ID NO: 143 |

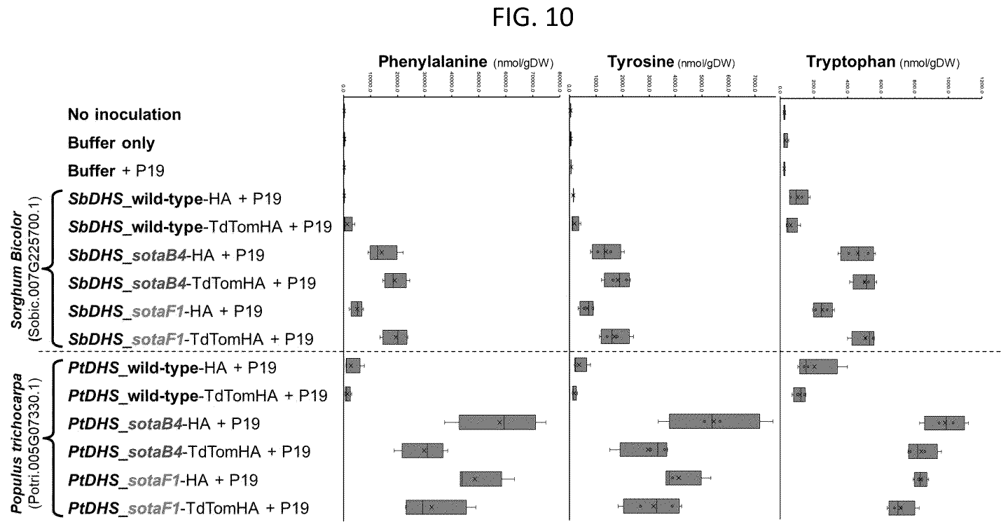

The levels of AAAs produced in Nicotiana benthamiana leaves that expressed the wild-type and sota mutant versions of these DHS proteins were measured via liquid chromatography-mass spectrometry (LC-MS), as described in Materials and Methods. As is shown in FIG. 10, expression of the mutant DHS proteins resulted in significantly elevated production of phenylalanine, tyrosine, and tryptophan in the tobacco plants as compared to the wild-type DHS proteins.

1. An engineered 3-deoxy-D-arabino-heptulosonate 7-phosphate synthase (DHS) polypeptide comprising at least one mutation at a position corresponding to amino acid residue 109, 114, 159, 240, 244, 245, 247, 248, 319, 322, or 348 of SEQ ID NO:1 (DHS1), wherein the polypeptide has at least 80% sequence identity to a polypeptide selected from SEQ ID NO:1-37.

2. (canceled)

3. The engineered polypeptide of claim 1, wherein the at least one mutation includes at least one mutation corresponding to P109S, P109L, G114R, L159F, A240V, A240T, G244R, G245S, A247V, A247T, A248T, D319N, S322F, or E348K in SEQ ID NO:1.

4. The engineered polypeptide of claim 1, wherein the engineered polypeptide has reduced inhibition by one or more tyrosine-associated compound or tryptophan-associated compound relative to the wild-type form of the polypeptide.

5. A polynucleotide encoding the engineered polypeptide of claim 1.

6-8. (canceled)

9. A construct comprising a promoter operably linked to the polynucleotide of claim 5.

10-12. (canceled)

13. A vector comprising the polynucleotide of claim 5.

14. A cell comprising the engineered polypeptide of claim 1.

15-16. (canceled)

17. A seed comprising the engineered polypeptide of claim 1.

18. A plant grown from the seed of claim 17.

19. A plant comprising the engineered polypeptide of claim 1.

20. The plant of claim 19, wherein the plant is selected from a tomato plant, a tobacco plant, a soybean plant, a cotton plant, a poplar plant, a sorghum plant, a rice plant, and a corn plant.

21. The plant of claim 19, wherein the plant:
(a) produces a greater quantity of aromatic amino acids as compared to a control plant;
(b) assimilates a greater quantity of carbon dioxide (CO₂) as compared to a control plant; or
(c) both (a) and (b).

22. (canceled)

23. The plant of claim 21, wherein the net CO₂ assimilation of the plant is at least 30% greater than that of a control plant.

24. A method for increasing production of aromatic amino acids in a plant, the method comprising: introducing the polynucleotide of claim 5 into the plant.

25. The method of claim 24, further comprising purifying aromatic amino acids or derivatives thereof from the plant.

26. A method for increasing the amount of CO₂ sequestered by a plant, the method comprising: introducing the polynucleotide of claim 5 into the plant.

27. The method of claim 24, wherein the plant is selected from a tomato plant, a tobacco plant, a soybean plant, a cotton plant, a poplar plant, a sorghum plant, a rice plant, and a corn plant.

28. A method for producing aromatic amino acids or derivatives thereof, the method comprising:
a) growing the plant of claim 19; and
b) purifying aromatic amino acids or derivatives thereof produced by the plant.

29. A method for sequestering CO₂, the method comprising growing the plant of claim 19.

30. The method of claim 29, further comprising harvesting part of the plant while leaving the roots of the plant in the soil.


SEQUENCE LISTING (143 sequences)

SEQ ID NO: 1 - protein, 525 aa, Arabidopsis thaliana
MALSNASSLSTRSIYGGDLSHRPSNRQSSFTFHPAVNTKPKSVNLVTAVHAAEPARNAVSVKESVASSSSGALKWTPESWKLKKALQLPDYPNANELESVLKTIEAFPPIVFAGEARNLEERLADAAVGKAFLLQGGDCAESFKEFNATNIRDTFRVLLQMSIVLTFGGQVPVIKVGRMAGQFAKPRSDAFEEKDGVKLPSYKGDNINGDTFDEKSRIPDPNRMIRAYTQSAATLNLLRAFATGGYAAIQRVTQWNLDFVEQSEQADRYQELANRVDEALGFMSACGLGTDHPLMTTTDFYTSHECLLLPYEQSLTRLDSTSGLYYDCSAHMVWCGERTRQLDGAHVEFLRGIANPLGIKVSNKMDPFELVKLVEILNPNNKPGRITVIVRMGAENMRVKLPHLIRAVRRSGQIVTWVCDPMHGNTIKAPCGLKTRAFDSILAEVRAFLDVHEQEGSHAGGIHLEMTGQNVTECIGGSRTVTYDDLSSRYHTHCDPRLNASQSLELAFIVAERLRKRRTGSQRVS

SEQ ID NO: 2 - protein, 507 aa, Arabidopsis thaliana
MVTLNASSPLTTKSFLPYRHAPRRPISFSPVFAVHSTDPKKSTQSASASVKWSLESWKSKKALQLPDYPDQKDVDSVLQTLSSFPPIVFAGEARKLEDKLGQAAMGQAFMLQGGDCAESFKEFNANNIRDTFRVLLQMGVVLMFGGQLPVIKVGRMAGQFAKPRSDPFEEKDGVKLPSYRGDNINGDAFDEKSRIPDPHRMVRAYTQSVATLNLLRAFATGGYAAMQRVSQWNLDFTQHSEQGDRYRELANRVDEALGFMGAAGLTSAHPIMTTTEFWTSHECLLLPYEQALTREDSTSGLYYDCSAHMLWVGERTRQLDGAHVEFLRGIANPLGIKVSDKMVPSELVKLIEILNPQNKPGRITVIVRMGAENMRVKLPNLIRAVRGAGQIVTWVSDPMHGNTIMAPGGLKTRSFDAIRAELRAFFDVHDQEGSFPGGVHLEMTGQNVTECVGGSRTITYNDLSSRYHTHCDPRLNASQSLELAFIIAERLRKRRLGSGNLPSSIGV

SEQ ID NO: 3 - protein, 527 aa, Arabidopsis thaliana
MALMNGSMNLSSVKSSMINHRQPNFSSAVSRPTSFRISAVQTDPKTPAASSASAATTTPATLTKPVGVNVGKGKWAPESWRTKKALQQPDYPDLAALEAVLETIEAFPPIVFAGEARLLEERLGQAAMGEAFLLQGGDCAESFKEFNANNIRDTFRILLQMGAVLMFGGQVPVVKVGRMAGQFAKPRSDSFEEKDGVKLPSYRGDNINGDAFDSKSRIPDPQRMIRAYCQSAATLNLLRAFATGGYAAMQRVTQWNLDFTERSEQGDRYRELANRVDEALGFMHAAGLTLDHPIMQTTDFWTSHECLLLPYEQSLTRLDSTSGLYYDCSAHMIWVGERTRQLDGAHVEFLRGVANPLGIKVSDKMDPKELVKLIEILNADNKPGRITIITRMGAENMRVKLPHLIREVRRAGQIVTWVSDPMHGNTIKAPCGLKTRPFDAILAEVRAFFDVHEQEGSHPGGIHLEMTGQNVTECIGGSRTVTFDDLGSRYHTHCDPRLNASQSLELSFIIAERLRKRRIKSQKAFAL

SEQ ID NO: 4 - protein, 462 aa, Mycobacterium tuberculosis
MNWTVDIPIDQLPSLPPLPTDLRTRLDAALAKPAAQQPTWPADQALAMRTVLESVPPVTVPSEIVRLQEQLAQVAKGEAFLLQGGDCAETFMDNTEPHIRGNVRALLQMAVVLTYGASMPVVKVARIAGQYAKPRSADIDALGLRSYRGDMINGFAPDAAAREHDPSRLVRAYANASAAMNLVRALTSSGLASLHLVHDWNREFVRTSPAGARYEALATEIDRGLRFMSACGVADRNLQTAEIYASHEALVLDYERAMLRLSDGDDGEPQLFDLSAHTVWIGERTRQIDGAHIAFAQVIANPVGVKLGPNMTPELAVEYVERLDPHNKPGRLTLVSRMGNHKVRDLLPPIVEKVQATGHQVIWQCDPMHGNTHESSTGFKTRHFDRIVDEVQGFFEVHRALGTHPGGIHVEITGENVTECLGGAQDISETDLAGRYETACDPRLNTQQSLELAFLVAEMLRD

SEQ ID NO: 5 - protein, 448 aa, Pseudomonas aeruginosa
MSQSWSPESWRAKPIQQQPEYPDAAHLARVEQTLAGYPPLVFAGEARELRRQFAEVTAGRAFLLQGGDCAESFAEFSAAKIRDTFKVLLQMAVVMTFAAGCPVVKVGRMAGQFAKPRSSGDETQNGVTLPAYRGDIVNGIGFDEKSRVPDPERLLQAYHQSTASLNLLRAFAQGGFADLHQVHRWNLDFIANSALAERYQQLADRIDETLAFMRACGLDSAPQLRETSFFTAHEALLLNYEEALTRRDSLTGEWYDCSAHMLWIGDRTRQIDGAHVEMLRGVGNPIGVKVGPSMDSEELIRLIDILNPDNDPGRLNLIVRMGADKVGDHLPRLIQAIQREGRQVLWSSDPMHGNTIKASSGYKTRDFARVLAEVRQFFEVHQAEGSYAGGIHIEMTGQNVTECIGGSRPITEDGLSDRYHTHCDPRLNADQSLELAFLIAETLKQVRR

SEQ ID NO: 6 - protein, 508 aa, Oryza sativa
MTCHSAMAALTVGHAAIVHATTRLEDARSTGRRRRRRGMITVRAAAAATSGWEPGSWRARPARQIPEYPDAAALEGAERELASFPPLVFAGEARKLEERLGDAAMGRAFLLQGGDCAESFKEFAANNIRDTFRLMLQMAVVLTFGGQMPTIKVGRMAGQFAKPRSNPTETIDGVTLPSYRGDIINSDGFDEKSRAPDPERLIRAYSQSASTLNLLRGFAHGGYADLQRVTQWNLDFLRDSTQGDRYMELSERVHDAIGFMVAAGLTPQHPIMTTAEFWTSHECLHLPYEQALTRVDSISGLYYDCSAHMLWVGERTRQLDGAHVEFLRGISNPLGVKVSDKLEPSELVKLCEILNPHNKPGRLTIITRMGAENTRVKLPHMIRAVRQAGLIVTWVSDPMHGNTISAPCGLKTRSFDAIRCELRAFFDVHEQEGSYPGGIHLEMTGQNVTECIGGSKTVTLDDLSSRYRTHCDPRLNASQSLELAFAIADRLRKKRDRAWNRLVYRAVA

SEQ ID NO: 7 - protein, 499 aa, Sorghum bicolor
MAPLATAPPPTPHAPRSLVPRVRPRRGLTAVRAASQGRTTDGWTPGSWRARPARQIPEYPDTAALEATERTLAEFPPLVFTGEVRKLEERLGEAAMGRAFLLQGGDCAESFREFNAQKIRDTFRLILQMAVVLTFGGQMPTIKVGRMGGQFAKPRSNPTETVDGVTLPSYRGDIINDQAFDEKSRVPDPERLIRAYTQSASTLNLLRAFAHGGFADLQRVTQWNLDFLRHSTQGDRYLELSQRVHEAIGFMVAAGLTPQHPIMTTAELWTSHESLHLPYEQALTREDSISGRYYDCSAHMLWVGERTRQLDGAHVEFLRGISNPLGIKVSDKLDPSELVKLCETLNPHNKPGRLTIITRMGAENMRVKLPQMIRAVRQAGMIVTWVSDPMHGNTISAPCGLKTRSFDSIMAELRAFFDVHGLEGSYPGGVHLEMTGQNVTECIGGSKAVTFDDLSARYHTHCDPRLNASQSLELAFAIADRLRNKRDKTWHNLTSRVVA

SEQ ID NO: 8 - protein, 495 aa, Sorghum bicolor
MAPLATAPPPTPHAPRSLVPRVRPRRGLTAVRAASQGRTTDGWTPGSWRARPARQIPEYPDTAALEATERTLAEFPPLVFTGEVRKLEERLGEAAMGRAFLLQGGDCAESFREFNAQKIRDTFRLILQMAVVLTFGGQMPTIKVGRMGGQFAKPRSNPTETVDGVTLPSYRGDIINDQAFDEKSRVPDPERLIRAYTQSASTLNLLRAFAHGGFADLQRVTQWNLDFLRHSTQGDRYLELSQRVHEAIGFMVAAGLTPQHPIMTTAELWTSHESLHLPYEQALTREDSISGRYYDCSAHMLWVGERTRQLDGAHVEFLRGISNPLGIKVSDKLDPSELVKLCETLNPHNKPGRLTIITRMGAENMRVKLPQMIRAVRQAGMIVTWVSDPMHGNTISAPCGLKTRSFDSIMAELRAFFDVHGLEGSYPGGVHLEMTGQNVTECIGGSKAVTFDDLSARYHTHCDPRLNASQSLELAFAIADSPDLKMVKRSVQPSK

SEQ ID NO: 9 - protein, 550 aa, Zea mays
MALATNAAAAAAISGGSASQPRRASPSLLLLRRRCAVGAVHAAEPSKNPGVVPAAAKASTPTVVPENDTVPAPSLGPNASLRARWAVDSWRTKKALQQPEYPNQTELEEAVNTIEKSPPIVFAGEARHLEERLADAAVGRAFLLQGGDCAESFKEFTSANIRDTFRVILQMAVVLTFGGQMPVVKVGRMAGQFAKPRSDAFEERGGVKLPSYRGDIINGEAFDEKSRVPDPQRMLKAYYQSASKLNLLRAFATGGYADIQRVTQWNLDFTKHSEQGDRYRELAHRVDDALGFMGAAGLTVDQPLTTMIEFWTSHECLLLPYEQALTRQDSTTGLFYDCSAHMLWVGERTRQLDGAHVEFLRGIANPLGIKVSDKMEPGELVKLIDILNPTNKPGRITVITRMGAENIRVKLPHLIRAVRQAGQSVTWITDPMHGNTIKTPCGRKTRPFDSILAEVRAFFDVHDQEGSQPGGVHLEMTGQNVTECTGGSSNVTFDNLRDRYHTQCDPRLNASQSLELAFILAEKLRKRRMRRSSVASGLGGSILPLPPFGF

SEQ ID NO: 10 - protein, 555 aa, Oryza sativa
MSLATSSSMAGGAAVVPRSATATTASAFVTMKRRATAVRAVHAAEPSKNPPVGVPSAAKTSSPSVAAPEKAPVAAAPAPVAPAPAATKQVAPARWAVDSWRTKKALQLPEYPNAAELEAVLKTIEAFPPIVFAGEARHLEERLADAAMGRAFLLQGGDCAESFKEFNGNNIRDTFRVLLQMSAVLTFGGQMPVIKVGRMAGQFAKPRSEAFEERDGVKLPSYRGDNINGDAFNEKSRIPDPQRMVRAYAQSAATLNLLRAFATGGYAAMQRVTQWNLDFTQHSEQGDRYRELAHRVDEALGFMSAAGLTVDHPLMTSTDFWTSHECLLLPYEQSLTRQDSTTGHFYDCSAHMLWVGERTRQLDGAHVEFLRGVANPLGIKVSDKMNPTELVKLIEILNPSNKPGRITIITRMGAENMRVKLPHLIRAVRHAGQIVTWITDPMHGNTIKAPCGLKTRPFDSILAEVRAFFDVHDQEGSHPGGVHLEMTGQNVTECIGGSRTVTFDDLGDRYHTHCDPRLNASQSLELSFIIAERLRRKRIRSSKLNNMLPLPPFGV

SEQ ID NO: 11 - protein, 551 aa, Sorghum bicolor
MALSTNAAAAAAAISGGSASASQPARRTPSSLLPLTRRRRAVVRAVHAAEPSKNPGVVVPAAAKASSPTTVAPENDAAPAPAPARAPAKWAVDSWRTKKALQLPEYPNPAELEAVLKTIEAFPPIVFAGEARHLEERLADAAMGRAFLLQGGDCAESFKEFNSNNIRDTFRVLLQMSAVLMFGAQMPVVKVGRMAGQFAKPRSDPFEVRDGVKLPSYRGDNINGEAFDEKSRVPDPQRMIRAYAQSAATLNLLRAFATGGYAAMQRVTQWNLDFTEHSEQGDRYRELAHRVDEALGFMSAAGLTPDHPLTTTTEFWTSHECLLLPYEQALTRQDSTSGLFYDCSAHMLWVGERTRQLDGAHVEFLRGIANPLGIKVSDKMNPSDLVKLIDILNPTNKPGRITVITRMGAENMRVKLPHLIRAVRQAGQIVTWITDPMHGNTIKAPCGLKTRPFDNILAEVRAFFDVHEQEGSHPGGVHLEMTGQNVTECIGGSRTVTFDDLSDRYHTHCDPRLNASQSLELAFIIAERLRKRRIRSSSGLNNILPLPPFGF

SEQ ID NO: 12 - protein, 537 aa, Oryza sativa
MALATNSAAVSGGAAAAASSAPQPRLAATFLPMRRRTVSAVHAADPAKSNGPVQAAAKASSPSTVAAPEKKPVGLGKWTVDSWKAKKALQLPEYPSQEELDSVLKTIETFPPVVFAGEARHLEERLADAAMGRAFVLQGGDCAESFKEFNANNIRDTFRILLQMGAVLMFGGQMPVVKVGRMAGQFAKPRSDSFEERDGVKLPSYRGDNINGDTFDEKSRVPDPQRMIRAYAQSVATLNLLRAFATGGYAAMQRVTQWNLDFMDHSEQGDRYRELAHRVDEALGFMTAAGLTVDHPIMTTTDFWTSHECLLLPYEQSLTREDSTSGLFYDCSAHMLWVGERTRQLDGAHVEFLRGVANPLGIKVSDKMNPRDLVKLIEILNPSNKPGRITIITRMGAENMRVKLPHLIRAVRNSGQIVTWITDPMHGNTIKAPCGLKTRPFDSILAEVRAFFDVHDQEGSHPGGIHLEMTGQNVTECIGGSRTVTFDDLSDRYHTHCDPRLNASQSLELAFIIAERLRRRRMRSGVNSNLPLPPLAF

SEQ ID NO: 13 - protein, 356 aa, Oryza sativa
MAGQFAKPRSDSFEERDGVKLPSYRGDNINGDTFDEKSRVPDPQRMIRAYAQSVATLNLLRAFATGGYAAMQRVTQWNLDFMDHSEQGDRYRELAHRVDEALGFMTAAGLTVDHPIMTTTDFWTSHECLLLPYEQSLTREDSTSGLFYDCSAHMLWVGERTRQLDGAHVEFLRGVANPLGIKVSDKMNPRDLVKLIEILNPSNKPGRITIITRMGAENMRVKLPHLIRAVRNSGQIVTWITDPMHGNTIKAPCGLKTRPFDSILAEVRAFFDVHDQEGSHPGGIHLEMTGQNVTECIGGSRTVTFDDLSDRYHTHCDPRLNASQSLELAFIIAERLRRRRMRSGVNSNLPLPPLAF

SEQ ID NO: 14 - protein, 537 aa, Zea mays
MALATNSAAAAAAVSGGVASQPRRAAVFLPLKRRTISAIHAADPSKNNGPAVPAAAGANSSASAVATPEKKPAAHGKWAVYSWRSKKALQLPEYPNAEELDAVLKTIETFPPVVFAGEARRLEERMAEAAMGRAFVLQGGDCAESFKEFHANNIRDTFRVLLQMGVVLMFGGQMPVVKVGRMAGQFAKPRSEPFEEKDGVKLPSYRGDNVNGDDFTEKSRVPDPQRMIRAYAQSVATLNLLRAFATGGYAAMQRVTQWNLDFMDHNEQGDRYRELAHRVDDALGFMTASGLTVDHPIMTTTDFWTSHECLLLPYEQALTREDSTSGLFYDCSAHMLWVGERTRQLDGAHVEFLRGVANPLGIKVSDKMNPSELVKLIDILNPSNKPGRITIITRMGAENMRVKLPHLIRAVRNAGLIVTWITDPMHGNTIKAPCGLKTRPFDSILAEVRAFFDVHDQEGSHPGGVHLEMTGQNVTECIGGSRTVTFDDLSDRYHTHCDPRLNASQSLELAFIIAERLRKRRMRSGLNSSLPLPPLAF

SEQ ID NO: 15 - protein, 540 aa, Sorghum bicolor
MALATNSAAAAAAAAAVSGGASSQPRRAAVFLPLKRRTISAIHAADPSKNNGPAVPAAAAAKSSASAVATPEKKPAAPGKWAVDSWKSKKALQLPEYPNQEELDTVLKTIETFPPVVFAGEARHLEERMAEAAMGRAFVLQGGDCAESFKEFHANNIRDTFRILLQMGAVLMFGGQVPVVKVGRMAGQFAKPRSEPFEERDGVKLPSYRGDNVNGDDFTEKSRVPDPQRMIRAYAQSVATLNLLRAFATGGYAAMQRVTQWNLDFMDHSEQGDRYRELAHRVDEALGFMTAAGLTVDHPIMTTTDFWTSHECLLLPYEQALTREDSTSGLFYDCSAHMLWVGERTRQLDGAHVEFLRGVANPLGIKVSDKMNPSDLVKLIEILNPSNKPGRITIITRMGAENMRVKLPHLIRAVRNAGLIVTWITDPMHGNTIKAPCGLKTRPFDSILAEVRAFFDVHDQEGSHPGGIHLEMTGQNVTECIGGSRTVTFDDLSDRYHTHCDPRLNASQSLELAFIIAERLRKRRMRSGLNNSLPLPPLAF

SEQ ID NO: 16 - protein, 538 aa, Zea mays
MALATNSAAAAAAAVSGGAASQPHRAATFLPLKRRTISAIHAADPSKNNGPAVPAAAAAKSSASAVATPEKNPAAPVKWAVDSWKSKKALQLPEYPNQEELDTVLKTIETFPPVVFAGEARHLEERMAEAAMGRAFILQGGDCAESFKEFHANNIRDTFRILLQMGAVLMFGGQVPVVKVGRMAGQFAKPRSEPLEERDGVKLPSYRGDNVNGDDFTEKSRVPDPQRMIRAYSQSVATLNLLRALATGGYAAMQRVTQWNLDFMDHSEQGDRYRELAHRVDEALGFMTAAGLTVDHPIMTTTDFWTSHECLLLPYEQSLTRKDSTSGLFYDCSAHMLWVGERTRQLDGAHVEFLRGVANPLGIKVSDKMNPSDLVKLIEILNPSNKPGRITIITRMGAENMRVKLPHLIRAVRNAGLIVTWITDPMHGNTIKAPCGLKTRPFDSILAEVRAFFDVHDQEGSHPGGIHLEMTGQNVTECIGGSRTVTFDDLSDRYHTHCDPRLNASQSLELAFIIAERLRKRRMRSGLNNSLPLPPLAF

SEQ ID NO: 17 - protein, 531 aa, Populus trichocarpa
MALSTLSSKSLYSNANNLFQQNQQQQSPTFSLVPGSKTLKSKSNLSISAVHAAEPTKNSVSVKESAAPVAAPGTGKWSLESWKTKKALQLPEYPDANELETVLKTIETFPPIVFAGEARSLEENLAEAAMGNAFLLQGGDCAESFKEFSANNIRDTFRILLQMGVVLMFGGQVPVIKVGRMAGQFAKPRSDPFEEKDGVKLPSYKGDNINGDAFNEKSRIPDPQRLIRAYCQSAATLNLLRAFATGGYAAMQRVTQWNLDFAQHSEQGDRYQELANRVDEALGFMAAAGLTVDHPIMTTTEFWTSHECLHLPYEQSLTRLDSTSGLYYDCSAHMLWCGERTRQLDGAHVEFLRGISNPIGIKVSNKMDPNELVKLIEIFNSNNKPGRITIIVRMGAENMRVKFPHLIRAVRRAGQIVTWVCDPMHGNTIKAPCGLKTRPFDAILAEVRAFFDVHEQEGSHPGGIHLEMTGQNVTECIGGSRTVTFDDLSSRYHTHCDPRLNASQSLELAFIIAERLRKRRIGTQRLFSLGQ

SEQ ID NO: 18 - protein, 432 aa, Populus trichocarpa
MALSTLSSKSLYSNANNLLKQNQQQPPTFSFVPGNKTHMSKSHLSISAVAAEPTKNSVAVKEYAAPGTGKWSLESWKTKKALQLPEYPDANELQSVLKTIEAFPPIVFAGEARSLEERLAEAAMGNAFLLQGGDCAESFKEFSAINIRDTFRIMLQMSVVLMFGGQMPIIKKSRIPDPQRLIRAYNQSAATLNLLRAFATGGYAAMQRVTQWNLDFAQHSEQGDRYQELAHRVDEALGFMAAAGLTVDHPIMTTTEFWTSHECLHLPYEQSLTRLDSTSGLYYDCSAHMLWCGERTRQLDGAHVEFLRGISNPIGIKVSNKMDPNELVKLIEILNSNNKPGRISIIVRMGAENMRVKLPHLIRAVRRAGQIVTWICDPIHGNTIKAPCGLKTRPFDAILAEGRAFFDVHEQEGSHPGGIHLEMTGQNVTECI

SEQ ID NO: 19 - protein, 505 aa, Oryza sativa
MPLAPCPSPPLPSSPWPARAPRRGGLLRARAVRAAPRPPSKWSLGSWRSLTALQQPEYPDKAELDEVLRTVEAFPPIVFAGEARKLEERLAEAAVGRAFLLQGGDCAESFKEFNANNIRDTFRVLLQMSVVLMFGGQMPIIKVGRMAGQFAKPRSDGFEERDGVKLPSYRGDNINGDSFDEKSRLPDPHRMIRAYSQSAATLNLLRAFATGGYAAMQRVTQWNLDFTEHSEQGDRYMELAHRVDEALGFMAAAGLTMDHPIMTTTEFWTSHECLLLPYEQALTREDSTSGLYYDCSAHFLWVGERTRQLDCAHVEFLRGIANPLGIKVSDKMDPKELVKLIDILNPQNKPGRITIITRMGPENMRVKLPHLIRAVRGAGQIVTWVTDPMHGNTMKAPCGLKTRSFDRILAEVRAFFDVHEQEGSHPGGVHLEMTGQNVTECIGGSRTVTFDDLGSRYHTHCDPRLNASQSLELAFIIAERLRKRRIASWQLNKNSHLGNIPSLGL

SEQ ID NO: 20 - protein, 508 aa, Sorghum bicolor
MPLAPSTPALPPNPALPSPCRGQGRGRPGRGALLRARAVRAAPRPPSQWSVGSWRGRPALQQPEYPDKADLNEVLRTVEAFPPIVFAGEARTLEERLAEAAVGRAFLLQGGDCAESFKEFNANNIRDTFRVLLQMSVVLMFGGQMPVVKVGRMAGQFAKPRSDGFEERDGVKLPSYRGDNINGDAFDEKSRLPDPHRMISAYSQSAATLNLLRAFATGGYAAMQRVTQWNLDFTEHSEQGDRYMELAHRVDEALGFMSAAGLTLDHPIMTTTEFWTSHECLLLPYEQALTREDSTSGLYYDCSAHFLWVGERTRQLDGAHVEFLRGIANPLGIKVSDKMDPAELVRLIDILNPENRAGRITIIARMGPENMRVKLPHLIRAVRGAGQIVTWVTDPMHGNTMKAPCGLKTRSFDRILAEVRAFFDVHEQEGSHPGGVHLEMTGQNVTECIGGSRTVTFDDLGSRYHTHCDPRLNASQSLEMAFIIAERLRKRRIASSPLYTNQLGSIRL

SEQ ID NO: 21 - protein, 511 aa, Zea mays
MALAPSPPALPPNPALPLPWWGRAGRGALLGARARVRAVRAALRPPSQWSVGSWRGRPAQQQPEYPDKADLEDVLRTVGTFPPIVFAGEARTLEERLAEAAVGRAFLLQGGDCAESFKEFNANNIRDTFRVLLQMSVVLMFGGQMPVVKVGRMAGQFAKPRSDGFEERNGLKLPSYRGDNINGDAFNGESRLPDPHRMIRAYSQSAATLNLLRAFATGGYAAMQRVAQWNLDFTENSEQGDRYMELAHRVDEALGFMSAAGLPLDHPIMTTAEFWTSHECLLLPYEQALTREDSTTGLYYDCSAHFLWVGERTRQLDGAHVEFLRGIANPLGIKVSDKMDPAELVRLIDILNPENRAGRITIITRMGPENMRVKLPHLIRAVRGAGQIVTWVTDPMHGNTMKAPCGLKTRSFDRILGEVRAFFDVHEQEGSHPGGVHLEMTGQNVTECIGGSRTVTFDDLGSRYHTHCDPRLNASQSLEMAFIIAERLRKRRMASSPLYTNQLGSIPSMGL

SEQ ID NO: 22 - protein, 517 aa, Gossypium raimondii
MVVAIPGSANLTASKPSLGLGGTTTRLSLSRPIPNLHVTKKPNSFIVASVSASSSPSTGQTNWSLDSWKSKPAQQLPEYPDADELDSVLKTIGSFPPIVFAGEARRLEDKLASAAVGKAFLLQGGDCAESFKEFNGTNIRDTFRVLLQMGIVLTFGAQMPVITVGRMAGQFAKPRSDPFEIKDGVKLPSYRGDNINGDAFDEKSRVPDPQRLIRAYLQSVGTLNLLRAFATGGYAAMQRVSQWNLDFVRNSEQGDRYMELAQRVDEALGFMAAAGITIGDPQMNTVDFWTSHECLHLPYEQALTREDSTTGLYYDCSAHLLWVGERTRQLDGAHVEFLRGISNPLGIKVSDKMDPKDLVKLCEILNPRNKPGRLTIITRMGADNMRIKLPHLIRAVRQAGLIVTWVSDPMHGNTIKAPCGLKTRPFDAIRSELRAFFDVHEQEGSYPGGVHLEMTGQNVTECIGGSNTVTFDDLNSRYHTHCDPRLNASQSLELAFAISERLRKRRLKSAKELCNDN

SEQ ID NO: 23 - protein, 511 aa, Solanum lycopersicum
MALSNTLSLSSSKSLVQSHLLHNPTPQPRFSLFPTTQHGRRHPISAVHAAEPSKTAVKQGKWSLDSWKTKKALQLPEYPDEKELESVLKTLEMNPPLVFAGEARSLEEKLGEAALGKAFLLQGGDCAESFKEFNANNIRDTFRILLQMSVVLMFGGQVPVIKVGRMAGQFAKPRSDPFEEINGVKLPSYKGDNINGDTFDEKSRIPDPHRLIRAYMQSAATLNLLRAFATGGYAAMQRVTEWNLDFVENSEQGDRYQELAHRVDEALGFMAAAGLTVDHPIMSTTDFWTSHECLLLPYEQALTREDSTSGLFYDCSAHMVWVGERTRQLDGAHVEFLRGVANPLGIKVSQKMDPKELIKLIDILNPANKPGRITVIVRMGAENMRVKLSHLVRAVRGAGQIVTWVCDPMHGNTIKAPCGLKTRAFDSILAEVRAFFDVHEQEGSHPGGIHLEMTGQNVTECIGGSRTVTYDDLGSRYHTHCDPRLNASQSLELSFIVAERLRRRRMSSQRL

SEQ ID NO: 24 - protein, 517 aa, Nicotiana benthamiana
MALSNTLSLSSSKSLVQSHILHNPSLPQPRIPLFHNPQHGRRHPISAVHAAEPGKTATASQPLKKTQWSLDSWKSKKALQLPEYPDEKELESVLKTLESNPPLVFAGEARNLEEKLGEAALGKAFLLQGGDCAESFKEFNANNIRDTFRILLQMSVVLMFGGQVPVIKVGRMAGQFAKPRSDPFEEINGVKLPSYKGDNINGDTFDEKSRIPDPHRLIRAYMQSAATLNLLRAFATGGYAAMQRVTEWNLDFVENSEQGDRYQELAHRVDEALGFMAAAGLTVDHPIMATTDFWTSHECLLLPYEQALTREDSTSGLFYDCSAHMIWVGERTRQLDGAHVEFLKGVANPLGIKVSQKMDPNELVKLIDILNPTNKPGRITVIVRMGAENMRVKLCHLIRAVRGAGQIVTWVCDPMHGNTIKAPCGLKTRAFDSILAEVRAFFDVHEQEGSHPGGIHLEMTGQNVTECIGGSRTVTYDDLGSRYHTHCDPRLNASQSLELSFIVAERLRKRRMASQRL

SEQ ID NO: 25 - protein, 537 aa, Populus trichocarpa
MALSTTLSTARPSISPLKPQNFTSTTRNPNLKPKTTSVFASLRSTPTSKPTISSNWALDSWKSKPARQLPDYPDPVELHSVLQTLTNFPPIVFAGEARKLEERIASAAVGGAFLLQGGDCAESFKEFNANNIRDTFRVLLQMGVVLTFGAQMPIIKVGRMAGQFAKPRSDPFEEKDGVKLPSYRGDNINADAFDKKSRTPDPQRLIRAYLQSVGTLNLLRAFATGGYAAMQRVSQWNLDFVEHSEQGDRFMELARRVDEALGFMAAAGLTIDHPVMNTTEFWTSHECLHLPYEQALTREDSTTGLYYDCSAHMLWVGERTRQLDGAHVEFLRGVSNPLGIKVSDKMDPKELVKLCEILNPHNRPGRLTIITRMGADNMRIKLPHLIRAVRHAGLIVTWVSDPMHGNTIKAPCGLKTRPFDSIRAELRAFFDVHDQEGSYPGGVHLEMTGQNVTECVGGSKTITFDDLNSRYHTHCDPRLNASQSLELAFAISERLRKKRLRAGDGILSGHNTGSVAHTLGGFELETVAKSSAEQKLI

SEQ ID NO: 26 - protein, 531 aa, Nicotiana benthamiana
MALTAATSSSSSRGGSALPNSCLQTLKLSPLQKPTFISSFPTTTATPSNSKIKPRINKPISAVQSPPSTTTKTAQNPDGKWSLESWKLKPAFQLPEYPDKVELESVLKTLSTYPPIVFAGEARNLEEKLGEAALGNAFLLQGGDCAESFKEFSANNIRDTFRVILQMGVVLMFGGQMPVIKVGRMAGQFAKPRSDPFEEKDGVKLPSYRGDNVNGDAFDEKSRLPDPDRMIRAYAQSVATLNLLRAFATGGYAAMQRVNQWNLDFTDHSEQGDRYRELAHRVDEAMGFMSAAGITVDHPIMTTTDFWTSHECLLLPYEQALTREDSTSGLYYDCSAHMIWVGERTRQLDGAHVEFLRGIANPLGIKVSQKMDPDELVKLIDILNPQNRPGRITVIARMGADNMRVKLPHLIRAVRGAGQIVTWVSDPMHGNTTKAPCGLKTRSFDSIRAELRAFFDVHDQEGSYPGGVHLEMTGQNVTECVGGSRTITYNDLSSRYHTHCDPRLNASQALELAFLIAERLRKRRLGPKFRL

SEQ ID NO: 27 - protein, 516 aa, Glycine max
MAATASNSLLCTKTLVPTLPSSSNNLNNSNLCSSFPRPRPRPIQALNNSVTVITDKKCEAKWVVDSWKSKKALQLPEYPEQEKLQSVLKSLEAFPPIVFAGEARNLEERLAQASMGNAFLLQGGDCAESFKEFHANNIRDTFRLLLQMSVVTMFGAQVPVIKVGRMAGQFAKPRSEAFEEKDGVKLPSYRGDNVNGDAFEEKTRIPDPERMIRSYSQSAATLNLLRAFATGGYAAMQRVSQWNLDFTQHSEQGDRYLELARRIDETLGFMAAAGLTMDHPIMKTTEFWTSHECLLLPYEQSLTRLDSTSGLYYDCSAHMLWVGERTRQLDGAHVEFLRGIANPLGIKVSDKIDPKELIRLIEILNPQNKPGRITVITRMGAENTRAKLPHLIRGVRNAGLIVCWVSDPMHGNTIKAPCGLKTRPFHSIMEEVKSFFDVHEQEGSHPGGVHLEMTGQNVTECIGGSKMVTFDDLSSRYNTHCDPRLNASQSLELAFIISERLKKNRIGSNLNNVFSL

SEQ ID NO: 28 - protein, 495 aa, Glycine max
MAVASSSSLITLKVKPCIFGSPRRSAVVRNCAKSTAGTISTSWSLDSWRAKKALQLPEYPDANELDLVLQTLSSFPPIVFAGEARNLEEKLAQAAMGNAFLLQGGDCAESFKEFTANNIRDTFRVILQMGVVLMFGGQMPVIKVGRMAGQFAKPRSDSFEEKNGVTLPSYRGDNVNGDAFDAASRIPDPQRMIRAYCQSVSTLNLLRAFATGGYAAMQRVNQWNLDFMEHSEQGDRYRELAHRVDEALGFMNVAGLTADHPIMSTTDFWTSHECLLLPYEQALTREDSTTGLHYDCSAHMLWVGERTRQLDGAHVEFLRGVANPLGIKVSDKMVPDELVKLIDILNPKNKPGRITVIVRMGAENMRVKLPHLIRAVRRAGQIVTWVSDPMHGNTIKAPSGLKTRSFDAIRAELRAFFDVHDQEGSYPGGVHLEMTGQNVTECVGGSRTITYDDLSSRYHTHCDPRLNASQSLELAFNIADRLRKRRLNSTRSLGI

SEQ ID NO: 29 - protein, 516 aa, Gossypium raimondii
MALTSPSFLASKSPLLPRRHLLPPSLKPITAVHSADPTKSTKSMAAAASTSSPSIPTQWSLESWKSKKALQLPEYPDQNDLVSVLETLSTFPPVVFAGEARSLEEKLGQAALGNAFLLQGGDCAESFKEFDANNIRDTFRVLLQMGVVLMFGGQMPIIKVGRMAGQFAKPRSDPFEEKDGVKLPSYRGDNINGDSFDEKERVPDPHRMIRAYCQSVATLNLLRAFATGGYAAIQRVTQWNLDFTEHSEQGDRYCELAHRVDEAMGFMAAAGLSVGHPVMTTTDFWTSHECLLLPYEQALTREDSTTGLYYDCSAHMLWVGERTRQLDGAHVEFLRGVANPLGIKVSDKMDPSELVRLIEILNPRNKPGRITVIVRMGAENMRVKLPHLIRAVRGAGQVVTWVSDPMHGNTIKAPCGLKTRSFDAIRAEVRAFFDVHDDEGSHPGGIHLEMTGQNVTECLGGSRTITYNDLGSRYHTHCDPRLNASQSLELAFIIAERLRRRRLASRNPLASSRSSI

SEQ ID NO: 30 - protein, 531 aa, Populus trichocarpa
MSLTSSSVIPTKSLIPSNKPHHPCFFTTKPSRSILHISAVHSADPSKSPSPIKTPAATSTKAATVAPTTNVGTGKWTVESWKSKKALQLPEYPNKEDLDSVLETLDAFPPIVFAGEARNLEERLAEAAMGNAFLLQGGDCAESFKEFNANNIRDTFRILLQMGAVLMFGGQMPVIKVGRMAGQFAKPRSDPLEEKNGVKLPSYRGDNVNGDAFDEKSRIPDPQRMIRAYCQSAATLNLLRAFATGGYAAMQRVTQWNLDFTEHSEQGDRYRELAHRVDEALGFMAAAGLTVDHPIMTTTEFWTSHECLLLPYEQSLTRLDSTSGLYYDCSAHFLWAGERTRQLDGAHVEFLRGVANPLGIKVSDKMDPNELVKLIEILNPQNKPGRITIITRMGAENMRVKLPHLIRAVRRAGQIVTWVSDPMHGNTIKAPCGLKTRPFDAIRAEVRAFFDVHEQEGSHPGGVHLEMTGQNVTECIGGSRPVTFDDLSSRYHTHCDPRLNASQSLELAFIIAERLRKRRIGLQPSIAPALL

SEQ ID NO: 31 - protein, 531 aa, Populus trichocarpa
MSLTSSSIIPSKSLIPSNKPHQPCFFTTKPSRSTLRISAVHSADPSKSPPPIKTPAATSTKATTVAPTVNVGAGKWTVESWKSKKALQLPEYPDKEDFDSVLKTLDAFPPIVFAGEARSLEEKLAEAAMGNAFLLQGGDCAESFKEFNANNIRDTFRILLQMGVVLMFGGQMPVIKVGRLAGQFAKPRSDPFEEKDGVKLPSYRGDNVNGDAFDEKSRIPDPQRMIRAYCQSAATLNLLRAFATGGYAAMQRVTQWNLDFTEHSEQGDRYRELAHRVDEALGFMAAAGLTVDHPIMTTTEFWTSHECLLLPYEQSLTRLDSTSGLYYDCSAHFLWVGERTRQLDGAHVEFLRGVANPLGIKVSDKMNPNELVKLIEIFNPQNKPGRITIITRMGAENMRVKLPHLIRAVRRAGQIVTWVSDPMHGNTIKAPCGLKTRPFDSIRAEVRAFFDVHEQEGSHPGGVHLEMTGQNVTECIGGSRTVTFDDLSSRYHTHCDPRLNASQSLELAFIIAERLRRRRIGSQSTVAPALL

SEQ ID NO: 32 - protein, 535 aa, Nicotiana benthamiana
MALSTSSTNSLLPNKSLIQIQPLLHSPKHALPTNSTKPVRPISAVYSPDSSKTPKPSPPAATSSSTAAAAVTGTKTDKVSKKWSVDSWRSKKALQLPEYPNKDDLESVLKTIEDFPPIVFAGEARSLEERLGEAAMGKAFLLQGGDCAESFKEFNAVNIRDTFRIILQMGAVLMFGGQMPVVKVGRMAGQFAKPRSDPSEEKDGVKLPSYRGDNVNGDAFDEKSRTPDPQRMIRAYCQSAATLNLLRAFAKGGYAAMQRINQWNLDFTEHSEQGDRYRELAHRVDEALGFMAAAGLTTDHPIMKTTEFWTSHECLLLPYEQSLTRLDSTSGLYYDCSAHFLWAGERTRQLDGAHVEFLRGIANPLGIKVSDKMDPNELVKLIDILNPNNKPGRITIITRMGAENMRVRLPHLIRAVRRAGQIVTWVSDPMHGNTIKAPCGLKTRPFDAIRAEVRAFFDVHEQEGSHPGGVHLEMTGQNVTECIGGSRTVTFDDLSSRYHTHCDPRLNASQSLELAFIIAERLRKRRLGFQSSLSY

SEQ ID NO: 33 - protein, 541 aa, Solanum lycopersicum
MALSTNTTTNSLLSNKSLLQNQPLLSSPSKNAFFSNKSTKTVRFVQPIAAVHSSDSNKNPIVSDKPSKSSPPAATATTAPAPAVTKTEWAVDSWKSKKALQLPEYPDQEELRSVLKTIDEFPPIVFAGEARSLEERLGEAAMGRAFLLQGGDCAESFKEFNANNIRDTFRILLQMGAVLMFGGQMPVIKVGRMAGQFAKPRSDSFEEKDGVKLPSYRGDNVNGDAFDVKSRTPDPQRLIRAYCQSAATLNLLRAFATGGYAAMQRINQWNLDFTEHSEQGDRYRELASRVDEALGFMTAAGLTMDHPIMKTTEFWTSHECLLLPYEQSLTRRDSTSGLHYDCSAHFLWVGERTRQLDGAHVEFLRGIANPLGIKVSDKMDPSALVKLIEILNPQNKAGRITIITRMGAENMRVKLPHLIRAVRRAGQIVTWVSDPMHGNTIKAPCGLKTRPFDSIRAEVRAFFDVHDQEGSHPGGVHLEMTGQNVTECIGGSRTVTFDDLSSRYHTHCDPRLNASQSLELSFIIAERLRKRRLGSQSTLGQ

SEQ ID NO: 34 - protein, 542 aa, Nicotiana benthamiana
MALSSSSTTNSLLSDKSQLVQNQFLLLSPLKNASFTTNSTKAVRFIQPISAVHSSDSSKNPIVSDKPSPKPSPPAATVTAAAATVTKTEWALGSWKSKKALQLPEYPNQEELQSVLKTIEEFPPIVFAGEARSLEERLGEAAMGRAFLLQGGDCAESFKEFNANNIRDTFRILLQMGAVLMFGGQMPVIKVGRMAGQFAKPRSDNYEEKNGVKLPSYRGDNVNGDAFDAKSRTPDPQRLIRAYCQSAATLNLLRAFATGGYAAMQRINQWNLDFTEHSEQGDRYRELANRVDEALGFMAAAGLTVDHPIMKTTEFWTSHECLLLPYEQSLTRLDSTSGLYYDCSAHFLWVGERTRQLDGAHVEFLRGVANPLGIKVSDKMDPSALVKLIEILNPDNKAGRITIITRMGAENMRVKLPHLIRAVRRAGQIVTWVSDPMHGNTIKAPCGLKTRPFDSIRAEVRAFFDVHEQEGSHPGGVHLEMTGQNVTECIGGSRTVTFDDLSSRYHTHCDPRLNASQSLELAFIIAERLRKRRLGSRSVLGQ

SEQ ID NO: 35 - protein, 539 aa, Gossypium raimondii
MALTNSSILPTNSILQSQPSTPFSKAKPLSVLRISAVHSSDSPKNPVVSDKSVKQPATAPLAATKSATAPTTAPPKNAVPGKWTVDSWKSKKALQLPEYPDQAELESVLKTLDAFPPIVFAGEARSLEEKLGEAAMGNAFLLQGGDCAESFKEFNANNIRDTFRILLQMGAVLMFGGQMPVIKVGRMAGQFAKPRSDPFEEKNGVKLPSYRGDNVNGDSFDEKSRIPDPQRMIRAYCQSAATLNLLRAFATGGYAAMQRVTQWNLDFTEHSEQGDRYRELASRVDEALGFMAAAGLTVDHPIMTTTEFWTSHECLLLPYEQSLTRLDSTSGRYYDCSAHFLWAGERTRQLDGAHVEFLRGVANPLGIKVSDKMDPNELVKLIEILNPQNKAGRITVITRMGAENMRVKLPHLIRAVRRAGQIVTWVSDPMHGNTIKAPCGLKTRPFDSIRAEVRAFFDVHEQEGSHPGGVHLEMTGQNVTECIGGSRTVTFDDLGSRYHTHCDPRLNASQSLELAFIIAERLRKRRINSPQLQPLITGL

SEQ ID NO: 36 - protein, 532 aa, Glycine max
MAISSTSNSLIPTKSLIPQSHPLIPNTRPALRPKPGPSPSIFAVHAAEPAKNPVVTDKPKPQAQQPPPASARATKWAVDSWKSKKALQLPEYPSQEELESVLKTLEAFPPIVFAGEARTLEEHLAEAAMGNAFLLQGGDCAESFKEFNANNIRDTFRIILQMSVVMMFGGQMPVIKVGRMAGQFAKPRSDSFEEKNGVKLPSYRGDNINGDSFDEKSRIPDPQRMIRAYCQAAATLNLLRAFATGGYAAMQRVTQWNLDFTDHSEQGDRYRELANRVDEALGFMAAAGLTVDHPIMRTTEFWTSHECLLLPYEQSLTRLDSTSGLYYDCSAHMIWVGERTRQLDGAHVEFLRGVANPLGIKVSDKMDPNELVKLIEILNPQNKAGRITVITRMGAENMRVKLPHLIRAVRRAGQIVTWVSDPMHGNTIKAPCGLKTRPFDSIRAEVRAFFDVHEQEGSHPGGVHLEMTGQNVTECIGGSRTVTFDDLSSRYHTHCDPRLNASQSLELAFIIAERLRKSRIRSQQPLAPLGVL

SEQ ID NO: 37 - protein, 403 aa, Glycine max
MGNAFLLQGGDCAESFKEFNANNIRDTFRIILQMSVVMMFGGQMPVIKVGRMAGQFAKPRSDSFEEKNGVKLPSYRGDNINGDSFDEKSRIPDPQRMIRAYCQAAATLNLLRAFATGGYAAMQRVTQWNLDFTDHSEQGDRYRELANRVDEALGFMAAAGLTVDHPIMRTTEFWTSHECLLLPYEQSLTRLDSTSGLYYDCSAHMLWVGERTRQLDGAHVEFLRGVANPLGIKVSDKMDPNELVRLIEILNPQNKPGRITVITRMGAENMRVKLPHLIRAVRRAGQIVTWVSDPMHGNTIKAPCGLKTRPFDFIRAEVRAFFDVHEQEGSHPGGVHLEMTGQNVTECIGGSRTVTFDDLSSRYHTHCDPRLNASQSLELAFIIAERLRKSRIRSQQPLAPLGV

SEQ ID NO: 38 - DNA, 1578 bp, Arabidopsis thaliana
  source 1..1578: mol_type = other DNA
atggctctttccaacgcctcctctctctccaccagatctatctacggcggtgatctttctcaccgtccaagcaaccgtcaatcatccttcacatttcatcccgccgtcaacaccaagcctaaatccgttaacctcgtcacggctgtacacgcggctgagccagctagaaacgctgtttcagtcaaagaatccgttgcgtcttcttcgtctggtgcattgaaatggactccagagagctggaaactgaagaaggctctgcaacttcctgattatcctaacgccaatgagcttgagtctgtgcttaaaacgatagaggcttttcctccgattgtgttcgccggagaagctaggaatcttgaagagagattggctgatgctgctgttggtaaagcttttcttctccaaggaggtgattgtgctgagagtttcaaggagttcaatgctactaatatcagagacaccttcagggttctgcttcagatgagtattgttcttacattcggtggccaggttccggtcattaaggttgggaggatggctggtcaatttgcaaagcctagatctgatgcatttgaggagaaggatggagtgaaattaccaagctacaagggagataacatcaatggtgatacttttgatgagaaatccaggattcctgatcctaataggatgattcgagcttacactcaatctgcagctactttgaatcttcttagagcctttgccactggaggttacgctgctattcaaagagttactcaatggaaccttgattttgttgaacaaagtgagcaagctgacaggtaccaggaattagctaatagggttgatgaggccttggggttcatgtctgcatgtggacttggcacggatcatccacttatgactacaactgatttctacacatcccatgagtgtttgcttctgccttatgaacagtcccttacaaggttggattcaacttctggtctctactatgattgttctgcacacatggtctggtgcggagagcgtaccagacaattggatggcgctcatgtcgagtttctcagggggattgctaatcctctgggcattaaggtgagcaacaaaatggatccctttgagctggttaagctagtagaaatcctgaatcctaataacaagcctggaagaatcactgtaattgtgagaatgggtgccgagaacatgagagttaagcttccccacctgatcagagcagtccgcagatcaggccagattgtgacatgggtctgcgatccaatgcatggaaacaccatcaaagcaccttgcggtcttaaaacacgtgcctttgactcaatactggctgaagtccgagcattccttgatgtgcacgagcaagaaggaagccacgcgggcggtatccatctcgagatgacaggtcagaacgtgacagaatgcattggagggtcccgcactgtgacctacgatgacctcagttctcgctaccacacacactgtgacccaaggctgaacgcgtcacagtctcttgaacttgccttcattgttgcagaacggctcaggaagagaagaacgggtagccagcgtgtgtcttga

SEQ ID NO: 39 - DNA, 1524 bp, Arabidopsis thaliana
  source 1..1524: mol_type = other DNA
atggtgactctaaacgcttcttctcctctcacgaccaagtcgttcctcccctaccgtcacgctcctcgccgtccaatctctttctcccctgtcttcgccgttcattcgactgaccccaagaaatctacccaatcagcctccgcttcggttaaatggagtctagagagttggaagtcgaagaaagctttgcaattgccggattatcctgatcagaaggatgttgattcggttctacagacgctttcttcttttcctcctatagttttcgctggtgaggctaggaaactagaggataagcttggtcaagcggctatgggtcaagcctttatgcttcaaggtggtgattgtgctgagagtttcaaggaatttaacgctaataacattagagacacctttagggttcttcttcagatgggtgttgttctcatgttcggtggccagttaccagttatcaaggtgggaagaatggctggtcagtttgcgaagccgagatcagacccatttgaggagaaagatggtgtgaagctgccgagttacagaggagataacataaatggtgatgcttttgatgagaaatcgaggattcctgatcctcataggatggttagagcgtacacacagtctgtggctacgttgaatctcttgagagcatttgctactggaggttatgcagctatgcagagagttagccagtggaaccttgatttcacgcaacatagtgaacagggtgacaggtaccgtgaattggctaatagagttgatgaggctttgggattcatgggtgcagctggacttactagtgctcacccgatcatgactactactgagttttggacatcccatgagtgtttgttattgccttatgagcaagcactcacaagagaggattcaacatctggactttactatgattgctctgcgcacatgctttgggttggagaacgaactcgccaacttgatggtgctcatgttgagtttctgaggggaatcgctaaccccctcggaatcaaggtgagtgataaaatggtccctagtgaactggttaagctgatagagatactaaaccctcagaacaagcctggaaggattacggttatagtgagaatgggagctgagaatatgcgggtcaagcttcctaatttgatcagagcagtccgtggagccggtcagattgtgacttgggttagtgatccaatgcacggaaacacaatcatggctcctggtgggctaaaaactcgttctttcgatgcaatcagggcggaattgagagcgttcttcgacgtccatgatcaagaagggagtttccctggcggggttcatttagaaatgactggtcaaaacgtgacagaatgtgtcggagggtcacgcaccatcacttacaacgatctaagctcacgctaccacactcactgtgacccaagactcaacgcatctcagtctctggagcttgcattcatcattgcagagcgtctgcgaaagagaaggcttggttccgggaatcttccgtcatctattggagtctag

SEQ ID NO: 40 - DNA, 1584 bp, Arabidopsis thaliana
  source 1..1584: mol_type = other DNA
atggcgcttatgaatggaagcatgaatctatcatcggtcaaatcatcgatgatcaaccaccgtcaaccaaacttctcctccgccgtctccagacccacttctttccggatctccgccgtgcaaaccgacccaaaaacaccagctgcttcatcggcgtcggcggcgacgacgactccggcaacgttgacgaagcccgttggagttaatgtgggtaaaggaaaatgggcaccagagagttggaggacgaagaaagctttacagcaaccggactatccagatttagctgcgcttgaagctgtgctagagacgatcgaagcttttcctccgatcgtgttcgccggagaagctagattgcttgaggaacgattaggacaagcagcgatgggtgaagcgtttctgttacaaggaggagattgtgcagagagtttcaaagagtttaatgcgaataacattcgtgatactttcaggattcttcttcagatgggtgctgtgttgatgttcggaggtcaagtccctgttgttaaggttgggagaatggcgggtcaatttgcgaaaccaagatcggattcgtttgaggagaaagatggagtgaaattgcctagttacagaggagataacattaatggagatgcgtttgattcgaaatctaggattcctgatccacagaggatgattagagcttattgtcaatctgcagctactttgaatcttttgagagcttttgctactggtggttatgctgctatgcagagagttactcaatggaatcttgatttcactgaacgtagtgagcaaggagacaggtaccgtgaactagctaaccgtgttgatgaagcgcttggtttcatgcacgctgctggattaacgctcgatcatcccataatgcaaacaaccgacttctggacttctcatgaatgtttgctcttgccttatgaacaatcactaacaaggcttgactcaacctctggtctctactacgattgttctgcccatatgatttgggtcggtgagcgaaccagacagctagatggtgcccatgtcgagttcttaagaggtgttgctaatcctcttggaatcaaggtgagtgataaaatggatccaaaggagctagtgaagttgattgagatcttgaatgctgacaataagcctggaaggattacaataatcacaagaatgggagctgagaatatgagagtgaagcttcctcatttgatcagagaagtgcgacgtgctggacagattgtgacttgggtcagtgatccaatgcacggaaacaccattaaggctccttgtggtctcaagactcgtccctttgacgccatcttggctgaagtgagagctttcttcgacgtgcacgagcaagaaggtagccaccccggaggaattcacctagaaatgactggtcagaacgtgaccgagtgcatcggtggttcacgcacagtcacattcgacgacttgggttcacgttaccacacgcactgtgatcctcgccttaatgcttcacagtctcttgagctctccttcatcatcgccgaacgtcttagaaagagacgcatcaagtctcaaaaggcttttgccctctaa

SEQ ID NO: 41 - DNA, 1389 bp, Mycobacterium tuberculosis
  source 1..1389: mol_type = other DNA
atgaactggaccgtcgacataccgatcgaccagctcccgtcgcttccgccgctgccgactgacctgcgaacacgactggacgccgcgctggccaagccggcggcccagcaacccacctggccggccgaccaggcgttggcgatgcgcacggtgttggagagcgtgccacccgtgacggtgccgtccgagatcgtccggctgcaagagcagctggcccaggtggcaaagggtgaggctttcctgctgcagggcggcgactgcgctgagacattcatggacaacaccgaaccccatattcggggcaatgtccgtgccttgttgcagatggccgtggtgctgacctacggcgccagcatgccagtggtgaaggtggcccgcatcgccggtcagtacgcgaagcctcggtcagccgacattgacgcgctgggtctgcggtcttaccgcggcgacatgatcaacggcttcgccccggacgccgcggcgcgcgagcacgacccgtcgcggctggtccgggcttacgctaacgccagtgcggcgatgaacttggtgcgtgcgctgacttcgtcgggcctggcgtcgctgcatctggttcacgactggaaccgggaattcgtccggacctcgccggccggcgcgcgctatgaggcgctggccaccgagatcgatcgtgggctgcggttcatgagtgcctgtggggtggccgaccgcaatctacagaccgccgaaatctatgccagccatgaagctttggtgctcgactacgagcgcgccatgttgaggttgtccgacggtgacgacggtgaaccacaactgtttgacctgtccgcacacaccgtgtggatcggcgagcggacccgacaaatcgatggcgcgcacatcgcgtttgcccaggtgattgctaatccggtcggggtcaagttgggccccaacatgaccccggaactggccgtggagtacgtcgagcggctcgacccgcacaataagccgggccggctgacgttggtgagcaggatgggcaaccacaaggtccgcgatctgttgccaccgatcgtggagaaggtccaggccaccgggcatcaggtgatctggcaatgcgacccgatgcacggcaacacccacgagtcgtccacggggttcaagacccgtcacttcgatcgcattgtcgatgaagtgcagggttttttcgaggtgcatcgtgccctcggaacccatccgggcggcatccatgtcgagattaccggcgagaacgtcaccgaatgtcttggtggcgcgcaagacatttcggagacggacttggccggccgctatgagacggcatgtgatccgcggctgaacacccagcagtcgctggagttggcgttcctggtcgcggagatgctgcgggactag

SEQ ID NO: 42 - DNA, 1347 bp, Pseudomonas aeruginosa
  source 1..1347: mol_type = other DNA
atgagccagtcctggagccccgagagctggagggccaagccgatccagcaacaacccgagtaccccgatgccgcgcacctggccagggtcgagcagaccctcgccggatacccgccgctggtcttcgccggcgaggctcgcgagctgcgccggcagttcgccgaagtcaccgccggacgcgccttcttgctccagggcggggattgcgcggaaagcttcgccgaattttccgcggcgaagatccgcgataccttcaaggtactgttgcagatggcggtggtaatgaccttcgccgccggctgcccggtagtgaaggtcgggcgcatggccggccagttcgccaagccgcgctcctccggcgacgaaacgcagaacggcgtgaccctgcccgcctatcgcggcgacatcgtcaacggcatcggcttcgacgagaagagccgcgtgccggacccggagcgcctgttgcaggcctaccaccagtccaccgccagcctcaacctgttgcgcgccttcgcccagggcggcttcgccgacctgcaccaggtgcaccgctggaacctcgatttcatcgccaactcggcgctggccgagcgctaccagcaactcgccgaccgcatcgacgagaccctcgccttcatgcgcgcctgcggcctggacagtgcgccgcagctccgcgaaaccagcttcttcaccgcccacgaagcgctcctgctgaactacgaggaagccctgacccgccgcgacagcctgaccggcgagtggtacgactgctcggcgcacatgctctggatcggcgaccgcacccgccagatcgacggcgctcatgtcgagatgctgcgcggcgtgggcaatccgatcggggtcaaggtcgggccgagcatggacagcgaggagctgatccgcctgatcgatattctcaacccggacaacgatccgggccgcctcaacctgatcgtgcggatgggtgccgacaaggtcggcgaccacctgccgcgcctgatccaggccatccagcgcgagggccgccaggtgctgtggagttccgatcctatgcacggcaataccatcaaggcctccagcggctacaagacccgcgacttcgcccgcgtgctcgccgaagtccgccagttcttcgaggtgcaccaggccgaaggcagctacgccggcggcatccacatcgagatgaccgggcagaacgtcaccgagtgcatcggcggttcgcgaccgatcaccgaagacggcctcagcgaccgctaccacacccactgcgacccgcggctgaacgccgaccagtcgctggagctggccttcctgatcgccgaaaccctgaagcaggtgcgccgctga

SEQ ID NO: 43 - DNA, 1527 bp, Oryza sativa
  source 1..1527: mol_type = other DNA
atgacgtgtcactctgccatggcggctctcaccgttgggcacgcggcgatcgtccatgccaccacgcgtctcgaggacgcccggtccaccggccgtcggcggcggcggcgggggatgatcacggtgcgcgcggcggcggcggcgacgagcgggtgggagccgggcagctggagggcgcggccggcgaggcaaatcccggagtacccggacgcggcggcgctggagggcgcggagcgcgagctggcgtcgttcccgccgctggtgttcgccggggaggcgcggaagctggaggagcggctcggggacgccgccatggggcgggccttcctcctgcagggcggcgactgcgccgagagcttcaaagagttcgccgccaacaacatccgcgacacctttcgcctcatgctccagatggccgtcgtcctcacctttggcggccagatgcccaccatcaaggttggaaggatggctggccaatttgcaaagccaagatcaaacccaactgagactatagatggagtgacacttccttcctatcgaggtgatattattaacagcgatggttttgatgagaagtcgcgtgcaccagatcctgaaaggttaattagagcctacagccagtccgcgagcaccctgaatcttctgagaggatttgctcatggagggtatgcagatctgcagagagtcacccagtggaatcttgacttcttgagggacagcacgcaaggggacaggtatatggagctgtccgagagggttcacgatgccatcggatttatggttgctgctggtctgactcctcagcatcccatcatgacgacggctgaattctggacatctcatgagtgccttcatttgccatatgagcaagcattgactagggtggactccatttctgggctttactacgattgctctgctcatatgctttgggttggggagaggactcgacaactggatggtgctcatgttgaattccttcgtggtatttccaaccctctaggtgtaaaggtgagtgacaagctcgaaccttcagagcttgttaaattgtgtgagattttgaatcctcacaacaagcccggaagactgacaatcatcacaagaatgggtgctgagaacacgcgcgttaagctcccacatatgatcagagcagtccgccaagctgggttgattgtcacttgggtcagtgatcccatgcatgggaacaccatcagcgcaccatgtggtctcaagacaagatcattcgacgcgatcaggtgcgagctgagggctttcttcgatgtccatgagcaagagggaagctaccctggagggattcacctggagatgacagggcagaacgttacagagtgcattggtggatccaagacggtgacccttgatgatctcagctctcgctaccgcacgcactgcgaccccaggctgaatgcatcgcagtcgcttgaactggctttcgccattgctgacaggctaaggaagaaacgagacagggcttggaataggttggtgtacagggcggtagcttaa

SEQ ID NO: 44 - DNA, 1500 bp, Sorghum bicolor
  source 1..1500: mol_type = other DNA
atggcgcctctcgccaccgcgccgcctcctactccccatgccccgcgctctctcgttccacgggtaaggccgcggcgagggctgacggcagtgcgcgcggcctcacagggaagaacaacggatgggtggacgccgggaagctggcgtgcgcgccctgcgcggcagatcccggagtaccccgacacggcggcgctggaggctacagagcgaactctggcggagtttccaccgctagtgttcacgggtgaggtgcggaagctggaggagcggctcggggaggcggccatgggtcgggcattcctcctgcagggcggcgactgcgccgagagctttagggagttcaatgcacagaaaatccgggacaccttccgactcatactgcagatggccgtcgttctcaccttcggcggacagatgcccaccatcaaggtgggaaggatgggtggtcaatttgcgaagccaagatcgaacccaactgagactgttgatggggtaacccttccctcctatagaggggatatcatcaacgatcaggcttttgatgagaaatctcgtgtaccagatcctgaaaggttgattagagcctatacccagtctgcgagcaccctgaatcttctcagagcatttgcccatggaggttttgcggatcttcagagagtcacccagtggaatctcgactttttgaggcacagcacacaaggagacaggtatctggagctttctcagagggttcatgaagccattgggtttatggttgccgctgggctgacccctcaacaccccatcatgaccacagctgagttatggacatcccatgagtcccttcacttaccatacgagcaagcactgactagggaggattccattagtggccggtactatgactgttccgcacacatgctttgggtcggtgagaggactcggcagctggatggtgctcatgttgaattccttcgtggtatttcaaatcctctgggtataaaggtaagtgataagcttgacccatcagagcttgtgaagttgtgtgagactctgaatcctcacaacaagcctgggcgattgacgattatcacaagaatgggggctgagaacatgcgtgttaagcttccacaaatgatcagagcagtgcgccaagctgggatgattgttacttgggttagtgatcctatgcacggaaacactatcagtgcaccatgcggactgaagacaagatcatttgattcaatcatggctgaactcagggctttcttcgatgttcatgggctagagggaagctaccctggaggagttcacctggagatgacggggcagaatgttacagagtgtatcgggggatctaaggcggtgacatttgatgatctcagtgcccgctaccacacacactgtgaccccagactgaacgcgtcgcagtccctcgagctggctttcgccattgctgacaggttaaggaacaagagagacaagacatggcataatttgacatccagagttgttgcttaa

SEQ ID NO: 45 - DNA, 768 bp, Sorghum bicolor
  source 1..768: mol_type = other DNA
atgcctccgctgctcacctcacccgcctccgcctcctcccctctcctcctcgcctcccgcgtcggcgctctccaccaccgcgccgccgcctcggtccacttcctcgcgccgcgcaggaagacccgcccgagcccgaccccgggccccgcgatgagctggcttgggaagctgggcctgagcgggctgggcggcagcccgcgcgcgtcggaggcgtcggcggcgctggcgcaggggcccgacgaggaccagcccgcgcccgggaacgagttcgcgcagttcggcgccggctgcttctggggcgtggagctcgcgttccagcgcgtgccgggagtcacgcgcaccgaggtcggctacagccagggcaacctccacgagccgacctacgaggacgtctgcaccggcgccacgaaccacaacgaggtcgtccgcgtgcagtacgaccccgccgcctgcaagttcgacgacctcctcgacaccttctgggcgcgccacgaccccaccacgcccaaccgacagggcaatgatgttggaacccagtataggtcagggatctacttctacaccccggagcaagagaaggcggcaagagaatccctggagaagcagcagaagcttctgaaccggacaatcgtcactgagattctaccggcgaagaggttctatagagcagaagagtaccatcagcagtacctggagaagggtggccgcttcggcttcaggcagtctgcatccaagggctgcaatgaccccatccgttgctacggatga

SEQ ID NO: 46 - DNA, 1653 bp, Zea mays
  source 1..1653: mol_type = other DNA
atggccctcgccaccaacgccgccgccgcagccgccatctccggagggtccgcctcacagccccgccgcgcgtcgccgtcgctgctcctgctcaggcgccgctgcgccgtcggcgccgtacacgccgcggagccgtcgaagaaccccggcgtcgtcccggccgccgcgaaggcctcgacgccgacggtcgtgcccgagaacgacaccgtcccggccccgtcccttggcccgaacgcgtcgttgcgcgcgaggtgggcggtggacagctggaggacgaagaaggccctgcagcagccagaatacccgaaccagacagagctcgaggaggcggtcaacacgatcgagaagtctccgccgatcgtctttgccggggaggcgcgtcacctggaggagcgcctcgccgacgccgccgtcggccgcgcattcctcctccagggcggcgactgcgccgaaagttttaaagagttcaccagtgctaatatccgcgacaccttccgcgtcatcctgcagatggccgtcgtgctgacgttcggcggccaaatgcccgttgtcaaggtcggtaggatggccggccaattcgcgaagccgaggtcggacgcgttcgaggagaggggcggcgtgaagctgcccagctaccggggcgacatcatcaacggcgaggctttcgacgagaagagccgcgtccccgacccacagaggatgctcaaggcgtactaccaatccgcttccaagctcaacctgctccgcgccttcgccaccggtggctacgcggacatccaacgcgtgacacagtggaaccttgacttcactaaacacagcgagcagggcgacaggtaccgtgaattggcacatcgggttgatgatgcccttggattcatgggtgcagctgggctaacagtggaccagcctttgacgacgatgatcgagttctggacctcacatgagtgcctcctcctaccttacgagcaagccttaacccggcaagactccaccaccggtcttttctacgactgctccgcccacatgctctgggtcggcgagcgcacccggcagcttgatggcgcccatgtggagttcctgagggggatcgccaacccccttggcatcaaggtgagcgacaagatggagcccggcgagctggtgaagctgatcgacatactgaacccgacgaacaagcccgggaggatcaccgtcatcacaaggatgggggcagagaacatcagggtcaagttacctcaccttatccgcgcggtccgccaggctggacagagtgtcacctggatcactgacccgatgcacgggaacaccatcaagactccttgcggacgaaagactcggccatttgactccattctggccgaggtacgggccttcttcgatgtgcacgaccaagagggaagccagcctggaggtgtccaccttgagatgaccggtcagaacgtgaccgaatgcacgggcgggtcaagcaacgtcaccttcgacaacctgagagaccgctaccacacgcaatgcgaccccaggctgaacgcgtcccagtccctggagctcgccttcatactcgccgagaagcttaggaagcggaggatgcggcggtcgtcggtggcgtctgggctcggcggcagcatcttgcccttgccgccctttggcttttga

SEQ ID NO: 47 - DNA, 1668 bp, Oryza sativa
  source 1..1668: mol_type = other DNA
atgtcgctcgccaccagctcctcgatggccggtggggcggcggtggttccacgcagcgcgacggcgacgacggcctcggcgttcgtcacgatgaagcggcgcgccacggccgtgcgcgccgtacacgcggcggagccgtcgaagaacccgcccgtcggcgtcccgtccgcggcgaagacgtcgtcgccgtcggtggcggcgccggagaaggcccccgtggcggccgcgcccgcgcctgtggctccggctccggcggccacgaagcaggtggcgccggcgaggtgggcggtggacagctggaggacgaagaaggcgctgcagctgccggagtaccccaacgcggcggagctggaggcggtgctcaagacgatcgaggcgttcccgccgatcgtgttcgccggggaggcgaggcacctggaggagcgcctcgccgacgccgccatgggccgcgccttcctcctccagggcggcgactgcgccgagagcttcaaggagttcaacggcaacaacatccgcgacaccttccgcgtcctcctccagatgtccgccgtcctcaccttcggcggccaaatgcccgtcatcaaggttgggagaatggcggggcaattcgcgaagccgaggtcggaggcgttcgaggagagggacggggtgaagctgccgagctacaggggcgacaacatcaatggcgacgcgttcaacgagaagagccgcatccctgacccgcagaggatggtccgggcctatgcgcagtccgccgccacgctcaacctcctccgcgctttcgccaccggaggatacgccgccatgcagcgcgtgacgcagtggaacctcgatttcacccagcacagcgagcagggcgacaggtaccgtgaattggcacacagggttgatgaagcccttggctttatgtctgctgctgggctaacagtggaccacccgttgatgacaagtactgatttctggacctcacatgagtgccttctcctaccctatgaacaatctctgacccgtcaggactccacaactggtcatttctacgactgctctgcccacatgctatgggtgggcgaacgcactcgccagctcgatggtgctcatgttgagttcctcaggggtgtggccaaccctcttggcatcaaggtgagtgacaaaatgaacccaaccgagttggtgaagctgattgagattttgaacccatcaaacaagcctgggagaattaccatcatcacaaggatgggggcagagaacatgagggtcaagttacctcatcttatccgtgcggtccgccacgctggacaaattgtcacatggattactgatccaatgcacgggaacaccatcaaggctccttgtggtctgaagacacgcccctttgactccattctggccgaggtacgagcattcttcgatgtgcatgatcaagaaggaagccacccaggaggtgtccacctcgagatgactgggcagaacgtaaccgagtgcatcggcggatcccgaaccgtcaccttcgatgacctgggtgaccgctaccacacccactgcgacccgaggctgaacgcgtcccagtcgctggagctctccttcatcatcgccgagaggctgaggaggaagaggatccggtcgtcgaagctgaacaacatgttgccgttgccacccttcggtgtctga

SEQ ID NO: 48 - DNA, 1656 bp, Sorghum bicolor
  source 1..1656: mol_type = other DNA
atggcgctctccaccaacgccgccgccgccgccgccgccatctccggcgggtccgcgtccgcgtcgcagccggcccgtcgcacgccctcgtctctcctcccgctgacgcgccgccgccgcgccgtcgtccgcgccgtgcacgccgcggagccgtcgaagaaccccggcgtcgtcgtcccggccgccgccaaggcctcgtcgccgacgacggtcgcgcctgagaacgacgcggcccctgctccggccccggcccgtgcgccggcgaagtgggcggtggacagctggaggacgaagaaagcgctacagctgccggagtatccgaacccggcagagctagaggcggtgctcaagaccatcgaggcgttcccacccatcgtcttcgctggggaggcgcgccacctggaagagcgcctcgccgacgccgccatgggacgcgccttcctcctccagggcggcgactgcgctgagagcttcaaggagttcaacagcaacaacatccgcgacaccttccgcgtcctcctgcagatgtccgccgttctcatgttcggcgcccagatgcccgtcgtcaaggttggtaggatggccggccaattcgcgaagccaaggtcggatccgttcgaggttagagatggcgtcaagctgcccagctaccggggcgataacattaatggcgaggcattcgacgagaagagccgtgtccccgacccacagaggatgatccgcgcgtatgcgcagtctgccgcaacgctgaacttgctccgcgccttcgccaccggaggctacgctgccatgcagcgcgtcacgcagtggaacctcgatttcaccgaacacagcgagcagggcgacaggtaccgtgaattggcacatcgggttgatgaagcccttggcttcatgtctgcagctggcctaacaccggaccaccctttgacgacgaccactgagttctggacctcacatgagtgcctcctcctaccttacgagcaagctttaacccgtcaagactccacctctggtcttttctacgattgctctgcccacatgctctgggttggtgagcgcactcgccagcttgatggcgcccatgttgagttcctcaggggtattgccaaccctcttggcatcaaggtaagtgacaaaatgaaccccagcgacttggtgaagctgattgatatactgaacccaacaaacaagcccgggaggatcaccgttattacaaggatgggggcagagaacatgagggtgaagttacctcaccttatccgtgcggtccgccaggctggacaaattgtcacctggatcactgacccgatgcacggcaacaccatcaaggctccttgtggtctaaagactcgtccatttgacaatattctggctgaggtacgggccttctttgatgtgcacgagcaagaggggagccaccctggaggtgtccaccttgagatgactgggcaaaacgtaactgaatgcattggcgggtcacgcaccgtgaccttcgatgacctgagcgaccgctaccacacccactgtgaccccaggctgaacgcgtcccagtctctggagctcgcgttcattatcgccgagaggctgaggaagaggaggatccggtcatcgtccgggctcaacaacatcttgcccttgccgccttttggtttctga

SEQ ID NO: 49 - DNA, 1614 bp, Oryza sativa
  source 1..1614: mol_type = other DNA
atggcgctcgccaccaactccgccgctgtttccggtggcgctgcggccgcggcgtcgtcggcgccccagccgcggctcgccgccacgttcctcccgatgaggaggcgaaccgtctcggcggtccacgcggccgacccggcgaagagcaacgggcccgtgcaggccgcggcgaaggcctcgtccccgtcgacggtggcggcgccggagaagaagccggtggggttggggaagtggacggtggatagctggaaggcgaagaaggcgctgcagctgcccgagtacccgagccaggaggagctcgactccgtgctcaagacgatcgagacgttcccgcccgtggtgttcgccggggaggcgcgccacctcgaggagcgcctcgccgacgccgccatgggccgcgccttcgtcctccagggcggcgactgcgccgagagcttcaaggagttcaacgccaacaacatccgtgacaccttccgcatcctgctccagatgggcgccgtcctcatgttcggcggccagatgcccgtcgtcaaggtcgggaggatggctggccagttcgccaagccgaggtctgattcgttcgaggagagggacggggttaagctgccgagttacaggggagacaacatcaatggcgacaccttcgacgagaagagccgcgtgccggacccgcagcggatgatccgcgcgtacgcgcagtcggtggcaacgctcaacctgctccgcgccttcgccacgggagggtacgccgccatgcagcgcgtcacgcagtggaacctcgatttcatggatcacagcgaacaaggagacaggtaccgtgaattggcacaccgggtggatgaggcacttggcttcatgactgcagcagggctaacagtcgaccatccgataatgacaacaactgacttctggacatcccatgagtgcctcctcttaccatatgagcagtctcttacacgtgaggattccaccagtggcctcttctatgactgctcagcccacatgctatgggttggtgagcgcactcgccagctcgatggagctcatgttgaatttctccgtggtgttgccaaccctcttggcataaaggtgagtgacaaaatgaacccccgtgacttggtgaagctcattgagattttgaacccatcaaacaagcctggaaggataaccataattacaagaatgggggcagagaacatgagggtgaaattgcctcatcttatccgtgctgtccgcaactctggacagattgtcacatggattactgatcccatgcatggaaacaccatcaaggctccatgtggtctgaagactcgtccattcgattccattctggctgaagtacgtgcgttcttcgatgtccatgatcaagaaggtagccacccaggaggtatccacctggaaatgactgggcagaacgtgactgaatgcatcggtggatcaaggaccgtcaccttcgatgacctgagcgaccgctaccacacccactgtgacccaaggctgaacgcgtcgcaatccctggagctcgccttcatcatcgccgagagactgaggcggaggaggatgcggtccggggtcaacagcaacctgccattgcccccattggctttctaa

SEQ ID NO: 50 - DNA, 645 bp, Oryza sativa
  source 1..645: mol_type = other DNA
atggctgtgtcggtttcagaggagatggtgactatggcgaagtccggcctggtcacgagcttaagacgacactgtagtgggggcccacctggcaacttgggagggacaaaaacaaacttgggcgggctgggcctcttgggccaagaggcccacacgggagagggagggaggggaggtgctgggcgagatgagaggatttctcaggatctggtacagtggacgaatgtccggctcgacgtcccgttggtgcttcttctggtcgtccagtcggctctcgaggcctggctgcggcccgccgagttcgagagctcgcctgccccgtgcctgccagtgactgatgtccttgattcgccgtcagttccagccactgccgcctatgctgatggactcactatggatcaccctattatgacaacagaattctggacatcacatgagtgccttcttcttccctatgagcaagcattacctgaaaacattagagtgaaactccctcacctcatacgtgctgttcatgttgctgtccagatagtaacatgggttaccgacccgatgcatggtaacacaatgaaggctccttgtggcctcaagactcgctcctttgctacttttcagtatttgcttgttcctattacttctcagtag

SEQ ID NO: 51 - DNA, 1614 bp, Zea mays
  source 1..1614: mol_type = other DNA
atggcgctggccaccaactccgccgcagcagccgcggccgtgtccggtggcgtggcatcgcagccgcgccgcgcggccgtgttcctcccgctgaagaggcgtaccatctccgccatccacgccgccgacccgtccaagaacaacgggcccgcggtccccgcggccgcaggcgccaactcctccgcctcggcggtggccacgccggagaagaagccggcagcgcacgggaagtgggcggtctacagctggaggtcgaagaaggcgttgcagctccccgagtacccgaacgcggaggagctggacgcggtgctgaagaccatcgagacgttcccgccggtggtgttcgccggagaggcgcgccgcctcgaggagcgcatggccgaggccgccatgggccgcgccttcgtcctccagggtggcgactgcgccgagagtttcaaggagttccacgccaacaacatccgtgacaccttccgcgtccttctccagatgggcgtcgtgctcatgttcggtggccagatgccggtcgtcaaggtggggaggatggctggccagttcgccaagccaaggtctgagccgttcgaggagaaggacggagttaagctgccgagctacaggggcgacaacgtcaacggcgacgacttcaccgagaagagccgcgtgccggacccgcagaggatgatccgcgcctacgcacagtcggtggcgacactcaacctgctccgcgcgttcgccaccggagggtacgctgccatgcagcgcgtcactcagtggaacctcgatttcatggatcacaacgagcaaggtgataggtaccgtgaattggcccatagggtggatgatgctcttgggttcatgactgcatcggggcttacagtcgaccacccgataatgacgactactgacttctggacctcgcacgagtgccttctcttaccctacgagcaggctcttacccgtgaggattccaccagtggccttttctatgattgttcggcccacatgttgtgggttggtgagcgcactcgacaactcgatggagctcatgttgaattcctccgtggtgttgccaaccctctgggcataaaggtgagcgacaaaatgaaccccagtgagttggtgaagctgattgatattctgaacccttcaaacaaacctggaaggatcaccataattacaaggatgggggcagagaacatgagggtgaagttgcctcatctcatccgtgctgttcgcaatgctggactgattgtcacatggattactgatcctatgcatggaaacaccatcaaggccccttgtggcctgaagactcgtccatttgactccattctggctgaagtgcgtgccttcttcgatgtgcatgaccaagaaggaagccaccctgggggcgtccaccttgaaatgactgggcagaatgtgaccgagtgcatcggtggatcacggaccgtgaccttcgacgatctgagcgaccgctaccacacccactgcgacccaaggctgaatgcctcccagtccctggagctcgcctttatcatcgcagagaggctgaggaagaggaggatgcgatcggggctcaacagcagcctgccactgccgccactggctttctga

SEQ ID NO: 52 - DNA, 1623 bp, Sorghum bicolor
  source 1..1623: mol_type = other DNA
atggcgctggccaccaactccgccgccgccgccgcagcagcagcggccgtatccggtggcgcgtcatcccagccgcgccgcgcggccgtgttcctcccgctgaagaggcgcaccatctccgccatccacgccgccgacccgtcaaagaacaacggacccgccgtccccgcggcggccgctgccaagtcttccgcctcggcggtggccacgccggagaagaagccggcggctccggggaagtgggcggtcgacagctggaaatcgaagaaggcgctgcagctccccgaatacccgaaccaggaggagctggacacggtgctcaagaccatcgaaacgttcccgccggtcgtgttcgccggagaggcgcgccacctcgaggagcgcatggcagaggctgccatgggccgcgccttcgtcctccagggcggcgactgcgccgagagcttcaaggagttccacgccaacaatatccgtgacaccttccgtattctgctccagatgggcgccgtgctcatgttcggtggtcaggtgccggtcgtcaaggtggggaggatggctggccagttcgccaagccaaggtccgaaccgttcgaggagagggatggtgttaagctgccgagctacaggggtgacaacgtcaacggcgatgacttcaccgagaagagccgcgtgccagacccgcagaggatgatccgcgcctacgcgcagtcggtggcgacgctcaacctgctccgcgcgttcgccacaggagggtatgctgccatgcagcgtgtcacacaatggaacctcgacttcatggatcacagcgagcaaggtgataggtaccgtgaattggcccatagggtggatgaggctcttgggttcatgactgcagcaggacttaccgttgaccacccgataatgacgactactgacttctggacctcgcacgagtgccttctcttaccctatgagcaggctcttacccgtgaggactccaccagtggccttttctatgattgttcagcccacatgttgtgggttggtgagcgcactcgtcaacttgatggagctcatgttgaattcctccgtggtgtggccaaccctcttggcataaaggtgagtgacaaaatgaatcccagtgacttggtgaagctgattgagattctgaacccttcaaacaaacccggaaggatcaccataattacaaggatgggggcagagaacatgagagtaaagttgcctcatctcatccgtgctgtccgcaatgctggattgattgtcacatggattactgatcctatgcatggaaacaccataaaggccccttgtggcttgaagactcgtcctttcgattccattctggctgaagtgcgcgcattcttcgacgtgcatgaccaagaaggaagtcacccaggaggtatccaccttgaaatgactgggcagaacgtgaccgagtgcattggtggatcacggactgtgacctttgacgacctgagcgaccgctaccacacccactgtgacccaagactgaacgcctcccagtccctggagcttgccttcatcattgcagagaggctcaggaagaggaggatgcggtcagggctcaacaacagcctgccgctgccaccactggctttctga

SEQ ID NO: 53 - DNA, 1617 bp, Zea mays
  source 1..1617: mol_type = other DNA
atggcgctggccaccaactccgccgctgccgcagcagctgccgtatccggcggcgcggcatcccagccgcaccgcgcggccacgttcctcccgctgaagaggcgcaccatctccgccatccacgccgccgacccgtctaagaacaacgggcccgccgtccccgcggccgccgccgctaagtcatctgcctctgcggtggccacgccggagaagaatccggcggcgccggtaaagtgggcggtcgacagctggaagtcgaagaaggcactgcagctcccagagtacccgaaccaggaggagctggacacggtgctcaagaccatcgagacgttcccgccggtggtgttcgccggagaggcgcgccaccttgaggagcgcatggccgaggccgccatgggccgcgccttcatcctccagggcggcgactgcgccgagagcttcaaggagttccacgccaacaacatccgtgacaccttccgtatcctgctccagatgggcgccgtgctcatgttcggtggtcaggtgccggtcgtcaaggtggggaggatggctggccagtttgccaagccaaggtccgaaccgttggaggagagggacggcgtcaagctgccaagctacaggggcgacaacgtcaacggcgacgacttcaccgagaagagccgcgtgccagacccgcagaggatgatccgcgcctactcgcagtcggtggcgacgctcaacctgctccgcgcgttggcgaccggagggtacgctgccatgcagcgcgtcacacagtggaacctcgatttcatggatcacagcgagcaaggtgataggtaccgtgaattggcccatagggtggatgaggctcttgggttcatgactgcagcagggcttacagttgaccacccgataatgacgactactgacttctggacctcgcacgagtgccttctcttaccctacgagcagtctcttacccgtaaagactccaccagtggccttttctacgattgttcggcccacatgttgtgggttggtgagcgcactcgtcaactcgatggagcgcatgttgaattccttcgtggtgttgccaatcctcttggcataaaggtgagcgacaaaatgaaccccagtgacttggtgaagctgattgagattctgaacccttcaaacaaacctggaaggatcaccataattacaaggatgggggcagagaacatgagagtgaagttgcctcatctcatccgtgctgttcgcaatgctggattaattgtcacatggattactgatcctatgcatggaaacaccatcaaggccccttgtggcctgaagactcgtccattcgactcaattctggctgaagtgcgcgcattcttcgacgtgcatgatcaagaaggaagtcacccaggaggcatccaccttgaaatgactgggcagaacgtgaccgagtgcattggtggatcacggactgtgaccttcgatgacctgagcgaccgctaccacacccactgtgacccaaggctgaacgcctcccagtccctggagctcgccttcatcattgcagagaggctcaggaagaggaggatgcggtcggggctcaacaacagcctgcctctgccaccactggctttctaa

SEQ ID NO: 54 - DNA, 1596 bp, Populus trichocarpa
  source 1..1596: mol_type = other DNA
atggctctttcaactctctcttccaaatctctctacagcaatgccaacaatctctttcaacaaaaccaacaacaacaatcacccactttctctcttgtccctggaagcaaaacgctcaagtcaaaatccaacctttcaatctctgccgtccatgccgccgagcccaccaagaattcagtttccgtgaaggaatctgcggccccagttgctgcccctgggactggaaaatggtccttggagagctggaaaaccaagaaagcattgcagctacccgaataccctgatgctaatgaactcgagacagttttgaagactattgagacgtttccgccaatagtttttgctggagaagcaaggagtttagaggagaaccttgctgaggctgcaatggggaatgcttttttattgcaaggtggtgattgtgctgagagtttcaaggagtttagtgctaataatataagggatacttttaggattctgcttcagatgggtgttgtgcttatgtttggaggtcaagttcctgtcattaaggtgggaagaatggcaggtcaatttgcaaagccaagatcagatccatttgaggagaaggatggagtgaagctgccaagttacaaaggggacaatattaatggagatgcttttaatgagaaatcaagaattccagacccgcagagattgataagagcttattgccagtctgctgcaactctcaatcttctcagggcatttgccaccggagggtatgctgcaatgcagagagttacacagtggaatcttgactttgcacagcacagcgagcagggagataggtaccaagaacttgctaaccgggttgatgaggccttgggattcatggctgcagcaggactcacagtggatcatccaatcatgacaactactgaattttggacatcccacgagtgcttgcatcttccctacgagcaatctctcacaaggcttgattcaacttctggcctctattatgattgctctgctcacatgctttggtgtggagagcgtactcgccagctggatggtgcccacgtggagttccttagaggaatttccaaccctattggcatcaaggtgagcaacaaaatggatccaaatgagctagttaaactcattgaaatctttaattctaataacaagccaggaaggattacaatcattgttagaatgggtgctgagaatatgagagtgaagtttccccacttgatcagagctgttcgtagggcgggacaaatcgtgacatgggtctgtgatccaatgcatggaaacaccattaaggctccttgtggactgaaaacacgcccctttgatgctattttggctgaggtgcgagcattctttgatgtccatgagcaagaaggaagccatcctggaggaattcacctagagatgactgggcagaatgtgactgagtgcattggagggtctcggacagtaacatttgacgatttgagctcccgctatcacacgcattgtgacccaaggctcaatgcttctcaatcgcttgagcttgcatttatcattgccgagcgacttagaaaaagaaggataggaactcaacgtctgttttccttgggccagtag

SEQ ID NO: 55 - DNA, 1521 bp, Populus trichocarpa
  source 1..1521: mol_type = other DNA
atggctctttcaactctctcatcaaaatctctctacagcaatgccaacaacctccttaaacaaaaccaacaacaaccacccactttctcttttgtccctggaaacaaaacccacatgtcaaaatcccacctttcaatctctgccgtagctgccgagcccaccaagaattcagtggcagtgaaggaatatgctgccccagggactggaaaatggtccttggagagttggaaaacgaagaaagcattgcagctaccagaataccctgatgctaatgagcttcagtcagttttgaagactatcgaggcgtttcctccaatagtttttgctggagaagcaaggagtttagaggagaggcttgctgaggctgcaatggggaatgcttttttgttgcaaggtggtgattgtgctgagagtttcaaggagtttagtgccattaatataagggatacttttagaattatgcttcagatgagtgttgtgctaatgtttggaggtcaaatgcctatcattaaggtgggaagaatggcaggtcaatttgcaaagccaagatcagatccatttgaggataaggatggagtgaagctgccaaacccacagagattgataagggcttataaccagtctgccgcaactctcaaccttcttagggcatttgccactggagggtatgctgcaatgcagagagttacacagtggaatcttgacttcgcacagcacagcgagcagggggataggtaccaagaactagctcaccgggttgatgaggccttgggattcatggctgcagcaggactcacagtggatcacccaatcatgacaactactgaattttggacatcccatgagtgcttgcaccttccttatgaacaatccctcacaaggcttgattcaacttctggtctctattatgactgctctgctcacatgctttggtgtggagagcgcactcgccaactggatggtgcccacgtggagttcctgagaggaatttccaaccctattggcattaaggtgagcaacaagatggatccaaatgagctagttaaactcattgaaatcttgaattctaacaacaagccaggaaggatctcaatcatcgttagaatgggtgctgaaaatatgagagtgaagcttccccacttgatcagagctgtccgtagagcaggacaaattgtgacatggatctgtgatccaatacatggaaacaccatcaaggcaccttgtggactcaaaacacgcccctttgatgctattttggctgaggggcgagcattctttgatgtgcatgagcaagaaggaagccatcctggaggaattcatctagagatgactggccagaatgtgactgaatgcattggagggtctcggacagtaacttttgatgacttgagctctcgatatcacactcattgtgacccaaggctcaatgcttctcaatcacttgagctcgcttttatcattgctgagcgactcagaaatagaagggtaggaactcaacgtctgctttcctcgggcctgtag

SEQ ID NO: 56 - DNA, 1518 bp, Oryza sativa
  source 1..1518: mol_type = other DNA
atgcccctcgcgccatgcccctcgccgcccctcccctcctccccgtggccggcgcgcgccccgcgccggggcggcctcctccgcgcccgcgcggtgcgggcggcgccccggccgccgagcaagtggtcgctgggtagctggcgcagcctgacggcgctgcagcagccggagtaccccgacaaggcggagctggatgaggtgctccggacggtggaggcgttcccgccgattgtcttcgccggcgaggcgcgcaagctggaggagcggctcgcggaggccgccgtcgggcgcgcgttcctcctccagggcggcgactgcgccgagagcttcaaggagtttaacgcgaacaacatccgggacaccttccgtgtgctcctccagatgtccgtggtgctcatgtttggaggccagatgcctatcatcaaggtaggaagaatggcaggtcagtttgcaaagccaaggtcagatggctttgaggagagggatggagtgaagttgccaagctacagaggggataacattaatggggattcattcgatgagaaatcaagattgccagatccacaccgcatgatcagggcatactcacagtctgcagcaacactgaatttgctgcgggcttttgctactggaggttatgctgccatgcagagggtaacacaatggaaccttgacttcacagagcatagtgaacagggtgacaggtacatggagctggctcaccgagttgatgaggctttggggttcatggcagctgctggtctcactatggaccatcctattatgacaacaacagaattctggacatcacatgagtgccttcttcttccctatgagcaagcacttactcgcgaggattccacatctggcctctattacgactgttctgctcacttcctttgggttggagagcgtacacgtcagcttgattgtgcccatgtggagtttctccgaggaattgcgaaccctctgggtatcaaggtcagtgacaagatggacccaaaagaacttgtgaagttgattgatatcttgaatccccagaacaaaccagggagaattactatcattacaagaatgggacctgaaaacatgagagtgaaactccctcacctaatacgtgctgtccgtggtgctggccagatagtaacatgggttactgatccgatgcatggtaacacaatgaaggctccttgtggcctcaagactcgctcctttgatagaatcttggctgaggtgcgcgcattctttgatgtgcacgaacaagaaggcagccacccaggaggggtgcatctggagatgactggacaaaatgtgacagaatgcatcggcgggtcacgcacggtgacattcgacgatctgggctcacgataccacacacactgcgacccgaggctcaacgcatcgcagtctctggagttggcgttcatcatcgccgagcggctcagaaagaggaggatcgcctcatggcagttgaacaagaacagtcatctgggcaacatcccatctttggggctctga

SEQ ID NO: 57 - DNA, 1656 bp, Sorghum bicolor; identical to SEQ ID NO: 48
  source 1..1656: mol_type = other DNA

SEQ ID NO: 58 - DNA, 1521 bp, Zea mays
  source 1..1521: mol_type = other DNA
atggcactcgcgccatcacccccagcgctgccgcccaacccggcgctccctttgccgtggtgggggcgggccggccgcggtgcgctcctcggcgcccgcgcccgcgtccgcgccgtccgcgcggcgctacggcccccgagccagtggtccgtcgggagctggcggggccgcccggcgcagcagcagcccgagtacccggacaaggcggacctggaagacgtgctgcggacggtgggaacgttcccgcccatcgtcttcgccggcgaggcgcgcaccctcgaggagcgcctcgcggaggccgccgtcggccgggccttcctcctccagggcggcgactgcgccgagagcttcaaggagttcaacgccaacaacatcagggacaccttccgcgtcctcctgcaaatgtccgttgtgctcatgttcggaggccagatgcctgtcgtcaaggtgggaagaatggcagggcagtttgcgaagccaaggtcagatggttttgaggagcggaatggattgaagttgccaagctatagaggggacaatatcaatggggatgcattcaatggggagtcaaggttgccagatccacaccgcatgataagggcgtactcacagtctgcagcaacactgaatctgctgcgggcattcgcaactggaggttatgctgcaatgcagagggtagcacaatggaaccttgatttcacagagaacagcgaacaaggtgataggtacatggagttggctcaccgagttgacgaagctttggggttcatgtcagctgctgggctccctttagatcaccctataatgacaacagcagaattctggacgtcacatgagtgccttcttctaccttatgagcaagcgctcactcgtgaggattccaccacgggcctctattatgactgctctgcccacttcctatgggtcggagagcgcactcgccagcttgatggtgctcacgttgagttccttcgaggcattgccaaccctcttggtatcaaggttagtgacaagatggacccagcagaacttgtgcggttgattgatatattgaatcccgaaaacagggctgggagaataaccatcatcacaagaatgggacctgaaaacatgagggtgaaacttccacacctgatacgcgctgtccgtggggccggtcagatagtaacatgggttactgacccaatgcatgggaacactatgaaggccccttgcggactcaaaacccgctcgttcgacaggattttgatggccgctatgggcatcggcgactgcgccaccaccagccagcgcggcgacctggacatcccgtacccgttgagcatcgggcgcgggtgctaccactacacgggcgatcgagggcggggcgacatcacctgcaggcgcaccgcgcgcggcggctaccagcctaccaggccttcagcggcgagtccgttgaggtcatcgccatctccgtgcgccgcggccaggcgcgcgtctgcattctgcaacgacatccagacctggtgctacaacacctag

SEQ ID NO: 59 - DNA, 1554 bp, Gossypium raimondii
  source 1..1554: mol_type = other DNA
atggtggtcgcaatcccgggctccgctaatctcaccgcctcaaaaccctctctaggactgggaggtaccaccacacgcctctctctctcccgcccgatcccaaacctccacgtcaccaaaaaacccaactccttcattgtcgcttccgtttccgcttcctcttctccctccaccggtcaaaccaattggtctctcgattcctggaagtcgaagcctgcccaacaactccctgaatatccggacgctgatgagctcgattcggtactcaaaacgatcgggtccttcccgccaattgtgtttgctggggaggcaagaagactggaggataaactagccagcgccgccgtcgggaaagcgtttctgttacagggtggtgactgtgcagagagtttcaaagaattcaatgggaccaacattagagacactttcagggtccttttgcagatgggaattgtcctcacttttggtgcccaaatgcccgtcattactgtcgggaggatggcagggcaatttgcaaagccaaggtcagatccatttgagataaaagatggtgtaaagttaccgagttatcgaggagataacataaatggcgatgcttttgacgagaaatctagagttcccgaccctcaaaggttgatcagagcttatcttcaatctgttggtaccttgaatctccttagagcattcgctactggtggctatgctgctatgcaaagggtctcccaatggaatctcgattttgttagaaatagtgaacagggagacaggtacatggagcttgcacagagagttgatgaagctttgggattcatggctgcagctgggataaccattggtgatcctcaaatgaacacagttgacttttggacatcccatgaatgccttcatttaccatatgaacaagccttgactagagaagattcaacaaccggcctttattatgattgctctgctcacctgctttgggttggtgagaggactcggcaattagatggagcacatgtcgaatttctccggggtatttccaatcctctcggcataaaggtcagtgataaaatggacccgaaggaccttgtgaagttgtgtgaaattctcaaccctcggaacaaacctggaaggctgacaataatcacacgaatgggtgccgataacatgcgaataaagcttccacatcttataagggcagtgcgccaggctgggcttattgtcacttgggtgagtgatcccatgcatgggaacacaattaaagctccatgtggtctcaaaactcgtccatttgatgcaatcagatctgagttgagggcattcttcgatgttcatgaacaagaagggagctatcctggaggagttcatctggagatgacggggcagaacgtaacagagtgcatcggagggtcaaacaccgtcacttttgatgacttgaactctcggtatcacacacattgcgacccgagattaaatgcatcgcagtcattagagctagcctttgccatatcagaaaggttgaggaagagaaggctcaagtctgccaaggaactttgcaatgacaattaa

SEQ ID NO: 60 - DNA, 1536 bp, Solanum lycopersicum
  source 1..1536: mol_type = other DNA
atggctttatcaaacaccttatcattgtcatcatcaaaatcccttgttcaatctcatctgctccataatcctacaccccaacctcgtttttccctttttccgaccacccaacatgggcggcgccaccctatctccgccgtacacgcggcggagccatccaagactgcagttaagcaaggaaaatggagtcttgatagctggaaaacaaagaaagctttacaacttcctgaatacccagatgagaaagaacttgaatcggtgcttaaaacacttgaaatgaatccaccccttgtgtttgctggtgaggcaaggagtttggaagagaagcttggtgaggctgcattggggaaagcttttttgttacaagggggagactgtgctgagagttttaaggagtttaatgccaataatattcgtgatactttcaggattttgcttcagatgagtgttgttcttatgtttggtggacaagttcctgtcattaaggttggaagaatggccggtcagtttgcaaaaccaagatcagatccgtttgaggagataaatggagtgaagctaccaagttacaagggtgacaatatcaatggtgatacgttcgatgagaagtcaagaattccagatcctcataggcttattagggcatacatgcaatctgctgcgactcttaatcttcttagagcttttgctactggaggttatgctgcaatgcagagggtcactgaatggaatcttgattttgtggagaacagtgaacagggagataggtatcaggaactagctcacagggttgatgaagccttaggattcatggctgctgctggactcacagttgaccaccctatcatgtcaacaactgatttctggacatcccatgagtgcttgcttcttccttatgaacaagcacttacaagagaggattcaacttctggtcttttctacgattgttctgctcacatggtttgggttggggaaagaaccaggcaactagacggtgctcatgtcgagtttttgagaggagttgcgaaccctcttggaataaaggtgagtcaaaaaatggatccaaaagagctaattaagctcattgacatcctgaaccctgccaataagcctggaagaattactgtaattgttagaatgggtgccgagaacatgagagtaaaactttcccacttggtcagggctgtacggggcgctggtcagattgtgacatgggtttgtgatccaatgcacgggaataccatcaaggcaccatgtggactcaaaacccgtgctttcgattcaatcctggctgaggtccgagctttcttcgatgtgcatgagcaagaaggaagccaccctggtggtattcaccttgaaatgacagggcaaaatgtgactgaatgcattggtggatcacgaacagtaacctatgacgatttgggctctcgctaccacactcattgtgacccaagattgaatgcatctcagtctcttgaactctccttcatcgtagctgagcgactaagaagaagaagaatgtcatctcaacgtctgtag

SEQ ID NO: 61 - DNA, 1554 bp, Nicotiana benthamiana
  source 1..1554: mol_type = other DNA
atggctttatcaaacaccttatcattgtcatcatcaaaatctcttgttcaatctcacattctccacaacccctcattaccccaaccccgtattcctctttttcacaacccccaacatgggcggcgccaccccatctccgccgtacatgcggcggagcccggcaaaacagcaactgcttcacagccgttgaaaaaaacccaatggagtcttgattcttggaaaagtaaaaaggctttgcaattacctgaatacccagatgaaaaagaacttgaatctgtgcttaaaactcttgaatctaatcctccacttgtgtttgctggtgaagctaggaatttagaagagaaacttggtgaagctgctttaggaaaagcttttttattacaaggtggtgattgtgctgagagttttaaggaatttaatgctaataatattcgtgatacttttaggattctccttcagatgagtgtggttcttatgtttggtggtcaagttcctgttattaaggttggaagaatggcgggtcagtttgcgaaaccaagatcagatccgtttgaggagattaatggagtgaagctgccaagttacaagggtgataacattaatggtgatacatttgatgagaagtcaaggatcccagatcctcataggcttattagggcttacatgcaatctgctgcgactcttaaccttcttagagcttttgctaccggaggttatgctgcaatgcagagggtcaccgaatggaatcttgattttgtggagaacagtgagcaaggagataggtatcaagaactagctcatagggttgatgaagccttgggattcatggctgctgctggactcacagttgaccaccctatcatggcgacaactgatttctggacatcccacgagtgcttgcttcttccttatgaacaggcacttacaagggaggattcaacttctggtcttttctatgattgctccgctcacatgatttgggttggcgaacgaaccaggcaacttgatggtgctcatgttgagtttttgaaaggagtagcaaacccacttggcatcaaggtgagccaaaagatggatccaaatgagctagtgaaactcattgacatcctgaacccaaccaataagcctggaagaattactgtaattgtgagaatgggtgctgagaatatgagagtaaagctttgccacttgatcagggcagtgcgaggagctggacagattgttacctgggtttgtgacccgatgcacggcaacaccataaaggcaccatgcggactcaaaacccgtgctttcgattcaatcctggctgaggtccgagcgttcttcgatgtgcatgagcaagaagggagccaccctgggggtatccatctagaaatgacagggcaaaatgtgactgaatgcattggtggatcacgaacagtaacctacgacgatctgggctctcgctaccacacacattgtgacccaagattgaatgcttctcagtctcttgaactttccttcatcgtagctgaacgactaagaaaacgaagaatggcctctcaacgcctgtag

SEQ ID NO: 62 - DNA, 1551 bp, Populus trichocarpa
  source 1..1551: mol_type = other DNA
atggcactctcaaccactctcagcactgcaagaccctccatctcccctctcaaaccccaaaacttcacctcaacaaccagaaaccctaacctcaaacccaaaaccacttctgtttttgcctccctgcgctcaactccaacatcaaagcccacaatctcttccaactgggccttagacagctggaaatctaaaccagcacgccagcttcctgattacccggacccggttgaactgcactcggtgctccagacccttaccaatttcccgccaattgtgttcgcgggcgaggctaggaagcttgaggagagaattgcgagtgctgctgtgggaggtgcctttttattgcaaggtggagattgtgctgagagttttaaagagttcaatgccaataatattcgagatacttttcgagttttgctccagatgggtgttgtcctcacctttggtgctcaaatgcccatcatcaaggttggtaggatggcaggccaatttgcaaaacctagatctgacccctttgaggaaaaagatggcgtgaagctaccgagttatcgaggagataatattaacgccgatgcttttgataagaaatctaggacacctgatcctcaaaggttgatcagagcatacctccaatctgttggcaccttaaatctccttagagcttttgccactggtggatatgctgctatgcaaagagtgtcacaatggaatcttgactttgtagaacatagtgagcaaggagacagattcatggaacttgcacggagagttgatgaagctcttggcttcatggctgctgctggcctcactatagatcatcctgtaatgaacacaacagagttttggacatctcatgagtgtcttcatttaccatatgagcaggctctaaccagggaggactcaacaactggcctctattatgactgttctgctcacatgctctgggtaggtgagaggacccggcagttggatggagcacatgttgaattcctacgtggtgtatccaatcctcttggcatcaaggtgagtgataagatggatccaaaggagcttgtgaaattgtgtgaaattcttaacccgcacaacagacctggaagactgacaataattaccagaatgggagcagacaacatgcggattaaactcccccatctgataagagctgtgcgccacgctgggcttatagtcacatgggtcagtgatcccatgcatggtaatacgattaaagctccatgtggtctcaagacaaggccatttgattcaatcagggctgagttgagggcattctttgatgtccatgaccaagaaggtagctatcctggaggagtccacttagaaatgactgggcagaatgtaacggagtgtgttggggggtcaaagaccatcacttttgatgacttgaattcccgctatcacacacattgtgatcctagactgaatgcgtctcagtcgctggagctggcatttgccatatcagagaggctgaggaagaaaaggttaagagctggtgatggtattcttagcggacataacactggttctgtagcgtaa

SEQ ID NO: 63 - DNA, 1596 bp, Nicotiana benthamiana
  source 1..1596: mol_type = other DNA
atggctctgacagccgccaccagcagcagcagcagcagaggtggctcagctctccccaattcatgccttcaaaccctaaagctttcacctttacagaaacccaccttcatctcttccttccccacaaccacagccaccccttcaaactccaaaatcaagccaagaattaacaaaccaatctcagccgttcaatctccaccctcaacaacaacaaaaactgctcaaaacccagatggaaaatggagtcttgaaagctggaagttgaaaccagcttttcagctacctgagtaccctgataaagttgagctggaatcagttcttaagaccctttcaacttatcctccaattgtatttgctggggaagctaggaatcttgaagagaaattgggtgaagcagctcttggaaatgctttcttgttgcaagggggtgattgtgctgagagttttaaggagtttagtgctaataacattagggacacttttagagtcatattgcagatgggagttgtgcttatgtttggtggtcaaatgcctgttatcaaggtgggtagaatggcaggtcaatttgcgaagccaagatctgatccgtttgaagagaaggatggcgtgaagctaccaagttacaggggagacaatgtgaatggtgacgcttttgatgagaaatcaagacttcctgaccccgacaggatgattagggcatacgctcaatctgttgctaccttgaacctcctccgagcatttgctacagggggttatgctgccatgcagagggttaaccagtggaacctcgactttactgatcacagcgagcaaggtgacaggtatcgcgaactggctcaccgagtagacgaagccatgggcttcatgagtgctgctgggattacagttgaccacccaatcatgactacaacagacttctggacatcccatgagtgccttctcttgccttatgaacaagcacttacgagggaggattcaacttctggcctttattacgactgctccgctcacatgatttgggttggggagcgaacaaggcaattggatggtgctcatgttgagttcctgagaggaattgccaatccacttggtatcaaggtgagtcagaaaatggatccagatgaacttgtcaagcttattgacattcttaaccctcaaaatagaccaggaagaattacagtgatcgccaggatgggagctgataacatgagagtaaagcttccccatctgatcagggctgttcgcggagcaggtcaaattgtcacttgggtcagtgatcctatgcatggaaataccaccaaagccccctgtggactcaaaacgcgttcatttgactctatccgggctgagctaagagcattctttgatgtacacgatcaagaagggagctatccgggtggcgtgcatctggagatgacaggtcagaatgtaacggagtgcgtgggaggctctcggacaattacttacaatgatctaagctcgcgctaccatacacattgtgacccaagacttaatgcttctcaagcacttgaactggcctttcttattgccgagcgcctcagaaaaagaagactgggaccaaagttccggctctag

SEQ ID NO: 64 - DNA, 1551 bp, Glycine max
  source 1..1551: mol_type = other DNA
atggctgccactgcttccaactctctcctttgcaccaaaacgctagtcccaaccctcccttcttcctccaacaacctcaacaacagcaacctatgctcctcctttcccaggcccaggcccaggcccatccaggccctgaacaactccgtgactgtaataaccgacaagaagtgcgaggcaaagtgggtggtggacagctggaagtccaaaaaggctcttcaactcccggaatacccggagcaagagaagctccagtcggtgctcaagagcctggaagccttccctccgattgtgtttgccggagaggcccgcaacctcgaggagcgcctcgcccaggcttccatgggaaacgcgttcctcctccagggcggagactgcgctgagagcttcaaggagtttcatgcaaataacattcgtgacaccttcagactcctcctccaaatgagtgtcgtcaccatgttcggagcccaagtgcctgtcattaaggtgggaagaatggcgggtcaatttgcgaaaccgagatcggaagcgtttgaggagaaagacggggtgaaactcccaagttacagaggagataatgtgaatggagatgctttcgaggagaaaacgagaattcctgatcctgaaagaatgatcagatcatacagccaatctgctgcgactctcaatcttctgagagcatttgcgacgggaggatatgctgctatgcaaagggttagccaatggaatttggacttcacgcaacacagcgagcaaggagacaggtacctagagcttgctcgccgaattgatgagacccttgggttcatggctgctgctggcctcacaatggatcatcctataatgaaaacaaccgagttttggacttcacatgaatgtttgttattgccctatgaacaatcactcactaggttggattcaacttctggtctctactatgactgctccgcccatatgctttgggttggtgaacggactagacagctcgatggtgctcatgttgaatttctaagagggattgctaatccccttggaattaaggtaagtgataagatcgatccaaaggagcttattagactaatagagatcttgaatccccagaacaagccagggagaataactgtgataacgaggatgggagctgaaaatacgagggcgaaacttccacatctgatcaggggagtacgcaatgcgggactaattgtatgttgggtcagtgatcctatgcatggaaacaccataaaagctccctgtggactcaaaactcgccccttccattccatcatggaggaggtgaaatcattcttcgacgtgcatgaacaagaaggaagccacccaggtggggttcatctagagatgacaggtcagaatgtgactgagtgcattggtgggtcaaagatggtcacctttgatgatctcagctcacgctacaacactcactgtgaccccaggcttaatgcttcacagtctcttgagcttgctttcatcatttctgaacgcctcaaaaagaataggatcgggtcaaatctgaacaatgttttttctctatag

SEQ ID NO: 65 - DNA, 1488 bp, Glycine max
  source 1..1488: mol_type = other DNA
atggctgtggcgtcgtcatcatcccttatcacgttgaaggtgaaaccttgcattttcgggtctcctcggagatccgcggtggttcggaattgtgcgaagtcaacggcggggacaatatcgacgagttggagcctggacagctggagggcgaagaaggcgcttcagcttccggagtacccagatgcgaatgaattggacctagtcctccaaaccctctcttcttttcccccaatcgtcttcgccggcgaggcgaggaatctggaggagaagctcgctcaggctgccatggggaacgcttttctccttcagggcggtgattgcgccgagagcttcaaggaattcactgccaacaacatccgtgacaccttccgtgtcatccttcaaatgggtgtggtcctcatgttcggtggccaaatgcccgttatcaaggtggggagaatggcaggtcaatttgcaaagccgagatccgattcatttgaggagaagaatggagtgacgctcccgagttacaggggtgataatgtgaatggcgatgcatttgacgcggcatctagaatccccgatccacagaggatgataagagcctactgccaatctgtgtctactctgaaccttttgcgggcatttgccacgggaggttatgctgccatgcaaagggttaatcaatggaatcttgatttcatggagcatagtgaacagggagacaggtaccgtgaattagcccatagagtggatgaggctcttggcttcatgaatgttgctgggctcacagccgaccatcccatcatgagtacaacagacttttggacctcccatgagtgtttgcttctcccttatgagcaagcacttactagggaggattctactactgggcttcattatgattgctcagctcacatgctatgggttggggaacgtacccgccaacttgatggtgctcatgttgaattcttgagaggagttgctaatccacttggcatcaaggtgagtgataagatggttcccgatgaacttgttaagctgatagatattctgaaccctaaaaacaagcctggaagaattacagtcattgttagaatgggagctgagaatatgcgagtgaagcttccacatcttatcagggcagttcgcagagcaggtcaaattgtcacttgggttagtgaccccatgcatgggaacaccattaaagctccatctggacttaaaacccgctcttttgatgcaataagggctgagctgagggcattctttgatgtgcatgatcaagaaggaagctacccaggaggggttcatttagagatgacagggcagaacgtgacagaatgtgttggaggctcaaggactattacttatgatgacttgagctcacgctaccacacacattgtgatccaaggcttaatgcttctcaatctctcgagcttgcatttaatattgcggacaggttgcgcaagagaaggctcaattcaactcgatcacttggaatttag

SEQ ID NO: 66 - DNA, 1551 bp, Gossypium raimondii
  source 1..1551: mol_type = other DNA
atggctctgacatccccttcctttctcgcctccaaatcccctcttctcccccgccgccatctcctccctccaagtcttaaacccatcaccgccgtccattcggccgacccgactaagtcaaccaaatcaatggcggctgccgcctccaccagctccccttcgatccccacgcaatggagcctcgaaagctggaagtccaagaaggccctccagcttcccgaatacccagaccaaaacgacctagtatccgtcctcgagacgctctctacgttccctccggtcgtcttcgccggggaggctaggagcctggaagagaagctgggacaggctgctctcggcaacgctttcttgctgcaaggtggtgactgtgccgagagcttcaaggagttcgatgctaacaacattcgtgacactttcagggtcctcctacagatgggtgtagtcctcatgtttggtggccaaatgcctattatcaaggtaggaagaatggcaggtcagtttgcaaagccgagatcagatccgtttgaggagaaggatggggtgaagctcccaagttatagaggtgacaatataaatggtgattctttcgatgagaaagaaagagttccagatccccataggatgatcagggcctattgccaatctgtggcaactttgaaccttttgcgggcatttgctactggagggtatgcagccattcaaagagtcacacaatggaatctagacttcactgagcacagcgagcagggggacaggtactgtgaacttgctcaccgagttgatgaggcaatggggttcatggctgctgctggactctcagttggtcaccctgtcatgacaactacagatttttggacatctcatgagtgcttgctcttgccttatgagcaggcactcactagggaggattctactactgggctttactacgactgctcagctcacatgctttgggttggagaacgcacccgccaacttgatggtgctcatgttgaatttttaaggggagttgctaatcctcttggcataaaggttagcgataagatggatcctagtgaattagttaggctcattgaaattctaaatcctcggaacaagccaggcagaataacagtcattgtaaggatgggagctgagaatatgcgagtgaaacttccccatttgatcagagcggtccgtggagctgggcaggttgtaacttgggttagcgaccccatgcatgggaacaccattaaagctccttgcggactgaaaactcgttcctttgatgccattagggccgaagtgagagcattcttcgatgtacatgatgatgaaggtagtcatcccggaggaattcatttagagatgacggggcagaatgtgacagaatgtcttggaggctcaagaactataacctataacgacctgggttcacgctatcacacccattgtgatccaaggctcaatgcttctcaatctctggaactcgccttcattatcgcggagcgcttaaggagacgaaggcttgcatcacggaaccctctagcctcctcccgatcatctatttag

SEQ ID NO: 67 - DNA, 1596 bp, Populus trichocarpa
  source 1..1596: mol_type = other DNA
atgtctctaactagcagttccgtaatccctaccaaatccttgatcccctctaacaagccccatcatccttgtttcttcaccactaaaccttccagatccatcctccacatctccgctgtacactcagctgatccttccaaatcaccatcacccattaaaacacctgcagcaacatccactaaagcagcaaccgttgctccaacaaccaatgtgggtactgggaaatggactgttgaaagctggaaatccaagaaggccttgcaactccctgaatatcctaacaaagaagaccttgattcggttctcgaaaccctcgacgcctttcctccgattgttttcgctggtgaggccaggaatttagaggagaggctcgctgaggctgctatgggcaatgcgtttttgctgcaaggaggtgattgtgctgagagttttaaagagttcaatgctaataatattagggacactttcaggattcttcttcaaatgggcgctgttttgatgttcggtggtcaaatgcccgttatcaaggtggggaggatggcagggcagtttgcgaagccaagatcggatccactggaggaaaaaaatggagtgaaattaccaagttacagaggggataatgtgaatggagatgcttttgatgagaagtcaaggatacctgacccacagaggatgatcagggcatactgtcaatcagcagccactttgaatctcctcagggcgtttgctactggtggttatgcagctatgcagagggttactcagtggaatttggatttcaccgagcacagtgagcagggagataggtaccgggaacttgcccaccgggttgatgaggcccttggattcatggccgcagctggtctcactgttgatcatcctatcatgacaacaactgagttttggacatcccatgagtgcttgctattgccatatgagcaatcacttactaggctggattcaacctcaggtctttactatgactgctcagcccattttctttgggctggtgagcgtaccaggcagctagatggtgctcatgttgagtttttgagaggagtcgcaaatcccctaggaattaaggtcagtgataagatggatccaaatgagctagttaagctcatagaaattttgaatcctcaaaataaacctggcagaataacaatcattacaagaatgggagctgagaacatgagagtgaagcttcctcatctaataagggctgttcgcagggcaggacaaattgtcacatgggtcagtgatcctatgcatggaaacaccataaaggctccttgtgggctgaaaactcgcccattcgatgccatcagggcggaggtgagagccttctttgatgtgcatgagcaagaaggaagccacccaggaggcgttcatctagagatgactggtcagaatgtgacagagtgcattggtggatcacggcctgtgacatttgatgatttgagttcacgctaccacacccattgtgatcccaggctcaatgcttcacaatctctagagcttgcattcatcattgcagagcgtcttagaaagaggaggattggattacaaccttctattgcaccggcattgctgtag

SEQ ID NO: 68 - DNA, 1596 bp, Populus trichocarpa
  source 1..1596: mol_type = other DNA
atgtctctaactagcagttccataatcccttccaaatccttgatcccctccaacaaaccccatcagccttgtttcttcaccactaaaccttccaggtccaccctccgcatctccgctgtccactctgccgatccttccaaatcaccaccacccattaaaacacctgcagcaacatccactaaagcaacaaccgttgcaccaacagtcaatgtgggtgctggaaaatggacagttgaaagctggaaatcaaagaaggccttgcaactccctgaatatcctgacaaagaagattttgattcggttctcaaaactctcgatgcgttccctccaattgttttcgctggtgaggccaggagtttagaggagaagcttgctgaggctgctatgggcaatgcgtttttgttgcaaggaggtgattgtgctgagagttttaaagagttcaatgctaataatatcagggacactttcaggattcttcttcaaatgggtgttgttttgatgttcggtggtcaaatgcctgttatcaaggtggggaggttggcagggcagtttgcgaagccgagatcggatccatttgaggaaaaagatggagtgaaattgccaagttacaggggggacaatgtgaatggagatgcttttgatgagaagtcgaggatacctgaccctcagaggatgatcagggcttactgtcaatcagcagctactttgaatctacttagggcttttgctactggtggttatgcagctatgcaaagggttactcagtggaatttggatttcacagagcacagtgagcaaggagacaggtaccgggaacttgcccaccgggtcgatgaggcccttggattcatggctgcagctggtctcactgttgatcatcctatcatgacaactactgagttctggacatcccatgagtgcttgctattgccatatgagcaatcacttactaggctggattcaacttcgggtctctactatgactgctcagcccattttctctgggttggtgagcgtaccaggcagctagatggtgctcatgtcgagttcttgagaggagttgcaaatcccctcggaattaaggtcagtgataagatgaatccaaatgagctagttaagctcatagagattttcaacccccagaataaaccaggcagaataacaattattactagaatgggagctgagaacatgagagtgaagcttccccatctaatcagggctgttcgcagggctgggcaaattgtcacatgggtcagtgatcctatgcatggaaacaccataaaggctccatgcggtctgaaaactcgcccatttgattccatcagggcggaggtgagagccttctttgatgtgcatgagcaagaaggaagccacccaggaggagttcatctagagatgactggccagaatgtgacagagtgcattggtggatcacggactgtgacatttgatgatttgagttcacgctaccatacccactgtgatcccaggctcaacgcttcacaatctctagagcttgcattcatcattgcagagcgccttagaaggaggaggattggatcacaatccactgtcgcaccagcattgctgtag

SEQ ID NO: 69 - DNA, 1608 bp, Nicotiana benthamiana
  source 1..1608: mol_type = other DNA
atggctctttcaactagcagcaccaactccctcctaccaaataaatccttaattcaaattcaacctcttttacattctcccaaacatgcccttcccaccaactcaaccaaacccgttagaccgatctcagccgtctattctcccgactcctccaagacccctaaaccttcacccccggcagccacctcctcctccactgctgctgctgctgtcacggggacgaaaacagacaaagtttctaaaaaatggtcagttgacagctggagatcgaagaaagcacttcagcttccagagtacccaaataaagatgatctcgaatcagttcttaagaccattgaagatttccctccaattgtgtttgctggtgaggcgaggagccttgaagagcgacttggagaggctgcaatggggaaggcgtttctgctacaaggtggtgactgtgccgagagtttcaaggaattcaatgctgttaacataagggacactttcagaatcatccttcaaatgggtgctgttcttatgttcggtggtcagatgcccgttgtcaaggttggaagaatggctgggcaatttgcaaagccaagatcagacccatctgaagagaaagatggagtaaagctgccaagttacaggggagataatgtgaatggagatgcatttgatgagaaatccagaactccggatcctcaaaggatgatcagggcctattgtcaatctgcagctactttgaatctcttgagggcctttgctaaaggaggatatgctgccatgcagaggatcaaccagtggaacttagattttacagagcacagtgagcagggtgatcggtatcgtgaattagctcatagagtggatgaggcccttggtttcatggctgctgctggacttacaacggaccatcctatcatgaaaaccaccgagttttggacgtctcacgagtgcttacttttgccctatgagcagtcacttacaagattggattcaacttctggcctttactatgattgctctgcccattttctttgggctggggagagaaccagacaattggatggtgcccatgttgagttcttgagaggaattgccaacccccttgggataaaggtgagtgacaagatggatccaaatgagttagttaagctcatcgacattctgaaccctaacaacaaaccgggaaggattacaataattaccagaatgggagcagagaacatgagggttaggcttcctcatctaataagggcagtgagaagagctgggcaaattgttacatgggtatctgatcctatgcatggaaataccattaaagcaccttgtggtctgaaaactcgaccttttgatgccatcagggctgaagtgagagcattctttgacgtgcatgagcaagaaggaagccatccaggaggagttcacctagagatgacaggccaaaatgtaacagagtgcattggtggatcaaggaccgtgactttcgatgatctgagctcacgctatcacacccactgcgaccctaggctcaatgcatctcaatcccttgaactcgcttttattatcgctgaacgcctcagaaagaggaggcttggatttcaaagctcattaagttactaa

SEQ ID NO: 70 - DNA, 1626 bp, Solanum lycopersicum
  source 1..1626: mol_type = other DNA
atggctctttcaactaataccactaccaactctcttctttccaacaaatctttgcttcaaaatcaaccccttttgtcttctccttcaaagaatgcatttttctccaacaaatcaaccaaaacggttagatttgttcaacctatcgcagcggttcattcgtctgattcaaacaaaaaccccattgtttccgataagccctccaagtcttctccaccggcggccactgcaaccacggctccggctccggctgtgaccaaaactgaatgggcggtggatagctggaagtctaagaaggcgcttcagctaccggaatacccagatcaagaggagcttagatctgttcttaagacgatcgatgagttccctcctatcgtctttgctggtgaggctagaagccttgaagaacgacttggtgaggctgctatgggaagggcgtttttgttacaaggaggagattgtgccgagagtttcaaggaattcaatgctaataatattagggatactttcagaatccttcttcaaatgggtgctgttcttatgtttggtggtcagatgcctgtgatcaaggttggaagaatggctgggcaatttgcaaagccgagatcagattcatttgaggagaaggatggtgtaaagctgccgagttacaggggagacaatgtgaatggagatgcatttgatgtcaagtccaggactcctgaccctcagaggctgatcagggcctattgccaatctgcagctactttgaatctgttgagggcttttgctactggaggatatgctgccatgcagaggatcaatcagtggaacttggatttcacagagcatagtgagcagggtgataggtaccgtgaactagctagtagagtggatgaggcccttggtttcatgactgcagctggacttacaatggaccatcctattatgaaaaccactgagttctggacatctcatgagtgcttacttttgccctatgaacagtcactaacacgccgggattcaacttctggccttcactatgattgctctgctcatttcctttgggttggagagagaaccaggcagttggatggtgcccatgttgagttcttgagaggaattgccaaccctctcggtattaaggtgagtgacaagatggacccaagtgcattggtcaagctcattgagattttgaacccacaaaacaaagctggaaggattacaataattaccagaatgggagcagaaaacatgagggttaagcttcctcatcttatcagggcagtccgaagagcaggacaaattgtgacttgggtatctgatcctatgcacggaaataccatcaaagctccatgcggtctaaaaactcgaccttttgattccatcagggctgaggtaagagcattctttgatgttcatgatcaagaaggaagccacccaggaggagttcatcttgagatgacaggccaaaacgtcacagagtgcattggtggatcacgaactgtgacctttgatgatctgagctcacgttaccacactcactgtgatcctaggctcaatgcatctcaatcccttgagctctcattcattatcgcagaacgtttgaggaaaaggaggctcggatcgcaaagcacattagggcaatag

SEQ ID NO: 71 - DNA, 1629 bp, Nicotiana benthamiana
  source 1..1629: mol_type = other DNA
atggctctttcaagtagtagcactaccaactctctactttccgacaaatctcaattggttcaaaaccaattccttttactttctccattaaagaatgcatctttcaccaccaactcaaccaaagccgttagatttattcaaccaatctcagccgttcattcctctgactcttccaagaaccctattgtctccgacaagccttcccccaagccttcaccgccggcggccactgttacggcggcggctgcgacggtgacgaaaacagaatgggcattggggagctggaaatctaagaaggctcttcagttacccgaatacccaaatcaagaggagcttcaatctgttctcaagacgattgaggagttccctcctatcgttttcgctggtgaggcgaggagtcttgaagagcgtctcggtgaggctgctatgggccgggctttcttgttacaaggaggtgattgtgctgagagttttaaggaatttaatgccaataatattagggatacttttagaatccttcttcaaatgggtgctgttctcatgtttggtggtcagatgcctgttatcaaggttggaaggatggctgggcaatttgcaaagccaagatcagataattatgaggagaagaatggagtgaagctcccaagttacaggggagacaacgtgaacggagatgcatttgatgccaagtcaagaactcctgaccctcagaggttgatcagggcgtattgtcaatctgcagctactttgaatctattgagggcttttgctacaggaggatatgctgccatgcagaggatcaaccaatggaacttggatttcacagagcacagtgagcagggtgataggtatcgtgaactagctaatagagtggatgaggcccttggtttcatggctgctgctggacttacagtggatcatcctattatgaagactaccgagttctggacatctcatgagtgcttgcttttgccctatgagcagtcactaacacgattggattcaacttctggcctttactatgattgttccgcccattttctttgggttggagaaagaactaggcagttggatggtgcccatgttgagttcttgagaggagttgccaacccccttggtattaaggtgagtgacaagatggatccaagtgcattggtcaagctcattgagattttgaaccctgataacaaagctgggaggattacaataattaccagaatgggagcagagaacatgagggttaagcttcctcatcttatcagggcagtccgaagagcagggcaaatcgtcacttgggtatctgatcctatgcatgggaataccatcaaagctccttgcggtctaaaaactagacctttcgattccatcagggctgaagtaagagcattctttgatgttcatgagcaagaaggaagccacccaggaggagtacacctggagatgacaggccaaaacgtcactgagtgcatcggtgggtcacgaactgtgacctttgatgacctgagctcacgttaccacacccactgtgatcctaggctcaatgcatctcaatcccttgagctcgcgttcattattgcagaacgcctaagaaagaggaggcttggatcacgaagcgtattaggtcaatag

SEQ ID NO: 72 - DNA, 1620 bp, Gossypium raimondii
  source 1..1620: mol_type = other DNA
atggctctcaccaactcctccattcttcccaccaactccatccttcaatctcagccatcaacgccgttctctaaggctaagccattgtctgtccttcggatctccgcggtccattcttctgactcgcctaagaaccccgtcgtctccgacaaatccgtcaagcagcccgctactgctccactcgctgctaccaaatctgctaccgcgcccaccactgctccaccgaagaatgcggttcctgggaaatggaccgtggatagctggaagtcgaagaaggctttgcagttgccggagtatccggatcaggcggagctcgagtcggttctcaagactctcgatgctttccctccgattgtcttcgctggagaggctcggagtcttgaggagaagctcggggaagccgccatgggaaacgcttttctgttacaaggcggagattgtgccgagagtttcaaagagttcaacgctaataacattcgtgacactttccggatccttcttcagatgggtgccgttttgatgtttggtggtcaaatgcctgttatcaaggtgggaagaatggctgggcaatttgcgaagccgagatcggacccattcgaggaaaagaatggagtgaagctgccgagttacagaggagataatgtcaacggggattccttcgatgaaaagtctaggatccctgatccccaaagaatgattagagcttactgtcaatcagcagctactttgaatcttttgagagcttttgctactggaggttatgctgctatgcaaagggttactcagtggaatttggatttcaccgaacacagcgagcagggagataggtaccgtgaactagctagtcgtgtggatgaggccctcggattcatggctgcagctggacttacagttgaccatcctatcatgacaacaactgaattctggacttcccatgagtgcttgctcttaccttacgaacagtcacttacccggcttgattctacttcaggccgttactatgattgttctgctcatttcctgtgggccggggaacgaacaagacagttggatggtgcacatgttgagtttttgagaggagttgctaatccccttggaattaaggtgagtgataagatggatccgaatgaactagtaaagctgattgaaattttgaaccctcagaacaaagctggaaggattacagttatcacaagaatgggtgctgagaatatgagagtgaagcttccccatctaattagggcagtgcgtcgtgctgggcaaattgtcacttgggtcagtgaccctatgcatggaaacaccatcaaggctccttgtggtctcaaaactcgccccttcgattccataagggctgaagtgagagctttctttgacgtgcacgagcaagagggaagtcatcctggaggagttcatctagagatgactggacagaatgtgacagaatgcatcgggggatcccggactgttacattcgatgacttgggttcacgctaccatacccactgtgaccctcgtttgaatgcctcccaatctcttgagctcgccttcattatcgccgagcgcctcagaaagaggaggatcaactcaccgcaactgcaaccactaatcacagggctgtaa

SEQ ID NO: 73 - DNA, 1599 bp, Glycine max
  source 1..1599: mol_type = other DNA
atggcaatctcctccacttccaactccctcattcccaccaaatctctaatcccccaatcccaccccctcattcccaacaccaggcccgccctccggcccaagcccggcccatccccttccatcttcgccgttcacgccgccgagcccgccaaaaaccccgtcgtcaccgacaagcccaagccccaagcccaacaacctcccccggcctcggcccgggcaacgaaatgggccgtggacagctggaagtcaaagaaagccctgcagctgcccgaatacccgagccaggaggagctggagtccgtcctcaaaaccctcgaggcttttcctccaatcgtcttcgccggtgaggccaggacattggaggagcatctcgccgaggccgccatgggaaatgccttcctcctccagggcggagactgtgctgagagcttcaaggagttcaatgccaacaacatccgtgacaccttccgcatcatcctccagatgagcgtcgtcatgatgttcggcggccaaatgcctgtcatcaaggtggggagaatggcggggcaatttgcaaagcctcgttcggattcgtttgaggagaagaatggcgtgaagcttccgagttacagaggggataacattaacggagactctttcgacgagaagtcgaggattccggatccgcagaggatgattagggcttattgccaagccgcggccacgctgaatcttctcagagcttttgccaccggtggttatgctgctatgcagagggttactcagtggaatttggacttcacggatcacagcgaacagggagataggtaccgagagcttgctaaccgagttgatgaggcccttggattcatggctgctgctgggctcacggtggaccatcccataatgagaacaactgaattctggacatctcatgagtgcttattgttgccttatgaacaatccctcacaaggttggattcaacttctggtctctactatgactgttcagcccatatgatctgggttggagaacgaaccaggcagcttgatggtgcccatgttgagtttctaagaggagttgctaatcccttgggaattaaggtaagtgacaagatggatccaaatgagctagttaaactcattgagatcttgaatcctcaaaacaaagcaggaagaattactgtgatcacgaggatgggagctgaaaatatgagggtgaagcttccacatctcatcagggcagtgcgcagagcaggccaaattgtcacttgggtcagtgatcctatgcatggaaacaccattaaggctccctgtggtcttaaaactcgccccttcgattccatcagggccgaagtgagagcattcttcgacgtacacgagcaagaaggaagccacccaggaggggttcatctagagatgacgggtcagaatgtgaccgagtgcattggtgggtcaaggacggtcacatttgatgacttgagctcacgttaccacacacactgtgacccaaggctcaatgcttcacaatctcttgagcttgctttcatcatcgctgagcgtttgagaaagagcaggatcagatcgcagcaacctcttgcccctctaggagtactgtaa

SEQ ID NO: 74 - DNA, 1212 bp, Glycine max
  source 1..1212: mol_type = other DNA
atgggaaatgccttcctcctccagggcggagactgtgccgagagcttcaaggagttcaatgccaacaacatccgtgacaccttccgcatcatcctccagatgagcgtcgtcatgatgttcggcggccaaatgcccgtcatcaaggtggggagaatggcggggcaatttgcgaagccgaggtcggattcgtttgaggagaagaacggcgtgaagcttccgagttacagaggggacaacattaacggagactcctttgacgagaagtcgaggattccggatccgcagaggatgattagggcttattgccaagccgcggcgacgctgaatcttctcagagctttcgccaccggtggttatgctgctatgcagagggttactcagtggaatttggacttcacggatcacagcgaacagggagataggtaccgagagcttgctaaccgagttgatgaggctcttggattcatggctgctgctgggctcacagtggaccatcccataatgagaacaactgaattctggacatctcatgagtgcttattgttgccttatgaacaatccctcaccaggttggattcaacttctggtctctactatgactgttcagcccatatgctctgggttggggaacgaaccaggcagcttgatggtgcccatgtcgagtttctaagaggagttgctaatcccttgggaattaaggtaagtgacaagatggatccaaatgagcttgttagactcattgagatcttgaatccccaaaacaaaccagggagaataactgtgattacgaggatgggagctgaaaatatgagggtgaagcttccacatctcatcagggcagtgcgcagagcagggcaaattgtcacctgggtcagtgatcctatgcatggaaacaccattaaggctccatgtggtcttaaaactcgccccttcgatttcatcagggctgaagtgagagcattctttgatgtgcacgagcaagaaggaagccacccaggaggggttcatctagagatgacgggtcagaatgtgaccgagtgcattggtgggtcaaggacggtcacatttgatgacttgagctcacgttaccacacacactgtgacccaaggctcaatgcttcacaatctcttgagcttgctttcatcatcgccgagcgtttgagaaagagcaggatcagatcgcagcaacctcttgcccctctaggagtgtaa

SEQ ID NO: 75 - protein, 9 aa, synthetic construct
YPYDVPDYA

SEQ ID NO: 76 - DNA, 31 bp, synthetic construct
  source 1..31: mol_type = other DNA
gagcatttgctactggaggttatgcagctat

SEQ ID NO: 77 - DNA, 33 bp, synthetic construct
  source 1..33: mol_type = other DNA
atttgctactggaggttatgcagctatgcagag

SEQ ID NO: 78 - DNA, 31 bp, synthetic construct
  source 1..31: mol_type = other DNA
ttagagcctttgccactggaggttacgctgc

SEQ ID NO: 79 - DNA, 30 bp, synthetic construct
  source 1..30: mol_type = other DNA
gacacctttagggttcttcttcagatgggt

SEQ ID NO: 80 - DNA, 29 bp, synthetic construct
  source 1..29: mol_type = other DNA
tttgaatcttttgagagcttttgctactg

SEQ ID NO: 81 - DNA, 31 bp, synthetic construct
  source 1..31: mol_type = other DNA
atcgtgttcgccggagaagctagattgcttg

SEQ ID NO: 82 - DNA, 31 bp, synthetic construct
  source 1..31: mol_type = other DNA
gagcatttgctactggaagttatgcagctat

SEQ ID NO: 83 - DNA, 33 bp, synthetic construct
  source 1..33: mol_type = other DNA
atttgctactggaggttatacagctatgcagag

SEQ ID NO: 84 - DNA, 31 bp, synthetic construct
  source 1..31: mol_type = other DNA
ttagagcctttgccactagaggttacgctgc

SEQ ID NO: 85 - DNA, 30 bp, synthetic construct
  source 1..30: mol_type = other DNA
gacacctttagggttctttttcagatgggt

SEQ ID NO: 86 - DNA, 29 bp, synthetic construct
  source 1..29: mol_type = other DNA
tttgaatcttttgagaacttttgctactg

SEQ ID NO: 87 - DNA, 31 bp, synthetic construct
  source 1..31: mol_type = other DNA
atcgtgttcgccagagaagctagattgcttg

SEQ ID NO: 88 - DNA, 20 bp, synthetic construct
  source 1..20: mol_type = other DNA
ttttcaggtgggaagaatgg

SEQ ID NO: 89 - DNA, 20 bp, synthetic construct
  source 1..20: mol_type = other DNA
tgttgcgtgaaatcaaggtt

SEQ ID NO: 90 - DNA, 19 bp, synthetic construct
  source 1..19: mol_type = other DNA
gagttacagaggagataac

SEQ ID NO: 91 - DNA, 19 bp, synthetic construct
  source 1..19: mol_type = other DNA
acccatgaatcccaaagcc

SEQ ID NO: 92 - DNA, 21 bp, synthetic construct
  source 1..21: mol_type = other DNA
ttgaggagaaggatggagtga

SEQ ID NO: 93 - DNA, 21 bp, synthetic construct
  source 1..21: mol_type = other DNA
tcagcttgctcactttgttca

SEQ ID NO: 94 - DNA, 24 bp, synthetic construct
  source 1..24: mol_type = other DNA
ggtggtgattgtgctgagagtttc

SEQ ID NO: 95 - DNA, 34 bp, synthetic construct
  source 1..34: mol_type = other DNA
tggccaccgaacatgagaacaacacccatctcta

SEQ ID NO: 96 - DNA, 26 bp, synthetic construct
  source 1..26: mol_type = other DNA
cctgatccacagaggatgattagagc

SEQ ID NO: 97 - DNA, 29 bp, synthetic construct
  source 1..29: mol_type = other DNA
catagcagcataaccaccagtagcaagag

SEQ ID NO: 98 - DNA, 23 bp, synthetic construct
  source 1..23: mol_type = other DNA
ggactatccagatttagctgcgc

SEQ ID NO: 99 - DNA, 25 bp, synthetic construct
  source 1..25: mol_type = other DNA
cgttcctcaagcaatctagcagatc

SEQ ID NO: 100 - DNA, 18 bp, synthetic construct
  source 1..18: mol_type = other DNA
gcggagagcgtaccagac

SEQ ID NO: 101 - DNA, 22 bp, synthetic construct
  source 1..22: mol_type = other DNA
gatccattttgttgctcacctt

SEQ ID NO: 102 - DNA, 20 bp, synthetic construct
  source 1..20: mol_type = other DNA
caatgcacggaaacacaatc

SEQ ID NO: 103 - DNA, 19 bp, synthetic construct
  source 1..19: mol_type = other DNA
acgtcgaagaacgctctca

SEQ ID NO: 104 - DNA, 20 bp, synthetic construct
  source 1..20: mol_type = other DNA
caagactcgtccctttgacg

SEQ ID NO: 105 - DNA, 19 bp, synthetic construct
  source 1..19: mol_type = other DNA
gggtggctaccttcttgct

SEQ ID NO: 106 - DNA, 23 bp, synthetic construct
  source 1..23: mol_type = other DNA
tcctacttcatgtagcgcaggac

SEQ ID NO: 107 - DNA, 23 bp, synthetic construct
  source 1..23: mol_type = other DNA
tcctccagaataagggctatccg

SEQ ID NO: 108 - DNA, 33 bp, synthetic construct
  source 1..33: mol_type = other DNA
ctttgccactagaggttacgctgctattcaaag

SEQ ID NO: 109 - DNA, 32 bp, synthetic construct
  source 1..32: mol_type = other DNA
gcgtaacctctagtggcaaaggctctaagaag

SEQ ID NO: 110 - DNA, 35 bp, synthetic construct
  source 1..35: mol_type = other DNA
gctactggaagttatgcagctatgcagagagttag

SEQ ID NO: 111 - DNA, 34 bp, synthetic construct
  source 1..34: mol_type = other DNA
ctgcataacttccagtagcaaatgctctcaagag

SEQ ID NO: 112 - DNA, 33 bp, synthetic construct
  source 1..33: mol_type = other DNA
ggaggttatacagctatgcagagagttagccag

SEQ ID NO: 113 - DNA, 33 bp, synthetic construct
  source 1..33: mol_type = other DNA
gcatagctgtataacctccagtagcaaatgctc

SEQ ID NO: 114 - DNA, 34 bp, synthetic construct
  source 1..34: mol_type = other DNA
tagggttctttttcagatgggtgttgttctcatg

SEQ ID NO: 115 - DNA, 36 bp, synthetic construct
  source 1..36: mol_type = other DNA
cccatctgaaaaagaaccctaaaggtgtctctaatg

SEQ ID NO: 116 - DNA, 34 bp, synthetic construct
  source 1..34: mol_type = other DNA
ctttgaatcttttgagaacttttgctactggtgg

SEQ ID NO: 117 - DNA, 34 bp, synthetic construct
  source 1..34: mol_type = other DNA
ccaccagtagcaaaagttctcaaaagattcaaag

SEQ ID NO: 118 - DNA, 35 bp, synthetic construct
  source 1..35: mol_type = other DNA
cgtgttcgccagagaagctagattgcttgaggaac

SEQ ID NO: 119 - DNA, 35 bp, synthetic construct
  source 1..35: mol_type = other DNA
ctagcttctctggcgaacacgatcggaggaaaagc

SEQ ID NO: 120 - DNA, 19 bp, synthetic construct
  source 1..19: mol_type = other DNA
attttgccgatttcggaac

SEQ ID NO: 121 - DNA, 20 bp, synthetic construct
  source 1..20: mol_type = other DNA
tcctcctttggcgcaaccgc

SEQ ID NO: 122 - DNA, 22 bp, synthetic construct
  source 1..22: mol_type = other DNA
gcgtcacgaattgcgatccagc

SEQ ID NO: 123 - DNA, 740 bp, synthetic construct
  source 1..740: mol_type = other DNA
gaattcatggggcattacaattaattagtaatactaattttaataacactaaatcatattagtctctataggtataatatataactctggagtcactatctgtatttgggatcaacagttacaaaggtacgagtaaagtctctgatccgacaaacaatccgtatatcctaccttcccgaaatttcatgtataaaattatactaaaatattatttttcttgtattatccgattgaaaattatgcgaattcaacataaaactctaaaaaaagaaaataatgaaaattacaacttaaaagagtgtctaagttggaatcatggtattacataaagttgaggacaatataagcaagcaaacaagtactctagctatcaatttactttggactactatatgataaatatttcaacccccctcctcccccaccaaaaaaaaattaaaataacaagttgaaggactcaaaaaaaaaaaaaaaaaaaactcaaaaccaacctcaatcatacattcatatcctcttcctacccccatcttggatgagataagattaacgaggtgcttacacgtgtcacctctattgtggtgacttaaaaaaaattccaacctttcatatgtagatattaagtaattgtataatgttatcaagaaccacataacatatcaaaaaccttatcatttcattatataaaaggatagtggacatcaaaaggttcatattgaaccaaaaaaagagagaagaagcaa

SEQ ID NO: 124 - DNA, 177 bp, synthetic construct
  source 1..177: mol_type = other DNA
atggcttcttctatgctttcttctgctgctgttgttgctactcgtgctagtgctgctcaagctagtatggttgctccttttactggacttaagtctgctgcttcttttcctgttactagaaagcaaaacaaccttgatattacttctattgctagtaacggaggaagagttcgagca

SEQ ID NO: 125 - DNA, 1434 bp, synthetic construct
  source 1..1434: mol_type = other DNA
atgctccgcgcccgcgccgtccgcgcggcgccgcggcccccgagccagtggtccgtcggcagctggcggggccgcccggcgctgcagcagcccgaatacccggacaaggcggacctgaacgaggtgctgcggacggtggaggcgttcccgcccatcgtgtttgccggcgaggcgcgcaccctcgaggagcgcctcgcggaggccgccgtcggcagagctttcctcctccagggcggtgactgcgctgagagcttcaaggagttcaacgccaacaatattagggacacctttcgcgtcctcctgcaaatgtctgttgtgctcatgttcggaggccagatgcctgtcgtcaaggtgggaagaatggcaggtcagtttgcaaagccaagatcagatggttttgaggagcgggatggagtgaagttgccaagctatagaggggacaatatcaatggggatgcatttgatgagaagtcaagattgccagatccacaccgcatgataagtgcctactcacagtctgcagcaacgctgaatctgctgcgggcgttcgcaactggaggttatgctgcaatgcagagggtaacacaatggaaccttgatttcacagagcatagtgaacaaggggataggtacatggaattggctcaccgagttgacgaagctttggggttcatgtcagctgctggactcactttagatcaccctataatgacgacaacagaattctggacgtcacatgagtgccttcttctgccttatgagcaagcgctcacccgtgaggactccaccagtggcctctattatgattgctctgctcacttcctatgggttggagagcgcactcgccagcttgatggtgctcatgtggagttccttcgaggcatcgccaaccctcttggtatcaaggttagtgacaagatggacccagcagaacttgtgcggttgattgatatattgaatcccgaaaacagggctgggagaataaccatcatcgcaagaatgggacctgaaaacatgagggtgaaacttccacacctgatacgcgccgtccgtggggccggccagatagtaacatgggtcactgacccaatgcatgggaacaccatgaaggccccttgcggactcaaaacccgctcattcgacagaattttggccgaggtgcgtgcgttctttgatgtccacgaacaagaagggagctaccctggaggagtgcatctagagatgactgggcaaaatgttacagagtgcattggagggtcacgtaccgtgacatttgatgatctgggctcacgctaccacacgcactgcgacccaaggctcaacgcctcacagtctctggagatggcattcattatagcggagcgccttaggaaaaggaggattgcctcgtcgcctttgtacacgaaccagctgggttccattcgtctgggttcg

SEQ ID NO: 126 - DNA, 1434 bp, synthetic construct
  source 1..1434: mol_type = other DNA
atgctccgcgcccgcgccgtccgcgcggcgccgcggcccccgagccagtggtccgtcggcagctggcggggccgcccggcgctgcagcagcccgaatacccggacaaggcggacctgaacgaggtgctgcggacggtggaggcgttcccgcccatcgtgtttgccggcgaggcgcgcaccctcgaggagcgcctcgcggaggccgccgtcggcagagctttcctcctccagggcggtgactgcgctgagagcttcaaggagttcaacgccaacaatattagggacacctttcgcgtcctcctgcaaatgtctgttgtgctcatgttcggaggccagatgcctgtcgtcaaggtgggaagaatggcaggtcagtttgcaaagccaagatcagatggttttgaggagcgggatggagtgaagttgccaagctatagaggggacaatatcaatggggatgcatttgatgagaagtcaagattgccagatccacaccgcatgataagtgcctactcacagtctgcagcaacgctgaatctgctgcgggcgttcgcaactaggggttatgctgcaatgcagagggtaacacaatggaaccttgatttcacagagcatagtgaacaaggggataggtacatggaattggctcaccgagttgacgaagctttggggttcatgtcagctgctggactcactttagatcaccctataatgacgacaacagaattctggacgtcacatgagtgccttcttctgccttatgagcaagcgctcacccgtgaggactccaccagtggcctctattatgattgctctgctcacttcctatgggttggagagcgcactcgccagcttgatggtgctcatgtggagttccttcgaggcatcgccaaccctcttggtatcaaggttagtgacaagatggacccagcagaacttgtgcggttgattgatatattgaatcccgaaaacagggctgggagaataaccatcatcgcaagaatgggacctgaaaacatgagggtgaaacttccacacctgatacgcgccgtccgtggggccggccagatagtaacatgggtcactgacccaatgcatgggaacaccatgaaggccccttgcggactcaaaacccgctcattcgacagaattttggccgaggtgcgtgcgttctttgatgtccacgaacaagaagggagctaccctggaggagtgcatctagagatgactgggcaaaatgttacagagtgcattggagggtcacgtaccgtgacatttgatgatctgggctcacgctaccacacgcactgcgacccaaggctcaacgcctcacagtctctggagatggcattcattatagcggagcgccttaggaaaaggaggattgcctcgtcgcctttgtacacgaaccagctgggttccattcgtctgggttcg

SEQ ID NO: 127 - DNA, 1434 bp, synthetic construct
  source 1..1434: mol_type = other DNA
atgctccgcgcccgcgccgtccgcgcggcgccgcggcccccgagccagtggtccgtcggcagctggcggggccgcccggcgctgcagcagcccgaatacccggacaaggcggacctgaacgaggtgctgcggacggtggaggcgttcccgcccatcgtgtttgccggcgaggcgcgcaccctcgaggagcgcctcgcggaggccgccgtcggcagagctttcctcctccagggcggtgactgcgctgagagcttcaaggagttcaacgccaacaatattagggacacctttcgcgtcctcttccaaatgtctgttgtgctcatgttcggaggccagatgcctgtcgtcaaggtgggaagaatggcaggtcagtttgcaaagccaagatcagatggttttgaggagcgggatggagtgaagttgccaagctatagaggggacaatatcaatggggatgcatttgatgagaagtcaagattgccagatccacaccgcatgataagtgcctactcacagtctgcagcaacgctgaatctgctgcgggcgttcgcaactggaggttatgctgcaatgcagagggtaacacaatggaaccttgatttcacagagcatagtgaacaaggggataggtacatggaattggctcaccgagttgacgaagctttggggttcatgtcagctgctggactcactttagatcaccctataatgacgacaacagaattctggacgtcacatgagtgccttcttctgccttatgagcaagcgctcacccgtgaggactccaccagtggcctctattatgattgctctgctcacttcctatgggttggagagcgcactcgccagcttgatggtgctcatgtggagttccttcgaggcatcgccaaccctcttggtatcaaggttagtgacaagatggacccagcagaacttgtgcggttgattgatatattgaatcccgaaaacagggctgggagaataaccatcatcgcaagaatgggacctgaaaacatgagggtgaaacttccacacctgatacgcgccgtccgtggggccggccagatagtaacatgggtcactgacccaatgcatgggaacaccatgaaggccccttgcggactcaaaacccgctcattcgacagaattttggccgaggtgcgtgcgttctttgatgtccacgaacaagaagggagctaccctggaggagtgcatctagagatgactgggcaaaatgttacagagtgcattggagggtcacgtaccgtgacatttgatgatctgggctcacgctaccacacgcactgcgacccaaggctcaacgcctcacagtctctggagatggcattcattatagcggagcgccttaggaaaaggaggattgcctcgtcgcctttgtacacgaaccagctgggttccattcgtctgggttcg

SEQ ID NO: 128 - DNA, 1560 bp, synthetic construct
  source 1..1560: mol_type = other DNA
atgcggggttctcatcatcatcatcatcatggtatggctagcatgactggtggacagcaaatgggtcgggatctgtacgacgatgacgataaggatcatcccttcaccgttcatgcagcagaaccgaccaaaaatagcgttagcgttaaagaaagcgcagcaccggttgcagcacctggcaccggtaaatggtcactggaaagctggaaaaccaaaaaagcactgcagctgccggaatatccggatgcaaatgaactggaaaccgttctgaaaaccattgaaacctttccgcctattgtttttgccggtgaagcacgtagcctggaagaaaatctggccgaagcagcaatgggtaatgcattcctgctgcaaggtggtgattgtgcagaaagctttaaagaattttccgccaacaatatccgtgatacctttcgtattctgttacagatgggtgttgttctgatgtttggtggtcaggttccggttattaaagttggtcgtatggcaggtcagtttgcaaaaccgcgtagcgatccgtttgaagaaaaagatggtgttaaactgccgagctataaaggcgataacattaatggtgatgcctttaacgaaaaaagccgtattccggatccgcagcgtctgattcgtgcatattgtcagagcgcagcaaccctgaatctgctgcgtgcatttgcgaccggtggttatgcagcaatgcagcgtgttacccagtggaatctggattttgcacagcatagcgaacagggtgatcgttatcaagaactggcaaatcgtgttgatgaagcactgggttttatggcagcagcaggtctgaccgttgatcatccgattatgaccaccaccgaattttggaccagccatgaatgtctgcatctgccgtatgaacagagtctgacccgtctggatagcaccagcggtctgtattatgattgtagcgcacatatgctgtggtgtggtgaacgtacccgtcagctggatggtgcacatgttgaatttctgcgtggtattagcaatccgattggtattaaagtgagcaacaaaatggatccgaacgaactggttaaactgatcgaaatctttaacagcaacaataaaccgggtcgcattaccattattgttcgtatgggtgcagaaaacatgcgtgttaaatttccgcatctgattcgcgcagttcgtcgtgcaggtcagattgttacctgggtttgtgatccgatgcatggcaataccattaaagcaccgtgtggcctgaaaacccgtccgtttgatgcaattctggcggaagttcgtgcattttttgatgtgcatgaacaagagggtagccatcctggtggtattcatctggaaatgacaggtcagaatgtgaccgaatgtattggtggtagccgtaccgttacctttgatgatctgagcagccgttatcatacacattgcgatccgcgtctgaatgcaagccagagcctggaactggcatttatcattgcagaacgtctgcgtaaacgtcgtattggcacccagcgtctgtttagcctgggtcagggttcg

SEQ ID NO: 129 - DNA, 1560 bp, synthetic construct
  source 1..1560: mol_type = other DNA
atgcggggttctcatcatcatcatcatcatggtatggctagcatgactggtggacagcaaatgggtcgggatctgtacgacgatgacgataaggatcatcccttcaccgttcatgcagcagaaccgaccaaaaatagcgttagcgttaaagaaagcgcagcaccggttgcagcacctggcaccggtaaatggtcactggaaagctggaaaaccaaaaaagcactgcagctgccggaatatccggatgcaaatgaactggaaaccgttctgaaaaccattgaaacctttccgcctattgtttttgccggtgaagcacgtagcctggaagaaaatctggccgaagcagcaatgggtaatgcattcctgctgcaaggtggtgattgtgcagaaagctttaaagaattttccgccaacaatatccgtgatacctttcgtattctgttacagatgggtgttgttctgatgtttggtggtcaggttccggttattaaagttggtcgtatggcaggtcagtttgcaaaaccgcgtagcgatccgtttgaagaaaaagatggtgttaaactgccgagctataaaggcgataacattaatggtgatgcctttaacgaaaaaagccgtattccggatccgcagcgtctgattcgtgcatattgtcagagcgcagcaaccctgaatctgctgcgtgcatttgcgaccagaggttatgcagcaatgcagcgtgttacccagtggaatctggattttgcacagcatagcgaacagggtgatcgttatcaagaactggcaaatcgtgttgatgaagcactgggttttatggcagcagcaggtctgaccgttgatcatccgattatgaccaccaccgaattttggaccagccatgaatgtctgcatctgccgtatgaacagagtctgacccgtctggatagcaccagcggtctgtattatgattgtagcgcacatatgctgtggtgtggtgaacgtacccgtcagctggatggtgcacatgttgaatttctgcgtggtattagcaatccgattggtattaaagtgagcaacaaaatggatccgaacgaactggttaaactgatcgaaatctttaacagcaacaataaaccgggtcgcattaccattattgttcgtatgggtgcagaaaacatgcgtgttaaatttccgcatctgattcgcgcagttcgtcgtgcaggtcagattgttacctgggtttgtgatccgatgcatggcaataccattaaagcaccgtgtggcctgaaaacccgtccgtttgatgcaattctggcggaagttcgtgcattttttgatgtgcatgaacaagagggtagccatcctggtggtattcatctggaaatgacaggtcagaatgtgaccgaatgtattggtggtagccgtaccgttacctttgatgatctgagcagccgttatcatacacattgcgatccgcgtctgaatgcaagccagagcctggaactggcatttatcattgcagaacgtctgcgtaaacgtcgtattggcacccagcgtctgtttagcctgggtcagggttcg

SEQ ID NO: 130 - DNA, 1560 bp, synthetic construct
  source 1..1560: mol_type = other DNA
atgcggggttctcatcatcatcatcatcatggtatggctagcatgactggtggacagcaaatgggtcgggatctgtacgacgatgacgataaggatcatcccttcaccgttcatgcagcagaaccgaccaaaaatagcgttagcgttaaagaaagcgcagcaccggttgcagcacctggcaccggtaaatggtcactggaaagctggaaaaccaaaaaagcactgcagctgccggaatatccggatgcaaatgaactggaaaccgttctgaaaaccattgaaacctttccgcctattgtttttgccggtgaagcacgtagcctggaagaaaatctggccgaagcagcaatgggtaatgcattcctgctgcaaggtggtgattgtgcagaaagctttaaagaattttccgccaacaatatccgtgatacctttcgtattctgtttcagatgggtgttgttctgatgtttggtggtcaggttccggttattaaagttggtcgtatggcaggtcagtttgcaaaaccgcgtagcgatccgtttgaagaaaaagatggtgttaaactgccgagctataaaggcgataacattaatggtgatgcctttaacgaaaaaagccgtattccggatccgcagcgtctgattcgtgcatattgtcagagcgcagcaaccctgaatctgctgcgtgcatttgcgaccggtggttatgcagcaatgcagcgtgttacccagtggaatctggattttgcacagcatagcgaacagggtgatcgttatcaagaactggcaaatcgtgttgatgaagcactgggttttatggcagcagcaggtctgaccgttgatcatccgattatgaccaccaccgaattttggaccagccatgaatgtctgcatctgccgtatgaacagagtctgacccgtctggatagcaccagcggtctgtattatgattgtagcgcacatatgctgtggtgtggtgaacgtacccgtcagctggatggtgcacatgttgaatttctgcgtggtattagcaatccgattggtattaaagtgagcaacaaaatggatccgaacgaactggttaaactgatcgaaatctttaacagcaacaataaaccgggtcgcattaccattattgttcgtatgggtgcagaaaacatgcgtgttaaatttccgcatctgattcgcgcagttcgtcgtgcaggtcagattgttacctgggtttgtgatccgatgcatggcaataccattaaagcaccgtgtggcctgaaaacccgtccgtttgatgcaattctggcggaagttcgtgcattttttgatgtgcatgaacaagagggtagccatcctggtggtattcatctggaaatgacaggtcagaatgtgaccgaatgtattggtggtagccgtaccgttacctttgatgatctgagcagccgttatcatacacattgcgatccgcgtctgaatgcaagccagagcctggaactggcatttatcattgcagaacgtctgcgtaaacgtcgtattggcacccagcgtctgtttagcctgggtcagggttcg

SEQ ID NO: 131 - protein, 59 aa, synthetic construct
MASSMLSSAAVVATRASAAQASMVAPFTGLKSAASFPVTRKQNNLDITSIASNGGRVRA

SEQ ID NO: 132 - protein, 478 aa, synthetic construct
MLRARAVRAAPRPPSQWSVGSWRGRPALQQPEYPDKADLNEVLRTVEAFPPIVFAGEARTLEERLAEAAVGRAFLLQGGDCAESFKEFNANNIRDTFRVLLQMSVVLMFGGQMPVVKVGRMAGQFAKPRSDGFEERDGVKLPSYRGDNINGDAFDEKSRLPDPHRMISAYSQSAATLNLLRAFATGGYAAMQRVTQWNLDFTEHSEQGDRYMELAHRVDEALGFMSAAGLTLDHPIMTTTEFWTSHECLLLPYEQALTREDSTSGLYYDCSAHFLWVGERTRQLDGAHVEFLRGIANPLGIKVSDKMDPAELVRLIDILNPENRAGRITIIARMGPENMRVKLPHLIRAVRGAGQIVTWVTDPMHGNTMKAPCGLKTRSFDRILAEVRAFFDVHEQEGSYPGGVHLEMTGQNVTECIGGSRTVTFDDLGSRYHTHCDPRLNASQSLEMAFIIAERLRKRRIASSPLYTNQLGSIRLGS

SEQ ID NO: 133 - protein, 478 aa, synthetic construct
MLRARAVRAAPRPPSQWSVGSWRGRPALQQPEYPDKADLNEVLRTVEAFPPIVFAGEARTLEERLAEAAVGRAFLLQGGDCAESFKEFNANNIRDTFRVLLQMSVVLMFGGQMPVVKVGRMAGQFAKPRSDGFEERDGVKLPSYRGDNINGDAFDEKSRLPDPHRMISAYSQSAATLNLLRAFATRGYAAMQRVTQWNLDFTEHSEQGDRYMELAHRVDEALGFMSAAGLTLDHPIMTTTEFWTSHECLLLPYEQALTREDSTSGLYYDCSAHFLWVGERTRQLDGAHVEFLRGIANPLGIKVSDKMDPAELVRLIDILNPENRAGRITIIARMGPENMRVKLPHLIRAVRGAGQIVTWVTDPMHGNTMKAPCGLKTRSFDRILAEVRAFFDVHEQEGSYPGGVHLEMTGQNVTECIGGSRTVTFDDLGSRYHTHCDPRLNASQSLEMAFIIAERLRKRRIASSPLYTNQLGSIRLGS

SEQ ID NO: 134 - protein, 478 aa, synthetic construct
MLRARAVRAAPRPPSQWSVGSWRGRPALQQPEYPDKADLNEVLRTVEAFPPIVFAGEARTLEERLAEAAVGRAFLLQGGDCAESFKEFNANNIRDTFRVLFQMSVVLMFGGQMPVVKVGRMAGQFAKPRSDGFEERDGVKLPSYRGDNINGDAFDEKSRLPDPHRMISAYSQSAATLNLLRAFATGGYAAMQRVTQWNLDFTEHSEQGDRYMELAHRVDEALGFMSAAGLTLDHPIMTTTEFWTSHECLLLPYEQALTREDSTSGLYYDCSAHFLWVGERTRQLDGAHVEFLRGIANPLGIKVSDKMDPAELVRLIDILNPENRAGRITIIARMGPENMRVKLPHLIRAVRGAGQIVTWVTDPMHGNTMKAPCGLKTRSFDRILAEVRAFFDVHEQEGSYPGGVHLEMTGQNVTECIGGSRTVTFDDLGSRYHTHCDPRLNASQSLEMAFIIAERLRKRRIASSPLYTNQLGSIRLGS

SEQ ID NO: 135 - protein, 520 aa, synthetic construct
MRGSHHHHHHGMASMTGGQQMGRDLYDDDDKDHPFTVHAAEPTKNSVSVKESAAPVAAPGTGKWSLESWKTKKALQLPEYPDANELETVLKTIETFPPIVFAGEARSLEENLAEAAMGNAFLLQGGDCAESFKEFSANNIRDTFRILLQMGVVLMFGGQVPVIKVGRMAGQFAKPRSDPFEEKDGVKLPSYKGDNINGDAFNEKSRIPDPQRLIRAYCQSAATLNLLRAFATGGYAAMQRVTQWNLDFAQHSEQGDRYQELANRVDEALGFMAAAGLTVDHPIMTTTEFWTSHECLHLPYEQSLTRLDSTSGLYYDCSAHMLWCGERTRQLDGAHVEFLRGISNPIGIKVSNKMDPNELVKLIEIFNSNNKPGRITIIVRMGAENMRVKFPHLIRAVRRAGQIVTWVCDPMHGNTIKAPCGLKTRPFDAILAEVRAFFDVHEQEGSHPGGIHLEMTGQNVTECIGGSRTVTFDDLSSRYHTHCDPRLNASQSLELAFIIAERLRKRRIGTQRLFSLGQGS

SEQ ID NO: 136 - protein, 520 aa, synthetic construct
MRGSHHHHHHGMASMTGGQQMGRDLYDDDDKDHPFTVHAAEPTKNSVSVKESAAPVAAPGTGKWSLESWKTKKALQLPEYPDANELETVLKTIETFPPIVFAGEARSLEENLAEAAMGNAFLLQGGDCAESFKEFSANNIRDTFRILLQMGVVLMFGGQVPVIKVGRMAGQFAKPRSDPFEEKDGVKLPSYKGDNINGDAFNEKSRIPDPQRLIRAYCQSAATLNLLRAFATRGYAAMQRVTQWNLDFAQHSEQGDRYQELANRVDEALGFMAAAGLTVDHPIMTTTEFWTSHECLHLPYEQSLTRLDSTSGLYYDCSAHMLWCGERTRQLDGAHVEFLRGISNPIGIKVSNKMDPNELVKLIEIFNSNNKPGRITIIVRMGAENMRVKFPHLIRAVRRAGQIVTWVCDPMHGNTIKAPCGLKTRPFDAILAEVRAFFDVHEQEGSHPGGIHLEMTGQNVTECIGGSRTVTFDDLSSRYHTHCDPRLNASQSLELAFIIAERLRKRRIGTQRLFSLGQGS

SEQ ID NO: 137 - protein, 520 aa, synthetic construct
MRGSHHHHHHGMASMTGGQQMGRDLYDDDDKDHPFTVHAAEPTKNSVSVKESAAPVAAPGTGKWSLESWKTKKALQLPEYPDANELETVLKTIETFPPIVFAGEARSLEENLAEAAMGNAFLLQGGDCAESFKEFSANNIRDTFRILFQMGVVLMFGGQVPVIKVGRMAGQFAKPRSDPFEEKDGVKLPSYKGDNINGDAFNEKSRIPDPQRLIRAYCQSAATLNLLRAFATGGYAAMQRVTQWNLDFAQHSEQGDRYQELANRVDEALGFMAAAGLTVDHPIMTTTEFWTSHECLHLPYEQSLTRLDSTSGLYYDCSAHMLWCGERTRQLDGAHVEFLRGISNPIGIKVSNKMDPNELVKLIEIFNSNNKPGRITIIVRMGAENMRVKFPHLIRAVRRAGQIVTWVCDPMHGNTIKAPCGLKTRPFDAILAEVRAFFDVHEQEGSHPGGIHLEMTGQNVTECIGGSRTVTFDDLSSRYHTHCDPRLNASQSLELAFIIAERLRKRRIGTQRLFSLGQGS

SEQ ID NO: 138 - protein, 537 aa, synthetic construct
MASSMLSSAAVVATRASAAQASMVAPFTGLKSAASFPVTRKQNNLDITSIASNGGRVRAMLRARAVRAAPRPPSQWSVGSWRGRPALQQPEYPDKADLNEVLRTVEAFPPIVFAGEARTLEERLAEAAVGRAFLLQGGDCAESFKEFNANNIRDTFRVLLQMSVVLMFGGQMPVVKVGRMAGQFAKPRSDGFEERDGVKLPSYRGDNINGDAFDEKSRLPDPHRMISAYSQSAATLNLLRAFATGGYAAMQRVTQWNLDFTEHSEQGDRYMELAHRVDEALGFMSAAGLTLDHPIMTTTEFWTSHECLLLPYEQALTREDSTSGLYYDCSAHFLWVGERTRQLDGAHVEFLRGIANPLGIKVSDKMDPAELVRLIDILNPENRAGRITIIARMGPENMRVKLPHLIRAVRGAGQIVTWVTDPMHGNTMKAPCGLKTRSFDRILAEVRAFFDVHEQEGSYPGGVHLEMTGQNVTECIGGSRTVTFDDLGSRYHTHCDPRLNASQSLEMAFIIAERLRKRRIASSPLYTNQLGSIRLGS

SEQ ID NO: 139 - protein, 537 aa, synthetic construct
MASSMLSSAAVVATRASAAQASMVAPFTGLKSAASFPVTRKQNNLDITSIASNGGRVRAMLRARAVRAAPRPPSQWSVGSWRGRPALQQPEYPDKADLNEVLRTVEAFPPIVFAGEARTLEERLAEAAVGRAFLLQGGDCAESFKEFNANNIRDTFRVLLQMSVVLMFGGQMPVVKVGRMAGQFAKPRSDGFEERDGVKLPSYRGDNINGDAFDEKSRLPDPHRMISAYSQSAATLNLLRAFATRGYAAMQRVTQWNLDFTEHSEQGDRYMELAHRVDEALGFMSAAGLTLDHPIMTTTEFWTSHECLLLPYEQALTREDSTSGLYYDCSAHFLWVGERTRQLDGAHVEFLRGIANPLGIKVSDKMDPAELVRLIDILNPENRAGRITIIARMGPENMRVKLPHLIRAVRGAGQIVTWVTDPMHGNTMKAPCGLKTRSFDRILAEVRAFFDVHEQEGSYPGGVHLEMTGQNVTECIGGSRTVTFDDLGSRYHTHCDPRLNASQSLEMAFIIAERLRKRRIASSPLYTNQLGSIRLGS

SEQ ID NO: 140 - protein, 537 aa, synthetic construct
MASSMLSSAAVVATRASAAQASMVAPFTGLKSAASFPVTRKQNNLDITSIASNGGRVRAMLRARAVRAAPRPPSQWSVGSWRGRPALQQPEYPDKADLNEVLRTVEAFPPIVFAGEARTLEERLAEAAVGRAFLLQGGDCAESFKEFNANNIRDTFRVLFQMSVVLMFGGQMPVVKVGRMAGQFAKPRSDGFEERDGVKLPSYRGDNINGDAFDEKSRLPDPHRMISAYSQSAATLNLLRAFATGGYAAMQRVTQWNLDFTEHSEQGDRYMELAHRVDEALGFMSAAGLTLDHPIMTTTEFWTSHECLLLPYEQALTREDSTSGLYYDCSAHFLWVGERTRQLDGAHVEFLRGIANPLGIKVSDKMDPAELVRLIDILNPENRAGRITIIARMGPENMRVKLPHLIRAVRGAGQIVTWVTDPMHGNTMKAPCGLKTRSFDRILAEVRAFFDVHEQEGSYPGGVHLEMTGQNVTECIGGSRTVTFDDLGSRYHTHCDPRLNASQSLEMAFIIAERLRKRRIASSPLYTNQLGSIRLGS

SEQ ID NO: 141 - protein, 579 aa, synthetic construct
MASSMLSSAAVVATRASAAQASMVAPFTGLKSAASFPVTRKQNNLDITSIASNGGRVRAMRGSHHHHHHGMASMTGGQQMGRDLYDDDDKDHPFTVHAAEPTKNSVSVKESAAPVAAPGTGKWSLESWKTKKALQLPEYPDANELETVLKTIETFPPIVFAGEARSLEENLAEAAMGNAFLLQGGDCAESFKEFSANNIRDTFRILLQMGVVLMFGGQVPVIKVGRMAGQFAKPRSDPFEEKDGVKLPSYKGDNINGDAFNEKSRIPDPQRLIRAYCQSAATLNLLRAFATGGYAAMQRVTQWNLDFAQHSEQGDRYQELANRVDEALGFMAAAGLTVDHPIMTTTEFWTSHECLHLPYEQSLTRLDSTSGLYYDCSAHMLWCGERTRQLDGAHVEFLRGISNPIGIKVSNKMDPNELVKLIEIFNSNNKPGRITIIVRMGAENMRVKFPHLIRAVRRAGQIVTWVCDPMHGNTIKAPCGLKTRPFDAILAEVRAFFDVHEQEGSHPGGIHLEMTGQNVTECIGGSRTVTFDDLSSRYHTHCDPRLNASQSLELAFIIAERLRKRRIGTQRLFSLGQGS

SEQ ID NO: 142 - protein, 579 aa, synthetic construct
MASSMLSSAAVVATRASAAQASMVAPFTGLKSAASFPVTRKQNNLDITSIASNGGRVRAMRGSHHHHHHGMASMTGGQQMGRDLYDDDDKDHPFTVHAAEPTKNSVSVKESAAPVAAPGTGKWSLESWKTKKALQLPEYPDANELETVLKTIETFPPIVFAGEARSLEENLAEAAMGNAFLLQGGDCAESFKEFSANNIRDTFRILLQMGVVLMFGGQVPVIKVGRMAGQFAKPRSDPFEEKDGVKLPSYKGDNINGDAFNEKSRIPDPQRLIRAYCQSAATLNLLRAFATRGYAAMQRVTQWNLDFAQHSEQGDRYQELANRVDEALGFMAAAGLTVDHPIMTTTEFWTSHECLHLPYEQSLTRLDSTSGLYYDCSAHMLWCGERTRQLDGAHVEFLRGISNPIGIKVSNKMDPNELVKLIEIFNSNNKPGRITIIVRMGAENMRVKFPHLIRAVRRAGQIVTWVCDPMHGNTIKAPCGLKTRPFDAILAEVRAFFDVHEQEGSHPGGIHLEMTGQNVTECIGGSRTVTFDDLSSRYHTHCDPRLNASQSLELAFIIAERLRKRRIGTQRLFSLGQGS

SEQ ID NO: 143 - protein, 579 aa, synthetic construct
MASSMLSSAAVVATRASAAQASMVAPFTGLKSAASFPVTRKQNNLDITSIASNGGRVRAMRGSHHHHHHGMASMTGGQQMGRDLYDDDDKDHPFTVHAAEPTKNSVSVKESAAPVAAPGTGKWSLESWKTKKALQLPEYPDANELETVLKTIETFPPIVFAGEARSLEENLAEAAMGNAFLLQGGDCAESFKEFSANNIRDTFRILFQMGVVLMFGGQVPVIKVGRMAGQFAKPRSDPFEEKDGVKLPSYKGDNINGDAFNEKSRIPDPQRLIRAYCQSAATLNLLRAFATGGYAAMQRVTQWNLDFAQHSEQGDRYQELANRVDEALGFMAAAGLTVDHPIMTTTEFWTSHECLHLPYEQSLTRLDSTSGLYYDCSAHMLWCGERTRQLDGAHVEFLRGISNPIGIKVSNKMDPNELVKLIEIFNSNNKPGRITIIVRMGAENMRVKFPHLIRAVRRAGQIVTWVCDPMHGNTIKAPCGLKTRPFDAILAEVRAFFDVHEQEGSHPGGIHLEMTGQNVTECIGGSRTVTFDDLSSRYHTHCDPRLNASQSLELAFIIAERLRKRRIGTQRLFSLGQGS